(19) United States
(12) Patent Application Publication
(10) Pub. No.: US 2005/0239104 A1
(43) Pub. Date: Oct. 27, 2005

(54) Microarray controls

(76) Inventors: Tracy L. Ferea, Mountain View, CA (US); David P. Holden, Burlingame, CA (US); Gary Schroth, San Ramon, CA (US); Austin B. Tomaney, San Francisco, CA (US); Andrew Diamond, San Diego, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/982,541

(22) Filed: Nov. 4, 2004

Related U.S. Application Data
(60) Provisional application No. 60/517,506, filed on Nov. 4, 2003.
(60) Provisional application No. 60/519,077, filed on Nov. 10, 2003.

(51) Int. Cl. C12Q 1/68; C12M 1/34

(52) U.S. Cl. 435/6; 435/287.2

(57) ABSTRACT

The teachings relate to microarray controls.

This application claims the benefit of U.S. Provisional Application No. 60/517,506, filed Nov. 4, 2003, and U.S. Provisional Application No. 60/519,077, filed Nov. 10, 2003. U.S. Provisional Application No. 60/517,506 and U.S. Provisional Application No. 60/519,077 (in their entirety, including Tables A through I and Appendices A through E of those applications) are incorporated by reference herein.

### FIELD

Disclosed are assays for detecting or determining target molecules in a sample. In certain embodiments, the teachings relate to nucleic acid arrays and controls used in such arrays.

### BACKGROUND

Substrate-bound oligonucleotide arrays, also known as microarrays, enable one to test the hybridization of different nucleic acid sequences in a sample to different oligonucleotide probes. These microarrays can be composed of hundreds of thousands of probes deposited or synthesized within specific regions, defined as features, on a substrate such as a glass microscope slide or other materials. In some procedures, one may use target nucleic acid directly from a sample (as mRNA, for example). In some procedures, one may use target nucleic acid replicated or amplified from a sample (as cDNA, for example). Hybridization assays on such microarrays may be used, e.g., for profiling of gene expression levels, identification of genetic variants of infectious diseases, identification of genetic diseases, or any assay that identifies different nucleic acid sequences.

### SUMMARY

In certain embodiments, a microarray is provided comprising, an internal control set and at least one control element selected from: a spatial normalization control, a control ladder, a positive gene mismatch control, a hybridization control, a non-specific background control, a reverse transcription control, a buffer blank control, and an in vitro transcription control.

In certain embodiments, a microarray is provided comprising an internal control set and at least two control elements selected from: a spatial normalization control, a control ladder, a positive gene mismatch control, a hybridization control, a non-specific background control, a reverse transcription control, a buffer blank control, and an in vitro transcription control.

In certain embodiments, a microarray is provided comprising an internal control set and at least three control elements selected from: a spatial normalization control, a control ladder, a positive gene mismatch control, a hybridization control, a non-specific background control, a reverse transcription control, a buffer blank control, and an in vitro transcription control.

In certain embodiments, a microarray is provided comprising an internal control set and at least four control elements selected from: a spatial normalization control, a control ladder, a positive gene mismatch control, a hybridization control, a non-specific background control, a reverse transcription control, a buffer blank control, and an in vitro transcription control.

In certain embodiments, a microarray is provided comprising an internal control set and at least five control elements selected from: a spatial normalization control, a control ladder, a positive gene mismatch control, a hybridization control, a non-specific background control, a reverse transcription control, a buffer blank control, and an in vitro transcription control.

In certain embodiments, a microarray is provided comprising an internal control set and at least one control element selected from: a spatial normalization control, a control ladder, and a positive gene mismatch control.

In certain embodiments, a microarray is provided comprising an internal control set and at least two control elements selected from: a spatial normalization control, a control ladder, and a positive gene mismatch control.

In certain embodiments, a microarray is provided comprising an internal control set and: a spatial normalization control, a control ladder, and a positive gene mismatch control.

In certain embodiments, a microarray is provided comprising an internal control set and a spatial normalization control.

In certain embodiments, a microarray is provided comprising an internal control set and a control ladder.

In certain embodiments, a microarray is provided comprising an internal control set and a positive gene mismatch control.

In certain embodiments, a microarray is provided comprising a spatial normalization control.

In certain embodiments, a microarray is provided comprising a spatial normalization control and at least one control element selected from: a control ladder, a positive gene mismatch control, a hybridization control, a non-specific background control, a reverse transcription control, a buffer blank control, and an in vitro transcription control.

In certain embodiments, a microarray is provided comprising a spatial normalization control and at least two control elements selected from: a control ladder, a positive gene mismatch control, a hybridization control, a non-specific background control, a reverse transcription control, a buffer blank control, and an in vitro transcription control.

In certain embodiments, a microarray is provided comprising a spatial normalization control and at least three control elements selected from: a control ladder, a positive gene mismatch control, a hybridization control, a non-specific background control, a reverse transcription control, a buffer blank control, and an in vitro transcription control.

In certain embodiments, a microarray is provided comprising a spatial normalization control and at least four control elements selected from: a control ladder, a positive gene mismatch control, a hybridization control, a non-specific background control, a reverse transcription control, a buffer blank control, and an in vitro transcription control.

In certain embodiments, a microarray is provided comprising a spatial normalization control and at least one control element selected from a control ladder and a positive gene mismatch control.

In certain embodiments, a microarray is provided comprising a control ladder.

In certain embodiments, a microarray is provided comprising a control ladder and at least one control element selected from: a positive gene mismatch control, a hybridization control, a non-specific background control, a reverse transcription control, a buffer blank control, and an in vitro transcription control.

In certain embodiments, a microarray is provided comprising a control ladder and at least two control elements selected from: a positive gene mismatch control, a hybridization control, a non-specific background control, a reverse transcription control, a buffer blank control, and an in vitro transcription control.

In certain embodiments, a microarray is provided comprising a control ladder and at least three control elements selected from: a positive gene mismatch control, a hybridization control, a non-specific background control, a reverse transcription control, a buffer blank control, and an in vitro transcription control.

In certain embodiments, a microarray is provided comprising a control ladder and a positive gene mismatch control.

In certain embodiments, a microarray is provided comprising a positive gene mismatch control.

In certain embodiments, a microarray is provided comprising a positive gene mismatch control and at least one control element selected from: a hybridization control, a non-specific background control, a reverse transcription control, a buffer blank control, and an in vitro transcription control.

In certain embodiments, a microarray is provided comprising a positive gene mismatch control and at least two control elements selected from: a hybridization control, a non-specific background control, a reverse transcription control, a buffer blank control, and an in vitro transcription control.

In certain embodiments, a microarray is provided comprising at least one non-specific background control comprising a sequence selected for its dissimilarity to sequences in a database comprising sequences expected to be present in a test sample.

In certain embodiments, a microarray is provided comprising at least one non-specific background control comprising at least one sequence selected from: SEQ ID NOs: 37 to 134. In certain such embodiments, a microarray further comprises at least one control element selected from: an internal control, a spatial normalization control, a control ladder, a positive gene mismatch control, a non-specific background control, a reverse transcription control, a buffer blank control, and an in vitro translation control.

In certain embodiments, a microarray is provided comprising a hybridization control comprising at least one sequence selected from: SEQ ID NOS: 4, 5, and 6.

In certain embodiments, a microarray is provided comprising a hybridization mismatch control comprising at least one sequence containing one nucleotide substitution compared to a sequence selected from: SEQ ID NOS: 4, 5, and 6.

In certain embodiments, a microarray is provided comprising a reverse transcription control comprising at least one nucleic acid sequence selected from SEQ ID NOS: 22 to 36.

In certain embodiments, a microarray is provided comprising an in vitro transcription control comprising at least one nucleic acid sequence selected from SEQ ID NOS: 7 to 21.

In certain embodiments, a method is provided for normalizing signals from two or more experimental features on a microarray comprising:
attaching a first experimental probe to a first experimental feature on the microarray and attaching a second experimental probe to a second experimental feature on the microarray; wherein hybridization of target to experimental probe results in experimental signal;
attaching a first control probe and a second control probe to a first control feature on the microarray, wherein the first control probe comprises a fluorescent label and the second control probe comprises a chemiluminescent label;
attaching a first control probe and a second control probe to a second control feature on the microarray, wherein the first control probe attached to the second control feature is the same as the first control probe attached to the first control feature and the second control probe attached to the second control feature is the same as the second control probe attached to the first control feature;
contacting the microarray with a test sample; detecting experimental signals from the first and second experimental features, and detecting fluorescent and chemiluminescent signals from the first and second control features; and
using the fluorescent and chemiluminescent signals from the first and second control features to normalize experimental signals from the first and second experimental features.

In certain embodiments, a method is provided for normalizing signals from two or more experimental features on a microarray comprising:
attaching a first experimental probe to a first experimental feature on the microarray and attaching a second experimental probe to a second experimental feature on the microarray; wherein hybridization of target to experimental probe results in experimental signal;
attaching a first control probe and a second control probe to a first control feature on the microarray, wherein the first control probe is complementary to a first control target and the second control probe is complementary to a second control target;
attaching a first control probe and a second control probe to a second control feature on the microarray, wherein the first control probe attached to the second control feature is the same as the first control probe attached to the first control feature and the second control probe attached to the second control feature is the same as the second control probe attached to the first control feature;
contacting the microarray with a test sample comprising a first control target comprising a fluorescent label and a second control target comprising a chemiluminescent label;
detecting experimental signals from the first and second experimental features, and detecting fluorescent and chemiluminescent signals from the first and second control features; and
using the fluorescent and chemiluminescent signals from the first and second control features to normalize experimental signals from the first and second experimental features.

In certain embodiments, a kit is provided comprising: a microarray comprising a spatial normalization control probe and a spatial normalization control target.

In certain embodiments, a kit is provided comprising: a microarray comprising a hybridization control comprising at least one sequence selected from: SEQ ID NOS: 4, 5, and 6 and a hybridization control target.

In certain embodiments, a kit is provided comprising: a microarray comprising a hybridization mismatch control comprising at least one sequence containing one nucleotide substitution compared to at least one sequence selected from: SEQ ID NOS: 4, 5, and 6; and a hybridization mismatch control target.

In certain embodiments, a kit is provided comprising: at least one nucleic acid molecule comprising a nucleic acid sequence selected from SEQ ID NOS: 22 to 36; a reverse transcriptase; and a microarray comprising a reverse transcription control comprising at least one nucleic acid sequence selected from SEQ ID NOS: 22 to 36.

In certain embodiments, a kit is provided comprising: at least one nucleic acid molecule comprising a nucleic acid sequence selected from SEQ ID NOS: 7 to 21; a transcriptase; and a microarray comprising an in vitro transcription control comprising at least one nucleic acid sequence selected from SEQ ID NOS: 7 to 21.

In certain embodiments, a computer-implemented method is provided for processing data associated with one or more microarrays comprising: obtaining first data related to one or more spatial normalization controls and second data related to one or more experimental probes from the one or more microarrays; and normalizing the second data using the first data.

In certain such embodiments, the first data comprises signal values associated with the spatial normalization controls. In certain such embodiments, the second data comprises signal values associated with the experimental probes. In certain such embodiments, normalizing the second data using the first data comprises dividing signal values associated with the experimental probes with signal values associated with the spatial normalization controls. In certain such embodiments, the signal values comprise signal values associated with at least one of a chemiluminescent (CL) signal and a fluorescent (FL) signal.

In certain embodiments, a system is provided for processing data associated with one or more microarrays, comprising:
at least one memory storing program instructions;
a processor configured to execute the program instructions stored in the memory in order to perform a method comprising:
   obtaining first data related to one or more spatial normalization controls and second data related to one or more experimental probes from the one or more microarrays; and
   normalizing the second data using the first data.

In certain such embodiments, the first data comprises signal values associated with the spatial normalization controls. In certain such embodiments, the second data comprises signal values associated with the experimental probes. In certain such embodiments, the processor is further configured to divide signal values associated with the experimental probes with signal values associated with the spatial normalization controls. In certain such embodiments, the signal values comprise signal values associated with at least one of a chemiluminescent (CL) signal and a fluorescent (FL) signal.

In certain embodiments, a computer readable medium is provided containing instructions for controlling a computer system to perform a method for processing data associated with one or more microarrays, the method comprising: obtaining first data related to one or more spatial normalization controls and second data related to one or more experimental probes from the one or more microarrays; and normalizing the second data using the first data.

In certain embodiments, a computer-implemented method is provided for processing data from one or more microarrays comprising: obtaining data related to at least one first internal control probe that provides a chemiluminescent (CL) signal and at least one second internal control probe that provides a fluorescent (FL) signal; and correcting the data using the CL and FL signals.

In certain such embodiments, the CL and FL signals have associated signal values. In certain such embodiments, correcting the data comprises correcting the data using a ratio of the signal values associated with the CL and FL signals. In certain such embodiments, correcting the data comprises correcting the data using a ratio of the signal values associated with the CL and FL signals, wherein the data relates to background corrected signal values associated with the CL and FL signals. In certain such embodiments, correcting the data comprises correcting the data using a ratio of the signal values associated with the CL and FL signals, wherein the data relates to normalized CL signal values.

In certain such embodiments, the method further comprises calculating a predicted coefficient of variation per feature related to the corrected data using a calculated uncertainty measurement of the corrected data in combination with a coefficient of variation of a system estimated from high signal-to-noise (S/N) replicate controls on the one or more microarrays. In certain embodiments wherein correcting the data comprises correcting the data using a ratio of the signal values associated with the CL and FL signals, the data relates to associated CL and FL signal uncertainties.

In certain embodiments, a system is provided for processing data associated with one or more microarrays, comprising:
at least one memory storing program instructions;
a processor configured to execute the program instructions stored in the memory in order to perform a method comprising:
   obtaining data related to at least one first internal control probe that provides a chemiluminescent (CL) signal and at least one second internal control probe that provides a fluorescent (FL) signal; and
   correcting the data using the CL and FL signals.

In certain such embodiments, the CL and FL signals have associated signal values. In certain such embodiments, the processor is further configured to correct the data using a ratio of the signal values associated with the CL and FL signals. In certain embodiments in which the processor is further configured to correct the data using a ratio of the signal values associated with the CL and FL signals, the data relates to background corrected signal values associated with the CL and FL signals. In certain embodiments in which the processor is further configured to correct the data using a ratio of the signal values associated with the CL and FL signals, the data relates to normalized CL signal values. In certain embodiments in which the processor is further configured to correct the data using a ratio of the signal values associated with the CL and FL signals, the data relates to associated CL and FL signal uncertainties. In certain embodiments in which the processor is further configured to correct the data using a ratio of the signal values associated with the CL and FL signals, the processor is further configured to calculate a predicted coefficient of variation per feature related to the corrected data using a calculated uncertainty measurement of the corrected data in combination with a coefficient of variation of a system estimated from high signal-to-noise (S/N) replicate controls on the one or more microarrays.

In certain embodiments, a computer readable medium is provided containing instructions for controlling a computer system to perform a method for processing data associated with one or more microarrays, the method comprising: obtaining data related to at least one first internal control probe that provides a chemiluminescent (CL) signal and at least one second internal control probe that provides a fluorescent (FL) signal; and correcting the data using the CL and FL signals.

In certain embodiments, a computer-implemented method is provided for processing data associated with a microarray comprising: obtaining first data related to a first signal from gridding reference control features and second data related to a second signal from one or more experimental probes from the microarray, wherein the second signal is detectably different from the first signal; and associating coordinates associated with the one or more experimental probes using the gridding reference control features as reference points.

In certain such embodiments, the first signal is a fluorescent (FL) signal. In certain such embodiments, the second signal is a chemiluminescent (CL) signal.

In certain embodiments, a computer-implemented method is provided for processing data associated with a microarray comprising: obtaining first data related to a first signal from gridding reference control features and second data related to a second signal from one or more experimental probes from the microarray, wherein the second signal is detectably different from the first signal; and associating coordinates associated with the one or more experimental probes using the gridding reference control features as reference points; wherein associating coordinates comprises identifying the gridding reference control features on sub-grids of the microarray and mapping the identified gridding reference control features to x, y coordinates. In certain such embodiments, first signal is a fluorescent (FL) signal and the second signal is a chemiluminescent (CL) signal.

In certain embodiments, a system is provided for processing data associated with a microarray, comprising:
at least one memory storing program instructions;
a processor configured to execute the program instructions stored in the memory in order to perform a method comprising:
   obtaining first data related to a first signal from gridding reference control features and second data related to a second signal from one or more experimental probes from the microarray, wherein the second signal is detectably different from the first signal; and
   associating coordinates associated with the one or more experimental probes using the gridding reference control features as reference points.

In certain such embodiments, the first signal is a fluorescent (FL) signal. In certain such embodiments, the second signal is a chemiluminescent (CL) signal.

In certain embodiments, a system is provided for processing data associated with a microarray, comprising:
at least one memory storing program instructions;
a processor configured to execute the program instructions stored in the memory in order to perform a method comprising:
   obtaining first data related to a first signal from gridding reference control features and second data related to a second signal from one or more experimental probes from the microarray, wherein the second signal is detectably different from the first signal; and
   associating coordinates associated with the one or more experimental probes using the gridding reference control features as reference points;
wherein the processor is further configured to identify the gridding reference control features on sub-grids of the microarray and map the identified gridding reference control features to x, y coordinates. In certain such embodiments, the first signal is a fluorescent (FL) signal and the second signal is a chemiluminescent (CL) signal.

In certain embodiments, a computer readable medium is provided containing instructions for controlling a computer system to perform a method for processing data associated with a microarray, the method comprising:
obtaining first data related to a first signal from gridding reference control features and second data related to a second signal from one or more experimental probes from the microarray, wherein the second signal is detectably different from the first signal; and
associating coordinates associated with the one or more experimental probes using the gridding reference control features as reference points.

In certain embodiments, a computer-implemented method is provided for image processing of a microarray having a plurality features, the method comprising:
obtaining first images of the microarray using internal control probes that provide a fluorescent (FL) signal associated with features of the microarray;
obtaining second images of the microarray using internal control probes that provide a chemiluminescent (CL) signal associated with features of the microarray, wherein the FL and CL signals have associated signal values; and
detecting the features of the microarray using a combination of the FL and CL signal values of the first and second images.

In certain such embodiments, obtaining first images comprises obtaining a first FL image with exposure to excitation light and obtaining a second FL image without exposure to excitation light. In certain such embodiments, the method further comprises subtracting the signal values in the second FL image from the signal values associated with the FL signals in the first FL image to obtain a third corrected FL image. In certain such embodiments, the signal values are scaled signal values.

In certain embodiments in which the method further comprises subtracting the signal values in the second FL image from the signal values associated with the FL signals in the first FL image to obtain a third corrected FL image, the third corrected FL image is corrected for spectral-crosstalk from CL signals in the first FL image. In certain such embodiments, obtaining second images comprises obtaining a first CL image with a first exposure time and obtaining a second CL image with a second exposure time, wherein the first exposure time is greater than the second exposure time.

In certain such embodiments, the method further comprises identifying signal values in the first CL image having detector saturated signal values and replacing the identified detector saturated signal values with signal values in the second CL image multiplied by a factor to obtain a third CL image. In certain such embodiments, the factor is equal to the first exposure time divided by the second exposure time.

In certain embodiments in which the method further comprises identifying signal values in the first CL image having detector saturated signal values and replacing the identified detector saturated signal values with signal values in the second CL image multiplied by a factor to obtain a third CL image, the method further comprises calibrating the third FL image and the third CL image. In certain such embodiments, the method further comprises associating coordinates to the features of the microarray using the signal values of the calibrated third FL image and the calibrated third CL image. In certain such embodiments, the method further comprises: correcting background signal in the calibrated third FL image and the calibrated third CL image; integrating features of the microarray in the calibrated third FL image and the calibrated third CL image; and normalizing the integrated feature intensity obtained from the third CL image by the integrated feature intensity from the third FL image.

In certain embodiments in which the method further comprises associating coordinates to the features of the microarray using the signal values of the calibrated third FL image and the calibrated third CL image, normalizing the integrated feature intensity includes applying weights to the integrated feature intensity.

In certain embodiments, a system is provided for processing data associated with one or more microarrays, comprising:
at least one memory storing program instructions;
a processor configured to execute the program instructions stored in the memory in order to perform a method comprising:
   obtaining first images of the microarray using internal control probes that provide a fluorescent (FL) signal associated with features of the microarray;
   obtaining second images of the microarray using internal control probes that provide a chemiluminescent (CL) signal associated with features of the microarray, wherein the FL and CL signals have associated signal values; and
   detecting the features of the microarray using a combination of the FL and CL signal values of the first and second images.

In certain such embodiments, the processor is further configured to obtain a first FL image with exposure to excitation light and obtain a second FL image without exposure to excitation light. In certain such embodiments, the processor is further configured to subtract the signal values in the second FL image from the signal values associated with the FL signals in the first FL image to obtain a third corrected FL image. In certain such embodiments, the signal values are scaled signal values.

In certain embodiments in which the processor is further configured to subtract the signal values in the second FL image from the signal values associated with the FL signals in the first FL image to obtain a third corrected FL image, the third corrected image is corrected for spectral-crosstalk from CL signals in the first FL image. In certain such embodiments, the processor is further configured to obtain a first CL image with a first exposure time and to obtain a second CL image with a second exposure time, wherein the first exposure time is greater than the second exposure time. In certain such embodiments, the processor is further configured to identify signal values in the first CL image having detector saturated signal values and to replace the identified detector saturated signal values with signal values in the second CL image multiplied by a factor to obtain a third CL image. In certain such embodiments, the factor is equal to the first exposure time divided by the second exposure time.

In certain embodiments in which the processor is further configured to identify signal values in the first CL image having detector saturated signal values and to replace the identified detector saturated signal values with signal values in the second CL image multiplied by a factor to obtain a third CL image, the processor is further configured to calibrate the third FL image and the third CL image. In certain such embodiments, the processor is further configured to associate coordinates to the features of the microarray using the signal values of the calibrated third FL image and the calibrated third CL image. In certain such embodiments, the processor is further configured to correct background signal in the calibrated third FL image and the calibrated third CL image, to integrate features of the microarray in the calibrated third FL image and the calibrated third CL image, and to normalize the integrated feature intensity obtained from the third CL image by the integrated feature intensity from the third FL image.

In certain embodiments in which the processor is further configured to correct background signal in the calibrated third FL image and the calibrated third CL image, to integrate features of the microarray in the calibrated third FL image and the calibrated third CL image, and to normalize the integrated feature intensity obtained from the third CL image by the integrated feature intensity from the third FL image, the processor is further configured to apply weights to the integrated feature intensity.

In certain embodiments, a computer readable medium is provided containing instructions for controlling a computer system to perform a method for processing data associated with one or more microarrays, the method comprising:
obtaining first images of the microarray using internal control probes that provide a fluorescent (FL) signal associated with features of the microarray;
obtaining second images of the microarray using internal control probes that provide a chemiluminescent (CL) signal associated with features of the microarray, wherein the FL and CL signals have associated signal values; and
detecting the features of the microarray using a combination of the FL and CL signal values of the first and second images.

In certain embodiments, a computer-implemented method is provided for processing data associated with at least one microarray comprising:
obtaining first data related to signal values from non-specific background controls, wherein the non-specific background controls comprise at least one oligonucleotide sequence selected for its dissimilarity to sequences in a database comprising sequences expected to be present in a test sample;
correcting second data related to at least one experimental probe from the at least one microarray; and
normalizing the second data using the first data.

In certain such embodiments, the first data comprises the mean signal value calculated from the signal values of the non-specific background controls. In certain such embodiments, the second data comprises at least one signal value associated with the at least one experimental probe. In certain such embodiments, the correcting the second data using the first data comprises subtracting the mean signal value calculated from the signal values of the non-specific background controls from at least one of the at least one signal value associated with the at least one experimental probe.

In certain embodiments, a computer-implemented method is provided for processing data associated with at least one microarray comprising:
obtaining first data related to signal values from non-specific background controls, wherein the non-specific background controls comprise at least one oligonucleotide sequence selected for its dissimilarity to sequences in a database comprising sequences expected to be present in a test sample, wherein the first data comprises the median signal value calculated from the signal values of the non-specific background controls;
correcting second data related to at least one experimental probe from the at least one microarray; and
normalizing the second data using the first data.

In certain such embodiments, the second data comprises at least one signal value associated with the at least one experimental probe. In certain such embodiments, the correcting the second data using the first data comprises subtracting the median signal value calculated from the signal values of the non-specific background controls from at least one of the at least one signal value associated with the at least one experimental probe.

In certain embodiments, a system is provided for processing data associated with one or more microarrays, comprising:
at least one memory storing program instructions;
a processor configured to execute the program instructions stored in the memory in order to perform a method comprising:
   obtaining first data related to signal values from non-specific background controls, wherein the non-specific background controls comprise at least one oligonucleotide sequence selected for its dissimilarity to sequences in a database comprising sequences expected to be present in a test sample;
   obtaining second data related to at least one experimental probe from the at least one microarray; and
   correcting the second data using the first data.

In certain such embodiments, the first data comprises the mean signal value calculated from the signal values of the non-specific background controls. In certain such embodiments, the second data comprises at least one signal value associated with the at least one experimental probe. In certain such embodiments, the processor is further configured to subtract the mean signal value calculated from the signal values of the non-specific background controls from at least one of the at least one signal value associated with the at least one experimental probe.

In certain embodiments, a system is provided for processing data associated with one or more microarrays, comprising:
at least one memory storing program instructions;
a processor configured to execute the program instructions stored in the memory in order to perform a method comprising:
   obtaining first data related to signal values from non-specific background controls, wherein the non-specific background controls comprise at least one oligonucleotide sequence selected for its dissimilarity to sequences in a database comprising sequences expected to be present in a test sample, wherein the first data comprises the median signal value calculated from the signal values of the non-specific background controls;
   obtaining second data related to at least one experimental probe from the at least one microarray; and
   correcting the second data using the first data. In certain such embodiments, the second data comprises at least one signal value associated with the at least one experimental probe. In certain such embodiments, the processor is further configured to subtract the median signal value calculated from the signal values of the non-specific background controls from at least one of the at least one signal value associated with the at least one experimental probe.

In certain embodiments, a computer readable medium is provided containing instructions for controlling a computer system to perform a method for processing data associated with one or more microarrays, the method comprising: obtaining first data related to signal values from non-specific background controls, wherein the non-specific background controls comprise at least one oligonucleotide sequence selected for its dissimilarity to sequences in a database comprising sequences expected to be present in a test sample;
obtaining second data related to at least one experimental probe from the at least one microarray; and
correcting the second data using the first data.

In certain embodiments, a computer-implemented method is provided for processing data associated with at least one microarray comprising: obtaining data for a group of pixels related to chemiluminescent (CL) signals and fluorescent (FL) signals of a feature; and integrating the group of pixels to obtain a mean pixel signal value for the group of pixels by using a combination of the CL and FL signals.

In certain such embodiments, integrating the group of pixels includes: adding signal values of each pixel in the group multiplied by a weighting factor for each pixel to obtain a first sum; adding the weighting factor of each pixel to obtain a second sum; and dividing the first sum by the second sum to obtain the mean pixel signal value for the group of pixels. In certain such embodiments, the signal values of each pixel is the background corrected signal value for each pixel. In certain such embodiments, the weighting factor of each pixel is determined based on at least the high signal correlation of the CL and FL signals.

In certain embodiments, a computer-implemented method is provided for processing data associated with at least one microarray comprising: obtaining data for a group of pixels related to chemiluminescent (CL) signals and fluorescent (FL) signals of a feature, wherein the pixels have pixel values with high CL and FL signal correlation; and integrating the group of pixels to obtain a mean pixel signal value for the group of pixels by using a combination of the CL and FL signals.

In certain embodiments, a system is provided for processing data associated with one or more microarrays, comprising:
at least one memory storing program instructions;
a processor configured to execute the program instructions stored in the memory in order to perform a method comprising:
   obtaining data for a group of pixels related to chemiluminescent (CL) signals (CL) and fluorescent (FL) signals of a feature; and
   integrating the group of pixels to obtain a mean pixel signal value for the group of pixels by using a combination of the CL and FL signals.

In certain such embodiments, the processor is further configured to add signal values of each pixel in the group multiplied by a weighting factor for each pixel to obtain a first sum, add the weighting factor of each pixel to obtain a second sum, and divide the first sum by the second sum to obtain the mean pixel signal value for the group of pixels. In certain such embodiments, the signal values of each pixel is the background corrected signal value for each pixel.

In certain embodiments in which the processor is further configured to add signal values of each pixel in the group multiplied by a weighting factor for each pixel to obtain a first sum, add the weighting factor of each pixel to obtain a second sum, and divide the first sum by the second sum to obtain the mean pixel signal value for the group of pixels; the pixels have pixel values with high CL and FL signal correlation. In certain such embodiments, the processor is further configured to determine the weighting factor of each pixel based on at least the high signal correlation of the CL and FL signals.

In certain embodiments, a computer readable medium is provided containing instructions for controlling a computer system to perform a method for processing data associated with one or more microarrays, the method comprising:
obtaining data for a group of pixels related to chemiluminescent (CL) signals (CL) and fluorescent (FL) signals of a feature; and
integrating the group of pixels to obtain a mean pixel signal value for the group of pixels by using a combination of the CL and FL signals.

In certain embodiments, a microarray is provided comprising, an internal control set and at least one control element selected from: a spatial normalization control, a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, an internal control set and at least two control elements selected from: a spatial normalization control, a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, an internal control set and at least three control elements selected from: a spatial normalization control, a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, an internal control set and at least four control elements selected from: a spatial normalization control, a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, an internal control set and at least five control elements selected from: a spatial normalization control, a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, a spatial normalization control and at least one control element selected from: a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, a spatial normalization control and at least two control elements selected from: a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, a spatial normalization control and at least three control elements selected from: a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, a spatial normalization control and at least four control elements selected from: a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, a spatial normalization control and at least five control elements selected from: a control ladder, a positive gene mismatch control, a hybridization control, a non-specific background control, a reverse transcription control, a buffer blank control, an in vitro transcription control, and attachment control, and a contaminant control.

In certain embodiments, a microarray is provided comprising, a control ladder and at least one control element selected from: a positive gene mismatch control, a hybridization control, a non-specific background control, a reverse transcription control, a buffer blank control, an in vitro transcription control, and attachment control, and a contaminant control.

In certain embodiments, a microarray is provided comprising, a control ladder and at least two control elements selected from: a positive gene mismatch control, a hybridization control, a non-specific background control, a reverse transcription control, a buffer blank control, an in vitro transcription control, and attachment control, and a contaminant control.

In certain embodiments, a microarray is provided comprising, a control ladder and at least three control elements selected from: a positive gene mismatch control, a hybridization control, a non-specific background control, a reverse transcription control, a buffer blank control, an in vitro transcription control, and attachment control, and a contaminant control.

In certain embodiments, a microarray is provided comprising, a control ladder and at least four control elements selected from: a positive gene mismatch control, a hybridization control, a non-specific background control, a reverse transcription control, a buffer blank control, an in vitro transcription control, and attachment control, and a contaminant control.

In certain embodiments, a microarray is provided comprising, a control ladder and at least five control elements selected from: a positive gene mismatch control, a hybridization control, a non-specific background control, a reverse transcription control, a buffer blank control, an in vitro transcription control, and attachment control, and a contaminant control.

In certain embodiments, a microarray is provided comprising, a positive gene mismatch control and at least one control element selected from: a hybridization control, a non-specific background control, a reverse transcription control, a buffer blank control, an in vitro transcription control, and attachment control, and a contaminant control.

In certain embodiments, a microarray is provided comprising, a positive gene mismatch control and at least two control elements selected from: a hybridization control, a non-specific background control, a reverse transcription control, a buffer blank control, an in vitro transcription control, and attachment control, and a contaminant control.

In certain embodiments, a microarray is provided comprising, a positive gene mismatch control and at least three control elements selected from: a hybridization control, a non-specific background control, a reverse transcription control, a buffer blank control, an in vitro transcription control, and attachment control, and a contaminant control.

In certain embodiments, a microarray is provided comprising, a positive gene mismatch control and at least four control elements selected from: a hybridization control, a non-specific background control, a reverse transcription control, a buffer blank control, an in vitro transcription control, and attachment control, and a contaminant control.

In certain embodiments, a microarray is provided comprising, a positive gene mismatch control and at least five control elements selected from: a hybridization control, a non-specific background control, a reverse transcription control, a buffer blank control, an in vitro transcription control, and attachment control, and a contaminant control.

### BRIEF DESCRIPTION OF THE FIGURES

FIG. 1 shows the arrangement of the gridding of an exemplary microarray according to certain embodiments.

FIG. 2 shows an arrangement of exemplary microarray control elements according to certain embodiments. (In view of the size of the figure, the exemplary microarray is split into two figures. FIG. 2A shows the top image of such an exemplary microarray and FIG. 2B shows the bottom image of such an exemplary microarray).

FIG. 3 is an enlarged view of sub-grid 1A of FIG. 2.

FIG. 4 is an enlarged view of sub-grid 1C of FIG. 2.

FIG. 5 is an enlarged view of sub-grid 1D of FIG. 2.

FIG. 6 is an enlarged view of sub-grid 10F of FIG. 2.

FIG. 7 depicts certain embodiments in which one employs labeled experimental targets (a first signal), labeled internal control targets (second signal), and labeled internal control molecules that are attached to the feature (a third signal).

FIG. 8 shows a non-limiting exemplary image processing system 800 for obtaining images of microarrays according to certain embodiments.

FIG. 9 depicts an overview block diagram of a non-limiting exemplary computing system for processing images of a microarray.

FIG. 10 depicts a flow diagram of exemplary basic steps to process images of a microarray according to certain embodiments.

FIG. 11 depicts a detailed flow diagram of exemplary steps to perform image correction of FIG. 10 according to certain embodiments.

FIGS. 12A and 12B show an FL image and a corrected FL image after spectral-cross talk correction of the CL channel.

FIG. 13 depicts a detailed flow diagram of the steps to perform primary image analysis of FIG. 10 according to certain embodiments.

FIG. 14 shows illustrations of CL/FL feature pixels correlated to maximize the S/N of a final extracted intensity.

FIG. 15 depicts a flow diagram of the steps to perform feature quantification refinement of FIG. 10 according to certain embodiments.

FIG. 16 shows an FL image having spatial normalization correction.

FIG. 17 shows signal-to-noise of assay background controls.

FIG. 18 shows inter-microarray reproducability of detecting features.

FIG. 19 shows fold change confidence values.

FIG. 20 shows an error model.

FIG. 21 shows a plot of rejected features between two microarrays.

FIG. 22 shows features in images flagged with a quality metric.

FIG. 23 is a block diagram that illustrates a computer system, according to certain embodiments, upon which embodiments of the invention may be implemented.

FIG. 24 shows information for a row/column gridder according to certain embodiments as discussed in the Gridder Example.

FIG. 25 shows information for finding subgrid rows and columns according to certain embodiments as discussed in the Gridder Example.

### DETAILED DESCRIPTION OF CERTAIN EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose.

Certain Definitions

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2 ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar molecule, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C₁-C₆)alkoxyribose, 2′-(C5-C14)aryloxyribose, 2′,3′-didehydroribose, 2′-deoxy-3′-haloribose, 2′deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). Exemplary LNA sugar analogs within a polynucleotide include, but are not limited to, the structures:

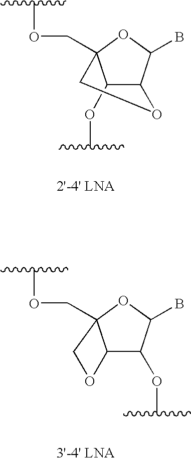

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 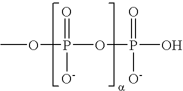
where a is an integer from 0 to 4. In certain embodiments, a is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog”, as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers that can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-60 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, “T” denotes thymidine or an analog thereof, and “U” denotes uridine or an analog thereof.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids include, but are not limited to, synthetic and in vitro transcription products.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 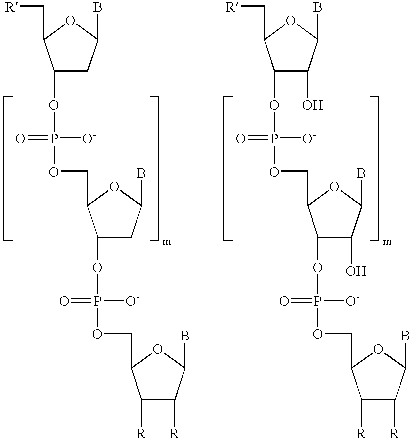
wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or 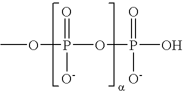
where a is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254:1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

The term “microarray” refers to one or more oligonucleotides attached to one or more features on a substrate.

The term “probe” refers to an oligonucleotide that is capable of binding to a complementary target sequence. In certain embodiments, at least one probe is attached to at least one feature on a microarray. The probe may include Watson-Crick bases or modified bases. Modified bases include, but are not limited to, the AEGIS bases (from Eragen Biosciences), which have been described, e.g., in U.S. Pat. Nos. 5,432,272; 5,965,364; and 6,001,983. Additionally, bases may be joined by a natural phosphodiester bond or a different chemical linkage. Different chemical linkages include, but are not limited to, a peptide bond or a Locked Nucleic Acid (LNA) linkage, which is described, e.g., in published PCT applications WO 00/56748; and WO 00/66604.

The term “target” refers to a nucleic acid molecule that is capable of hybridizing to a probe.

The term “experimental target” refers to a target, the presence, absence, or amount of which is sought to be determined. In certain embodiments, the experimental target is extracted from a biological sample, e.g., as RNA or DNA. In certain embodiments, the experimental target is replicated from a sample, e.g., such as cDNA which has been reverse transcribed from sample mRNA. In certain embodiments, the experimental target may be amplified from a sample.

The term “experimental probe” refers to a probe that is capable of hybridizing to an experimental target.

The term “control probe” refers to a probe that can be used to obtain a control signal. In certain embodiments, the control signal can be compared to a signal resulting from experimental target hybridized to an experimental probe. In certain embodiments, a control probe comprises a label. In certain embodiments, a control probe may hybridize to a control target.

The term “control target” refers to a target that is capable of hybridizing to a control probe.

The term “test sample” refers to a sample that is used in an assay. In certain embodiments, the test sample is assayed by contacting the test sample with a microarray. In certain embodiments, the test sample comprises one or more targets. In certain embodiments, the test sample includes one or more experimental targets. In certain embodiments, the test sample includes one or more control targets.

As used herein, an “affinity set” is a set of molecules that specifically bind to one another. Exemplary affinity sets include, but are not limited to, biotin and avidin, biotin and streptavidin, His6 tag and nickel, receptor and ligand, antibody and ligand, antibody and antigen, a polynucleotide sequence and its complement, a polynucleotide and a protein that specifically binds that polynucleotide, and affinity binding chemicals available from Prolinx™ (Bothell, Wash.) as exemplified, e.g., by U.S. Pat. Nos. 5,831,046; 5,852,178; 5,859,210; 5,872,224; 5,877,297; 6,008,406; 6,013,783; 6,031,117; and 6,075,126. As used herein, a ligand is any molecule that may be specifically bound by a receptor. Ligands may be proteinaceous or non-proteinaceous. Exemplary ligands include, but are not limited to, proteins, polypeptides, polysaccharides, and small molecules. As used herein, an antigen is any molecule that may be specifically bound by an antibody. Antigens may be proteinaceous or non-proteinaceous. Exemplary antigens include, but are not limited to, proteins, polypeptides, polysaccharides, polynucleotides, and small molecules.

The term “feature” refers to a single location on a microarray. In certain embodiments, a single feature may have probes that all have the same sequence. In certain embodiments, a single feature may include probes with different sequences. In certain embodiments, a single feature may include probes comprising labels. In certain embodiments, a single feature may include probes comprising one or more different labels. In certain embodiments, a feature comprises buffer, but no probe or label.

The term “label” refers to any molecule that can be detected. In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal. A complex encompasses more than one moiety associated by at least one covalent and/or at least one non-covalent interaction.

The term “signal value” refers to a value of the signal that is detected from a label. In certain embodiments, the signal value is the amount or intensity of signal that is detected from a label. Thus, if there is no detectable signal from a label, its signal value is zero (0). In certain embodiments, the signal value is a characteristic of the signal other than the amount or intensity of the signal, such as the spectra, wavelength, color, polarization, or lifetime of the signal.

The term “control element” refers to one or more features the signal from which is used to compare and/or verify results from the same and/or different features comprising experimental probes.

The term “verify” refers to using a signal to determine whether one or more steps of an experiment was successful.

The term “compare” refers to using a signal from one or more features to interpret signals from the same or one or more other features. The term “compare” includes, but is not limited to, using a signal to orient a microarray.

The term “internal control set” refers to a control element comprising at least one internal control molecule and at least one experimental probe at a single feature.

The term “spatial normalization control” refers to a control element comprising two or more features on a microarray, each of which produces at least one signal, and by comparing at least one signal of two or more of those features, one can normalize signals of the microarray in the regions of the compared features. In certain embodiments, a spatial normalization control feature produces at least two detectably different signals. In certain embodiments, a spatial normalization control feature produces both a fluorescent and chemiluminescent signal.

The term “control ladder” refers to a control element comprising at least two different features on a microarray that comprise labeled molecules in different concentrations.

The term “non-specific background control” refers to a control element comprising at least one non-specific background control probe which is designed based on its dissimilarity to any known target in a test sample.

The term “positive gene control” refers to a control element comprising at least one positive gene control probe that is complementary to at least a portion of a known gene without any mismatches, and the known gene is expected to be present in a test sample.

The term “positive gene mismatch control” refers to a control element comprising at least one positive gene mismatch control probe that comprises nucleotides that base pair with at least a portion of a known gene that is expected to be present in a sample and comprises at least one mismatch with the at least a portion of the known gene. Positive gene mismatch control probes may or may not hybridize to the at least a portion of the known gene.

The term “landmark fiducial” refers to a control element comprising a feature at a known location on a microarray and the landmark fiducial feature comprises at least one label.

The term “hybridization control” refers to a control element comprising at least one hybridization control probe that is complementary (and without any mismatches) to at least a portion of a labeled hybridization control target that is added to the test sample.

The term “hybridization mismatch control” refers to a control element comprising at least one hybridization mismatch control probe that comprises nucleotides that base pair with at least a portion of a labeled hybridization control target which is added to the test sample and comprises at least one mismatch with the at least a portion of the labeled hybridization control target. Positive gene mismatch control probes may or may not hybridize to the at least a portion of the labeled hybridization control target.

The term “reverse transcription control” refers to a control element comprising at least one reverse transcription control probe which is complementary to a control target derived from a reverse transcription reaction.

The term “in vitro transcription control” refers to a control element comprising at least one in vitro transcription probe which is complementary to a control target derived from an in vitro transcription reaction.

A buffer blank control is a control element feature that has buffer on the feature, and which has no probe or label attached to the feature.

The term “gridding” refers to the association of features with respect to the microarray layout and the feature locations in corresponding images.

The term “gridding reference control” refers to a control element comprising multiple features on a microarray that provide a first signal that can be used to determine where the features are represented in an image of that first signal, as well as where those features are represented in one or more additional images of one or more additional detectably different signals.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, and target-specific portions.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, and target-specific portions. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 70% of the particular identical sequences. In certain embodiments, “a substantial portion of the particular identical sequences” means at least 80% of the particular identical sequences. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, the more complex the composition, the more likely undesired sequences will hybridize. For example, in certain embodiments, with a given number of mismatches, a probe may more likely hybridize to undesired sequences in a composition with the entire genomic DNA than in a composition with fewer DNA sequences, when the same hybridization and wash conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, and target-specific portions.

Certain Exemplary Microarrays

In various embodiments, microarrays can be made of any suitable material. Suitable materials include, but are not limited to, silicon, nylon, glass, polymeric material, and shrinkable polymeric material.

In certain embodiments, target nucleic acid may be labeled. In certain embodiments, the label produces a signal or interacts with another moiety to produce a signal. In certain embodiments, the label can be a fluorescent molecule or a molecule that catalyzes a chemiluminescent reaction. In certain embodiments, the label can interact with another moiety to modify a signal of the other moiety. In certain embodiments, the label can bind to another moiety or complex that produces a signal or that interacts with another moiety to produce a signal.

Hybridization between a target and a probe may be detected by a signal at one or more features within the microarray. In certain embodiments, the amount of signal may be dependent on the amount of target available for hybridization, as well as the thermal stability of the probe-target hybrids. Thermal stability is a function of several factors. For example, the length of the hybridizing region, the accuracy of the match in hybridization, the total length of the oligonucleotides, as well as the actual sequence composition (A-T rich regions melt at lower temperatures than G-C rich areas), all factor into the specific melting temperature (Tm) for a probe-target hybrid.

In certain instances, an absence of signal for any given feature may be caused by a lack of a sufficient amount of that specific target in the test sample, or by a lack of sufficient probe bound to the feature. These two causes, which have profoundly different biological implications, in certain instances, cannot be distinguished. Further, in certain instances, comparison of signals from different regions of a microarray or signals from different microarrays may be difficult.

Certain embodiments are directed to the use of control elements in methods, compositions, and/or kits for the manufacture of the microarrays. Certain embodiments are directed to the use of control elements in methods, compositions, and/or kits that provide quality control for the manufacture of the microarrays. In certain embodiments, the teachings can provide quantification of signal to facilitate comparison between features within a microarray, comparison with features on other microarrays, and comparison of multiple features on multiple microarrays. Certain such embodiments provide reliable and consistent control signals against which an experimental signal can be compared.

The manufacture of high density nucleic acid microarrays, and methods of their use in diagnostic assays have been described, e.g., in U.S. Pat. Nos. 5,445,934; 5,552,270; 5,837,832; 6,040,138; and 6,045,996; and published PCT applications WO 00/39345 and WO 00/47767.

In various embodiments, probes and/or labeled molecules can be attached to the substrate in various ways. In certain embodiments, probes and/or labeled molecules are attached covalently to the substrate. In certain embodiments, probes and/or labeled molecules are attached by other methods. Exemplary methods include, but are not limited to, UV cross-linking, electrostatic attachment, polylysine coating of the substrate, hybridization to other nucleic acids on the substrate, and in situ synthesis of the nucleic acid on the substrate. In certain embodiments, probes and/or labeled molecules are attached with a linker molecule. Certain linker molecules include, but are not limited to, polyethylene glycol linker molecules, peptide linker molecules, and C6 linker molecules.

During manufacture, errors may occur during the depositing or synthesis of probes on features within the microarray. Thus, there may be no probe deposited at a particular feature, or there may be variation in the amount of probe deposited at different features within a microarray or variation at counterpart features in different microarrays. Such variation in probe deposition (including failure of probe attachment) may occur due to many factors. For example, the reactivity of the surfaces may vary from feature to feature and/or from microarray to microarray, different elements that are used for depositing or synthesis of probe at different features or on different microarrays may create variation in the amount and distribution of probe, and differing environmental conditions, such as humidity, can impact probe deposition. Since the attachment typically involves a chemical binding reaction with the probe or with linkers, variations in such reactions, or the absence of active reagents, can create variations in probe deposition. Variation may result during any method for attaching nucleic acid on a substrate, e.g., during in situ synthesis of probe on a substrate. Variation may often result when microarrays are made in different facilities, but also may result within the same facility.

An aim of certain embodiments is to account for such variation and to provide more accurate determination of amounts of sequences within an experimental sample. Thus, according to certain embodiments, the teachings allow one to determine whether sufficient probe is actually attached to at least one feature. In such embodiments, if no signal is detected for a given feature, the user can typically conclude that insufficient probe was bound to the microarray. Without such appropriate controls, when one uses the microarray, such a lack of signal may indicate that there was not sufficient complementary target in the sample, but it could also indicate that there was insufficient probe attached to the feature. The user would not be able to make a conclusion one way or the other. Thus, such embodiments provide an important control for the user.

Also, certain embodiments provide appropriate controls for manufacturing microarrays. In certain embodiments, one can determine whether probe has attached to each feature without running a hybridization reaction. In certain embodiments, controls can be used in hybridization reactions to test batches of microarrays to provide quality control for appropriate probe deposition.

Also, according to certain embodiments, the teachings provide more consistent controls for comparing experimental signals, which allows one to obtain an accurate ratio of experimental signal to control signal. That ratio allows one to more accurately compare a particular feature to other features on that microarray, or to features on other microarrays when one employs the same controls on such other microarrays.

Also, with different features on a microarray or on features of different microarrays, variation in intensity of signal may be due to variation in the amount of experimental target in a sample, or may be due to variation in the amount of probe in counterpart features. In certain embodiments, the teachings allow one to determine whether such variation is due to variation in the amount of probe, since the control signal is not dependent on the amount of experimental or control target in the sample. Certain such embodiments include more consistent controls, which allows one to obtain a more accurate ratio of experimental signal to control signal. In certain embodiments, that ratio allows one to more accurately compare that feature to other features on that microarray, or to features on other microarrays when one employs the same controls on such other microarrays.

Also, when microarrays are placed in an optical reader, small misalignments, e.g., caused by misplacement of the microarray in the reader or by misalignment during microarray manufacture, may result in errors in identifying the source of a signal. In some known microarrays, some features in the microarray are used to spot a label without probe in order to serve as a “landmark” in aligning the microarray. This results in fewer features available for experiments, and does not provide controls for the amount of probe deposited on the other features.

According to certain embodiments, a “landmark” is provided in features along with experimental probe. In such embodiments, the features can can serve as landmark fiducial as well as include experimental probes. According to certain embodiments, deposition of control label in easily identifiable patterns allows the features to provide a “landmark fiducial” function for aligning the microarray without sacrificing the number of features that can be used for detecting target in the assay. The landmark fiducial function also allows for easier identification of specific features within the microarray.

In some methods of array manufacture, features are deposited in a microarray on a polymeric substrate. According to certain embodiments, one can use polymeric film and methods of affixing nucleic acids such as those disclosed in published PCT application WO 99/53319. After attachment of the probes, the polymeric film is heated. In certain embodiments, it shrinks about twenty-five fold, to about four percent of its original size. After shrinking, the plastic substrate typically has folds on the surface approximately 10 microns across. The features typically are approximately 40 microns across. Such folding can create an irregular and uneven focal plane, and an indefinite depth of field.

According to certain embodiments, having a control signal at each feature typically allows one to correct for irregularities in the shape, size, and intensity of the feature, as well as in the focal plane and the depth of field. According to certain embodiments, placing the microarray on a larger surface which is shrunk allows for finer detail in depositing desired shapes for the feature, and greater regularity in probe density. Because one is initially depositing a larger feature that is later reduced in size, feature landmark fiducials typically become easier to shape to provide useful landmarks fiducials for aligning microarrays.

Also, according to certain embodiments, features are outlined with control signal. This allows one to scan features that are defined by the control signal, and disregard areas with no control signal. Areas without control signal would have no attached experimental probe. Thus, one can determine what part of the feature is background and that background should not be included in the quantitation of the features. This would make the reading of any experimental signal more accurate.

In certain embodiments, the identifiable pattern formed is a pattern of pixels, such as those read by an optical reader. In certain embodiments, an optical reader comprises an optical scanner. In certain embodiments, an optical reader comprises a CCD. Detection of a control signal within a pixel indicates that the corresponding probe is represented in that pixel. Those pixels with no control signal are then known to be background. One can then easily distinguish the pixels which are detecting a feature from the background, and scan for experimental signal only in those pixels which are detecting part of the feature. In certain embodiments, control elements can correct for vignetting resulting from certain optical readers. In certain instances, vignetting occurs toward the outside of an image.

Certain embodiments are directed to software that is used for analysis of the controls. For example, software can be used for quantitation and comparison of the various signals from different features and/or from different microarrays.

Certain Labels

In certain embodiments, labels are included. In various embodiments, use of labels can be accomplished using any one of a large number of known techniques employing known labels, linkages, linking groups, reagents, reaction conditions, and analysis and purification methods. The term “label” includes, but is not limited to, any moiety that can be attached to a nucleic acid and: (i) provides a detectable signal; (ii) interacts with a second label to modify the detectable signal provided by the second label, e.g., FRET (Fluorescent Resonance Energy Transfer); or (iii) provides a member of a binding complex or affinity set, e.g., affinity, antibody/antigen, ionic complexation, hapten/ligand, and biotin/avidin.

Exemplary, labels include, but are not limited to, light-emitting or light-absorbing compounds which generate or quench a detectable fluorescent, chemiluminescent, or bioluminescent signal (see, e.g., Kricka, L. in Nonisotopic DNA Probe Techniques (1992), Academic Press, San Diego, pp. 3-28). Fluorescent reporter dyes useful as labels include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein.

Exemplary labels also include, but are not limited to, quantum dots. “Quantum dots” refer to semiconductor nanocrystalline compounds capable of emitting a second energy in response to exposure to a first energy. Typically, the energy emitted by a single quantum dot always has the same predictable wavelength. Exemplary semiconductor nanocrystalline compounds include, but are not limited to, crystals of CdSe, CdS, and ZnS. Suitable quantum dots according to certain embodiments are described, e.g., in U.S. Pat. Nos. 5,990,479 and 6,207,392 B1, and in “Quantum-dot-tagged microbeads for multiplexed optical coding of biomolecules,” Han et al., Nature Biotechnology, 19:631-635 (2001).

Exemplary labels also include, but are not limited to, phosphors, luminescent molecules, fluorophores, radioisotopes, chromogens, enzymes, antigens, heavy metals, dyes, magnetic probes, phosphorescence groups, chemiluminescent groups, and electrochemical detection moieties. Exemplary fluorophores that are used as reporter groups include, but are not limited to, rhodamine, cyanine 3 (Cy 3), cyanine 5 (Cy 5), fluorescein, ViC™, LiZ™, Tamra™, 5-Fam™, 6-Fam™, and Texas Red (Molecular Probes). (ViC™, LiZ™, Tamra™, 5-Fam™, and 6-Fam™ are all available from Applied Biosystems, Foster City, Calif.) Exemplary radioisotopes include, but are not limited to, ³²P, ³³P, and ³⁵S Exemplary labels also include elements of multi-element indirect reporter systems, e.g., biotin/avidin, biotin/strepavidin, antibody/antigen, ligand/receptor, enzyme/substrate, and the like, in which the element interacts with other elements of the system in order to effect a detectable signal. One exemplary multi-element reporter system includes a biotin reporter group attached to a probe and an avidin or strepavidin conjugated with a fluorescent label.

The skilled artisan will appreciate that, in certain embodiments, one or more of the primers, probes, deoxyribonucleotide triphosphates, ribonucleotide triphosphates disclosed herein may further comprise one or more labels. Detailed protocols for methods of attaching labels to oligonucleotides and polynucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

Certain non-radioactive labeling methods, techniques, and reagents are reviewed in: Non-Radioactive Labelling, A Practical Introduction, Garman, A. J. (1997) Academic Press, San Diego.

In certain embodiments, at least one label is used to detect the presence of at least one probe, target, and/or other molecule. Any suitable label may be used. In certain embodiments, at least one label produces at least one fluorescent signal. In certain embodiments, at least one label produces at least one chemiluminescent signal. In certain embodiments, at least one label produces at least one fluorescent signal and at least one label produces at least one chemiluminescent signal. Use of chemiluminescent labels with microarrays has been described, e.g., in Published U.S. Patent Application No. 2003/0134286.

In certain embodiments, target nucleic acids and/or control target nucleic acids are labeled with a ligand which binds to a moiety or complex that is capable of binding the ligand and includes an enzyme capable of cleaving an enzyme labile group on a chemiluminescent substrate to produce a signal. Exemplary ligand/enzyme complex pairs which can be used include, but are not limited to, digoxigenin/anti-digoxigenin antibody:enzyme complex; biotin/strepavidin:enzyme complex; biotin/avidin:enzyme complex; and fluorescein/anti-fluorescein antibody:enzyme complex. Digoxigenin is also known as DIG. In certain embodiments, a ligand label is capable of binding to a fusion protein and the fusion protein is capable of interacting with a substrate to produce a signal.

In certain embodiments, target nucleic acids and/or control target nucleic acids are labeled with DIG which is capable of binding to an antiDIG:enzyme complex. In certain embodiments, the antiDIG:enzyme complex is an antiDIG:alkaline phosphatase complex. In certain such embodiments, the antiDIG:alkaline phosphatase complex bound to the captured nucleic acids labeled with DIG is exposed to a chemiluminescent substrate (e.g., a 1,2-dioxetane substrate) to produce a chemiluminescent signal.

In certain embodiments, prior to exposure to the chemiluminescent substrate, a ligand/enzyme complex bound to the captured nucleic acids is exposed to a chemiluminescent enhancing material. Certain exemplary chemiluminescent enhancing materials are described, e.g., in U.S. Ser. No. 10/462,742, filed Jun. 17, 2003.

In certain embodiments, target nucleic acids and/or control target nucleic acids are labeled with a ligand which binds to a moiety or a complex that includes another label and that is capable of binding the ligand. In certain embodiments, multiple labels bind to multiple complexes with labels. In certain embodiments, the multiple complexes can comprise additional labels that bind to additional complexes that include labels. Certain exemplary embodiments include branch DNA and dendrimers

Certain Exemplary Control Elements

1. Internal Control Set

In certain embodiments, an internal control set is provided. Certain internal control sets are described, e.g., in Published U.S. Patent Application No. 2002/0110828. An internal control set comprises at least one internal control molecule and at least one experimental probe at a single feature. In certain embodiments, at least one internal control molecule comprises at least one label. In certain embodiments, an internal control molecule comprises at least one internal control probe, which is complementary to an internal control target that comprises at least one label.

In certain embodiments, a signal from an internal control set can be used to confirm that experimental probe is present at a feature. In certain embodiments, a signal from an internal control set can be used to quantitate an experimental signal.

In certain embodiments, the signal from at least one internal control molecule can be used to define the shape of a feature. In certain embodiments, since the experimental probe and internal control molecule are added together, the area to which the internal control molecule is bound is the same as the area to which the experimental probe is bound. Thus, in certain embodiments, the outline of a feature may be used to identify where experimental probe is bound, and distinguish that area from background where no experimental probe is bound.

In certain embodiments, an internal control molecule is an internal control probe, which is complementary to an internal control target. In certain embodiments, an internal control probe may be used to determine whether hybridization occurs. In certain such embodiments, a feature includes an internal control probe that hybridizes to an internal control target. In certain such embodiments, detection of internal control target at the feature including internal control probe indicates that hybridization is occurring. In certain embodiments, an internal control probe can be used to determine differences in hybridization in different features of a microarray. For example, in certain instances a bubble or other artifact may interfere with hybridization in certain features of a microarray. In certain embodiments, the internal control probes can be used to detect such interference with hybridization.

In certain embodiments, an internal control probe is designed not to be complementary to any experimental target sequence. In certain such embodiments, one uses an internal control target that is complementary to the internal control probe and that is added to a test sample. Certain such internal control targets are less likely to compete for binding with experimental targets. In certain embodiments, one uses internal control probes that all have the same nucleic acid sequence and internal control targets that all have the same sequence that is complementary to the internal control probe sequence. In certain embodiments, all of the internal control set features on the microarray comprise internal control probes with the same sequence. Thus, in such embodiments, different internal control targets with different sequences need not be included for different features. In certain such embodiments, such a strategy reduces the number of synthesis reactions used to make the internal control targets for the assay. In certain embodiments, such a strategy results in less variation of hybridization to internal control probes and thus less variation in signal, than what may occur with different internal control probes with different sequences.

In certain embodiments, one uses different internal control probes that hybridize to different internal control targets. In certain embodiments, synthetic internal control targets that hybridize to the experimental probes on the microarray and that are labeled with a detectably different label than experimental targets are added to an experimental sample. In such embodiments, the experimental probes not only serve as experimental probes that hybridize to experimental targets, but also serve as internal control probes.

To decrease the chance of cross hybridization with experimental targets, in certain embodiments, internal control probes and internal control targets may include non-Watson-Crick bases. Such bases typically would not be included in experimental targets from a biological sample, and typically would not hybridize with the naturally occurring Watson-Crick bases in the experimental probes and experimental targets. Synthetic non-Watson-Crick bases, such as the AEGIS bases, are described, e.g., in U.S. Pat. Nos. 5,432,272; 5,965,364; and 6,001,983; and are available, e.g., from Eragen Biosciences, Inc.

In certain embodiments, an internal control target is not added to the test sample. In certain such embodiments in which the internal control target is not added to the test sample, the internal control probe can be complementary to a sequence that is expected to be present in the test sample. For example, the internal control probe may be complementary to a known gene that is expressed in the tissue from which the test sample was obtained.

In certain embodiments, an internal control molecule is attached to an experimental probe (e.g., an internal control probe and experimental probe can be synthesized as one oligonucleotide or an internal control molecule can comprise a label that is attached to an experimental probe). The contiguous experimental probe and internal control molecule are then attached to the microarray. The attachment to the microarray may occur through the internal control molecule, the experimental probe, or a linker that is attached to one of the internal control molecule or experimental probe. This method has an added advantage that the stochiometry of experimental probe and internal control molecule within a feature is the same. Thus, in certain of these embodiments, by determining signal from the internal control molecule, one can easily determine not only whether experimental probe has bound to the microarray, but also the amount of such binding.

In certain embodiments, an internal control molecule is not contiguous with the experimental molecule. In certain embodiments, an internal control molecule is bound to the microarray. In certain embodiments, an internal control molecule may be bound to the microarray with the same microarray binding reaction as an experimental probe. In certain embodiments, an internal control molecule is an internal control probe and the internal control probe is bound to the microarray with the same microarray binding reaction as an experimental probe. Certain of these embodiments provide a control for determining the amount of experimental probe that is attached to the feature. In certain embodiments, because the internal control probe and experimental probes bind to the microarray by the same chemical reaction, the amount of internal control probe that is detected at a feature should be representative of the amount of experimental probe that is also attached to that feature. Thus, failure to attach experimental probe, or variations in the amount of experimental probe bound to the microarray, can be detected by observing the amount of internal control signal.

In certain embodiments, a subset of experimental probes are associated with an internal control molecule, such that a specific percent of experimental probes bound to a feature are labeled. Thus, in certain such embodiments, the signal resulting from the internal control molecule can be used to calculate the amount of total experimental probe bound to a feature.

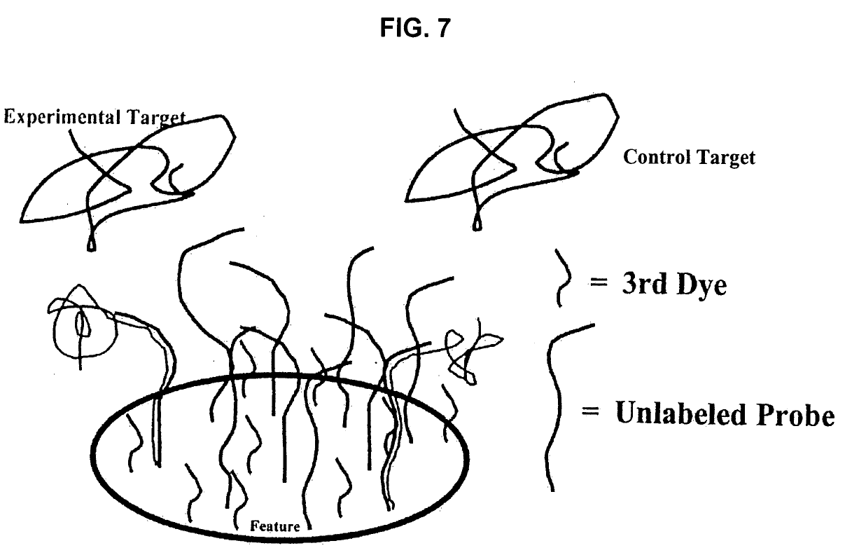

In certain embodiments, one employs labeled experimental targets (a first signal), labeled internal control targets (second signal), and labeled internal control molecules that are attached to the feature (a third signal) (See FIG. 7). In certain such embodiments, the third signal provides a control for accurately determining the amount and placement of probe that is bound to the feature. The amount of a given target in traditional biologically derived control samples is typically not known. Thus, it is possible that a given internal control target sequence is not present in the control sample. Thus, without the third signal, absence of internal control signal at a feature cannot be positively attributed to lack of internal control target sequence in the sample or to the lack of experimental probe on the feature. Also, since the amount of target in a biological sample typically is not known, one typically cannot accurately determine whether differences in signal intensity from feature to feature is due to different levels of target in the sample or is due to different levels of probe bound to the microarray.

In certain embodiments, experimental target and internal control target hybridize to the same probe. In certain embodiments, competitive hybridization is carried out with labeled experimental target (which provides a first signal if there is binding) and differently labeled internal control target (which provides a second signal if there is binding). Detection of second signal indicates presence of probe at the feature and confirms that hybridization can occur. Detection of first signal indicates the presence of experimental target in the test sample.

In certain embodiments, an internal control set comprises an internal control molecule and more than one experimental probe sequence. Certain such embodiments, may be employed for determining the amount of an experimental target in a sample. For example, this may be useful in instances in which variations in splicing result in different mRNA transcripts for the same gene, which transcripts have different overall sequences. In such cases, it may be desirable in certain embodiments to have different experimental probe sequences complementary to different portions of the gene located on the same feature. In certain embodiments, several experimental probes are created that are complementary to different regions of a transcript. In certain embodiments, these different experimental probes are deposited on the same feature. In certain embodiments, this would allow experimental target molecules representing a given gene a greater opportunity to hybridize to the feature. Any number of different experimental probes may be included in the same feature. Consequently, in certain embodiments, the experimental signal could be a more accurate indication of the levels of expression of a given gene in the sample, and only one feature is used to accomplish this result rather than multiple features. Of course, these multiple experimental probes can be directed to other nucleic acids, such as intergenic regions, introns, etc.

2. Spatial Normalization Control

In certain embodiments, a spatial normalization control is provided. The term “spatial normalization control” refers to a control element comprising two or more features on a microarray, each of which produces at least one signal, and by comparing the at least one signal of two or more of those features, one can normalize signals of the microarray in the regions of the compared features. In certain embodiments, a spatial normalization control feature produces at least two detectable different signals. In certain embodiments, a spatial normalization control feature produces both a fluorescent and chemiluminescent signal.

In certain embodiments, a spatial normalization control feature produces both a fluorescent and chemiluminescent signal. In certain embodiments, one determines the ratio of fluorescent signal to chemiluminescent signal at each of two or more features each comprising a spatial normalization control. In certain embodiments, one compares the ratio of fluorescent signal to chemiluminescent signal of two spatial normalization controls in two different regions of a microarray. In certain embodiments, if the ratios are substantially different at the two different features, the user normalizes the signals from experimental features from the two different regions. If the ratios are substantially the same, no adjustment of the experimental signals from the two regions is made. In certain embodiments, a given set of features is assigned to a given spatial normalization control for that region. In certain embodiments, algorithms are useful for normalizing signals from different features based on the fluorescent and chemiluminescent signals from a spatial normalization control.

In certain embodiments, the fluorescent and chemiluminescent signals are achieved by attaching labeled molecules to the two or more spatial normalization control features. In certain embodiments, those labeled molecules are labeled probes. In certain embodiments, unlabeled control probes are attached to the two or more spatial normalization control features and fluorescent and chemiluminescent labeled targets that are complementary to those unlabeled control probes are present in the sample. In certain embodiments, the labeled targets are added to an experimental sample. In certain embodiments, all of the control probes that hybridize to fluorescent labeled targets have the same sequence and all of the control probes that hybridize to chemiluminescent labeled targets have the same sequence. In certain such embodiments, the control probes that hybridize to fluorescent labeled targets have a different sequence than the control probes that hybridize to chemiluminescent labeled targets. In certain embodiments, the chemiluminescent labeled targets comprise chemiluminescent labels, e.g., labels that interact with a chemiluminescent substrate to produce chemiluminescent signal or labels that bind to a molecule or complex that interacts with a chemiluminescent substrate to produce chemiluminescent signal. In certain embodiments, a first probe or molecule comprising a label and a second probe lacking a label are attached to a spatial normalization control feature, and target complementary to the second probe and comprising a label is present in the test sample.

In certain embodiments, a spatial normalization control feature produces at least two detectably different signals. In certain embodiments, one determines the ratio of two detectably different signals at each of two or more features each comprising a spatial normalization control. In certain embodiments, one compares that ratio of the detectably different signals of two spatial normalization controls in two different regions of a microarray. In certain embodiments, if the ratios are substantially different at the two different features, the user normalizes the signals from experimental features from the two different regions. If the ratios are substantially the same, no adjustment of the experimental signals from the two regions is made. In certain embodiments, a given set of features is assigned to a given spatial normalization control for that region. In certain embodiments, algorithms are useful for normalizing signals from different features based on the detectably different signals from a spatial normalization control.

In certain embodiments, at least two detectably different signals are achieved by attaching labeled molecules to the two or more spatial normalization control features. In certain embodiments, those labeled molecules are labeled probes. In certain embodiments, unlabeled control probes are attached to the two or more spatial normalization control features and detectably different labeled targets that are complementary to those unlabeled control probes are present in the sample. In certain embodiments, the labeled targets are added to an experimental sample. In certain embodiments, all of the control probes have the same sequence. In certain embodiments, all of the control probes that hybridize to first labeled targets have the same sequence and all of the control probes that hybridize to detectably different second labeled targets have the same sequence. In certain such embodiments, the control probes that hybridize to first labeled targets have a different sequence than the control probes that hybridize to detectably different second labeled targets. In certain embodiments, a first probe or molecule comprising a label and a second probe lacking a label are attached to a spatial normalization control feature, and target complementary to the second probe and comprising a label is present in the test sample.

In certain embodiments the at least two detectably different signals result from at least two detectably different fluorescent labels. In certain embodiments the at least two detectably different signals result from at least two detectably different chemiluminescent labels. In certain embodiments the at least two detectably different signals result from a combination of fluorescent labels and chemiluminescent labels.

In certain embodiments, a spatial normalization control feature produces a signal. In certain embodiments, one compares the signals of two spatial normalization controls in two different regions of a microarray. In certain embodiments, if the signals are substantially different at the two different spatial normalization control features, the user normalizes the signals from experimental features from the two different regions. If the signals at the spatial normalization controls are substantially the same, no adjustment of the experimental signals from the two regions is made. In certain embodiments, a given set of features is assigned to a given spatial normalization control for that region. In certain embodiments, algorithms are useful for normalizing signals from different features based on the signals from spatial normalization controls.

In certain embodiments, the signals are achieved by attaching labeled molecules to the two or more spatial normalization control features. In certain embodiments, those labeled molecules are labeled probes. In certain embodiments, unlabeled control probes are attached to the two or more spatial normalization control features and labeled targets that are complementary to those unlabeled control probes are present in the sample. In certain embodiments, the labeled targets are added to an experimental sample. In certain embodiments, all of the control probes have the same sequence.

In certain embodiments, spatial normalization controls are used to normalize differences in detected signals due to vignetting. In certain instances, vignetting results in an image in which the signal intensity toward the center of the microarray is greater than the signal intensity toward the outside, for example in the outside corners, of the microarray.

In certain embodiments, spatial normalization controls are designed such that the measured signal values from two or more spatial normalization controls that are compared are substantially the same if no normalization is needed. In certain such embodiments, one compares the signals of two spatial normalization controls in two different regions of a microarray. In certain such embodiments, if the signals are substantially different at the two different spatial normalization control features, the user normalizes the signals from experimental features from the two different regions. If the signals at the spatial normalization controls are substantially the same, no adjustment of the experimental signals from the two regions is made. In certain embodiments, a given set of features is assigned to a given spatial normalization control for that region. In certain embodiments, algorithms are useful for normalizing signals from different features based on the signals from spatial normalization controls. In certain embodiments, spatial normalization control probes are the same and spatial normalization control targets are added at a given concentration to an experimental sample.

3. Control Ladder

In certain embodiments, the teachings provide for a control ladder, which is a control element comprising at least two different features on a microarray that comprise labeled molecules in different concentrations.

In certain embodiments, a control ladder comprises several features, each of which comprises labeled probe that is deposited at a different known concentration. In certain embodiments, a control ladder allows one to quantify results from a microarray. For example, in certain embodiments, one compares the signal from a labeled experimental target to the signal from the control ladder. For example, in certain embodiments, if the signal from an experimental target is substantially the same as a signal at a given concentration of the control ladder, one concludes that the label is present at the experimental feature at that given concentration. Thus, one can quantify the amount of experimental target present at the feature, because the amount of label per amount of target is known.

In certain embodiments, the labeled molecules of a control ladder comprise fluorescent labels. In certain such embodiments, the labeled molecule comprises a 5′-LIZ label. In certain embodiments, the labeled molecules of a control ladder comprise chemiluminescent labels, e.g., labels that interact with a chemiluminescent substrate to produce chemiluminescent signal or labels that bind to a molecule or complex that interacts with a chemiluminescent substrate to produce chemiluminescent signal. In certain such embodiments, the labels can be a 5′-DIG label.

In certain embodiments, a single feature of a control ladder comprises both labeled molecules comprising fluorescent labels and chemiluminescent labels. In certain embodiments, a microarray comprises more than one control ladder. In certain such embodiments, more than one control ladder may each comprise the same label or labels. In certain embodiments, more than one control ladders may comprise different labels. For example, in certain embodiments, a microarray may comprise at least one first control ladder comprising at least one fluorescent label and at least one second control ladder comprising at least one chemiluminescent label. In certain embodiments, each feature of a control ladder comprises both a fluorescent label, which is part of a fluorescent control ladder, and a chemiluminescent label, which is part of a chemiluminescent control ladder. In certain embodiments, a microarray comprises a fluorescent control ladder on separate features from a chemiluminescent control ladder.

In certain embodiments, labeled probe is provided on separate features of a microarray at ten-fold dilutions. In certain embodiments, the highest concentration of labeled probe for the control ladder is about 10 μM. In certain embodiments, the different concentrations of label for a control ladder are selected from: 10 μM, 1 μM, 0.1 μM, 0.01 μM, 1 nM, 0.1 nM, 0.01 nM, 1 pM, 0.1 pm, 0.01 pM, and so on.

In certain embodiments, a control ladder is used to identify the linear range of detection for a microarray assay. In certain embodiments, the upper end of the linear range is the signal value from a control ladder feature above which the detection method range becomes non-linear or plateaus. In other words, control ladder features above the upper end of the linear range will all have approximately the same signal value even though the concentrations of label vary. In certain embodiments, the lower end of the linear range is the signal value from a control ladder feature directly above the control ladder feature having the highest concentration of label that has a signal value no higher than background. In other words, below the lower end of the linear range, the detection method is not sufficiently sensitive to detect signal. In certain embodiments, one may choose to disregard signal values above or below the linear range of detection for a microarray assay.

In certain embodiments, the same control ladder is included in two or more regions of a microarray and the control ladders allow one to compare signals from the different regions. In certain embodiments, the same control ladder is included in two or more separate microarrays and the control ladders allow one to compare signals from different microarrays. In certain such embodiments, one may use linear regression analysis using the linear parts of the two control ladders to compare results.

In certain embodiments, a control ladder is used to assess sensitivity of an assay. For example, in certain embodiments, one notes the control ladder feature with the lowest concentration of label from which one obtains a signal. In certain embodiments, such information may be used for quality control.

In certain embodiments, control ladders are used to assess whether certain steps in an assay were successful. For example, even if the only positive signal detected from a microarray is from at least one control ladder, in certain embodiments, the user may nevertheless conclude that the optical reader was functioning. Absent such a positive signal, in certain instances, the user could not draw such a conclusion. Further, in certain embodiments, a positive signal from at least one control ladder indicates that the labeled molecules were successfully attached to the microarray. In certain embodiments, ultraviolet light is used to attach oligonucleotides to a microarray, and a positive signal from at least one control ladder indicates that labeled molecules were successfully attached to the microarray using ultraviolet light. In certain embodiments, if the labeled molecules and experimental probes were attached side by side in similar reactions, the user may conclude that the experimental probes were successfully attached. In certain embodiments, when chemiluminescent labels are used, a positive signal from at least one control ladder indicates that the chemiluminescent reaction was successful.

In certain embodiments, the person attaching probes to the microarray can determine the relative success of the attachment reaction by the results provided from the control ladder. For example, in certain such embodiments, the probes for the control ladder are applied to the microarray in different concentrations. Accordingly, in certain embodiments, the signal from different features with different concentrations of probes applied to them should provide different levels of signal. In certain such embodiments, if there is an insufficient difference in the signal from certain different features of a control ladder, a conclusion may be made that the attachment reaction did not occur in an acceptable manner.

4. Non-specific Background Control

In certain embodiments, a non-specific background control is provided. A non-specific background control is a control element comprising at least one non-specific background control probe which is designed based on its dissimilarity to any known target that is expected to be present in a test sample. In certain embodiments, the Basic Local Alignment Tool (BLAST) program is used to identify a sequence with low identity or homology to gene sequences in a database. In certain embodiments, the database comprises sequences from a particular species or tissue type. In certain embodiments, the database comprises sequences deposited in the National Center for Biotechnology Information (NCBI).

In certain embodiments, a non-specific background control is used to determine whether, or the degree to which, targets are bound to probes non-specifically in an assay. In certain embodiments, since one does not expect target complementary to the non-specific background probe to be present in the test sample, one expects the signal at the feature comprising such a probe to be low, relative to the signal from other features where a positive signal is expected. In certain embodiments, a signal from a non-specific background control similar to, or greater than, signals at one or more experimental features may indicate that target is bound non-specifically. In certain embodiments, a signal from a non-specific background control similar to, or greater than, signals at one or more control probes may indicate that target is bound non-specifically.

In certain embodiments, one may adjust the stringency of an assay based on results obtained from a non-specific background control. In certain embodiments, one may adjust the signals from experimental features by subtracting background using the signals obtained from the non-specific background controls. For example, in certain embodiments, one may subtract the median signal value obtained from non-specific background controls from signal values obtained from experimental features. For example, in certain embodiments, one may subtract the mean signal value obtained from non-specific background controls from signal values obtained from experimental features.

5. Positive Gene Control

In certain embodiments, a positive gene control is provided. A positive gene control is a control element comprising at least one positive gene control probe that is complementary to at least a portion of a known gene without any mismatches and the known gene is expected to be present in a sample. In certain embodiments, the known gene is typically present in samples from a target species from which the test sample was obtained.

In certain embodiments, a signal from a positive gene control indicates that the quality and/or quantity of the sample was adequate to provide a signal. For example, in certain embodiments, a signal from a positive gene control indicates that the sample was not degraded. In certain embodiments, a signal from a positive gene control indicates that target in the test sample was successfully labeled. In certain instances, absent a positive gene control, one might have difficulty distinguishing a negative finding (i.e., that an experimental target is not present in the target sample) from a failed assay due to degraded sample or unsuccessful labeling.

In certain embodiments, more than one positive gene control is included on a microarray. In certain embodiments, the signal values from more than one positive gene control are averaged. In certain embodiments, such an average positive gene control value is used to assess labeling of target in a test sample. In certain instances, an average positive gene control value may be more reliable than a single positive gene control, because the presence and/or amount of a particular positive gene control target may not be known with certainty. Although each positive gene control target is expected to be present in the test sample, it is possible that one or more positive gene control targets is not present or is present at lower than expected amounts. Thus, in certain instances, an average of more than one positive gene control improves reliability.

In certain embodiments, a microarray includes multiple different positive gene control probes for different portions of a given gene. In certain such embodiments, signals from the positive gene control probes may be used to make a conclusion about the quality of RNA in a sample and/or labeled DNA produced from it in a reverse transcription reaction. For example, in certain embodiments, a microarray includes multiple different positive gene control probes that are complementary to different portions in spaced intervals along a given gene.

In certain such embodiments, a reverse transcription reaction is used to make labeled DNA from a sample comprising RNA. In certain such embodiments, after the reaction, the material is exposed to the microarray. In certain such embodiments, one compares the signal from positive gene control probes directed to portions of the transcript of the given gene near or at the 3′ end to the signal from positive gene control probes directed to portions of the transcript of the given gene near or at the 5′ end of the given gene. In certain such embodiments, a ratio is determined between the signal from one or more positive gene control probe complementary to one or more portion of the transcript of the given gene near or at the 3′ end to the signal from one or more positive gene control probe complementary to one or more portion of the transcript of the given gene near or at the 5′ end of the given gene. In certain such embodiments, if such a ratio meets or exceeds a threshold level, a conclusion is made that the RNA was degraded and/or that the reverse transcription reaction did not produce suitable labeled DNA. In certain such embodiments, if such a ratio is below a threshold level, a conclusion is made that the RNA was not substantially degraded and that the reverse transcription reaction produced suitable labeled DNA.

In certain embodiments of microarrays that include multiple different positive gene control probes for different portions of a given gene, signals from the positive gene control probes may be used to make a conclusion about the quality of RNA in a sample and/or labeled RNA produced from it in a reverse transcription reaction, followed by an in vitro transcription reaction. For example, in certain embodiments, a microarray includes multiple different positive gene control probes that are complementary to different portions in spaced intervals along a given gene.

In certain such embodiments, a reverse transcription reaction, followed by an in vitro transcription reaction, is used to make labeled RNA from a sample comprising RNA. In certain such embodiments, after the reaction, the material is exposed to the microarray. In certain such embodiments, one compares the signal from positive gene control probes directed to portions of the transcript of the given gene near or at the 3′ end to the signal from positive gene control probes directed to portions of the transcript of the given gene near or at the 5′ end of the given gene. In certain such embodiments, a ratio is determined between the signal from one or more positive gene control probe complementary to one or more portion of the transcript of the given gene near or at the 3′ end to the signal from one or more positive gene control probe complementary to one or more portion of the transcript of the given gene near or at the 5′ end of the given gene. In certain such embodiments, if such a ratio meets or exceeds a threshold level, a conclusion is made that the RNA was degraded and/or that the reverse transcription reaction, followed by the in vitro transcription reaction, did not produce suitable labeled RNA. In certain such embodiments, if such a ratio is below a threshold level, a conclusion is made that the RNA was not substantially degraded and that the reverse transcription reaction, followed by the in vitro transcription reaction, produced suitable labeled RNA.

In certain embodiments, multiple positive gene control probes that are complementary to multiple different portions of a transcript of a gene can be used to determine the extent of degradation of RNA. For example, in certain embodiments, one may include 10 different positive gene control probes that are complementary to portions along a given gene transcript in the 3′ to 5′ direction. If signals from each of the 10 different positive gene controls are not significantly different (below a threshold difference), a conclusion can be made that there is not significant degradation. If signals from the first nine different positive gene controls are not significantly different (below a threshold difference), but the signal from the tenth positive gene control probe is significantly less than the other nine, a conclusion can be made that there is degradation past the region of the given gene that is complementary to the ninth positive gene control probe. In certain embodiments, one can draw conclusions about the degradation of other RNA in the sample from the results with the test RNA (the RNA analyzed by the multiple positive gene control probes). For example, in the discussion above with the ten illustrative positive gene control probes, a conclusion may be made that other RNA is not degraded if it has a length equal to or less than the length of the test RNA up to the region that is complementary to the ninth positive gene control probe.

In certain embodiments, there are two to 30, including any number in that range, multiple different positive gene control probes complimentary to different portions of a transcript of a given gene. In certain embodiments, multiple different positive gene control probes are directed to different portions of a gene that has a transcript length of 500 to 20,000, including any number in that range, nucleotides. In certain embodiments, multiple different positive gene control probes are directed to different portions of a gene that has a transcript length of at least 1000 nucleotides. In certain embodiments, multiple different positive gene control probes are directed to different portions of a gene that has a transcript length of at least 3000 nucleotides. In certain embodiments, multiple different positive gene control probes are directed to different portions of a gene that has a transcript length of at least 5000 nucleotides. In certain embodiments, multiple different positive gene control probes are complementary to different portions of a gene encoding apolipoprotein B. In certain embodiments, multiple different positive gene control probes are complementary to different portions of a gene encoding ubiquitin specific protease 34. In certain embodiments, multiple different positive gene control probes are complementary to different portions of a gene encoding nebulin. In certain embodiments, multiple different positive gene control probes are complementary to different portions of a gene encoding mitogen. In certain embodiments, multiple different positive gene control probes are complementary to different portions of a gene encoding activated protein kinase 4.

In certain embodiments, there are multiple sets of positive gene control probes directed to different genes, wherein each set includes multiple different positive gene control probes complementary to different portions of a given gene.

In certain embodiments, a positive gene control probe is complementary to a sequence that is expected to be in any ribosomal RNA sample irrespective of the source. Exemplary sequences include, but are not limited to, ribosomal RNA, repetitive sequences (for example, sines, lines, alus), and simple sequence repeats (for example, poly A, poly CA, and triplet repeats). In certain such embodiments, the signal from such positive gene control probes may be used to determine if the correct amount of RNA was used in a reverse transcription reaction or if the correct amount of product produced from a reverse transcription reaction has been added to a microarray. For example, in certain embodiments, a given signal from such a positive gene control is expected if the correct amount of RNA is used in a reverse transcription reaction and the correct amount of product is added to the microarray. If the signal varies from the given signal, a conclusion may be made that the incorrect amount of RNA was used in the reverse transcription reaction and/or the incorrect amount of product was added to the microarray.

In certain embodiments of microarrays that include positive gene control probes that are complementary to a sequence that is expected to be in any ribosomal RNA sample irrespective of the source, the signal from such positive gene control probes may be used to determine if the correct amount of RNA was used in a reverse transcription reaction or if the correct amount of product produced from a reverse transcription reaction, followed by an in vitro transcription reaction, has been added to a microarray. For example, in certain embodiments, a given signal from such a positive gene control is expected if the correct amount of RNA is used in a reverse transcription reaction and the correct amount of product is added to the microarray. If the signal varies from the given signal, a conclusion may be made that the incorrect amount of RNA was used in the reverse transcription reaction and/or the incorrect amount of product was added to the microarray.

6. Positive Gene Mismatch Control

In certain embodiments, a positive gene mismatch control is provided. A positive gene mismatch control is a control element comprising at least one positive gene mismatch control probe that comprises nucleotides that base pair with at least a portion of a known gene that is expected to be present in a sample and comprises at least one mismatch with the at least a portion of the known gene. Exemplary numbers of mismatches include, but are not limited to, one, two, three, four, five, six, seven, and eight mismatches.

In certain embodiments, one may monitor hybridization and/or wash conditions of an assay by including one or more positive gene mismatch controls. In certain embodiments, one may include a positive gene control and a series of positive gene mismatch control probes comprising one mismatch, two mismatches, three mismatches, and so on. Under appropriate hybridization and/or wash conditions, one expects signal to decrease as the number of mismatches increases. In certain embodiments, one may draw conclusions about the hybridization and/or wash conditions by comparing the results from such a series of positive gene mismatch controls. In certain embodiments, one may adjust hybridization and/or wash conditions to achieve a desired tolerance for mismatches.

In certain embodiments, one may use results from positive gene mismatch controls as quality control to allow comparison of results from different microarrays. For example, in certain embodiments, if one obtains the same or similar results from the same positive gene mismatch controls on different microarrays, one may conclude that stringency conditions were similar for those different microarrays. However, if one obtains different results from the same positive gene mismatch controls on different microarrays, one may conclude that stringency conditions were different on the two microarrays. In certain embodiments, one may choose to compare results from different microarrays only if the stringency conditions were similar.

7. Landmark Fiducial

In certain embodiments, a landmark fiducial is provided. A landmark fiducial is a control element comprising a feature that produces a signal at a known location on a microarray. The signal of a landmark fiducial may be used to orient the microarray. In certain embodiments, when microarrays are placed in an optical reader, small misalignments, e.g., caused by misplacement of the microarray in the reader or by misalignment during microarray manufacture, may result in errors in identifying the source of a signal. In certain embodiments, since the landmark fiducial produces a signal at a known location, that location can serve as a reference point for orienting the microarray and identifying the positions of the other signals relative to the landmark fiducial.

In certain embodiments, the signal at the landmark fiducial is achieved by attaching a labeled molecule to the feature. In certain embodiments, the labeled molecule of a landmark fiducial is a labeled probe. In certain embodiments, signal at the landmark fiducial is achieved by attaching unlabeled probe to the feature and adding labeled target complementary to that unlabeled probe to the sample.

In certain embodiments, the signal from one or more landmark fiducial feature may be used to identify a particular microarray and/or one or more regions of a microarray. For example, in certain embodiments, a given type of microarray includes one or more landmark fiducial feature that provides a unique signal, and/or that is in a unique position, compared to other types of microarrays. In certain such embodiments, the different types of microarrays may be for different species, for example, microarrays for human samples, rodent samples, or any other species. In certain embodiments, the different types of microarrays may be different microarrays for the same species.

In certain embodiments, a microarray includes one or more landmark fiducial features that may be used to identify different regions of a microarray. For example, in certain embodiments, a microarray may be arranged in two sections for two images and one or both of the sections includes one or more landmark fiducial features that is unique to that section. In certain embodiments, a microarray may be subdivided into multiple regions and each such region includes one or more landmark fiducial feature that is unique to that section. In certain embodiments, one or more landmark fiducial features is unique to a section by providing a unique signal and/or is in a unique position in that section.

In certain embodiments, landmark fiducial features can be used to determine if particular oligonucleotides have been placed in an incorrect position in a microarray. For example, in certain embodiments, if one or more sections do not include one or more landmark fiducial features that provide the correct unique signal and/or are not in the correct unique position in that section, a conclusion can be made that the placement of oligonucleotides in that section is incorrect.

In certain embodiments, a combination of multiple landmark fiducial features provide a unique pattern for a given microarray and/or for a given region of a microarray. In certain such embodiments, such a combination may include a given number of features that either provide a signal or are blank (do not provide a significant signal). In certain such embodiments, such a combination may include 1 to 10, including any number in that range, features that either provide a signal or are blank. For example, in certain such embodiments, the combination of landmark fiducial features includes seven features that either provide a signal or are blank. In such embodiments, up to 2⁷ or up to 128 different possible codes can be provided. The codes may be used for different microarrays and/or for different regions of microarrays. For example, 128 codes may be used for 128 different microarrays, 128 different regions of a microarray; or for 128 different combinations of microarrays and regions of microarrays (for example, four different microarrays with 32 different regions; or one or two different microarrays with 48 different regions). In certain embodiments, not all possible codes are used.

In certain embodiments, other control elements may serve as landmark fiducial features used to identify a type of microarray and/or different regions of a microarray. For example, in certain embodiments, another control element that is present in each of several different regions of a microarray can be placed in a unique position in each different region. For example, in certain embodiments, a microarray may be subdivided into four different regions and each different region includes a control ladder. In certain such embodiments: the first region may include the control ladder toward the upper left corner of the region; the second region may include the control ladder toward the upper right corner of the region; the third region may include the control ladder toward the lower left corner of the region; the fourth region may include the control ladder toward the lower right corner of the region.

In certain embodiments, landmark fiducial features may be identified in view of detectably different signals. In certain embodiments, landmark fiducial features may be identified in view of different intensities of signals.

8. Hybridization Control

In certain embodiments, a hybridization control is provided. A hybridization control is a control element comprising at least one hybridization control probe that is complementary (and without any mismatches) to at least a portion of a labeled hybridization control target that is added to the test sample. In certain embodiments, the sequence of a hybridization control probe, is designed based on its dissimilarity to the sequence of any known target that is expected to be present in the test sample. In certain instances, using such a sequence reduces the likelihood that competition for binding between the hybridization control target and sample nucleic acids will affect the results. In certain embodiments, a positive signal from a hybridization control indicates that the hybridization was successful and/or that signal can be detected.

In certain embodiments, different hybridization controls comprising hybridization control probes of different lengths are included. In certain instances, with shorter oligonucleotides, the melting temperature (Tm) is lower, which causes such shorter oligonucleotides to bind to their complements less efficiently than longer oligonucleotides. In certain instances, binding can be made more efficient by altering the chemical bonds between the bases. For example, nucleotide bases joined by peptide bonds, instead of the natural phosphodiester bonds, typically have higher melting temperatures. In certain embodiments, the hybridization control target is composed of short oligonucleotides of nine or ten bases in length, joined by peptide bonds or LNA linkages, such as those described in published PCT applications WO 00/56748; and WO 00/66604. Such targets have the Tm of longer oligonucleotides, allowing them to withstand certain stringent conditions that may be used for hybridization assays.

In certain embodiments, one uses hybridization control probes to monitor the stringency of an assay. In certain embodiments, a microarray comprises hybridization control probes that are the same length as experimental probes. In certain embodiments, a microarray comprises certain different experimental probes having different lengths. In certain such embodiments, a microarray comprises different hybridization control probes having different lengths, such that there is a hybridization control probe having the length of each of the different lengthed experimental probes.

9. Hybridization Mismatch Control

In certain embodiments, a hybridization mismatch control is provided. A hybridization mismatch control is a control element comprising at least one hybridization mismatch control probe that comprises nucleotides that base pair with at least a portion of a labeled hybridization control target that is added to the test sample and comprises at least one mismatch with the at least a portion of the labeled hybridization control target. Exemplary numbers of mismatches include, but are not limited to, one, two, three, four, five, six, seven, and eight mismatches.

In certain embodiments, one may monitor hybridization conditions of an assay by including one or more hybridization mismatch controls. In certain embodiments, one may include a hybridization control and a series of hybridization mismatch control probes comprising one mismatch, two mismatches, three mismatches, and so on. Under appropriate hybridization conditions, one expects signal to decrease as the number of mismatches increases. In certain embodiments, one may draw conclusions about the hybridization conditions by comparing the results from such a series of hybridization mismatch controls. In certain embodiments, one may adjust hybridization conditions to achieve a desired tolerance for mismatches.

In certain embodiments, one may use results from a hybridization mismatch control as quality control to allow comparison of results from different microarrays. For example, in certain embodiments, if one obtains the same or similar results from the same hybridization mismatch controls on different microarrays, one may conclude that stringency conditions were similar for those different microarrays. However, if one obtains different results from the same hybridization mismatch controls on different microarrays, one may conclude that stringency conditions were different on the two microarrays. In certain embodiments, one may choose to compare results from different microarrays only if the stringency conditions were similar.

10. Reverse Transcription Control

In certain embodiments, a reverse transcription control is provided. A reverse transcription control is a control element comprising at least one reverse transcription control probe that is complementary to a control target derived from a reverse transcription reaction.

Reverse transcription is a reaction in which cDNA is synthesized from template RNA. In certain embodiments, a user may use sample RNA to synthesize cDNA by reverse transcription and then use that cDNA in an assay using a microarray. In certain embodiments, one may include control RNA in the reverse transcription reaction, which will result in synthesis of a control cDNA. In certain such embodiments, the at least one reverse transcription control probe on the microarray is complementary to the control cDNA produced in the reverse transcription assay. In certain such embodiments, a reverse transcription control may allow the user to conclude that the reverse transcription assay was successful. For example, in certain embodiments, if the reverse transciption control gives a positive signal and an experimental sample gives no signal, the user may be able to conclude that the reverse transcription reaction was successful, and that the sequence corresponding to the experimental probe is not present in the original RNA sample.

11. In Vitro Transcription Control

In certain embodiments, an in vitro transcription control is provided. An in vitro transcription control is a control element comprising at least one in vitro transcription control probe that is complementary to a control target derived from an in vitro transcription reaction.

In vitro transcription is a reaction in which RNA is synthesized from template DNA. In certain embodiments, a user may use sample DNA to synthesize RNA by in vitro transcription and then use that RNA in an assay using a microarray. In certain embodiments, one may include control DNA in the in vitro transcription reaction, which will result in synthesis of a control RNA. In certain such embodiments, at least one in vitro transcription control probe is complementary to the control RNA produced in the in vitro transcription assay. In certain such embodiments, an in vitro transcription control may allow the user to conclude that the in vitro transcription assay was successful. For example, in certain embodiments, if the in vitro transciption control gives a positive signal and an experimental sample gives no signal, the user may be able to conclude that the in vitro transcription reaction was successful, and that the sequence corresponding to the experimental probe is not present in the original DNA sample.

12. Buffer Blank Control

In certain embodiments, a buffer blank control is provided. A buffer blank control is a control element feature that has buffer on the feature, and which has no probe or label attached to the feature. In certain embodiments, the buffer is applied to the microarray with the same device that is used to apply oligonucleotides, for example, probes to the microarray. In certain embodiments, if signal is detected on a buffer blank control, it is concluded that oligonucleotides, for example, probes left over from a previous application has contaminated the buffer blank control. In certain such embodiments, a manufacturer of the microarray uses the buffer blank control as a quality control. In certain embodiments, buffer blank controls can be spotted directly following application of a high concentration of oligonucleotide to the microarray. In certain embodiments, signal detected from a buffer blank control indicates that the buffer is producing a signal on its own. For example, in certain such instances, the buffer may autofluoresce. In certain such embodiments, the user may use such information to normalize other signals. In certain embodiments, if the user detects signal on a buffer blank control, the user concludes that there is spatial crosstalk, in other words, there is carry over of signal from another feature.

13. Blanks

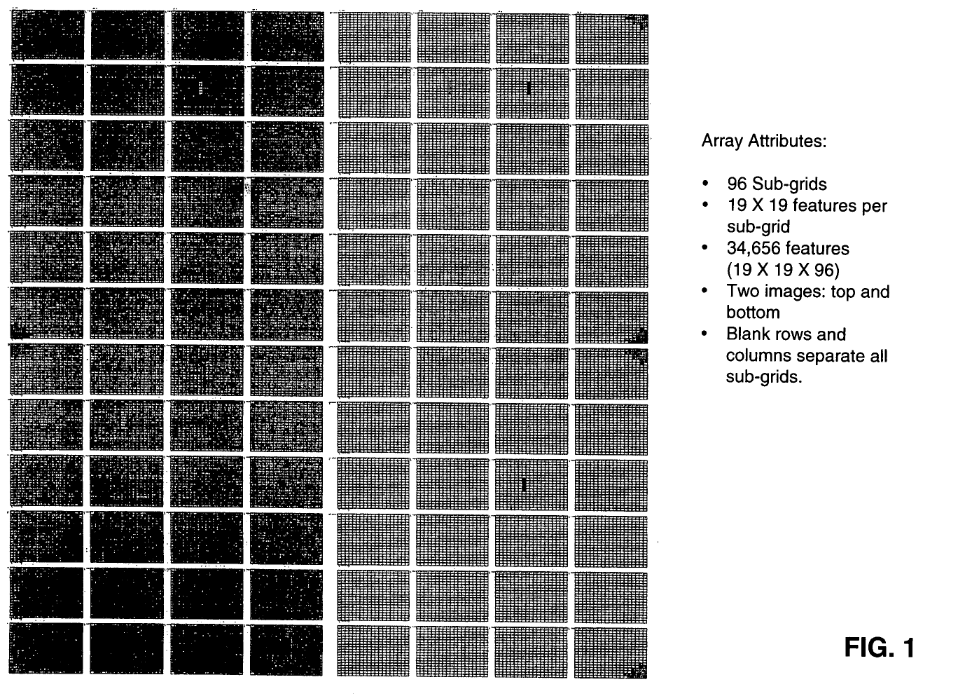

In certain embodiments; areas of the microarray are left blank. For example, in certain embodiments, rows and columns between sub-grids are left blank. See, for example, FIG. 1. In certain embodiments, if the user detects signal in a blank row or column, the user concludes that there is spatial crosstalk, in other words, there is carry over of signal from another feature. In certain embodiments, the blank rows and columns can be used to align the microarray for reading. In certain embodiments, the blank rows and columns can be used by gridding algorithms.

14. Gridding Reference Controls

In certain embodiments, a gridding reference control is provided. The term “gridding reference control” refers to a control element comprising multiple features on a microarray that provide a first signal that can be used to determine where the features are represented in an image of that first signal, as well as where those features are represented in one or more additional images of one or more additional detectably different signals. In certain embodiments, the first signal is provided by a label. In certain embodiments, the label is a fluorescent label. In certain embodiments, the label is a chemiluminescent label.

In certain embodiments, a microarray comprises multiple gridding reference control features that provide a first signal. In certain embodiments, the gridding reference control features further comprise other controls and/or experimental probes that provide a second detectably different signal. In certain embodiments, the gridding reference control features can be used to determine where those features are represented in an image of the first signal. In certain embodiments, that information can also be used to determine where those features are represented in an image of the second detectably different signal.

In certain instances, the presence and/or intensity of signal from experimental target on various features of a microarray may vary depending upon the make-up of the experimental sample. In certain instances, that variation may make it difficult to determine where features comprising experimental probe are represented in an image of the signal from the experimental target. In certain embodiments, a gridding reference control can assist in determining where features comprising experimental probe are represented in an image of the signal from the experimental target.

15. Attachment Control

In certain embodiments, an attachment control is provided. An attachment control is a control element comprising at least one attachment control oligonucleotide that does not include a chemical attachment group, which is a chemical group that is used to attach other oligonucleotides to the microarray.

In certain embodiments, an attachment control oligonucleotide is an attachment control probe that has a sequence that is the same as the sequence of a counterpart probe. In such embodiments, the counterpart probes, however, include a chemical attachment group. In certain embodiments, the signal from the counterpart probes is compared to the signal from the attachment control probes. In certain such embodiments, if the ratio of the signal from the counterpart probes to the signal from the attachment control probes meets or exceeds a threshold level, a conclusion is made that an acceptable level of specific attachment of probes over nonspecific attachment has occurred. When used to discuss attachment of probes to a microarray, specific attachment is attachment that occurs as a result of a chemical attachment group. When used to discuss attachment of probes to a microarray, nonspecific attachment is attachment that occurs not as a result of a chemical attachment group. In certain such embodiments, if the ratio of the signal from the counterpart probes to the signal from the attachment control probes is below a threshold level, a conclusion is made that an unacceptable level of specific attachment of probes over nonspecific attachment has occurred.

In certain embodiments, the threshold ratio of the signal from the counterpart probes to the signal from the attachment control probes is at least 1.5 and can be any higher number (including fractions). In certain embodiments, the threshold ratio of the signal from the counterpart probes to the signal from the attachment control probes is any number from 5 to 10. In certain embodiments, the threshold ratio of the signal from the counterpart probes to the signal from the attachment control probes is 10.

In certain instances, unacceptable specific attachment may be caused by one or more of the following: insufficient specific attachment of probes using the chemical group; insufficient washing after a chemical attachment reaction; and excessive nonspecific attachment of oligonucleotides to the microarray.

Exemplary chemical attachment groups include, but are not limited to, amino groups, thiol groups, biotin, avidin, streptavidin, and acrylamide groups. In certain embodiments, the chemical attachment group is an amino group.

In certain embodiments, the signal from the attachment control probes is detectably different from the signal from the counterpart probes. In certain embodiments, the attachment control probes are included on separate features than the counterpart probes. In certain such embodiments, the signals from the attachment control probes and the counterpart probes are not detectably different.

In certain embodiments, the sequence of the attachment control probes and the counterpart probes is complementary to at least a portion of a known gene without any mismatches and the known gene is expected to be present in a sample. In certain embodiments, the known gene is typically present in samples from a target species from which the test sample was obtained. In certain embodiments, the sequence of the attachment control probes and the counterpart probes is the same as the sequence of a positive gene control probe that is used for the microarray. In certain embodiments, the sequence of the attachment control probes and the counterpart probes is complementary (and without any mismatches) to at least a portion of a labeled attachment control target that is added to the test sample. In certain embodiments, the sequence of an attachment control probe is designed based on its dissimilarity to the sequence of any known target that is expected to be present in the test sample.

In certain embodiments, the attachment control probes and the counterpart probes are labeled without hybridization to another oligonucleotide. In certain such embodiments, one can test the acceptability of specific attachment of probes to a microarray without exposing the microarray to a sample.

16. Contaminant Control

In certain embodiments, a contaminant control is provided. A contaminant control is a control element comprising at least one contaminant control probe that is complementary to at least a portion of a contaminant gene that should not be present in a sample, but may be in a sample. In certain embodiments, the contaminant gene is from a source other than the source from which the sample was obtained. For example, in certain embodiments, the sample may be obtained from a human source and one or more contaminant genes may be selected from one or more sources selected from bacterial sources, yeast sources, fungal sources, viral sources, and mycoplasma.

In certain embodiments, multiple different contaminant control probes may be provided that are complementary to at least a portion of different contaminant genes from a single source. For example, in certain embodiments, more than one different contaminant probes may be complementary to different genes from a single species of bacteria. In certain embodiments in which multiple different contaminant control probes are provided that are complementary to at least a portion of different contaminant genes from a single source, one or more additional contaminant control probes may be used that are complimentary to one or more different contaminant genes from different sources.

In certain embodiments, a contaminant control is used to determine whether, or the degree to which, contaminants are contained in a sample. In certain embodiments, since one does not expect contaminants complementary to the contaminant probe to be present in the test sample, one expects the signal at the feature comprising such a probe to be low, relative to the signal from other features where a positive signal is expected. In certain embodiments, a signal from a contaminant control similar to, or greater than, signals at one or more experimental features may indicate that a contaminant is present in the sample. In certain embodiments, a signal from a contaminant control similar to, or greater than, signals at one or more control probes may indicate that a contaminant is present in the sample. In certain embodiments, a signal above a given threshold level from a contaminant control indicates that a contaminant is present in the sample.

In certain instances, a contaminant can change gene expression in a given sample. In certain embodiments, the user may use results from a contaminant control to evaluate the accuracy of results in a microarray for a given sample. In certain embodiments, a contaminant control is used to determine whether, or the degree to which, contaminants are contained in a sample obtained from a tissue source.

Certain Exemplary Multi-Functional Controls

In certain embodiments, multi-functional control features are provided. Multi-functional control features are features that serve as more than one control element. In certain embodiments, combining control functions on a feature allows the user to dedicate more features on the microarray to experimental probes.

In certain embodiments, a multi-functional control feature produces two or more different signals. In certain such embodiments, the two or more different signals serve as two or more control elements. For example, in certain embodiments, a single feature comprises both a labeled probe, which produces a first signal for use as a landmark fiducial, and a hybridization control probe, which is complementary to a hybridization target comprising a label that produces a second signal. Thus, in certain embodiments, a multi-functional control feature can serve as a landmark fiducial and a hybridization control.

In certain embodiments, a single signal from a multi-functional control feature serves more than one control function. For example, because the features of a control ladder produce a reliable signal at a known location on a microarray, such features may also serve as landmark fiducials. Thus, in certain such embodiments, a single signal from a multi-functional control feature serves two or more control functions.

In certain embodiments, multi-functional control features produce two or more signals, at least one of which serves more than one control function. For example, in certain embodiments, a multi-functional control feature comprises two probes: a labeled probe, which produces a first signal that is both part of a control ladder and also serves as a landmark fiducial; and a hybridization control probe, which is complementary to at least a portion of a hybridization target comprising a label that produces a second signal. Such a feature serves as part of a control ladder, as a landmark fiducial, and as a hybridization control. In certain embodiments, another feature comprising the same probes is present at another region of the microarray and each produces both a chemiluminescent and a fluorescent signal. Thus, in certain embodiments, such a multi-functional control feature is also part of a spatial normalization control.

In another non-limiting example of multi-functional features, in certain embodiments, internal control molecules may be included with experimental probes at certain features of a microarray in an easily identifiable pattern. In certain embodiments, such a strategy allows a feature to function as a landmark fiducial and as an internal control set.

In another non-limiting example of multi-functional features, in certain embodiments, an internal control molecule of an internal control set may act as a hybridization control probe. For example, the internal control molecule may be a probe that is complementary, without any mismatches, to a hybridization control target that has been added to the test sample. Such a probe acts as both an internal control molecule and a hybridization control probe. Certain such embodiments allow one to quantify the amount of experimental target in a test sample. For example, in certain such embodiments, one knows the amount of hybridization control target added to the test sample. Further, in certain embodiments, one knows the ratio of internal control probe to experimental probe that was added to the feature. In certain such embodiments, one can use the known amount of hybridization control target added to the test sample and the known ratio of internal control probe to experimental probe to calculate the amount of experimental target in a sample.

In certain embodiments, multi-functional control features may be employed in assays in which targets are labeled with two or more different labels. For example, in certain embodiments, an internal control set may comprise an experimental probe and a labeled internal control probe that also serves as a positive gene control. In certain such embodiments, labeled internal control probe (first signal) is complementary to a differently labeled internal control target (second signal) that is expected to be present in a test sample (and thus, also acts as a positive gene control). In certain embodiments, experimental target is labeled with a different label (a third signal). In certain such embodiments, the first signal provides a control for accurately determining the amount and placement of experimental probe that is bound to the feature and the second signal serves as a positive gene control signal.

Certain Exemplary Combinations of Control Elements

The skilled artisan will recognize that control elements, whether embodied on separate features or combined as multi-functional controls, may be used in various combinations on a microarray. Thus, in certain embodiments, all or any subset of the control elements discussed above are included on a microarray. Such control elements may also be included with additional controls in any combination.

Certain Exemplary Kits

In certain embodiments, kits are provided that comprise one or more control targets that interact with one or more control probes on the microarray. In certain embodiments, kits are provided that comprise one or more microarrays comprising one or more control elements. In certain embodiments, kits are provided that comprise (1) one or more microarrays comprising one or more control elements, and (2) one or more control targets that interact with one or more control probes on the microarrays. In certain embodiments, kits are provided that comprise microarray probes.

Certain Exemplary Analysis Methods

Certain nonlimiting embodiments are described in “Applied Biosystems 1700 Chemiluminescent Microarray Analyzer”, Ver. 1.1 User Guide. To the extent the definitions of terms in that document are not the same as the definitions in the rest of the specification, the definitions in the rest of the specification control for the entire specification, including that document. Any other definitions in that document that are not explicitly provided in the rest of the specification apply only to the embodiments discussed in that document.

### EXAMPLE

The following example does not limit the scope of the invention.

### Example 1

This example describes a microarray according to certain embodiments. FIG. 1 shows the arrangement of the gridding of a microarray according to certain embodiments. The microarray in FIG. 1 includes 96 sub-grids, with 19×19 features per sub-grid. Thus, the microarray has 34,656 features (19×19×96). The microarray shown in FIG. 1 has two images (the top half of the microarray with 48 sub-grids (6×8)) and the (the bottom half of the microarray with 48 sub-grids (6×8)). The microarray shown in FIG. 1 includes blank rows and columns separating the sub-grids.

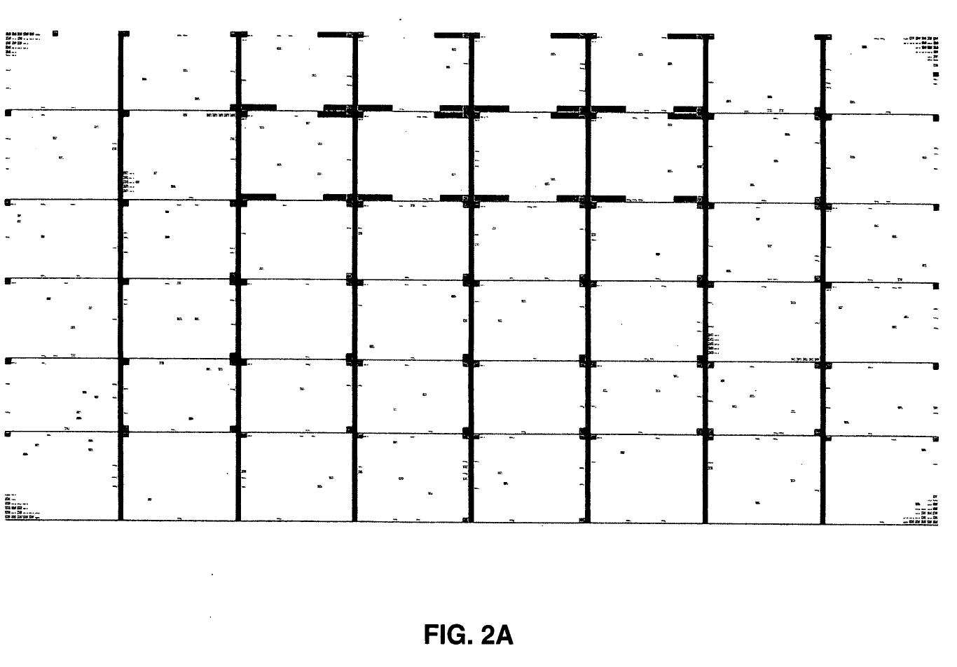

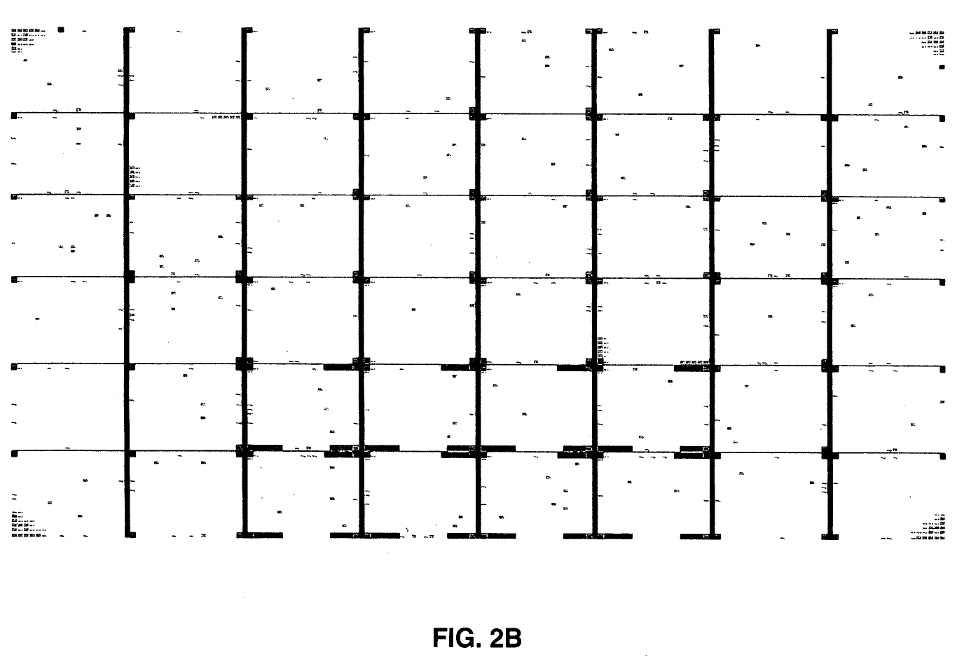

FIG. 2 shows an arrangement of microarray control elements according to certain embodiments of a microarray having the gridding pattern of the microarray of FIG. 1. (In view of the size of the figure, the exemplary microarray is split into two figures. FIG. 2A shows the top image of such an exemplary microarray and FIG. 2B shows the bottom image of such an exemplary microarray.)

FIG. 2 shows in purple the blank rows and columns separating the sub-grids. In this nonlimiting example, the sub-grids in FIG. 2 are designated with consecutive numbering from top to bottom and consecutive lettering from left to right. Thus, the top left hand corner sub-grid (in FIG. 2A) is designated 1A, the top right hand corner sub-grid (in FIG. 2A) is designated 1H, the bottom left hand corner sub-grid (in FIG. 2B) is designated 12A, and the bottom right hand corner sub-grid (in FIG. 2B) is designated 12H. In this non-limiting example, all or most of the features in FIG. 2 that do not include a control element designation include probes for detecting experimental target nucleic acids.

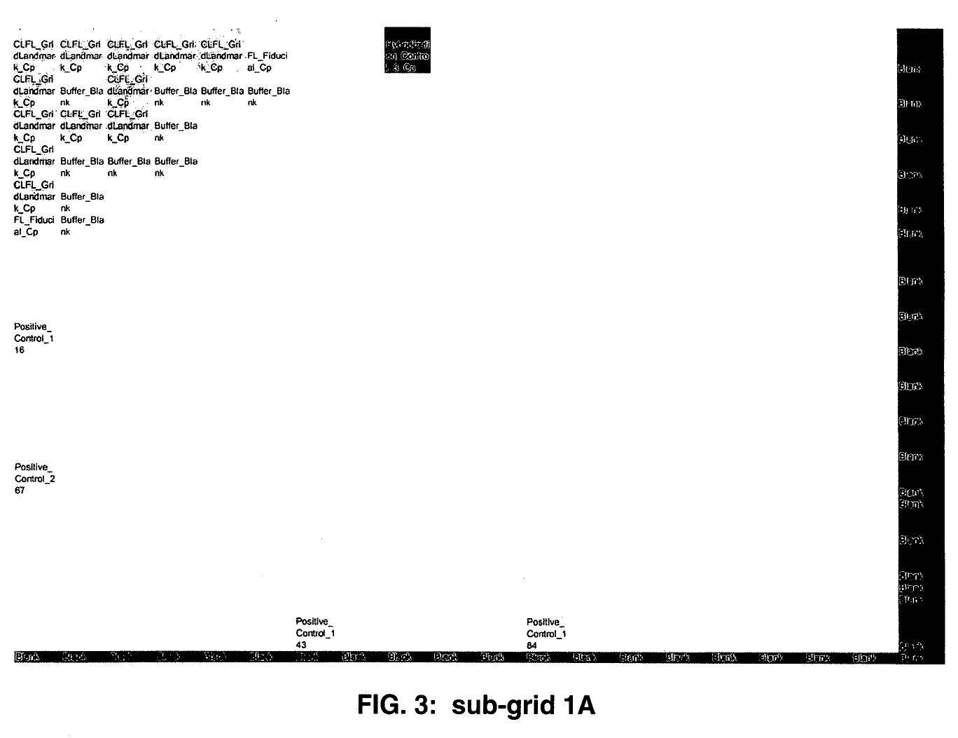

In this non-limiting example, there are landmark fiducial features in the four corner sub-grids of each image of the microarray. For example, FIG. 2 shows certain embodiments in which there are 12 landmark fiducial features in each such corner to which are attached oligonucleotides labeled with moieties that are used for both chemiluminescent and fluorescent signal detection. See, e.g., FIG. 2, the pink landmark fiducial features in sub-grids 1A, 1H, 6A, 6H, 7A, 7H, 12A, and 12H. FIG. 3 is an enlarged view of sub-grid 1A of FIG. 2. FIG. 2 also shows certain embodiments in which there are 2 landmark fiducial features in each corner of each image of the microarray to which are attached oligonucleotides labeled with moieties used for fluorescent signal detection. See, e.g., FIG. 2, the yellow landmark fiducial features near the pink landmark fiducials in sub-grids 1A, 1H, 6A, 6H, 7A, 7H, 12A, and 12H. Such yellow landmark fiducial features are more easily seen in the enlarged view of sub-grid 1A in FIG. 3. In certain embodiments, the labels used for fluorescent signal detection are LIZ and the labels used for chemiluminescent signal detection are DIG. In this non-limiting example, the landmark fiducial oligonucleotides have the sequences set forth in Tables B and C.

In this non-limiting example, there are 65 different positive gene control probes provided throughout the microarray having the sequences set forth in Table H. Each of the 65 different positive gene control probes is complementary to at least a portion of a known gene without any mismatches and the known gene is expected to be in the sample.

Also in this example, there are five different positive gene mismatch control probes related to each of the 65 different positive gene control probes. In other words, each of the five different positive gene mismatch control probes comprises nucleotides that base pair with the at least a portion of the known gene and comprises at least one mismatch with the at least a portion of the known gene. Thus, there are 325 (5×65) different positive gene mismatch control probes in this nonlimiting example having the sequences set forth in Table H.

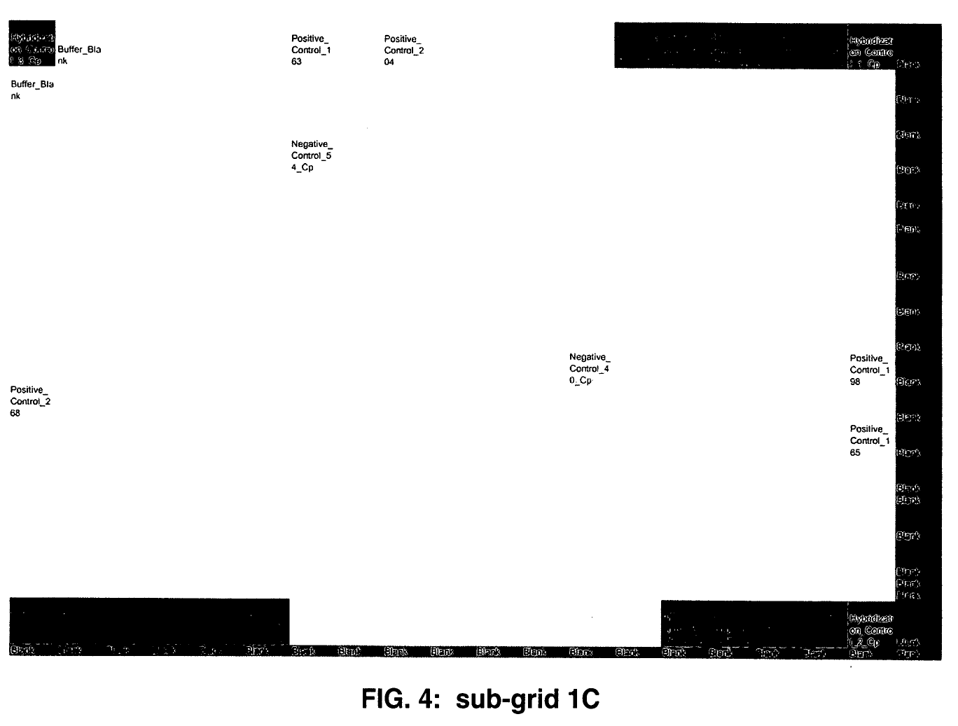

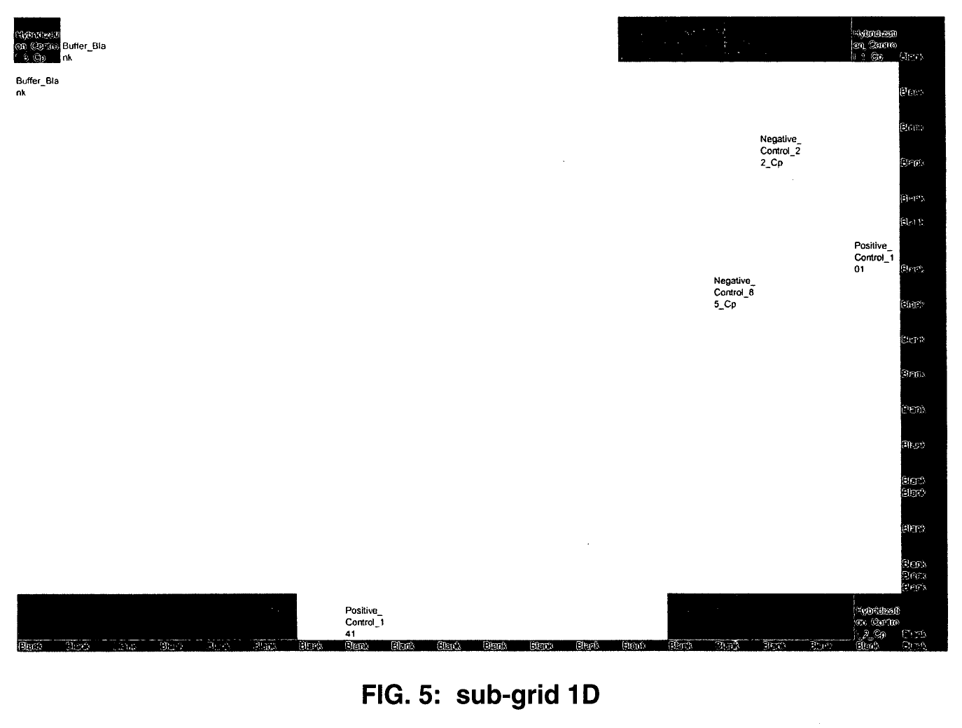

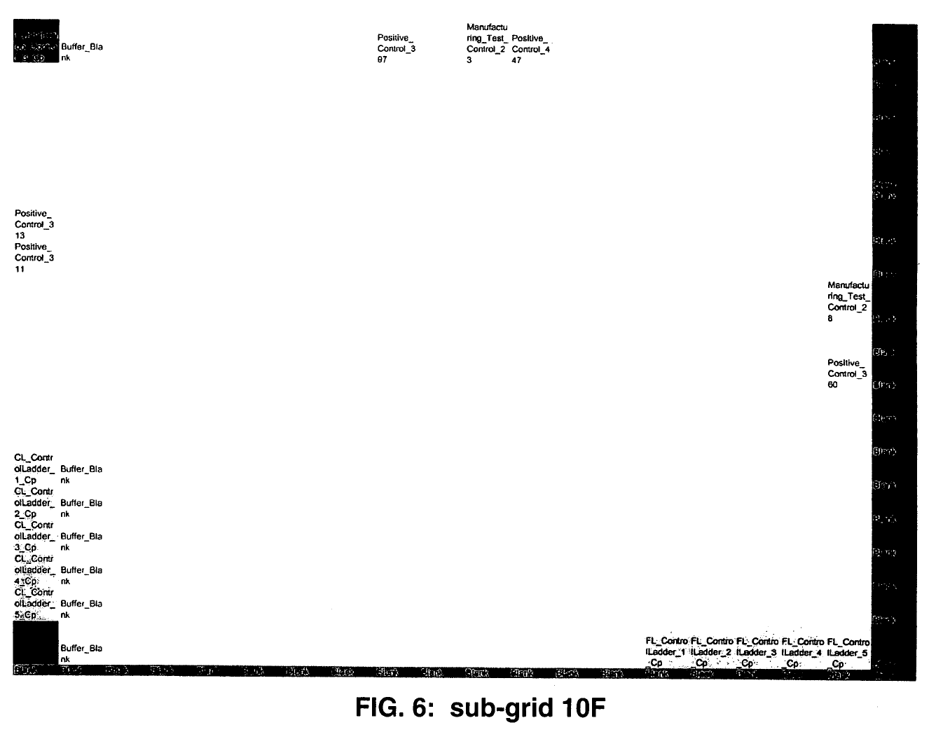

FIG. 2 shows certain embodiments in which certain of the yellow features on the outside perimeter of the sub-grids are positive gene controls and positive gene mismatch controls. In FIG. 2, there are 65 different positive gene control features, each with different positive gene control probes, and 325 different positive gene mismatch control features, each with different positive gene mismatch control probes. See also FIG. 3 (an enlarged view of sub-grid 1A), FIG. 4 (an enlarged view of sub-grid 1C), FIG. 5 (an enlarged view of sub-grid 1D), and FIG. 6 (an enlarged view of sub-grid 10F).

In this non-limiting example, there are 98 different non-specific background control probes provided in 98 different features throughout each image of the microarray having the sequences set forth in Table G. Thus, there are two replicates of each of the 98 different non-specific background control features (196 features).

FIG. 2 shows certain embodiments in which the yellow features in the interior of the sub-grids are non-specific background controls. See also FIG. 4 (an enlarged view of sub-grid 1C) and FIG. 5 (an enlarged view of sub-grid 1D). (FIGS. 4 and 5 refer to non-specific background controls as “negative controls.”)

In this non-limiting example, there are two fluorescent control ladders in each image and two chemiluminescent control ladders in each image. In this non-limiting example, each fluorescent ladder includes five features, each with a LIZ-labeled oligonucleotide attached in a five-fold dilution series (1×, 5×, 25×, 125×, and 625×). In this non-limiting example, the five different concentrations of the LIZ-labeled oligonucleotide on the five different features of the ladder are 50; 10; 2; 0.4; and 0.08 micromolar in the spotting solution. In this non-limiting, example each chemiluminescent control ladder includes five features, each with a DIG-labeled oligonucleotide attached in a five-fold dilution series (1×, 5×, 25×, 125×, and 625×). In this non-limiting example, the five different concentrations of the DIG-labeled oligonucleotide on the five different features of the ladder are 5; 1; 0.2; 0.04; and 0.008 micromolar in the spotting solution. FIG. 2 shows a possible arrangement of the four fluorescent control ladders (each of the control ladders having five features) and the four chemiluminescent control ladders (each of the control ladders having five features) according to certain embodiments. See the pink chemiluminescent control ladder features and the light blue fluorescent control ladder features in sub-grids 2B, 4G, 8B, and 10F). See also FIG. 6, which is an enlarged view of sub-grid 10F of FIG. 2. In this non-limiting example, the ladder oligonucleotides have the sequences set forth in Tables B and C.

In this non-limiting example, there are probes for three different bacterial genes that are used for reverse transcription control features. The genes are dap, lys, and phe. In this non-limiting example, there are five different 60-nucleotide reverse transcription control probes for each of the three genes. Thus, there are fifteen different reverse transcription control probes having the sequences set forth in Table F. In this non-limiting example, each of the different reverse transcription control probes is included in features four times per image. FIG. 2 shows certain embodiments in which the dark green features are reverse transcription control features (see sub-grids 1D, 1F, 2D, 2F, 10D, 10F, 11D, 11F, 12D, and 12F). FIG. 5 is an enlarged view of sub-grid 1D of FIG. 2 and shows 15 different reverse transcription control features with the 15 different reverse transcription control probes (probes 1 through 5 for each of the genes dap, lys, and phe). FIG. 2 shows certain embodiments in which sub-grids 1F, 2D, 2F, 11D, 11F, 12D, and 12F each have the same pattern of reverse transcription control features as sub-grid 1D. FIG. 2 shows certain embodiments in which sub-grids 10D and 10F each include a single reverse transcription control feature in the bottom left corner which includes lys 1 reverse transcription control probes.

In this non-limiting example, the user adds the reverse transcription control genes as single stranded RNA into a reverse transcription reaction to produce cDNA and that cDNA is used in an assay using the microarray.

In this non-limiting example, there are probes for three different bacterial genes that are used for in vitro transcription control features. The genes are bioB, bioC, and bioD. In this non-limiting example, there are five different 60-nucleotide in vitro transcription control probes for each of the three genes. Thus, there are fifteen different reverse transcription control probes having the sequences set forth in Table E.

In this non-limiting example, each of the different reverse transcription control probes is included in features four times per image. FIG. 2 shows certain embodiments in which the dark blue features are in vitro transcription control features (see sub-grids 1C, 1E, 2C, 2E, 10C, 10E, 11C, 11E, 12C, and 2E). FIG. 4 is an enlarged view of sub-grid 1C of FIG. 2 and shows 15 different in vitro transcription control features with the 15 different in vitro transcription control probes (probes 1 through 5 for each of the genes bioB, bioC, and bioD). FIG. 2 shows certain embodiments in which sub-grids 1E, 2C, 2E, 11C, 11E, 12C, and 12E each have the same pattern of in vitro transcription control features as sub-grid 1C. FIG. 2 shows certain embodiments in which sub-grids 1C and 10E each include a single in vitro transcription control feature in the bottom left corner which includes bioC1 in vitro transcription control probes.

In this non-limiting example, the user adds the in vitro transcription control genes as double-stranded DNA into an in vitro transcription reaction to produce RNA and that RNA is used in an assay using the microarray.

In certain embodiments, there is a reverse transcription kit (RT kit). In certain such embodiments, the reverse transcription control genes dap, lys, and phe are provided in the RT kit. In certain such embodiments, the user adds those three reverse transcription control genes to the reverse transcription reactions. In certain embodiments, there is a reverse transcription-in vitro transcription kit (RT-IVT kit). In certain such embodiments, the reverse transcription control genes dap, lys, and phe and the in vitro transciption control genes bioB, bioC, and bioD are provided in the RT-IVT kit. In certain such embodiments, the user adds those three reverse transcription control genes to the reverse transcription reactions and adds those three in vitro transcription control genes to the in vitro transcription reactions. In certain embodiments, the levels of reverse transcription control genes provided in the kits are about 100 times higher in the RT kit than in the RT-IVT kit.

In this non-limiting example, there are 60 first hybridization control features (30 per image) that each include 60-nucleotide first hybridization control probes having the sequence set forth in Table D (see HYB_Control_{—}1_Cp).

FIG. 2 shows certain embodiments in which the first hybridization control features are shown in orange in the upper right hand corners of 30 sub-grids per image. See also FIGS. 4 and 5, which are enlarged views of sub-grids 1C and 1D, respectively, of FIG. 2.

In this non-limiting example, there are 60 second hybridization control features (30 per image) that each include 60-nucleotide second hybridization control probes having the sequence set forth in Table D (see HYB_Control_{—}2_Cp).

FIG. 2 shows certain embodiments in which the second hybridization control features are shown in orange in the lower right hand corners of 30 sub-grids per image. See also FIGS. 4 and 5, which are enlarged views of sub-grids 1C and 1D, respectively, of FIG. 2.

In this non-limiting example, there are 117 third hybridization control features that each include 60-nucleotide third hybridization control probes having the sequence set forth in Table D (see HYB_Control_{—}3 Cp).

FIG. 2 shows certain embodiments in which the third hybridization control features are shown in red. See also FIGS. 4 and 5, which are enlarged views of sub-grids 1C and 1D, respectively, of FIG. 2. FIG. 2 shows certain embodiments in which the third hybridization control features are shown in certain corners of each of the sub-grids. FIG. 2 shows certain embodiments in which sub-grids 1A, 1H, 7A, and 7H also include third hybridization control features on the outside perimeter and not in a corner.

In certain embodiments, the first, second, and third hybridization control probes hybridize to three different DIG labeled hybridization control targets (1, 2, and 3) that are added to the sample. The sequences of the three different hybridization control targets are

(1)
CGACATGAAACTTGGTTTGTGCCCAGTAGCGACAGAATCACGTATCGGTT
TACGCCGTCA,
(2)
CTCGAGAGTAATTATGACACGTAAGGTTTAAGAGCCCGCCGGACTTGGAT
CCGTCCTACT,
and
(3)
TGAACTGGTTTTGCTAGCCCACTCAACGGTCACGCATCTAAGGGATATGC
CGATTCAGGA.

In certain embodiments, hybridization control targets 1 and 2 are added to the sample at 0.25 μM concentration. In certain such embodiments, hybridization control target 3 is added to the sample at 1.0 μM concentration.

In this non-limiting example, there are 170 buffer blank features. FIG. 2 shows certain embodiments in which the buffer blank features are depicted by print on a white background. The buffer blank features can also be seen in FIGS. 3, 4, and 5, which are enlarged views of certain sub-grids of FIG. 2.

In this non-limiting example, there is an internal control probe in each feature of the microarray that includes a 60-nucleotide probe. Those 60-nucleotide probes include expression probes for detecting target in a sample, as well as control probes such as hyridization control probes, non-specific background control probes, positive gene control probes, positive gene mismatch control probes, reverse transcription control probes, and in vitro transcription control probes.

In this non-limiting example, the internal control probe is a 24 nucleotide probe having the sequence: 5′-TTCGGCTGTGAGMCGATCACGCA-3′. In this non-limiting example, internal control target having sequence: 5′-TGCGTGATCGTTCTCACAGCCGAA-3′ labeled at the 5′ end with LIZ is added to the sample. Thus, if labeled control target hybridizes to internal control probe on a feature of the microarray, a fluorescent signal is provided at that feature. In certain embodiments, the fluorescent signal is used in gridding or aligning the features by one or more algorithms. For example, in certain embodiments, the fluorescent signals are used by an algorithm to determine which features or pixels to use in quantification. In certain embodiments, the fluorescent signal provides an indication that expression probes have been successfully attached to a given feature of the microarray. In certain embodiments, the fluorescent signal is used in focusing the optical reader.

In this non-limiting example, features that contain both an internal control probe and a hybridization control probe 3 operate as a spatial normalization control. Thus, in this non-limiting example, at such features, one determines the ratio of the fluorescent signal, which results from hybridization to the internal control probe to the chemiluminescent signal, which results from hybridization to the hybridization control probe 3.

In this non-limiting example, target nucleic acids are labeled with DIG. In this non-limiting example, after the target nucleic acids and control nucleic acids are applied to the microarray, antiDIG:alkaline phosphatase complex is applied and chemiluminescent enhancing material such as a quarternary amine containine polymer such as poly (benzyl tripentyl ammonium chloride) is applied. In this non-limiting example, the chemiluminescent substrate (e.g., a 1,2-dioxetane substrate) is then applied to produce a chemiluminescent signal.

Exemplary Systems According To Certain Embodiments

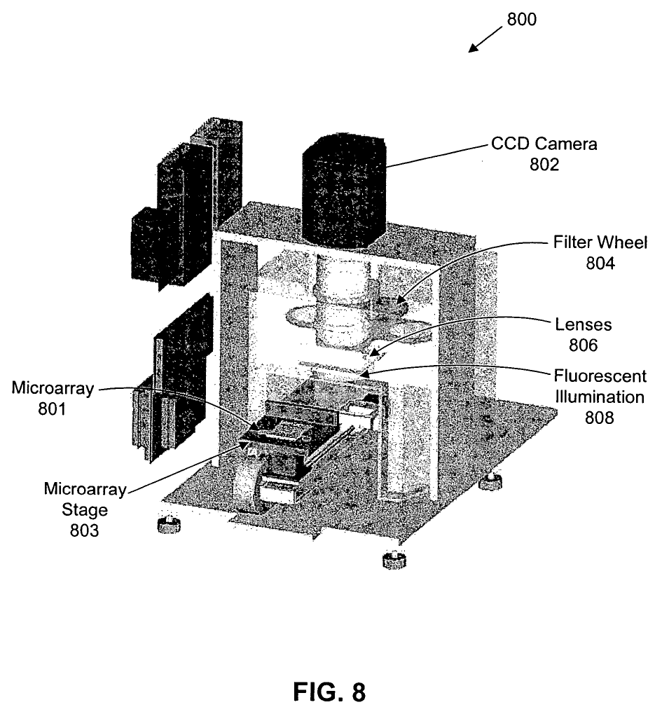

FIG. 8 shows a non-limiting exemplary image processing system 800 for obtaining images of microarrays according to certain embodiments. Image processing system 800 includes a microarray 801 on a microarray stage 803. In certain embodiments, microarray 801 includes a plurality of features that provide a fluorescent (FL) signal and a chemiluminsescent (CL) signal. Both FL and CL signals are detected from microarray 801 and such signals can be used for identifying features. In certain embodiments, the identified features can be used for gene expression analysis. In certain embodiments, signals and data related to the features can be corrected or normalized using a combination of both the CL and FL signals.

Image processing system 800 also includes a charge-coupled device (CCD) camera 802 that can obtain images of the microarray 801 using a filter wheel 804 and lenses 806. FIG. 8 also shows certain embodiments of a system that include a fluorescent illumination 808 for obtaining images. In certain embodiments, the CCD camera 802 can obtain images in two channels by detecting the CL and FL signals, which results in CL and FL images, respectively. The CL and FL signals can produce a noticeable spot or indication on or in the microarray 801 that appear in the CL and FL images. The noticeable spot or indication can be used to identify a feature in the images.

In certain embodiments, to take an image of these spots, the image processing system 800 illuminates light on the spots, which can exhibit varying light intensities. In certain embodiments, the CCD camera 802 includes an array of miniature optical sensors, i.e., pixels, that detect the light intensity from the spots and appear in an image. Each sensor senses light intensities for its corresponding pixel. In certain embodiments, a feature can be represented by a plurality of pixels where each pixel can have light intensity values that can range from 0 to 64,356 count values. The higher count values represent brighter colors and the lower count values represent darker colors. In certain embodiments, the CCD camera 802 can obtain images of of controls, such as landmark fidicual controls and spatial normalization controls that produce a noticeable indication in the images.

In certain embodiments, the CCD camera 802 includes a plurality of optical sensors, i.e., pixels, that detect light intensity from a particular location of microarray 801. For example, the pixels can detect light intensity from CL and FL signals of a feature on or in the microarray 801. In certain embodiments, the obtained CL and FL images can be represented by a plurality of pixels that form a two-dimensional array and provide light intensity values corresponding to the CL and FL signals. For example, in certain embodiments, the image includes an array of 2048×2048 pixels, wherein each pixel can be identified by x, y coordinates. The light intensity values can be referred to as “signal values.” In certain embodiments, features shown in the CL and FL images are represented by any number of pixels that can have varying signal values.

In certain embodiments, the CL and FL images show features that form a two dimensional grid in the images. That is, the features can be arranged to form rows and columns of features in the images. The features can also form a plurality of subgrids of rows and columns of features. In certain embodiments, the microarray 801 includes 19×19 subgrids of features such that blank rows and columns are shown in between subgrids of features in the corrected images.

In certain embodiments, the CCD camera 802 can obtain CL and FL images of subgrids of features. For example, the CCD camera 802 can take a first image and a second image of the top and bottom halves of features of microarray 801. In certain embodiments, the image processing system 800 including the CCD camera 802 can be implemented according to methods described in “Applied Biosystems 1700 Chemiluminescent Microarray Analyzer”, Ver. 1.1 User Guide.

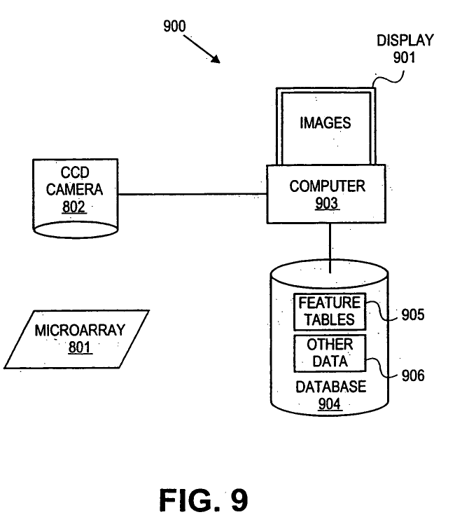

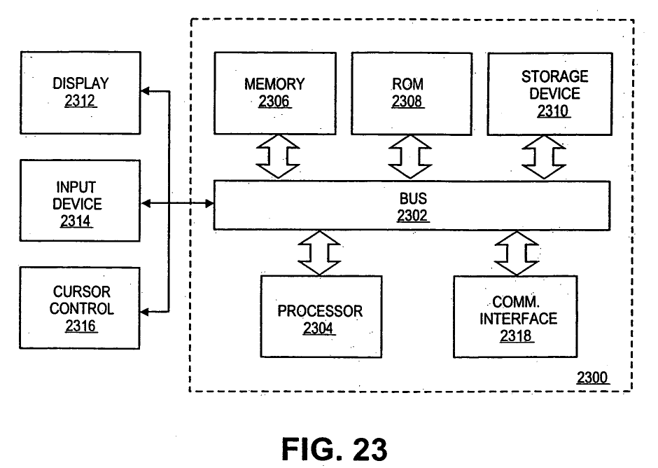

FIG. 9 depicts an overview block diagram of a non-limiting exemplary computing system 900 for processing images of microarray 801 from the CCD camera 802 according to certain embodiments. The computing system 900 includes a computer 903 with a display 901 for displaying images. The computer 903 is coupled with the CCD camera 802 and a database 904. In various embodiments, the computer 903 also includes any combination of hardware components (e.g., as shown in FIG. 23) to implement software for communicating with the CCD camera 802 and the database 904, such as the Windows® 2000 operating system and the Oracle® 9i server software.

In certain embodiments, the computer 903 runs application software used for obtaining images of the microarray 801 and analyzing data from the images, non-limiting examples of which include, but are not limited to, the “AB Navigator” software for the Applied Biosystems 1700 Chemiluminescent Microarray Analyzer™ described in “Applied Biosystems 1700 Chemiluminescent Microarray Analyzer”, Ver. 1.1 User Guide. In certain embodiments, such software assists a user of computer 903 to set up experiments, operate the image processing system 800 including the CCD camera 802, analyze images and data from the CCD camera 802, including signal values related to features, and perform primary analysis of the data and signals from the images of the microarray 801 or other microarrays.

In certain embodiments, the computer 903 processes data or signal values associated with CL signals, FL signals, and/or data or signal values associated with features to generate quantification data. In certain embodiments, the quantification data can include information regarding detected signals associated with features (“feature signals”), feature signal uncertainties, and quality metrics and confidence values associated with the detected feature signals. In certain embodiments, such quantification data can be stored in the database 904 as feature tables 905. Non-limiting examples of the feature tables 905 are disclosed in the Quantification Output Table below.

Quantification Output Table

| | AB1700 | 2^{nd} | |
|---|---|---|---|
| | SIGNAL | ANALYSIS | |
| API | TABLE | EXPORT | DESCRIPTION |
| CLinorm | ICP | Gene | This is the fully corrected signal (see |
| | Normalized | Signal | #1 below) associated with the probe |
| | Signal | | or control in counts. The CL signal is |
| | | | normalized by the FL signal over the |
| | | | feature integration aperture. To |
| | | | preserve the approximate feature CL |
| | | | counts the CL/FL ratio is multiplied by |
| | | | the median FL signal (aperture |
| | | | integrated) for all features containing |
| | | | ICP (i.e. gene probes) quantified at a |
| | | | particular array position. |
| CLinormsdev | ICP | — | Represents the estimated |
| | Normalized | | measurement uncertainty of CLinorm. |
| | Signal Error | | Error estimates are primarily from |
| | | | propagation of background |
| | | | subtraction and signal integration |
| | | | uncertainties. |
| CLinormS2N | ICP | Gene S/N | Equals the ratio of |
| | Normalized | | CLinorm/CLinormsdev. This metric |
| | S/N | | expresses the confidence of feature |
| | | | “detectability”. A S/N of 3 represents |
| | | | a 3 SDEV confidence (99.9%) that |
| | | | the measurement is real. Users can |
| | | | use this to make a Presence/Absence |
| | | | assignment at their preferred |
| | | | confidence level. |
| CLinormCV | ICP | Gene CV | The CV is fractional uncertainty in the |
| | Normalized | | signal (coefficient of variation) which |
| | CV | | includes both the fractional |
| | | | measurement uncertainty, |
| | | | determined by the S/N, and the |
| | | | system precision. The system |
| | | | precision (CVprec) is the limiting |
| | | | quantitative accuracy determined by |
| | | | the intrinsic spread of feature signals |
| | | | independent of S/N and is measured |
| | | | for each array using the fully |
| | | | normalized high S/N replicate |
| | | | controls. The Gene CV determines |
| | | | the individual feature reproducibility |
| | | | and is directly involved in a GEx Fold |
| | | | change confidence calculation. Since |
| | | | there are no replicates for a given |
| | | | gene, this is a predicted CV derived |
| | | | from an Error Model. CLinorm × CLinormCV |
| | | | represents the total probe signal variation |
| | | | that might otherwise be estimated analyzing a |
| | | | gene signal across multiple replicate |
| | | | (globally normalized) arrays. |
| Ginorm | Assay | Assay | This is a single value: the median of |
| | Normalizing | Normalizing | all valid probe CLinorm signals on the |
| | Signal | Signal | array. The ratio of CLinorm/Ginorm |
| | | | and CLinormsdev/Ginorm gives a |
| | | | simple “global normalization” of both |
| | | | signal and signal error between |
| | | | arrays, which normalizes out such |
| | | | things as differences in exposure time |
| | | | or sensitivity differences between |
| | | | instruments. This is especially useful |
| | | | if a user wishes to filter data based on |
| | | | a particular signal threshold across a |
| | | | number of arrays. Note that |
| | | | CLinormS2N and CLinormCV are |
| | | | independent of any array to array |
| | | | normalization. |
| FLAGS | FLAGS | FLAGS | A numeric code for each feature that |
| | | | identifies conditions given in the |
| | | | Appendix 1 table. Values <100 are |
| | | | considered valid quantification. |
| | | | Values in the range 100-10000 have |
| | | | quality issues. Values in the range |
| | | | 10000-100000 - are recommended to |
| | | | be rejected from analysis. These are |
| | | | based on quality issues captured by |
| | | | QCmetric2 below an internally |
| | | | defined threshold as well as low S/N |
| | | | in the FL channel. Values above |
| | | | 1000000 represent failures or invalid |
| | | | quantification. Users can filter data |
| | | | based on these FLAGS values. |
| QCmetric1 | QCmetric1 | — | A feature quality metric given by the |
| | | | CL/FL feature pixel correlation. Users |
| | | | can filter out problematical data |
| | | | based on their own preferred |
| | | | threshold. However, since low |
| | | | correlation can also reflect low S/N on |
| | | | the feature with otherwise valid |
| | | | quantification it is recommended |
| | | | users exercise caution with this |
| | | | potential filter. There is no internally |
| | | | defined threshold for triggering a |
| | | | FLAGS state for this metric. |
| QCmetric2 | QCmetric2 | — | A feature quality metric that captures |
| | | | the fraction of usable pixels used in |
| | | | quantification. The selection criterion |
| | | | is based on a Chi2 test of CL/FL pixel |
| | | | ratios and can be used to filter out |
| | | | problematic measurements likely to |
| | | | be associated with artifacts on the |
| | | | array that are corrupting |
| | | | quantification. A FLAGS state is set |
| | | | for features falling below an internally |
| | | | defined threshold of ⅔. |
| CLssum | CL Signal | — | Background corrected integrated CL |
| | | | feature signal (with no feature |
| | | | refinements listed in #1). |
| CLssig | CL Signal | — | Background corrected integrated CL |
| | Error | | feature signal uncertainty (with no |
| | | | feature refinements listed in #1). |
| FLssum | FL Signal | — | Background corrected integrated FL |
| | | | feature signal (with no feature |
| | | | refinements listed in #1). |
| FLssig | FL Signal | — | Background corrected integrated FL |
| | Error | | feature signal uncertainty (with no |
| | | | feature refinements listed in #1). |
| CLraw | CL Raw | — | The integrated CL signal with no |
| | Signal | | background correction. The |
| | | | integrated CL background is given by |
| | | | CLraw-CLssum. Note: this BG, in |
| | | | addition to the CLraw, excludes the |
| | | | arbitrary CCD bias offset corrected for |
| | | | in the preprocessing step (see note |
| | | | #1) which is not associated with |
| | | | actual BG signal. |
| FLraw | FL Raw | — | The integrated FL signal with no |
| | Signal | | background correction. The |
| | | | integrated FL background is given by |
| | | | FLraw-FLssum. Note: this BG, in |
| | | | addition to the FLraw, excludes the |
| | | | arbitrary CCD bias offset corrected for |
| | | | in the preprocessing step (see note |
| | | | #1) which is not associated with |
| | | | actual BC signal. |
| X | | | |
| Y | | | |

| | |
|---|---|
| 1. | Corrections can be considered to divide into two classes: |

a. Image Corrections

| | |
|---|---|
| i. | CCD BIAS offset subtraction |
| ii. | CCD BIAS image subtraction |
| iii. | CCD DARK image subtraction |
| iv. | FLAT-FIELD image division (CCD pixel to pixel gain variations) |
| v. | FL ILLUM_FL image division (non-uniform LED illumination) |
| vi. | BADPIXEL masking |
| vii. | FL spectral cross-talk correction (FL minus scaled FX image) |
| viii. | CL-CL-short image combine to replace detector saturated pixel in the |
| | CL-long image with scaled unsaturated pixels from the CL-short image |
| ix. | FL to CL subpixel image alignment. |

b. Feature Corrections

| | |
|---|---|
| i. | CL and/or FL spatial cross-talk (SXT) correction to signals and signal |
| | errorbars. [TBD depending on final quantification method selected during |
| | optimization.] |
| ii. | Feature (ICP) Normalization - the CL/FL ratio normalizes spotting |
| | variability, algorithmic issues (centroid variability, differences in integration |
| | apertures) as well as optical vignetting which affects both channels equally |
| iii. | Spatial Normalization (SPN) correction - controls are used to normalize out |
| | systematic trends in CL/FL ratios across the array. |
| | Note: optical vignetting seen in the corners of the image divides out |
| | in the CL/FL ratio. Also non-uniform LED FL illumination is addressed with |
| | the ILLUM_FL correction. This correction removes any residual large scale |
| | variations. |
| iv. | Assay Background (ABG) correction - subtracts the median fully normalized |
| | signal CL signal of negative controls to remove the statistical bias of assay |
| | noise from the final CLinorm signal. All CLinorm* table values are corrected |
| | for ABG by default. |

In certain embodiments, other data 906 may also be stored in the database 904 such as images and parameters for correcting the images.

Certain Exemplary System Operations

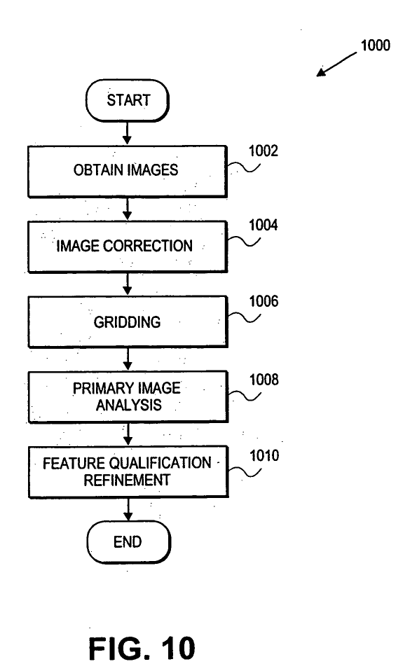

FIG. 10 depicts a flow diagram 1000 of exemplary basic steps to process images of microarray 801 from the CCD camera 802 according to certain embodiments.

Initially, in certain embodiments, images are obtained of the microarray 801 from the CCD camera 802 (step 1002). In certain embodiments, the CCD camera 801 takes different types of CL and FL images. For example, in certain embodiments, the CCD camera 802 takes a first FL image (“FL image”) with exposure to excitation light and a second FL image without exposure to excitation light (“FX image”). In certain embodiments, spots or indications of features can be easily seen in the FL image. In certain embodiments, the FX image can show bleed through of CL signals into the FL channel of the FL image.

In certain embodiments, the CCD camera 802 takes a first CL image with a first exposure time, e.g., 25 seconds (“CL long image”), and a second CL image with a second exposure time, e.g., 5 seconds (“CL short image”). In certain embodiments, the CL long image can ensure that faint features have an adequate signal-to-noise (S/N) ratio, and the CL short image can ensure that features are below saturation, especially bright features. In certain embodiments, the combination of these FL and CL images can improve detection of features and correct and normalize feature signals.

In certain embodiments, the computer 903 performs image correction on the obtained images that can include the FL and FX images and the CL long and CL short images (step 1004). In this step, according to certain embodiments, a number of corrections can be applied to obtain a corrected FL image and a corrected CL image in order to improve feature detection in the images. Exemplary non-limiting image corrections are described in further detail below.

In certain embodiments, the computer 903 assigns x, y coordinates for pixels corresponding to features in the corrected FL and CL images and grid row, column coordinates with an identification ID to those features, which is a non-limiting example of gridding (step 1006). In certain embodiments, features in the corrected FL image can appear with more noticeable intensity than features in the corrected CL image. Thus, in certain embodiments, gridding can be first performed on the corrected FL image where the coordinates can then be transformed or mapped to the corrected CL image.

In certain embodiments, gridding locates landmark fidicual controls at known locations in the corrected FL image. In certain embodiments, the landmark fidicual controls can provide a reference point to associate row and column coordinates and ID to the features in the corrected FL image. In certain embodiments, gridding can also associate x, y coordinates of pixels in the corrected FL image corresponding to those features. In certain embodiments, gridding can provide a geographical layout of coordinates for each spot or feature of the corrected FL image. Detailed exemplary steps of gridding according to certain embodiments can be found in the Gridder Example below.

In certain embodiments, the gridding coordinates from the corrected FL image can be transformed onto the CL plane to obtain the exact coordinates for corresponding features in the corrected CL image. For example, in certain embodiments, the column, row coordinates and ID and x, y pixel coordinates for features in the corrected FL image can be transformed and aligned onto the corrected CL image to produce gridding coordinates for the corrected CL image.

In certain embodiments, the computer 903 performs primary image analysis using the corrected CL and FL images (step 1008). In certain embodiments, in this step, the computer 903 can integrate feature signal values and normalize the feature signal values using the combination of the CL and FL signals in the CL and FL images. Non-limiting exemplary image analysis steps are described in further detail below.

In certain embodiments, the computer 903 performs feature quantification refinement (step 1010). In certain embodiments, in this step, the computer 903 can correct for systematic trends for signal values in the corrected images and provides quality assignments for each feature in the corrected images. Non-limiting exemplary feature quantification refinement steps are described in further detail below.

1. Image Correction

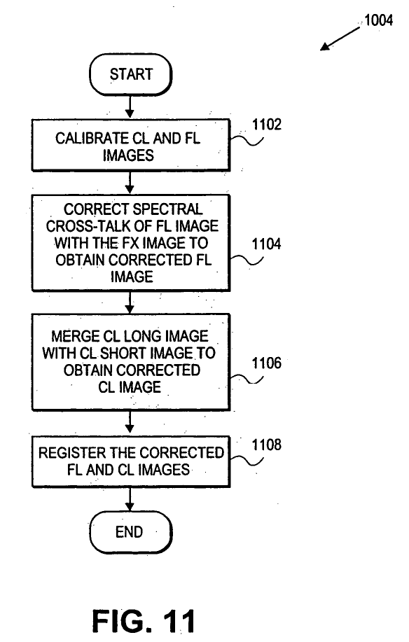

FIG. 11 depicts a detailed flow diagram of exemplary steps to perform image correction (step 1004) of FIG. 10 according to certain embodiments.

Initially, according to certain embodiments, the computer 903 calibrates the FL images and CL images, which can include the FL image, FX image, CL long image, and CL short image (step 1102). In certain embodiments, such images are referred to as “experimental images.” In certain embodiments, artifacts in the experimental images associated with the CCD camera 802 can be corrected using the following calibration images:
1. SBIAS: This calibration image can represent the residual effects of the CCD bias in the CCD camera 802. The image can be subtracted from the CL and FL images. In certain embodiments, the signal values associated with each pixel in this calibration image is subtracted from corresponding signal values of each pixel in the experimental images.
2. SDARK_{—}100s: This calibration image can be corrected for the CCD dark current artifacts caused by the CCD camera 802. This calibration image can be scaled to the experimental images exposure time and subtracted from the experimental images. In certain embodiments, the signal values associated with each pixel in this calibration image can be subtracted from corresponding signal values of each pixel in the experimental images.
3. SPIXFLAT: This calibration image can be applied to the experimental images to correct for small scale pixel-to-pixel CCD gain variations after the SDARK correction. In certain-embodiments, the signal values associated with each pixel in the experimental images can be divided by the corresponding signal values of each pixel in this calibration image.
4. ILLUM_FL: This calibration image can be applied only to the FL image to normalize the non-uniform LED illumination. In certain embodiments, the signal values associated with each pixel in the FL image can be divided by the corresponding signal values of each pixel in this calibration image.
5. BADPIXEL: This may be a binary image or a list of pixel coordinates that identifies known bad pixel signal values in the CCD camera. These pixels can then ignored, flagged, or interpolated over by an algorithm quantifying signals from experimental images ensuring more accurate and robust results.

In certain embodiments, the parameters of the CCD camera 802 can also be used to make corrections. In certain embodiments, artifacts in the images can be offset based on parameters of CCD camera 802 such as gain, read noise, saturation, and CCD bias parameters, accordingly. For example, in certain embodiments, the CCD camera 802 bias offset associated with each image can be estimated and subtracted from all images to further improve the images for subsequent analysis. In certain embodiments, this bias offset can be estimated from the target image itself, a separate “zero-second” dark exposure associated with the experimental image. In certain embodiments, the bias offset may be solved from a linear regression fit to the CL long and CL short exposures.

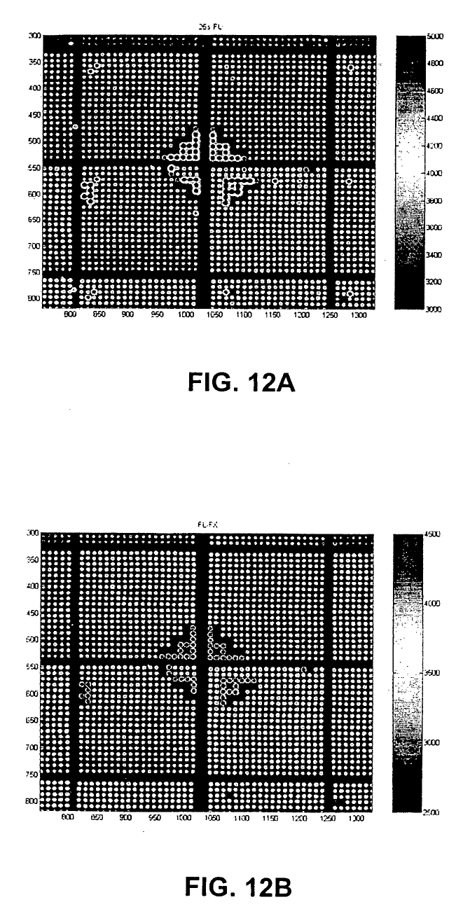

In certain embodiments, the computer 903 corrects spectral cross-talk of the FL image with the FX image to obtain a corrected FL image (step 1104). In certain instances, the CL signals in the CL channel of the microarray 801 can bleed through into the FL channel that causes artifacts (i.e., spectral cross-talk) in the FL image. In this step, according to certain embodiments, the FX image (scaled by relative FL/FX exposure times) is subtracted from the FL image by subtracting the signal values associated with each pixel in the FL image with corresponding signal values of each pixel in the FX image to correct for spectral cross-talk. The resulting image [FL−FX] can be referred to as the “corrected FL image.” FIGS. 12A and 12B show results of spectral cross-talk correction in the FL image according to certain embodiments.

In certain embodiments, computer 903 merges the CL long image with the CL short image to obtain a corrected CL image (step 1106). In certain embodiments, in this step, saturated features in the CL long image are replaced with unsaturated features in the CL short image corresponding to the saturated features. In certain embodiments, the signal values of pixels associated with the saturated features in the CL long image can be replaced by a product of multiplying the signal values of the pixels associated with the unsaturated features in the CL short image by a factor, which in certain embodiments, can be a ratio of CL long and short exposure times, to obtain a corrected CL image.

In certain embodiments, the computer 903 registers the corrected FL and CL images. In certain embodiments, in this step, the coordinates of features in the corrected FL image can be mapped or aligned with the same features in the corrected CL image and can be stored in the database 904 for further processing and analysis. In certain embodiments, pixels in the corrected FL image can be aligned with the same pixels in the corrected CL image using a calibrated transformation matrix due to a small magnification difference between the corrected FL and CL images. In certain embodiments, the coordinate information can be registered and stored in the database 904 for further processing and analysis.

2. Primary Image Analysis

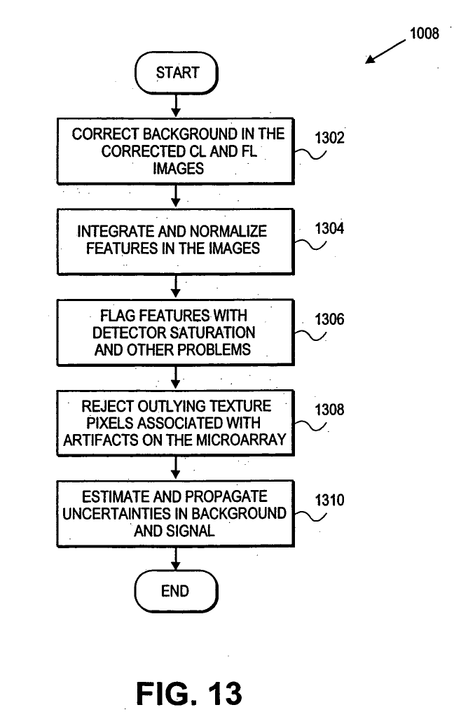

FIG. 13 depicts a detailed flow diagram of the steps to perform primary image analysis (step 1008) of FIG. 10 according to certain embodiments. These steps use the corrected FL and CL images and associated coordinate information regarding features in those images.

Initially, in certain embodiments, computer 903 can perform background correction on the corrected FL and CL images. In certain embodiments, areas of the microarray 801 are intended to be blank with no signal, and these areas can be referred to as the “background.” For example, in certain embodiments, the areas in the rows and columns between sub-grids are intended to be blank with no signals. In certain embodiments, however, areas in the background near neighboring features can have detected signals as a result of spatial crosstalk from signals in those features. In other words, a signal from a neighboring feature is carried over into the background near the feature.

To correct for such crosstalk in the background, in certain embodiments, the computer 903 can correct the signal values for pixels in the background with crosstalk near those features by subtracting the mean of signal values from the background with no crosstalk. In certain embodiments, the computer 903 can interpolate signal values in the background with no crosstalk with those signal in the background with crosstalk near a feature.

In certain embodiments, the computer 903 integrates and normalizes features using the corrected FL and CL images (step 1304). In certain embodiments, the computer 903 can integrate the signal values of pixels associated with each feature in both the corrected FL and CL images. In certain embodiments, the ratio of integrated FL and CL signal values for each feature can be used to provide a normalized signal for each feature. In certain embodiments, the normalized signal can be used as the fully corrected signal for the feature. In certain embodiments, there can be further refinements as well. In certain embodiments, the FL signals for features in the FL image can be used to identify the location of features in the CL image for integration purposes.

In certain embodiments, each feature in the FL and CL images can be represented by a group of pixels (aperture) and can be integrated by adding up the signal values of the pixels multiplied by a weighting factor and then divided by the sum of the weighting factors to obtain an integrated or mean signal value \overline{I} for all the pixels of the feature:

$$\tilde{I} = \frac{{\sum }_{i} {w}_{i} {I}_{i}}{{\sum }_{i} {w}_{i}} ,$$

where Iᵢ is the background-corrected signal value of a pixel of a feature and wᵢ is the weight assigned its pixel in the group.

In a certain embodiments, all pixels associated with a feature can have the same weight (e.g. 1) except for pixels outside the region of interest [ROI] of the feature where they have a value of zero. Since, in certain instances, the signal from a feature typically falls off as a function of distance from its center the ROI is chosen as a balance between integrating significant signal from a feature and minimizing the additional noise weaker signal pixels will contribute to the integrated signal. In a certain embodiments, the ROI may be determined by the average feature morphology.

In certain embodiments, the above algorithm can then sum up the background corrected pixel intensities within a set aperature centered on a feature. A number of factors can be used to determine an appropriate size of the integration aperature. For example, in certain instances, an integration aperature chosen to include more pixels can be outweighed by the addition of more pixels with noise. In certain embodiments, the high correlation between the CL and FL feature pixel intensities can be used to determine the feature integration aperature that deals with noise.

In certain embodiments, the control FL signal for each feature can be configured to give very high signal-to-noise S/N. In certain embodiments, the high S/N can indicate the CL signal morphology, irrespective of whether CL signal can be seen in the image. According to certain embodiments, as detailed below, the FL signal can provide a guide on choosing weights, w, that maximize the S/N of the extracted CL feature intensity such that ROI can be expanded without paying a S/N penalty. In certain embodiments, such weights can be based on the combination of the underlying CL/FL correlation and a noise model that can allow individual pixels to be tested for consistency with the FL feature signal and rejected from the integrated sum. In certain embodiments, by using weights in the above algorithm, the above algorithm can be more robust in view of artifacts on the microarray that might otherwise impact quantification.

In certain embodiments, because the CL and FL pixel intensities can be highly correlated, any ROI can be chosen for the feature to obtain the same CL-normalized value. For example, in certain embodiments, a spot for a feature can have a wide range of shapes with varying amounts of deposited probe and can have the same deposited. CL/FL ratio. In certain embodiments, the above image corrections can improve the underlying CL and FL pixel correlation for each feature. Furthermore, in certain embodiments, the combination of the CL/FL signals can thus improve feature integration and normalization.

In certain embodiments, less weight is given to pixels at the edge of the group and more weight is given to the pixels in the center of the group or in the center of the feature. In certain embodiments, the weight wᵢ can be the inverse variance for each pixel of the feature. In certain embodiments, the variance can be calculated from a noise model for each pixel. In certain embodiments, the noise model for each pixel can be determined by an estimated feature signal uncertainty for the pixel. In certain embodiments, by using the weights to perform the integration, the integration algorithm can give an optimal and unbiased estimate of the integrated sum \overline{I}.

In certain embodiments, within a feature, the initial estimate of the feature signal pixel uncertainty can be determined by the quadrature sum of background noise and the feature Shot noise, separately for both the FL and CL images:
σᵢ²=σ_{BGpix}²C²Iᵢ/g
where the background pixel standard deviation standard deviation σ_{BGpix}² can be estimated from blank regions in a local annulus. Standard mathematical techniques can be used to calculate the standard deviation. The Iᵢ is the background corrected pixel intensity, and the term g represents the CCD gain in electrons per count which comes from the CCD camera 802 calibration. The term C can account for the correlation of pixel to pixel variability within the integration aperture. This term is estimated from the statistics of integrated background pixels (typically measured in the blank background around the perimeter of the microarray). It is the factor by which the integrated background noise exceeds SQRT(N pixels)*σ_{BGpix}² that would be expected in the case of uncorrelated BG noise (where N is the number of pixels used in the sum).

In certain embodiments, the feature FL signal-to-noise (S/N) ratio significantly exceeds its S/N in the CL image. In certain embodiments, the FL feature morphology can be treated as the true profile of the feature in the CL image. The background subtracted feature pixel intensities of the FL image can be normalized by the integrated FL flux to give a feature profile pixel values, p_i.

In certain embodiments, an initial estimate of the total CL feature flux can be determined by the sum of the background corrected pixels of the CL image, G. In certain embodiments, the predicted CL pixel variance can be given by a modified equation for the CL channel:
Vᵢ=σᵢ²=σ_{BGpix}²C²+Gpᵢ/g

In certain embodiments, the measurements can be error-weighted and combined for an average feature pixel signal. In certain embodiments, the optimal error weight, wᵢ, can be given by the inverse variance:

$${w}_{i} = \frac{1}{{V}_{i}}$$

and the new estimate of the averaged feature pixel signal, G, can be given by:

$$G = \frac{{\sum }_{i} {M}_{i} {w}_{i} {p}_{i} {I}_{i}}{{\sum }_{i} {M}_{i} {w}_{i} {p}_{i}^{2}}$$

where the term Mᵢ can be set to zero for rejected measurements.

In certain embodiments, the (variance) uncertainty can then be determined as:

$${σ}_{G}^{2} = \frac{{\sum }_{i} {M}_{i} {p}_{i}}{{\sum }_{i} {M}_{i} {w}_{i} {p}_{i}^{2}}$$

In certain embodiments, the outlier pixels in the evaluation of G can be detected and rejected in an iterative process where each pixel intensity within the feature is tested against the latest estimate of G for that feature:
(I_{CL}_{ᵢ}−Gpᵢ)²>zₜₕᵣ²Vᵢ

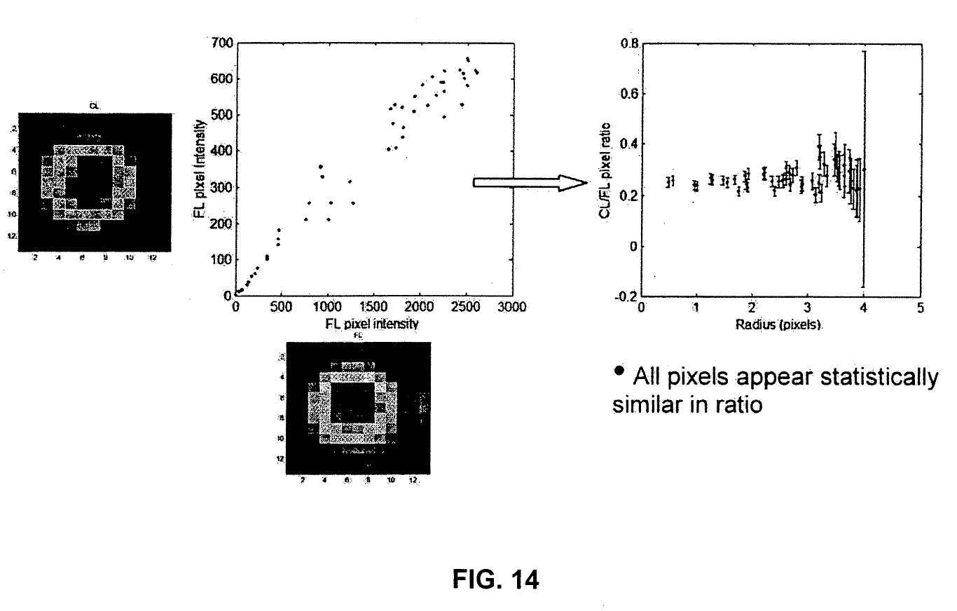

Thus, in certain embodiments, at each iteration if any pixel within the feature exceeds the z-score threshold the largest of these outliers is rejected (i.e. its Mᵢ value is set to zero), the pᵢ factors are re-evaluated and a new estimate of G across is made until all pixels are consistent within the specified z-score threshold. In certain embodiments, the FL signal and its uncertainty is extracted using the same weights as derived in the CL channel. In certain embodiments, the final CL-normalized feature combines the extracted CL signal G and sigma-G with the FL signal and FL signal uncertainties through error propagation. As shown in FIG. 14, in certain embodiments, the above algorithm can use feature CL/FL pixel correlation to maximize the S/N of the final extracted intensity, as well as provide robust quantification in the case of outlying pixels that might be associated with artifacts.

In certain embodiments, features with detector saturation and other problems are then flagged (step 1306). In certain embodiments, the consistency of pixel ratios using statistical data can be used for determining bad pixels that are flagged. In certain embodiments, the outlying feature pixels associated with artifacts of the microarray are rejected (step 1308). In certain embodiments, uncertainties in the background and signal are estimated and the uncertainties in the background and signal are propagated (step 1310).

3. Feature Quantification Refinement

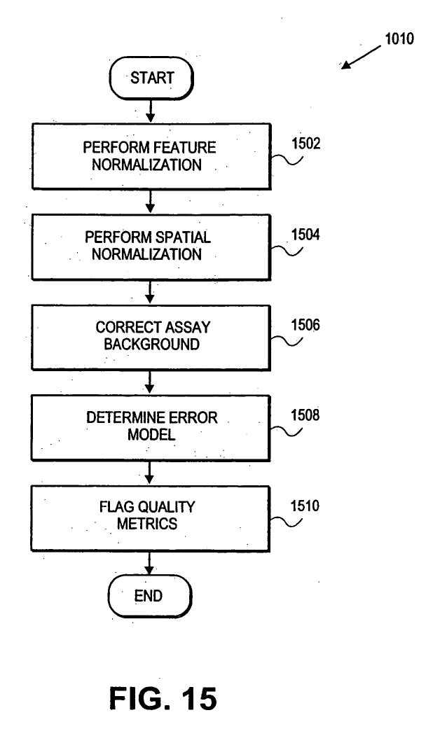

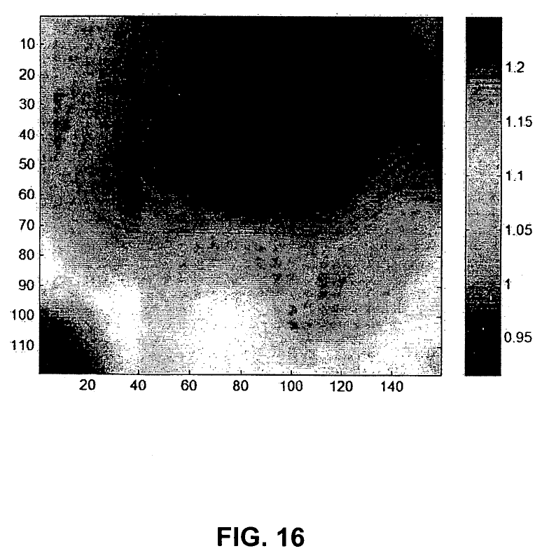

FIG. 15 depicts a flow diagram of steps to perform feature quantification refinement (step 1010) of FIG. 10 according to certain embodiments. In certain embodiments, feature quantification refinement uses the output of primary image analysis for correcting systematic trends and other identifiable variations in the images and for assigning quality values to the quantification of each feature.

In certain embodiments, during feature quantification refinement, feature normalization (e.g., the CL/FL ratio is calculated) is performed (step 1502); spatial normalization is performed (step 1504); the assay background is corrected (step 1506); the error model is determined (step 1508); and the quality metrics are determined and features flagged with various error conditions or warnings (step 1510). Examples of each of these steps is described in more detail below.

In certain embodiments, feature normalization can normalize a feature signal to obtain a fully corrected signal for a feature (I_{CL-INORM}) in count values. In certain embodiments, the I_{CL-INORM} value can be normalized by the FL signal over the feature integration aperture. In certain embodiments, to preserve the approximate feature CL counts, the CL/FL ratio is multiplied by the median FL signal (aperture integrated) for all features containing internal control probes quantified at a particular array position. In certain embodiments, the normalization of the CL intensities by the FL channel can compensate for (1) spotting variations associated with amount of probe deposited, (2) sub-optimal feature centroids and pixelation effects of the finite quantification aperture, (3) morphology differences between the same probe on different microarrays, (4) optical trends in the data. This is a background subtractive CL to FL ratio that, in certain embodiments, provides optimal intensity of a feature and noise associated with it. In certain embodiments, there are a number of metrics for each spot, e.g., I_{CL}, I_{FL}, to obtain I_{CL-NORM} as shown below in the following algorithm:

$${I}_{CL \text{-} INORM} = ( \frac{{I}_{CL}}{{I}_{FL}} ) 〈 {I}_{FL} 〉$$

In certain embodiments, the measurement uncertainty variance in the final I_{CL-INORM} signal is given approximately by:

$$\begin{matrix}{σ}_{CL \text{-} INORM}^{2} = ( {( \frac{{σ}_{CL}}{{I}_{CL}} )}^{2} + {( \frac{{σ}_{FL}}{{I}_{FL}} )}^{2} ) {I}_{CL \text{-} INORM}^{2} \\ = ( {CV}_{CL}^{2} + {CV}_{FL}^{2} ) {I}_{CL \text{-} INORM}^{2}\end{matrix}$$

where the ratio of the normalized CL signal and SIGMA CL-INORM is the feature S/N ratio and this measurement can determine the detectability of that feature based on all the known noise, i.e., is that feature really present in the microarray. In certain embodiments, such measurements can be stored in feature tables.

In certain embodiments, spotting variability may be apparent in the FL images of the microarray. In certain embodiments, normalizing the FL provided from internal control processes can compensate for this variability.

In certain embodiments, spatial normalization can remove and compensate for any systematic spatial trends in feature ratios across the microarray. In certain embodiments, spatial normalization can use spatial normalization (SPN) controls that are placed in a grid throughout the microarray at the same frequency of the subgrids. In certain embodiments, the SPN controls can provide known amounts of both FL and CL signals. In certain embodiments, to correct for I_CL-INORM value trends, a normalized image can be made using the SPN controls and interpolated under every feature then divided into the I_CL-INORM values. Thus, in certain embodiments, with spatial normalization all or most features of the microarray may have mostly the same or the same ratio of CL/FL. In certain embodiments, other normalization processes can be performed such as global normalization that scales signals for comparison of one microarray to another.

Detailed steps of non-limiting exemplary global normalization are discussed in this paragraph as follows. Global normalization scales signals for comparison of one microarray to another. Such scaling can compensate for slight differences in instrument response, exposure time, hybridization conditions, and so on. The median normalized CL signal value (GINORM) is calculated for both microarray positions. The CL-INORM and CL-INORM-SDEV columns are divided by GINORM to populate the GINORM and GINORMSDEV output columns. Global normalization is especially useful to filter data based signal thresholds across arrays. In certain embodiments, downstream analysis packages (for example, GeneSpring® and Spotfire®) can also be used to address global normalization between arrays. However, in certain embodiments, the Gene CV and Gene S/N are not scaled by any kind of global normalization of the data.

In certain embodiments, non-specific background controls can be used on a microarray. In certain embodiments, non-specific background control probes can be designed not to cross-hybridize against the genome in a sample. In certain embodiments, these controls can be used to estimate non-specific signal on each feature—i.e. “assay background” (ABG) which can lie above the optical background of the array addressed earlier. In certain instances, this ABG can be a combination of any non-specific signal and possibly a sequence dependent signal due to cross-hybridization unique to the control probe. In certain embodiments, the median I_CL-INORM signal of these controls can be subtracted from all features to statistically correct signals for ABG signal.

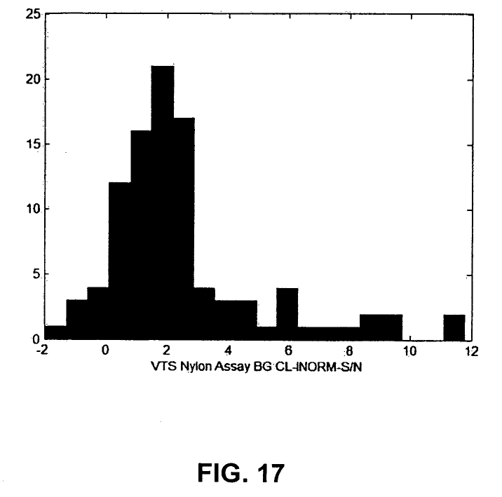

In certain embodiments, the statistical significance of assay background (ABG) measurements can be given by the S/N plot shown in FIG. 17, which has a median S/N of 2 to 1. Referring to FIG. 17, the median value can be small compared to other measurement errors in the system. In certain instances, high S/N points could be single bad measurements or an ABG control with large cross-hybridization. In certain embodiments, although the ABG for an individual probe is difficult to estimate, the median can be useful statistical correction of the average ABG bias of all probe signals. In certain embodiments, CL normalized output columns (CL-INORM, CL-INORM-SDEV, CL-INORM-S/N and CL-INORM-CV) can include the ABG correction.

In certain instances, background noise can be the result of structure in the background fluorescence for FL and spatial cross-talk between features for CL (major); shot noise associated with non-specific chemical signal in the CL channel and with background fluorescence; scattered light in the FL channel; and read-noise from the CCD camera. In certain instances, structure in the background fluorescence for FL and spatial cross-talk between features for CL dominate background noise at the exposure times used for the CL and FL channels. Both noise and signal increase linearly with exposure (or number of images).

In certain embodiments, feature S/N ratio is the ratio of signal to signal standard deviation (SDEV), wherein SDEV is the estimated measurement of uncertainty of the signal on the microarray from all uncertainties associated with the final normalized signal estimate. In certain embodiments, the S/N ratio can express the number of standard deviations above average. In certain embodiments, the S/N ratio metric can be the confidence of the measurement “detectability” above all known sources of noise.

In certain embodiments, based on the S/N ratio of features, genes can be marked “Present” or “Absent” at a desired level of confidence using a probability table for a normal distribution. For example, in certain embodiments, signals with a S/N ratio equal to 3 can have a 99.9% confidence the measurement is real. In certain embodiments, signals with a S/N ratio equal to 2 can have a confidence of 97.7%, a higher false positive rate.

Because certain microarray experiments typically measure differences in gene expression level among different samples, in certain embodiments, the metric can be used to express these differences is the expression ratio. In certain embodiments, gene expression measurements from one microarray can be divided by the measurements from another microarray.

In certain embodiments, a confidence value for the expression ratio or fold change can be calculated from the measurement errors associated with the gene signals. In certain embodiments, the calculation involves finding system precision, the intrinsic spread of the normalized signals independent of measurement errors.

In certain embodiments, the system precision can be estimated by measuring the coefficient of variation (CV), the fractional variation in a signal, for high S/N ratio replicate controls on the microarray. In certain embodiments, system precision for a microarray is typically 5 to 7%. In certain embodiments, gene CV can be calculated using the following algorithm:

$$CLinormCV = \sqrt{{( \frac{1}{CLinormS2N} )}^{2} + {CV}_{prec}^{2}}$$

In certain embodiments, the above algorithm can be the predicted CV derived from an error model. In certain embodiments, this can be equivalent to the CV that would otherwise be estimated experimentally from multiple replicate microarrays.

In certain embodiments, a predicted gene CV can allow the fold change confidence value to be calculated between two or more microarrays. In certain embodiments, the scatter in the ratio measurements can be close to log-normal (the scatter expected for the ratio of two normal distributions with large uncertainties). In certain embodiments, the algorithm in natural log becomes:

$$R = LOG ⅇ ( \frac{{I}_{CL \text{-} INORM1}}{{I}_{CL \text{-} INORM2}} )$$

which has a natural log standard deviation uncertainty given approximately by:
σ_{R}=\sqrt{CV_{CL-INORM1}²+CV_{CL-INORM2}²}

In certain embodiments, Sigma_R can represent the fractional uncertainty in the ratio distribution. The fold change confidence can be given by the z-score:
z=R/σ_{R}
In certain embodiments, the probability can then be found by looking in a normal distribution table.

The following are non-limiting examples of fold change calculations using the above algorithms.

### Example 1

### Calculating a Fold Change Confidence Using Output Globally Normalized Signals

An example gene includes:

| ARRAY | CL-GINORM | CL-INORM-CV |
|---|---|---|
| 1 | 20 | 0.3 |
| 2 | 30 | 0.25 |

Fold Change=20/20=1.5×
SIGMA-R=SQRT(0.3\hat{ }2+0.25\hat{ }2)=0.3905
1/SIGMA-R=2.5607 standard deviations
Z-SCORE probability—98.5% (from probability table)

The gene on Array 2 can have a fold increase of 1.5× at 98.5% confidence over Array 1.

### EXAMPLE 2

### Calculating a Fold Change Confidence Using Normalized Signals and an Independently Determined Global Normalization

An example gene includes:

| ARRAY | CL-GINORM | CL-INORM-CV |
|---|---|---|
| 1 | 2000 | 0.35 |
| 2 | 3000 | 0.25 |

In this example, the separately determined global normalization factor is 1.2 to normalize all signals from Array 2 to Array 1.
Fold Change=3000×1.2/2000=1.8×
SIGMA-R=SQRT(0.35\hat{ }2+0.25\hat{ }2)=0.4301
1/SIGMA-R=2.3250 standard deviations
Z-SCORE probability=97.33% from probability table)

The gene on Array 2 can have a fold increase of 1.8× at 97.3% confidence over Array 1.

### EXAMPLE 3

### Calculating an Upper Limit on Fold Change Confidence if a Gene is Absent (has Low or Negative S/N) on one Microarray

In certain embodiments, a gene can have very low S/N ratio on one microarray and be considered undetectable. The signal and S/N ratio can thus be negative. However, in certain embodiments, the standard deviation of a weak signal can still be used to calculate a statistically meaningful upper or lower limit on fold change.

An example gene includes:

| ARRAY | CL-GINORM | CL-INORM-CV |
|---|---|---|
| 1 | −200 | 250 |
| 2 | 3000 | 0.15 |

The global normalization factor is 1.2, to normalize all signals from Array 2 to Array 1. Array 1 has CL-INORM1-S/N of −0.8. The two SDEV (94.6% confidence) upper limit on this signal would be 2×CL-INORM-SDEV or 500 counts. The equivalent CV of this S/N=2 signal is given by the following equation using a CV precision of 0.07: SQRT ((½)\hat{ }2+0.07\hat{ }2)=0.51(essentially 1/(S/N) for S/N=2).
Fold Change (lower limit)=3000×1.2/(2×250)=7.2×
SIGMA-R=SQRT(0.51\hat{ }2+0.15\hat{ }2)=0.53
1/SIGMA-R=1.8868 standard deviations
Z-SCORE probability=93.27% (from probability table)

The gene on Array 2 can be expressed as 7.2× over Array 1 if Array 1 had a S/N of 2 at 93.3% confidence. However, because a two SDEV upper limit is on Array 1, the gene can have a 93.3% confidence lower limit on fold increase from Array 1 to Array 2 of 7.2×.

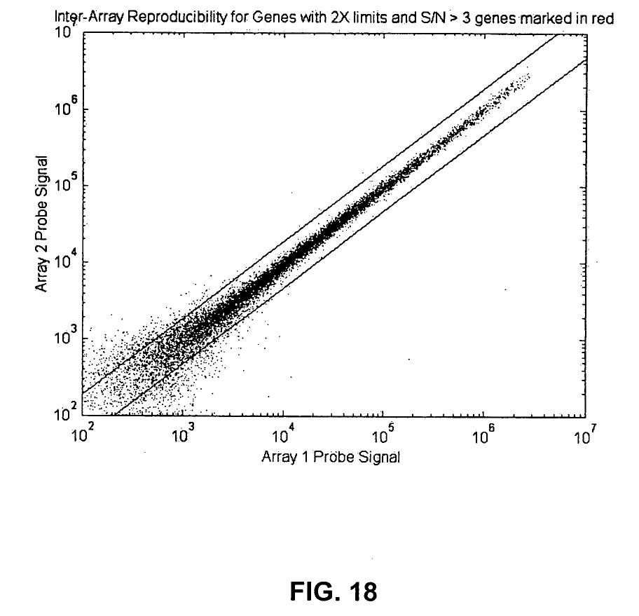

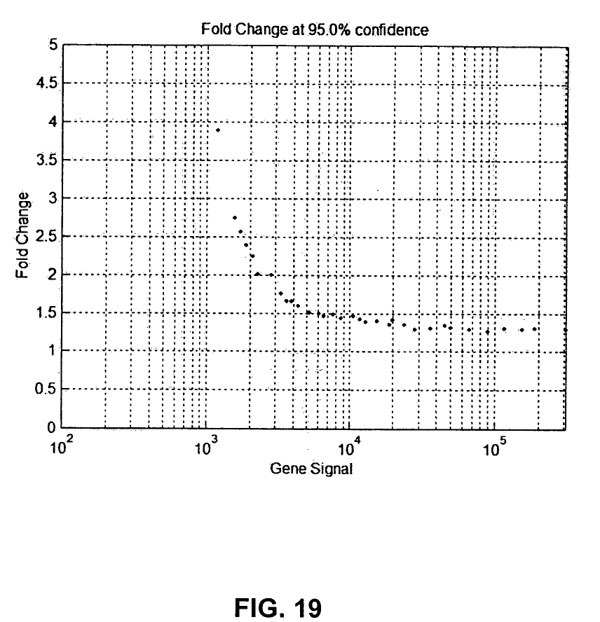

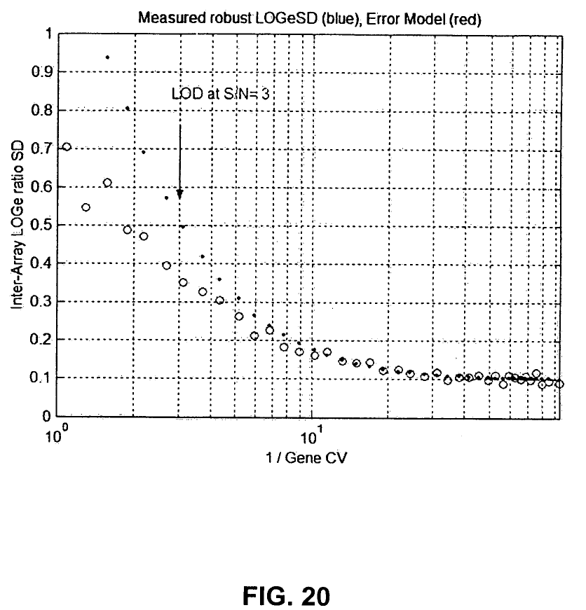

FIGS. 18 to 20 illustrate various plots that compare inter-array reproducibility and show predictions of reproducibility based on predicted CV. FIG. 18 illustrates inter-array reproducibility (for a Strategen UHR sample) showing genes with S/N>=3 in red. FIG. 19 illustrates empirically measured fold change confidence. Each point can represent 2% of genes on the microarray. Fold change confidence can be a function of signal intensity and can be determined empirically by determining the fold change that encompasses 95% of the points in an individual intensity bin. FIG. 20 illustrates that the empirical measure of scatter that can be compared against predictions from the system error model. The error model can determine the feature CV, tracks the empirical measure, and slightly overset images the scatter at S/N=3.

In certain embodiments, flagging quality metrics can include identifying metrics, providing warnings, and indicating failures. Non-limiting exemplary quality metrics for an individual feature can be QC metric1 and QCmetric2.

Qcmetric1 is a feature quality metric given by the CL/FL feature pixel correlation. Users can filter out problematic data based on their own preferred threshold. However, since low correlation can also reflect low S/N on the feature with otherwise valid quantification, it is recommended that users exercise caution with this potential filter.

Qcmetric2 is a feature quality metric that captures the fraction of usable pixels used in quantification. The selection criterion is based on a Chi2 test of CL/FL pixel ratios and can be used to filter out problematic measurements likely to be associated with artifacts on the microarray that are corrupting quantification.

In certain embodiments, in addition, a number of error and warning states can be conveniently encoded in a binary number (FLAGS) where each bit can be defined to flag an individual state. In this nonlimiting example, the FLAGS value correspond to states of increasing severity, for example:
0 or 1: feature has no issues with quantification;
2-200: feature has a problem but should not affect quality of quantification, e.g., problems such as partial feature saturation and rejected pixels;
1000-10000: feature has a problem that affects the quality of quantification, including corrupted neighbors or features outside the optimal focus region of the microarray; and
10000-100000: feature fails due to low FL S/N ratio<5 or rejection of more than ⅓ of feature pixels; and
   >10\hat{ }6: feature fails due to heavy saturation or not being identified during gridding, in other words, invalid quantification.

In certain embodiments, the users can filter data based on these FLAGS values. More details of exemplary non-limiting feature quantification flags are described in the following table.

Feature Quantification Flags Table

| BIT | VALUE | ASSIGNED |
|---|---|---|
| 1 | 1 | Gene NOT detected (at gene S/N threshold of <3 [P. detects2nthr]) |
| 2 | 2 | Feature centroid is from interpolated Grid position |
| 3 | 4 | Feature uses scaled pixels from CLs image |
| 4 | 8 | Partial saturation of feature CL quant pixels (, half [P. maxsatfrac]). |
| 5 | 16 | Partial saturation of feature FL quant pixels (, half [P. maxsatfrac]). |
| 6 | 32 | Partial saturation of pixels (1 or more) in local BG CL or FL annulus. |
| 7 | 64 | Feature has pixels rejected in fit |
| 8 | 128 | Null |
| 9 | 256 | Null |
| 10 | 512 | Null |
| 11 | 1024 | Feature is outside optimal position limits (P/optquantlims) |
| 12 | 2048 | Feature has FL neighbor with quantification problem |
| 13 | 4096 | Feature has CL neighbor with quantification problem |
| 14 | 8192 | Feature has poor CL-Fl correlation (no chosen threshold yet . . . ) |
| 15 | 16384 | Feature has low FL S/N (<5 [P. FLs2nthr]) |
| 16 | 32768 | Feature has poor fit (Qcmetric2 < 0.66) |
| 17 | 65536 | Null |
| 18 | 131072 | Null |
| 19 | 262144 | Null |
| 20 | 524288 | Null |
| 21 | 1048576 | Quantification returned a NaN value for a CLinorm* field |
| 22 | 2097152 | All pixels in local BG (CL or FL) saturated |
| 23 | 4194304 | Full saturation of feature FL quant pixels (>half [P. maxsatfrac]) |
| 24 | 8388608 | Full saturation of feature CL quant pixels (>half [P. maxsatfrac]) |
| 25 | 1677216 | Feature has no Grid position |

Bits 0-5 are informational.
Bits 10-15 are quality issues.
Bits 20-24 are failures in quantification.
Features with FLAGS bits higher than 13 should not be used.
Increasing FLAGS values are associated warnings or error of increasing severity.
FLAGS=0 indicated a measurement with no known errors or issues. In the case of a gene, it indicates a detection (Present) above either the internally specified S/N threshold (defaulted to 3 at present) or a threshold specified externally to the algorithm in the parameter Params.detects2 nthr.
Multiple flags are additive. FLAGS=1028 equals 1024+4+0 (10000000100 in binary) and decodes as “has replaced pixels from CLs image”, is “outside optimal quantification limits” and “detected” (i.e., “Present” if feature is a gene).

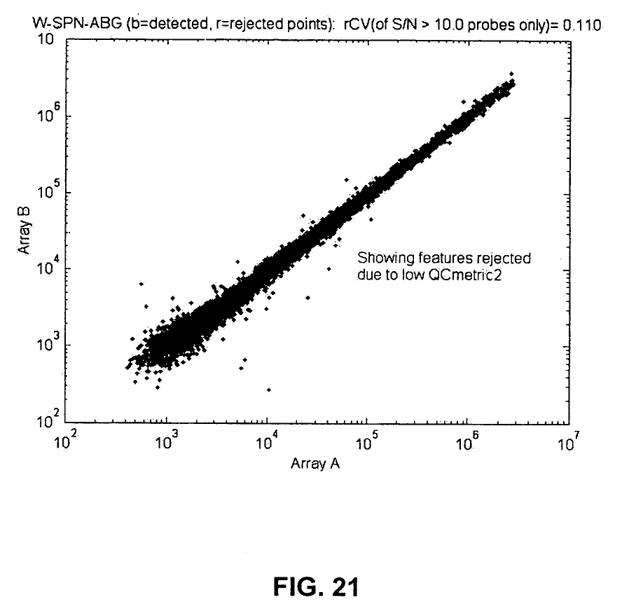

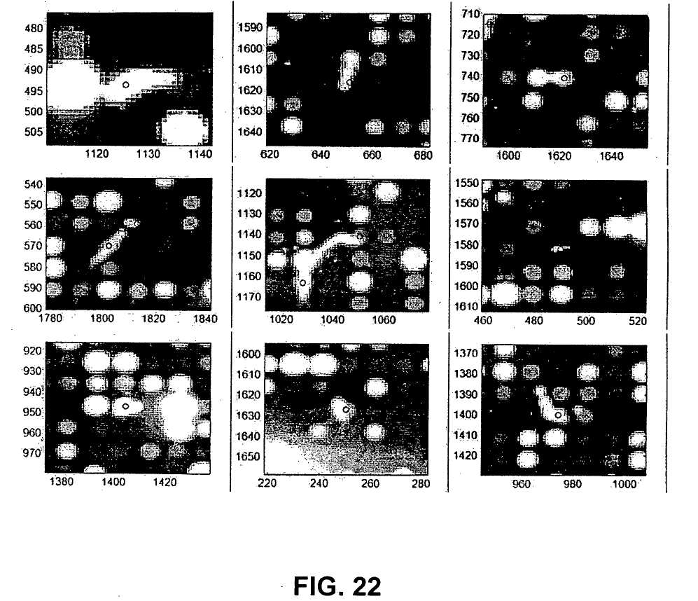

FIG. 21 shows features rejected due to low metrics. Referring to FIG. 21, the data for a gene S/N ratio>3 on both microarrays and filtered by flags>10000. FIG. 22 also shows features rejected due to low metrics. Referring to FIG. 22, features on a single microarray are flagged by a low QCmetric2, which tests the feature morphology integrity.

FIG. 23 is a block diagram that illustrates a computer system 2300, according to certain embodiments, upon which embodiments of the invention may be implemented. In certain embodiments, computer system 2300 includes a bus 2302 or other communication mechanism for communicating information, and a processor 2304 coupled with bus 2302 for processing information. In certain embodiments, computer system 2300 also includes a memory 2306, which can be a random access memory (RAM) or other dynamic storage device, coupled to bus 2302 and instructions to be executed by processor 2304. In certain embodiments, memory 2306 also may be used for storing temporary variables or other information during execution of instructions to be executed by processor 2304. In certain embodiments, computer system 2300 further includes a read only memory (ROM) 2308 or other static storage device coupled to bus 2302 for storing static information and instructions for processor 2304. In certain embodiments, a storage device 2310, such as a magnetic disk or optical disk, is provided and coupled to bus 2302 for storing information and instructions.

In certain embodiments, computer system 2300 may be coupled via bus 2302 to a display 2312, such as a cathode ray tube (CRT) or liquid crystal display (LCD), for displaying information to a computer user. In certain embodiments, an input device 2314, including alphanumeric and other keys, is coupled to bus 2302 for communicating information and command selections to processor 2304. Another type of user input device is cursor control 2316, such as a mouse, a trackball or cursor direction keys for communicating direction information and command selections to processor 2304 and for controlling cursor movement on display 2312. In certain embodiments, this input device typically has two degrees of freedom in two axes, a first axis (e.g., x) and a second axis (e.g., y), that allows the device to specify positions in a plane.

In certain embodiments, computer system 2300 implements the above algorithms and image processing and analysis techniques for control signals of microarrays. In certain embodiments, computer system 2300 implements the methods described in “Applied Biosystems 1700 Chemiluminescent Microarray Analyzer”, Ver. 1.1 User Guide for processing and analyzing microarrays in response to processor 2304 executing one or more sequences of one or more instructions contained in memory 2306. In certain embodiments, such instructions may be read into memory 2306 from another computer-readable medium, such as storage device 2310. In certain embodiments, execution of the sequences of instructions contained in memory 2306 causes processor 2304 to perform the process states described herein. In certain embodiments, hard-wired circuitry may be used in place of or in combination with software instructions to implement the invention. Thus implementations are not limited to any specific combination of hardware circuitry and software.

The term “computer-readable medium” as used herein refers to any media that participates in providing instructions to a processor for execution. Such a medium may take many forms, including but not limited to, non-volatile media, volatile media, and transmission media. Exemplary non-volatile media includes, but is not limited to, optical or magnetic disks, such as storage device 2310. Exemplary volatile media includes, but is not limited to, dynamic memory, such as memory 2306. Exemplary, transmission media includes, but is not limited to, coaxial cables, copper wire, and fiber optics, including the wires that comprise bus 2302. In certain embodiments, transmission media can take the form of acoustic or light waves, such as those generated during radio-wave and infra-red data communications.

Common forms of computer-readable media include, but are not limited to, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, a carrier wave as described hereinafter, or any other medium from which a computer can read.

In certain embodiments, various forms of computer readable media may be involved in carrying one or more sequences of one or more instructions to processor 2304 for execution. For example, in certain embodiments, the instructions may initially be carried on magnetic disk of a remote computer. In certain embodiments, the remote computer can load the instructions into its dynamic memory and send the instructions over a telephone line using a modem. In certain embodiments, a modem local to computer system 2300 can receive the data on the telephone line and use an infra-red transmitter to convert the data to an infra-red signal. In certain embodiments, an infra-red detector coupled to bus 2302 can receive the data carried in the infra-red signal and place the data on bus 2302. In certain embodiments, bus 2302 carries the data to memory 2306, from which processor 2304 retrieves and executes the instructions. In certain embodiments, the instructions received by memory 2306 may optionally be stored on storage device 2310 either before or after execution by processor 2304.

In various embodiments, methods may be implemented with both object-oriented and non-object-oriented programming systems.

### GRIDDER EXAMPLE

This non-limiting example describes gridding according to certain embodiments. Given an arrangement of features that form a two dimensional rectangular grid on some substrate and an image of those features, in this non-limiting example, a gridder assigns image pixel x,y coordinates and grid row, column coordinates to those features. In this non-limiting example, a microarray has 239×159 features and the system uses two overlapping images to image the entire array; one top and one bottom. This results in each of the two images having at least the top or bottom 119×159 features as well as additional features that are in the overlap area.

In this non-limiting example, the signals from features include both a fluorescent (FL) signal from internal control probes and a chemiluminescent (CL) signal. In this non-limiting example, except for a small amount of specified features, all features provide a FL signal from internal control probes while the CL signal varies per experiment. In this nonlimiting example, each array position is imaged in two channels: fluorescent (FL) and chemiluminescent (CL). This results in an FL image and a CL image for each array half. In this non-limiting example, almost all features will appear with significant intensity in the FL image, and many features will not appear with significant intensity in the CL image. In this non-limiting example, gridding is performed on the FL channel. In this non-limiting example, the optics use a linear transform applied to the FL grid coordinates to produce the CL grid coordinates.

In this non-limiting example, the microarray includes 96 sub-grids (12×8), with 19×19 features per sub-grid, and it includes blank rows and columns separating the sub-grids.

In this non-limiting example, the gridding system is comprised of 3 separate gridders (in other words, 3 gridding algorithms) providing double redundancy. In this non-limiting example, images are processed by the first gridder, and if it succeeds, the gridding system is complete; otherwise the next gridder attempts to grid it and so on for the subsequent gridders.

Exemplary Gridder Elements

In this non-limiting example, aspects of gridding algorithms can be generalized as follows:
Estimate Feature Centroids: Obtain the centroids of every blob in the image that might be a feature. Some of these may be just noise.
Estimate the Region Of Interest (ROI): Find the portion of the image that contains the microarray features.
Estimate the Initial Grid: This contains the main differences between gridders. Create an initial estimate of the entire grid using observed image characteristics taken in the context of a priori information about the expected appearance of the array in the FL image.
   Array layout.
   Corner fiducials.
   Expectation of straight rows and columns of features & blanks.
Match Features to Estimated Grid: Replace the estimated feature coordinates contained in the initial grid estimate with the real feature centroids as found in “Estimate Feature Centroids.”
   Exemplary Gridder Elements: Estimating Feature Centroids

In this non-limiting example, all of the gridders create an initial list of possible feature centroids to match against their estimated grid:
Normalized cross-correlation against a model feature pdf is used to create an enhanced feature image.
Quadratic fitting is used to determine the feature centroids in the enhanced feature image.

In this non-limiting example, very faint features are found but noise is also incorrectly found as features. In this non-limiting example, by rectifying these coordinates to the estimated grid, real from false features can be determined.

Exemplary Gridder Elements: Region Of Interest (ROI) Determination

In this non-limiting example, ROI determination is used by the Row/Column Gridder and the Blank Gridder (which are discussed below) to find the area of the image that has the array data. If corners were reliable, the Corner Gridder (which is discussed below), would have worked, and thus, the Row/Column Gridder and the Blank Gridder assume that corners can't be trusted.

In this non-limiting example, ROI determination is used to find the high density region of previously found centroids, which defines the ROI.

Gridding Algorithm 1: Corner Gridder

In this non-limiting example, the corner gridder attempts to take advantage of a design feature of the microarray which incorporates a specific landmark fiducial feature pattern at the four corners of each half of the array to find the grid corners. In this non-limiting example, the corner gridder estimates feature centroids, finds corner landmark fiducial features, interpolates between grid corners to provide an estimated initial grid, and matches the actual feutres to the estimated grid. By interpolating between the corners of the grid, the corner gridder gives a reasonable initial estimate of the feature locations in the image. In this non-limiting example, further common processing attempts to ascertain the actual feature coordinates.

Gridding Algorithm 2: Row/Column Gridder

In this non-limiting example, the row/column gridder does not use the corner fiducials. Rather, the row/column gridder attempts to find entire rows and columns of features assuming that they are reasonably straight lines. In this non-limiting example, the row/column gridder estimates feature centroids, estimates ROI, and creates lines to estimate rows and columns of features. In this non-limiting example, an initial grid estimate is created by computing the intersections of these lines, and the actual features are then matched to the estimated grid.

Creating Line Feature Row/Column Line Estimates

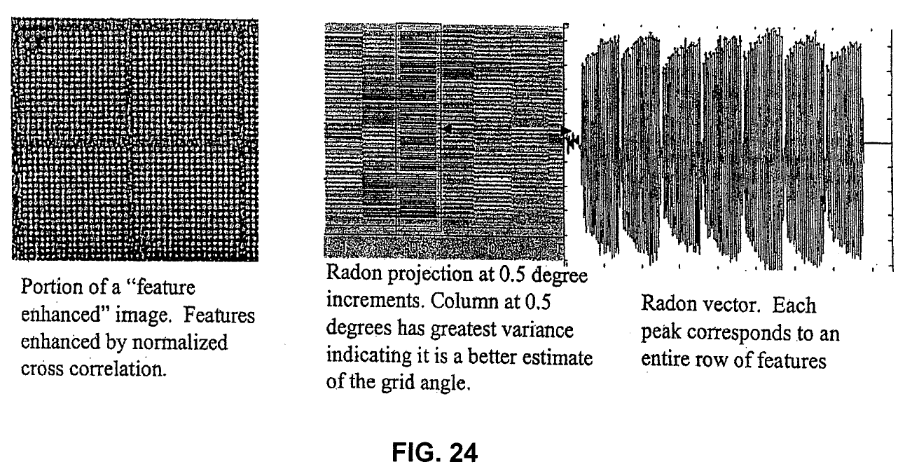

In this non-limiting example, the grid will have some rotation w/r to the image axis. The algorithm uses an optimizer on the variance of the radon transform of the feature enhanced image to determine the angle of this rotation to <0.01 degrees. In this non-limiting example, the radon projection w/r to the computed optimal grid rotation angle and the corresponding perpendicular angle yields two radon vectors. The peaks in the vectors should correspond to the rows and columns of features. See, for example, FIG. 24.

Gridding Algorithm 3: Blank Gridder

In this non-limiting example, the blank gridder does not use corner fiducials. Rather, the blank gridder attempts to find the blank rows and columns that separate the subgrids. In this non-limiting example, the blank gridder estimates feature centroids, estimates ROI, and creates lines to estimate blank rows and columns. In this non-limiting example, the positions of the corners of each subgrid are estimated by the intersection of those lines. In this non-limiting example, interpolating between these subgrid corners yields the initial grid estimate, and the actual features are then matched to the estimated grid.

Creating Blank Line Feature Row/Column Line Estimates

In this non-limiting example, the blank gridder uses the same technique as the row/column gridder to find grid angle. In this non-limiting example, the blank gridder searches optimal radon vectors for indications of blank lines rather than feature lines, and computes the intersection of blank lines.

Exemplary Gridder Elements: Matching Features to the Estimated Grid

In this non-limiting example, all three gridding algorithms create an initial grid estimate, albeit in different ways, but the coordinates are not those of real features. In this non-limiting example, all three gridding algorithms create a list of initial feature centroid estimates but they lack grid row/column coordinate information. In this non-limiting example, the gridding algorithms return a grid of real feature coordinates, and this is accomplished by matching the features to the estimated grid centroids. In this non-limiting example, each subgrid has its own positional bias (in other words, shared by all of its features.) Therefore, the matching of feature centroids to the grid is performed on a subgrid by subgrid basis. In this non-limiting example, some subgrids match easily to their features: the feature centroids and estimated grid points match. However, some subgrids may not and so more processing is performed to “recover” the subgrids.

In this non-limiting example, the following steps are performed:

1. Match estimated grid points against apparent/found feature centroids. Match Criteria:
1. Must be within ⅓ feature distance in both x and y directions.
2. A grid point can only match one feature centroid.

2. Re-estimate each subgrid using matched points.
1. If the vast majority of points in a subgrid matched, then use the real feature centroids to re-estimate the subgrid.
2. Otherwise, attempt to recover the subgrid. That is, re-estimate the subgrid from scratch just that portion of the image.

3. Repeat step 1 using the new grid estimate.

4. Substitute matched real feature centroids for estimated grid points.

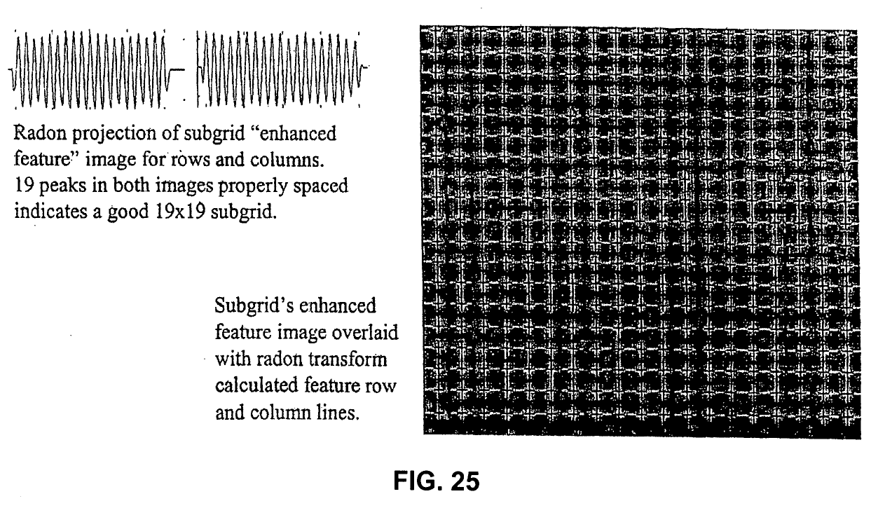

In this non-limiting example, radon transform is used to find subgrid rows and columns. See, for example, FIG. 25.

TABLE A
Internal Control Probe (ICP) Sequence

| | |
|---|---|
| 5′-amino-TTCGGCTGTGAGAACGATCACGCA-3′ | SEQ ID NO: 1 |

TABLE B

Sequence of Fluorescent Control Probe used in
Control Ladders and Landmark Fiducials

SEQ ID NO: 2

5′-LIZ-CGAATCGTTTGAGCAAGACCCACG-3′amino

TABLE C

Sequence of Chemiluminescent Control Probe used in
Control Ladders and Landmark Fiducials

SEQ ID NO: 3

5′-DIG-CGAATCGTTTGAGCAAGACCCACG-3′amino

TABLE D
Sequences of Three Hybridization Control Probes

| | | |
|---|---|---|
| HYB_Control_1_Cp | CGACATGAAACTTGGTTTGTGCCCAGTAGCGACAGAATCACGTATCGGTTTACGCCGTCA | SEQ ID NO: 4 |
| HYB_Control_2_Cp | CTCGAGAGTAATTATGACACGTAAGGTTTAAGAGCCCGCCGGACTTGGATCCGTCCTACT | SEQ ID NO: 5 |
| HYB_Control_3_Cp | TGAACTGGTTTTGCTAGCCCACTCAACGGTCACGCATCTAAGGGATATGCCGATTCAGGA | SEQ ID NO: 6 |

TABLE E

Sequences of IVT Control Gene Probes.
Five Probes each target control genes BioB, BioC, and BioD.

| | | |
|---|---|---|
| IVT_BIOB_1_Cp | CGCGGCGTGGAAGAATCCCCACGAACGCGATATGCCGTACCTGGAACAAATGGTGCAGGG | SEQ ID NO: 7 |
| IVT_BIOB_2_Cp | GACACCTCGCCGGAGTTTTACGGCAATATCATCACCACACGCACTTATCAGGAACGCCTC | SEQ ID NO: 8 |
| IVT_BIOB_3_Cp | GGTCGCGCGGATCATGATGCCAACCTCTTACGTGCGCCTTTCTGCCGGACGCGAGCAGAT | SEQ ID NO: 9 |
| IVT_BIOB_4_Cp | ACCTCTTACGTGCGCCTTTCTGCCGGACGCGAGCAGATGAACGAACAGACTCAGGCGATG | SEQ ID NO: 10 |
| IVT_BIOB_5_Cp | TTGAACAGGCGCTGATGACCCCGGACACCGACGAATATTACAACGCGGCAGCATTAGAGG | SEQ ID NO: 11 |
| IVT_BIOC_1_Cp | ACGCCTTACTGGCAATGCTTCCACAGCGTAAATACACCCACGTACTGGACGCGGGTTGTG | SEQ ID NO: 12 |
| IVT_BIOC_2_Cp | CGCAGACCATTATCTGGCGGGAGATATCGAATCCCTGCCGTTAGCGACTGCGACGTTCGA | SEQ ID NO: 13 |
| IVT_BIOC_3_Cp | AACTGCATCAGGCGTGGCAGGCGGTGGACGAGCGTCCGCATGCTAATCGCTTTTTACCGC | SEQ ID NO: 14 |
| IVT_BIOC_4_Cp | TTACCGCCAGATGAAATCGAACAGTCGCTGAACGGCGTGCATTATCAACATCATATTCAG | SEQ ID NO: 15 |
| IVT_BIOC_5_Cp | GCTGTGGTTTGATGATGCGCTCAGTGCCATGCGTTCGCTGAAAGGCATCGGTGCCACGCA | SEQ ID NO: 16 |
| IVT_BIOD_1_Cp | AGTGGGGAAAACTGTCGCCAGTTGTGCACTTTTACAAGCCGCAAAGGCAGCAGGCTACCG | SEQ ID NO: 17 |
| IVT_BIOD_2_Cp | GGATTACGCAACAGTAAATCCTTACACCTTCGCAGAACCCACTTCGCCGCACATCATCAG | SEQ ID NO: 18 |
| IVT_BIOD_3_Cp | GCCGGTGATACTGGTAGTTGGTGTGAAACTCGGCTGTATTAATCACGCGATGTTGACTGC | SEQ ID NO: 19 |
| IVT_BIOD_4_Cp | ATCACGCGATGTTGACTGCACAGGTAATACAACACGCCGGACTGACTCTGGCGGGTTGGG | SEQ ID NO: 20 |
| IVT_BIOD_5_Cp | GTTACGCCTCCGGGAAAACGTCACGCTGAATATATGACCACGCTCACCCGCATGATTCCC | SEQ ID NO: 21 |

TABLE F

Sequences of RT Control Gene Probes.
Five Probes each target control genes Dap, Lys, and Phe.

| | | |
|---|---|---|
| RT_DAP_1_Cp | ACATACAAAAATTGCATTAGAGCACGGAGTCCGTCCAGTTGTCGGAACAACCGGTTTCTC | SEQ ID NO: 22 |
| RT_DAP_2_Cp | GACCAGAAGCTTGACGCACCAAGCGGAACTGCGCTTAAAACAGCGGAAATGATTTCAGAA | SEQ ID NO: 23 |
| RT_DAP_3_Cp | ATTCTTATAACCGTGCTTCTTTCATGTCAGGCGTTAAACTGTCAGTCGAACAAGTCATGA | SEQ ID NO: 24 |
| RT_DAP_4_Cp | CAACCGGAACCACAGGGTTGAAAATTCATGAGGCGACAGGTCTTCAAATTGAACGTTTTC | SEQ ID NO: 25 |
| RT_DAP_5_Cp | AGGATTCCGTTTCTACTCCTCTGACGAATGGGTATATTGAAATCGTTGAAGCGAGAGAAA | SEQ ID NO: 26 |
| RT_LYS_1_Cp | TTCTTTAGATGTCGTATCCGGAGGAGAGCTATATACGGCTGTTGCAGCAGGCTTTCCGGC | SEQ ID NO: 27 |
| RT_LYS_2_Cp | GAAATCGCGCTTCTTGAAGACCTATGTAAAGAAACGGGTCACTCCATCGATGTTCTTCTT | SEQ ID NO: 28 |
| RT_LYS_3_Cp | TCAGCTGCTGOGTGTCCATTGCCATATCGGCTCGCAAATCTTTGATACGGCCGGTTTTGT | SEQ ID NO: 29 |
| RT_LYS_4_Cp | AAGAAAATGCTTCCCGTTACGGTTTTGACATTCCGGAAATTTGGATCGAACCGGGCCGTT | SEQ ID NO: 30 |
| RT_LYS_5_Cp | AAAAGAAGTGGATAAGCTGTACAATCGTTTCATCATTCGGCGTGCGAATTAAGGATCCAA | SEQ ID NO: 31 |
| RT_PHE_1_Cp | CTTACCGCACCATTCCGGAGTGTATAGATGCAGCCGTTGCAGGCGAAGTTGATTTTGCTT | SEQ ID NO: 32 |
| RT_PHE_2_Cp | TTTACTCACATTCACACGCGATTGCGCAATGCCATAAATTTCTTCATCGACATTTTCCTT | SEQ ID NO: 33 |
| RT_PHE_3_Cp | GGGACAATCATACAAGATTTATTATCCTGTCTCCCGATGAAAACATATCTTTTGAAGTGA | SEQ ID NO: 34 |
| RT_PHE_4_Cp | TCAAAAATTGAGTCACGTCCGACTAAAACCGGATTAGGCCATTATTTCTTTATTATTGAT | SEQ ID NO: 35 |
| RT_PHE_5_Cp | CCAGGGGCCATGCAGGAGCTTGAAGCACTCGGCTGCAAAGTGAGGCTTCTGGGTGCATAC | SEQ ID NO: 36 |

TABLE G
Non-Specific Background Control Probe Sequences

| | | |
|---|---|---|
| Background_1_Cp | CAACTTCCTTCAACACTTGAGCGGAGTCGGTGCATCCGAACAATGGAAGCTTCCACATTG | SEQ ID NO: 37 |
| Background_2_Cp | CTCACGGTACACAAATCCGTGCTCTAACTCGAATTCAACACAAGGAATCCACTTGTTGCG | SEQ ID NO: 38 |
| Background_3_Cp | GAATTTTCCGCATTTGATTGGTTCTTGTAAGTATGAGCCGGTTAGGATTAGGCCTCGCCG | SEQ ID NO: 39 |
| Background_4_Cp | CGGTGGATCAGTTTTACTTCGATGGTAGTTTATTAATGGACCAATCAGAATGTTCTTATT | SEQ ID NO: 40 |
| Background_5_Cp | AACGAGTTTTGCTGTATGAAATATCTATCGATGACTCAACAGGTTTTGATCATGATCATA | SEQ ID NO: 41 |
| Background_6_Cp | CACGCGTCCGCAAAACCATCTTAAAGCAACTACACAAATCTTGAAATTTTCTCATATTTT | SEQ ID NO: 42 |
| Background_7_Cp | ACAAGATTACTCTGCTAGAGGAGCTTCAAGAAAAGACCGAGGAAGATGAGGAGAACAAGC | SEQ ID NO: 43 |
| Background_8_Cp | AAGATGAGGAGAACAAGCCTAGTGTCATCGAAAAGCTTCACCGATCCAACAGCTCTTCTT | SEQ ID NO: 44 |
| Background_9_Cp | AAAAGTCAACGTACGATGACGTTTTCACTTGCGTCACTCTCATGATTTCATTTATTCTTG | SEQ ID NO: 45 |
| Background_10_Cp | TTCACCAATGATGACTCTGCTTGTGCTGACAGTTCGATTTAGCTAACCACTCTTGTTGTG | SEQ ID NO: 46 |
| Background_11_Cp | CAGGAACTTGTTATGTCGTGCTTCTTGATGCAGCAATGACAGATCATTTTCGATTGGGCC | SEQ ID NO: 47 |
| Background_12_Cp | TTTCATCACGTTCCTAACTTAACCGGGTGCCGTATCTGAACCAAGCGGTGCTCGGACAAG | SEQ ID NO: 48 |
| Background_13_Cp | TAGGCCACAACGAGTAACACTGCGGTCGAGAAAGACCTACGAAGAACATTATATTTGGAA | SEQ ID NO: 49 |
| Background_14_Cp | GAACCACGATACAGCTGTCGGGCCCACCGTGATCTTTCTGGGAGCTGGAAGCTATTACCA | SEQ ID NO: 50 |
| Background_15_Cp | GTTGTTCTGCGATGGTGGACACCGTATAAGCCGACAAAGGCCTCGGAGGGTGCTCCCGGG | SEQ ID NO: 51 |
| Background_16_Cp | CCCTAACTCATGATGAGGGTACCATACCGTATACTACCGTGTAACCCCTTAGTTTTGATG | SEQ ID NO: 52 |
| Background_17_Cp | TCAGAAACATAGCGTAATAAACGAACAGCGGTCCATTATCCCTATAGCTCTGGCGCGTTT | SEQ ID NO: 53 |
| Background_18_Cp | GGTTCGTGTATTGACAAGATATGACTTACATAGTTCCGTCAGTCAGTGGTGCATGGTCTG | SEQ ID NO: 54 |
| Background_19_Cp | TGGAGCGTAGGCGGTTTCTTAAGCTAATCGTCTCACCTAGCGATAAGATTTTGCGGGCGG | SEQ ID NO: 55 |
| Background_20_Cp | TGTCTTTTCGACCTAATGCTATGACGCTAGTAATTAGCCCACGCCTGAATCTGTCGCCGC | SEQ ID NO: 56 |
| Background_21_Cp | ATAAATGGTGCAGTGTATGAACCTTTTCCAACCAGTGCTAGCACCCTACGTCTTTTTGCC | SEQ ID NO: 57 |
| Background_22_Cp | CTGTGATTACCGTGCAATTCAATCTTATGGTAAACCTACGGACGTACGAGGAACAAGCTC | SEQ ID NO: 58 |
| Background_23_Cp | CGTACGAGGAACAAGCTCGACTAGCTTAACACAATTGCCAGTCTGGACCATACTTGTGCA | SEQ ID NO: 59 |
| Background_24_Cp | TATGCCAGGTCTATATCCTCGTTCCGTGGAAATACACCGCCGTAGCACAAATGCGAGCGA | SEQ ID NO: 60 |
| Background_25_Cp | TGGTGCAGGTACTCTTCTGCAATAGCGTCGTATCGTAACTAGTCTATGGAGCACTAGAAT | SEQ ID NO: 61 |
| Background_26_Cp | CGATGACACTGATTAAGGCAACCGGGTGGAGGTATATGCTATCCAACTAGGCATGTACCA | SEQ ID NO: 62 |
| Background_27_Cp | CCCCATAGGCCATATCCTATTATCATCGCTTACGACGCTAACGACCGTTAAATCATATCC | SEQ ID NO: 63 |
| Background_28_Cp | GATGCTCCTATCGTTGCTGAATGCGAAGGCTAGGGTGCACCCGACGCGGCAGTGTAGGTT | SEQ ID NO: 64 |
| Background_29_Cp | TAGCCGCGTTGATAGGAGATAACTATGTTACATAAAGCGAACGGTTACTCTTAATGAGGG | SEQ ID NO: 65 |
| Background_30_Cp | GGTTACTCTTAATGAGGGCCGGCAGATAATCCGGAATTTTGGCGCTCAAGTTCTTGCACC | SEQ ID NO: 66 |
| Background_31_Cp | CGCGGTCCCTAGCCGCGTCCGGACCAATCTCACCTATGCAAGCCAATTCTGTCGCGCGCG | SEQ ID NO: 67 |
| Background_32_Cp | CGACCCCGGTCTTATAGCCACGATTACGCTTGCATTAGTTTGATAAATAGAACGCGCCCC | SEQ ID NO: 68 |
| Background_33_Cp | CCATAAATGACAAGAGTTCCCCTCGGTAAACAGGGTATATCTGAAACTACTAGGAAAAGA | SEQ ID NO: 69 |
| Background_34_Cp | GAGCCTAACTATCGTTCATCGAGACCGACTATGCAACAACTCCTGCGTTGCTGAGTAGGG | SEQ ID NO: 70 |
| Background_35_Cp | TACAATGATAGGTCGCTTATTAACAGTTTGCGAGTCGCGTGTTGTAGTGAACAAAGCCTT | SEQ ID NO: 71 |
| Background_36_Cp | AGCAACGGGCCTATGCGATATCTGATAGTCAGGGTTGACACATATTCGATTATACCCTTC | SEQ ID NO: 72 |
| Background_37_Cp | TATTCGATTATACCCTTCCTATCACGTCATCTCATGTGCCTTCGCTCATAGTATATAAAC | SEQ ID NO: 73 |
| Background_38_Cp | ACTAACCCGCTGTTTAGAGTTCAGCTAGTGACCCTTTGACATACCACACATGCCAACTAG | SEQ ID NO: 74 |
| Background_39_Cp | CACACTTAGGTGCACAAACCTGATGATTCAAGCATAGCGGACTGATCTTTGTAACACACA | SEQ ID NO: 75 |
| Background_40_Cp | CGAAGTCGAATGACAGAATGTCAAAGCTTGTAGGCACTCCGCGACTAGGGTTATTTAGTC | SEQ ID NO: 76 |
| Background_41_Cp | TGCGGTATAGATGCCATTGTGCGACTTTTTTGTAAATCGCATGAATGGGCAAGGGTAAGC | SEQ ID NO: 77 |
| Background_42_Cp | CCCTATAGGGACCGTTGAACGCGTCTAGGCAGCATACGCCCAGACTACATGGCGCGCACA | SEQ ID NO: 78 |
| Background_43_Cp | TTTATCACGGAGAGTTACCTCGCGCTATAGAAGCAAATATGTCAGGATTCGTATGTGTAC | SEQ ID NO: 79 |
| Background_44_Cp | GCCAACCACTGTGTAAAAACATTGCGGGTAATTTTAGCACCGAAAGTCCAAAGCATCTGA | SEQ ID NO: 80 |
| Background_45_Cp | CTAATGGCGGGACCAGACCCAAAAGAGTTGCAAGAGTTTGACTCACTGCCGTTCGGTGAG | SEQ ID NO: 81 |
| Background_46_Cp | CCGTGAATTACGGTCTGTTCGTGTCTCTGCATGTCAGCGATGTGAACACGTGGCTGATCG | SEQ ID NO: 82 |
| Background_47_Cp | GGTCTGTTCAGATAACGCTATGCAAGTGTTGGCAATGGCGCTAGTAGCAACTCTGCTCCG | SEQ ID NO: 83 |
| Background_48_Cp | AGTAGCAACTCTGCTCCGGATTTCGTTGTAGGAAGTCATGCTCGTATCGAACTAACTACA | SEQ ID NO: 84 |
| Background_49_Cp | GCGCTAAGAAGTTACCCCATGGGTTAAAATTAGGACCCGTGTTGTAGACTCAAAGAGGAG | SEQ ID NO: 85 |
| Background_50_Cp | GTCAACAGTGACATGCCATCCTATATACTCAACAGCAGTATCAGTCAGATGAAAACATGA | SEQ ID NO: 86 |
| Background_51_Cp | AGTCAGATGAAAACATGACGACGTGGATCCCGACGTAGTAGCATAGGGTTGTAGAGCGGG | SEQ ID NO: 87 |
| Background_52_Cp | CGGCTGCATTTGTAGTAGCACCCATATCTACGATTTCAGCGGGTGTGAACGTCCAAACAA | SEQ ID NO: 88 |
| Background_53_Cp | CTTGACCTGATAGCTATTTCGGAGTTAGGTCCCAACCCGTCAGAATGTCACGTGCCAGTC | SEQ ID NO: 89 |
| Background_54_Cp | CCACACTCCGTAATCGTTGTCGCGCTTCTGGGATTGAGTCAGAAACGGCGGGCTGACACT | SEQ ID NO: 90 |
| Background_55_Cp | TCGTAGCCGATAAGCAAACTTGCGTGCTTGAGGCCATTGTACGTGGGAGACACTTCGTAC | SEQ ID NO: 91 |
| Background_56_Cp | GAGACCAGTATGAGCGAAACGTCCTCAGAAATGTTACGGGTACCTAGCTGCGAAACCCCA | SEQ ID NO: 92 |
| Background_57_Cp | ATTATCACTCTGCGATGGTTCGGAGATACTGTCATCGGTGCCCGCATTTGTCGGAATGGA | SEQ ID NO: 93 |
| Background_58_Cp | TTCTCTAACAATAGCCGATTAATCTTGTGCGGAGCTCTAGGCAGCTACCTAGTAGTGAGA | SEQ ID NO: 94 |
| Background_59_Cp | CGCTAGTCGGCCGAAATTGTAGGGATTCGGAGGTATATGATAAGTTCGAAAATTCCTCCC | SEQ ID NO: 95 |
| Background_60_Cp | GCCCAATAGGGTCCATAGTGCCGATGTTACGTTTGGATTAGGGCATTCTACCTTCTTGCC | SEQ ID NO: 96 |
| Background_61_Cp | ATCCCCCGATAAAATATGGCTAAGCACAGGTTCCATGCGGAGTGAGTCTGAGGCGTTATG | SEQ ID NO: 97 |
| Background_62_Cp | TGAGTCTGAGGCGTTATGCCTTCGATTGCCTTTACCGTACCCACGCTGCCAATTGTTTTG | SEQ ID NO: 98 |
| Background_63_Cp | ACCGGTTTACGTCATAACTACTTCGTCAGTTACTCTCACGACTTTCTTAATCGCTAGTAG | SEQ ID NO: 99 |
| Background_64_Cp | ACTTATTTGCGTCCAAATGACTAGATACAAGAGGTTAACGGCGTTGCCGACTGCTAACGC | SEQ ID NO: 100 |
| Background_65_Cp | ACGGCAATTGTTCTTTGTGACGTTTAAGGGCCATGCAGCAGATACGGGAATATAATACCA | SEQ ID NO: 101 |
| Background_66_Cp | TGTGCGTCAGGATTCACGCGCATGTCACAAAGCAGTTACGTAGCGGTGTCCCAATCTGTC | SEQ ID NO: 102 |
| Background_67_Cp | CTGTATATCTCTGCAAGGCATGAACGCAATGTGCTCTTTACTCCTACCCCTTTAACACCT | SEQ ID NO: 103 |
| Background_68_Cp | CCTACCCCTTTAACACCTGTTACCATTAGTTGACGTGAGGGCGGCAAAAAGATGATTGGA | SEQ ID NO: 104 |
| Background_69_Cp | GGCCGTTGTTTGCACACTGTAGTATCTGAGTGTCACAATCCCACCGTTTCACAATATTAA | SEQ ID NO: 105 |
| Background_70_Cp | ACCGTTTCACAATATTAAAGTCCCCCGAAAATGACCTAAATCCACATGTCCTAATTTATT | SEQ ID NO: 106 |
| Background_71_Cp | AAGGGAGTACAGACTCTACCCTTGTGATCCGTGTATCAAATTTTCTAAACGCTTACCAGC | SEQ ID NO: 107 |
| Background_72_Cp | GTTGAGGCTACATAGGAACATCGGCCGTATAAACAGAGGAAACTTATCCTACATACAGGG | SEQ ID NO: 108 |
| Background_73_Cp | GGATTTACTTAGGTCTACACGTTGTCACCTTAGTGATAGAAACAGACACTGATGAATATA | SEQ ID NO: 109 |
| Background_74_Cp | AACCGTTCAGTAAAGGTAGGATAGCCCATGTGATCAACTCTATTTGCAGCAATGCACGTT | SEQ ID NO: 110 |
| Background_75_Cp | TTTCAGTGTGCCATTCTTGATTAATACAGCACGAGATGAATCGACCGTCTTCGCCCGTAA | SEQ ID NO: 111 |
| Background_76_Cp | AACCAAGTACTAAGCATAGTCAATGTACGAGTGTTACAGGTCTCATTGGTACGGGTCGGG | SEQ ID NO: 112 |
| Background_77_Cp | AAAGGGTAGCCGGCACAAGTTACGGACTCGAAATATCGGTCAACCATCGGGCAAGTCGGG | SEQ ID NO: 113 |
| Background_78_Cp | GACAGTTGTAACATTGCGGTAATACTTGACTTACGACTGAGCTTTCAATTGAGACTAACC | SEQ ID NO: 114 |
| Background_79_Cp | ACCAAGAGGCTCTTAGAGGATCAGGGTTAGCTATTTAAGCCTTCGAATCCTAGCGGCGTG | SEQ ID NO: 115 |
| Background_80_Cp | CCAACGGCACGTTAACAGACGGTGAAGCCTTCTCGTCAGATCTTAGAGGCGGATTCGACA | SEQ ID NO: 116 |
| Background_81_Cp | ACCAACCTATGTAGCACGCGAGATTTGGAAATAAGGCCAACGAATCATCATGTTACGAAG | SEQ ID NO: 117 |
| Background_82_Cp | GTGACGCGCTCAGCAATTGCTATACTAGCGTTCTGCAAACCTTTCAGGTGTATTAGCAGA | SEQ ID NO: 118 |
| Background_83_Cp | CTCCTTTATTGAGTCCGAGCGTTACAACGACACATTTCAGTTGAAGCGCTTGACACCACA | SEQ ID NO: 119 |
| Background_84_Cp | CTGGATCCGACTAATAGCCAGCGTCAGCGGATGGCCTATCTGTGCGTTACGTTCGCCACA | SEQ ID NO: 120 |
| Background_85_Cp | ACCAGGACCTTGCCCGGTCTCTAAATTTTCTTAATACGTAAACTACCCAAATGTCCCAGC | SEQ ID NO: 121 |
| Background_86_Cp | CATTCATCCAACCGCGGCTCGATGGGCATTCCTCTCATCGTTCTGTCCCATAGACCTATC | SEQ ID NO: 122 |
| Background_87_Cp | CACCCAATCGATGTGTTTATGTTTGTCCTGACGAGATCCGCTGCTTGTTCAAAAGAAGAT | SEQ ID NO: 123 |
| Background_88_Cp | CTAGAAGCTCGCAATAGTCAGGGACCTACCATGTTGTGAAGGCCATACGTATCGCCCGCG | SEQ ID NO: 124 |
| Background_89_Cp | GGTAGCGTTCGGTAGTTATAATATCTATTCTCGCGTAAACTTGTTCCCAGCGCGCAAGGG | SEQ ID NO: 125 |
| Background_90_Cp | TAGGCGTGTGAACCGGATCGCCGTAAGAGCCGCACAACACTCCTAAAGCAGCGTGTCTAT | SEQ ID NO: 126 |
| Background_91_Cp | CAATTCATATAAAATCTTACGTTTCACGCGCTTATACTAGCTGAGGCTAGCCCACCCTCA | SEQ ID NO: 127 |
| Background_92_Cp | CGGACAAATATACTATTCCAATGTGCCTCGTATTCAATGTCGTTCTCAGTGGAACCACAC | SEQ ID NO: 128 |
| Background_93_Cp | GCCGGTTTTCACTGAACTTACTCTGCCTGCAGATCACCAGTCAGTTTCTACGAGGGTGGG | SEQ ID NO: 129 |
| Background_94_Cp | CCATGTCGCACGTACAGCTCGTGGACAAAATAGCTTTTGCGGGCAGTAAAAATTCATTCT | SEQ ID NO: 130 |
| Background_95_Cp | GCAGTAAAAATTCATTCTGATTCGGTTACGCCTTAGACTCCTCCGGCGCGATCACCACAA | SEQ ID NO: 131 |
| Background_96_Cp | CAAAGAAGTCTGCTATGTTTAGTTACCCAATCGTGGCAAAGACAATCCACATTTGGCTGG | SEQ ID NO: 132 |
| Background_97_Cp | GCAACCGTTTCGCCATGGTATTCTCGGGCCCGTGACACTTTGGTTCCAATGGATTAATAC | SEQ ID NO: 133 |
| Background_98_Cp | CCGAGGTGCACATCTACTCCGTATTCACATTAGACTTCAAGACTGACTGGCATACAAGGG | SEQ ID NO: 134 |

TABLE H
Positive Gene Control Probe and Positive Gene Mismatch Control Probe Sequences

| | | |
|---|---|---|
| PosControl_101 | CAACTATGAAGAATTCGTACAGATGATGACTGCAAAATGAAGACCTACTTTCAACTCCTT | SEQ ID NO: 135 |
| PosControl_101m5 | GAATTACGAGGACTTGGTGCATATCATAACTGCAAAATGAAGACCTACTTTCAACTCCTT | SEQ ID NO: 136 |
| PosControl_101m3 | CAACTATGAAGAATTCGTACAGATGATGACGGCGAATTGCAGTCCCACGTTGAATTCTTT | SEQ ID NO: 137 |
| PosControl_101m4 | CAACTATGAAGAATTTGTTCAAATCATTACAGCGAATTGCAGACCTACTTTCAACTCCTT | SEQ ID NO: 138 |
| PosControl_101m2 | GAACTAAGAAGAGTTCGTTCAGATAATGACCGCAAAGTGAAGCCCTACATTCAATTCCTT | SEQ ID NO: 139 |
| PosControl_101m1 | CACTATGAGAATCGTACGATGAGACTGAAAATAAGACTACTTCAACTCTTTTTTTTTTTT | SEQ ID NO: 140 |
| PosControl_102 | AACATTGCTGTTCAAAGAAATTACAGTTTACGTCCATTCCAAGTTGTAAATGCTAGTCTT | SEQ ID NO: 141 |
| PosControl_102m1 | ACTTCTTTAAGAAATTACAGTTTACGTCCATTCCAAGTTGTAAATGCTAGTCTTTTTTTT | SEQ ID NO: 142 |
| PosControl_102m2 | AACATGTGTCAAAATTACAGTTTACGTCCATTCCAAGTTGTAAATGCTAGTCTTTTTTTT | SEQ ID NO: 143 |
| PosControl_102m3 | AACATTGCTTTAAGAATACGTTTACGTCCATTCCAAGTTGTAAATGCTAGTCTTTTTTTT | SEQ ID NO: 144 |
| PosControl_102m4 | AACATTGCTGTTCAAGAATAAGTTCGTCCATTCCAAGTTGTAAATGCTAGTCTTTTTTTT | SEQ ID NO: 145 |
| PosControl_102m5 | AACATTGCTGTTCAAAGAATTCATTACTCATCCAAGTTGTAAATGCTAGTCTTTTTTTTT | SEQ ID NO: 146 |
| PosControl_103 | AGAACAAGAGCTAGAGCGATTAAGAAGCGAAAATAAGGATATTGAAAATCTGAGAAGAGA | SEQ ID NO: 147 |
| PosControl_103m1 | AGAACTTTTTTCTTTGCGATTAAGAAGCGAAAATAAGGATATTGAAAATCTGAGAAGAGA | SEQ ID NO: 148 |
| PosControl_103m2 | AGAACAAGAGCTAGATTTTCCTTTTAGCGAAAATAAGGATATTGAAAATCTGAGAAGAGA | SEQ ID NO: 149 |
| PosControl_103m3 | AGAACAAGAGCTAGAGCGATTAAGATTTTTTTTCTAGGATATTGAAAATCTGAGAAGAGA | SEQ ID NO: 150 |
| PosControl_103m4 | AGAACAAGAGCTAGAGCGATTAAGAAGCGAAAATATTTTCTCCTTAAATCTGAGAAGAGA | SEQ ID NO: 151 |
| PosControl_103m5 | AGAACAAGAGCTAGAGCGATTAAGAAGCGAAAATAAGGATATTGATTTCTCTTTTAGAGA | SEQ ID NO: 152 |
| PosControl_104 | ACACATTGTTACAGCTAGAGTGTGAAAAATACAAATCCGTCCTTGCAGAAACAGAAGGAA | SEQ ID NO: 153 |
| PosControl_104m1 | TTTTTTTGTTACAGCTAGAGTGTGAAAAATACAAATCCGTCCTTGCAGAAACAGAAGGAA | SEQ ID NO: 154 |
| PosControl_104m2 | TTTTTACACATTGTTTAGAGTGTGAAAAATACAAATCCGTCCTTGCAGAAACAGAAGGAA | SEQ ID NO: 155 |
| PosControl_104m3 | TTTTTACACATTGTTACAGCTAGAGAAAATACAAATCCGTCCTTGCAGAAACAGAAGGAA | SEQ ID NO: 156 |
| PosControl_104m4 | TTTTTACACATTGTTACAGCTAGAGTGTGAAAAATTCCGTCCTTGCAGAAACAGAAGGAA | SEQ ID NO: 157 |
| PosControl_104m5 | TTTTTACACATTGTTACAGCTAGAGTGTGAAAAATACAAATCCGTCAGAAACAGAAGGAA | SEQ ID NO: 158 |
| PosControl_105 | ACGCCAGATGCGTGAAATGGAAGAGAACTTTGCCGTTGAAGCTGCTAACTACCAAGACAC | SEQ ID NO: 159 |
| PosControl_105m1 | TTTTTACGCCCGTGAAATGGAAGAGAACTTTGCCGTTGAAGCTGCTAACTACCAAGACAC | SEQ ID NO: 160 |
| PosControl_105m2 | TTTTTACGCCAGATGCGTGAAAGAGAACTTTGCCGTTGAAGCTGCTAACTACCAAGACAC | SEQ ID NO: 161 |
| PosControl_105m3 | TTTTTACGCCAGATGCGTGAAATGGAAGAGTGCCGTTGAAGCTGCTAACTACCAAGACAC | SEQ ID NO: 162 |
| PosControl_105m4 | TTTTTACGCCAGATGCGTGAAATGGAAGAGAACTTTGCCGGCTGCTAACTACCAAGACAC | SEQ ID NO: 163 |
| PosControl_105m5 | TTTTTACGCCAGATGCGTGAAATGGAAGAGAACTTTGCCGTTGAAGCTGCACCAAGACAC | SEQ ID NO: 164 |
| PosControl_106 | CAGGATGTTGACAATGCGTCTCTGGCACGTCTTGACCTTGAACGCAAAGTGGAATCTTTG | SEQ ID NO: 165 |
| PosControl_106m1 | CAGGATGTTAACAACGCGTTTCTGGCACGTCTTGACCTTGAACGCAAAGTGGAATCTTTG | SEQ ID NO: 166 |
| PosControl_106m2 | CAGGACGTCGATAACGCATCCCTGGCACGTCTTGACCTTGAACGCAAAGTGGAATCTTTG | SEQ ID NO: 167 |
| PosControl_106m3 | CAGGATGTTGACAATGCGTCTCTGGCACGTCTTGACCTTGAACGCAAAGTGGAATCTTTG | SEQ ID NO: 168 |
| PosControl_106m4 | TAGAATATTAACGATACGCCTTTGACATGTTTTAACCTTGAACGCAAAGTGGAATCTTTG | SEQ ID NO: 169 |
| PosControl_106m5 | CGGAACGCTAATAGTACATTTTTAGTATGCCTTGACCTTGAACGCAAAGTGGAATCTTTG | SEQ ID NO: 170 |
| PosControl_107 | CCTACTTTGTCTTCAGCAGTTAGCTAAATTACAAGATCGAGAATGGTTAACAGAACTTTT | SEQ ID NO: 171 |
| PosControl_107m1 | TTCGTCCCACCTTCAGCAGTTAGCTAAATTACAAGATCGAGAATGGTTAACAGAACTTTT | SEQ ID NO: 172 |
| PosControl_107m2 | CCTACTTTGTTCCTGATGACTAGCTAAATTACAAGATCGAGAATGGTTAACAGAACTTTT | SEQ ID NO: 173 |
| PosControl_107m3 | CCTACTTTGTCTTCAGCAGTCTATCGGGCCACAAGATCGAGAATGGTTAACAGAACTTTT | SEQ ID NO: 174 |
| PosControl_107m4 | CCTACTTTGTCTTCAGCAGTTAGCTAAATTGTGGAGCTAGGAATGGTTAACAGAACTTTT | SEQ ID NO: 175 |
| PosControl_107m5 | CCTACTTTGTCTTCAGCAGTTAGCTAAATTACAAGATCGAAGGCAACCGGCAGAACTTTT | SEQ ID NO: 176 |
| PosControl_108 | TTCCAGAGTCCGGAGGCAGCAGACACGCCCTCTTAGTAGGGACTTAATGGGCCGGTCGGG | SEQ ID NO: 177 |
| PosControl_108m1 | TTCCTTTTCTCGGAGGCAGCAGACACGCCCTCTTAGTAGGGACTTAATGGGCCGGTCGGG | SEQ ID NO: 178 |
| PosControl_108m2 | TTCCAGAGTCCGGATTTTTTAGACACGCCCTCTTAGTAGGGACTTAATGGGCCGGTCGGG | SEQ ID NO: 179 |
| PosControl_108m3 | TTCCAGAGTCCGGAGGCAGCAGACTTTTTTTCTTAGTAGGGACTTAATGGGCCGGTCGGG | SEQ ID NO: 180 |
| PosControl_108m4 | TTCCAGAGTCCGGAGGCAGCAGACACGCCCTCTTTTCTTTGACTTAATGGGCCGGTCGGG | SEQ ID NO: 181 |
| PosControl_108m5 | TTCCAGAGTCCGGAGGCAGCAGACACGCCCTCTTAGTAGGGACTCTTCTTGCCGGTCGGG | SEQ ID NO: 182 |
| PosControl_109 | GGTGGCGGAGACCCGCAAGCGCAAGGGCCTGAAAGAAGGCATCCCTGCCCTGGACAACTT | SEQ ID NO: 183 |
| PosControl_109m1 | GGTGGCGGAGACCCAGCGCAAGGGCCTGAAAGAAGGCATCCCTGCCCTGGACAACTTTTT | SEQ ID NO: 184 |
| PosControl_109m2 | GGTGGCGGAGCCCACGCAAGGGCCTGAAAGAAGGCATCCCTGCCCTGGACAACTTTTTTT | SEQ ID NO: 185 |
| PosControl_109m3 | GGTGGCGGGCCCACCAAGGGCCTGAAAGAAGGCATCCCTGCCCTGGACAACTTTTTTTTT | SEQ ID NO: 186 |
| PosControl_109m4 | GGTGGCGGCCCACCAGGCCTGAAAGAAGGCATCCCTGCCCTGGACAACTTTTTTTTTTTT | SEQ ID NO: 187 |
| PosControl_109m5 | GGTGCGGCCCACCAGGCCTGAAAGAAGGCATCCCTGCCCTGGACAACTTTTTTTTTTTTT | SEQ ID NO: 188 |
| PosControl_110 | GACATCCAGAAGGACCTAAAAGACCTGTGACTAGTGAGCTCTAGGCTGTAGAAATTTAAA | SEQ ID NO: 189 |
| PosControl_110m1 | TTTTCTCAGAAGGACCTAAAAGACCTGTGACTAGTGAGCTCTAGGCTGTAGAAATTTAAA | SEQ ID NO: 190 |
| PosControl_110m2 | GACATCCAGTTTTTTCTAAAAGACCTGTGACTAGTGAGCTCTAGGCTGTAGAAATTTAAA | SEQ ID NO: 191 |
| PosControl_110m3 | GACATCCAGAAGGACCTAATTTTTTTGTGACTAGTGAGCTCTAGGCTGTAGAAATTTAAA | SEQ ID NO: 192 |
| PosControl_110m4 | GACATCCAGAAGGACCTAAAAGACCTGTGTTCTTCGAGCTCTAGGCTGTAGAAATTTAAA | SEQ ID NO: 193 |
| PosControl_110m5 | GACATCCAGAAGGACCTAAAAGACCTGTGACTAGTGAGCCTCTTTCTGTAGAAATTTAAA | SEQ ID NO: 194 |
| PosControl_111 | TGAAAGTGCCTATGAAGTCATCAAGCTAAAAGGATATACCAACTGGGCTATTGGATTAAG | SEQ ID NO: 195 |
| PosControl_111m1 | TGCCTATGAAGTCATCAAGCTAAAAGGATATACCAACTGGGCTATTGGATTAAGTTTTTT | SEQ ID NO: 196 |
| PosControl_111m2 | TGAAAGTGCAGTCATCAAGCTAAAAGGATATACCAACTGGGCTATTGGATTAAGTTTTTT | SEQ ID NO: 197 |
| PosControl_111m3 | TGAAAGTGCCTATGAAGTCCTAAAAGGATATACCAACTGGGCTATTGGATTAAGTTTTTT | SEQ ID NO: 198 |
| PosControl_111m4 | TGAAAGTGCCTATGAAGTCATCAAGCTAAATACCAACTGGGCTATTGGATTAAGTTTTTT | SEQ ID NO: 199 |
| PosControl_111m5 | TGAAAGTGCCTATGAAGTCATCAAGCTAAAAGGATATAGGGCTATTGGATTAAGTTTTTT | SEQ ID NO: 200 |
| PosControl_112 | AATCTGGTGCAGAGAAATGTTAATGTCTTCAAATTCATTATTCCTCAGATCGTCAAGTAC | SEQ ID NO: 201 |
| PosControl_112m1 | AATCAGAGAAATGTTAATGTCTTCAAATTCATTATTCCTCAGATCGTCAAGTACTTTTTT | SEQ ID NO: 202 |
| PosControl_112m2 | AATCTGGTGCAGAGTAATGTCTTCAAATTCATTATTCCTCAGATCGTCAAGTACTTTTTT | SEQ ID NO: 203 |
| PosControl_112m3 | AATCTGGTGCAGAGAAATGTTAATAAATTCATTATTCCTCAGATCGTCAAGTACTTTTTT | SEQ ID NO: 204 |
| PosControl_112m4 | AATCTGGTGCAGAGAAATGTTAATGTCTTCAAATTTCCTCAGATCGTCAAGTACTTTTTT | SEQ ID NO: 205 |
| PosControl_112m5 | AATCTGGTGCAGAGAAATGTTAATGTCTTCAAATTCATTATTCCCGTCAAGTACTTTTTT | SEQ ID NO: 206 |
| PosControl_113 | CAGCAGATTTTGGCAGGTGAATATGACCATCTCCCAGAACAGGCCTTCTATATGGTGGGA | SEQ ID NO: 207 |
| PosControl_113m1 | TGGCAGGTGAATATGACCATCTCCCAGAACAGGCCTTCTATATGGTGGGATTTTTTTTTT | SEQ ID NO: 208 |
| PosControl_113m2 | CAGCAGATTTATATGACCATCTCCCAGAACAGGCCTTCTATATGGTGGGATTTTTTTTTT | SEQ ID NO: 209 |
| PosControl_113m3 | CAGCAGATTTTGGCAGGTGACTCCCAGAACAGGCCTTCTATATGGTGGGATTTTTTTTTT | SEQ ID NO: 210 |
| PosControl_113m4 | CAGCAGATTTTGGCAGGTGAATATGACCATAGGCCTTCTATATGGTGGGATTTTTTTTTT | SEQ ID NO: 211 |
| PosControl_113m5 | CAGCAGATTTTGGCAGGTGAATATGACCATCTCCCAGAACTATGGTGGGATTTTTTTTTT | SEQ ID NO: 212 |
| PosControl_114 | GACCGTGTCCCGTGCACGGAAAATACAGCGTTTCTTGTCTCAGCCATTCCAGGTTGCTGA | SEQ ID NO: 213 |
| PosControl_114m1 | GACCCGTGCACGGAAAATACAGCGTTTCTTGTCTCAGCCATTCCAGGTTGCTGATTTTTT | SEQ ID NO: 214 |
| PosControl_114m2 | GACCGTGTCCCGTGAAATACAGCGTTTCTTGTCTCAGCCATTCCAGGTTGCTGATTTTTT | SEQ ID NO: 215 |
| PosControl_114m3 | GACCGTGTCCCGTGCACGGAAAATTTTCTTGTCTCAGCCATTCCAGGTTGCTGATTTTTT | SEQ ID NO: 216 |
| PosControl_114m4 | GACCGTGTCCCGTGCACGGAAAATACAGCGTTTCCAGCCATTCCAGGTTGCTGATTTTTT | SEQ ID NO: 217 |
| PosControl_114m5 | GACCGTGTCCCGTGCACGGAAAATACAGCGTTTCTTGTCTCAGCAGGTTGCTGATTTTTT | SEQ ID NO: 218 |
| PosControl_115 | CTGTGCCACTGTCCCCCCAGCCATTCACTCCTACTGATGAGACAAGATGCGGTGATGACA | SEQ ID NO: 219 |
| PosControl_115m1 | CTGTGCCCAGCCATTCACTCCTACTGATGAGACAAGATGCGGTGATGACATTTTTTTTTT | SEQ ID NO: 220 |
| PosControl_115m2 | CTGTGCCACTGTCCCCACTCCTACTGATGAGACAAGATGCGGTGATGACATTTTTTTTTT | SEQ ID NO: 221 |
| PosControl_115m3 | CTGTGCCACTGTCCCCCCAGCCATTGATGAGACAAGATGCGGTGATGACATTTTTTTTTT | SEQ ID NO: 222 |
| PosControl_115m4 | CTGTGCCACTGTCCCCCCAGCCATTCACTCCTACTGATGCGGTGATGACATTTTTTTTTT | SEQ ID NO: 223 |
| PosControl_115m5 | CTGTGCCACTGTCCCCCCAGCCATTCACTCCTACTGATGAGACAATGACATTTTTTTTTT | SEQ ID NO: 224 |
| PosControl_116 | TCGTCATGGGTGTGAACCATGAGAAGTATGACAACAGCCTCAAGATCATCAGCAATGCCT | SEQ ID NO: 225 |
| PosControl_116m1 | TCGTCATGGGTGTGGACCATGAGAAGTATGACAACAGCCTCAAGATCATCAGCAATGCCT | SEQ ID NO: 226 |
| PosControl_116m2 | TCGTCATGGGTGCGGATCATGAGAAGTATGACAACAGCCTCAAGATCATCAGCAATGCCT | SEQ ID NO: 227 |
| PosControl_116m3 | TCGTCATGAGCGCGGATCATGAGAAGTATGACAACAGCCTCAAGATCATCAGCAATGCCT | SEQ ID NO: 228 |
| PosControl_116m4 | TCGTCACGAGCGCGGATCGTGAGAAGTATGACAACAGCCTCAAGATCATCAGCAATGCCT | SEQ ID NO: 229 |
| PosControl_116m5 | TCGTTACGAGCGCGGATCGTAAGAAGTATGACAACAGCCTCAAGATCATCAGCAATGCCT | SEQ ID NO: 230 |
| PosControl_117 | GGAAGGTGAAGGTCGGAGTCAACGGATTTGGTCGTATTGGGCGCCTGGTCACCAGGGCTG | SEQ ID NO: 231 |
| PosControl_117m1 | AGGAAGCGGAGGTCGGAGTCAACGGATTTGGTCGTATTGGGCGCCTGGTCACCAGGGCTG | SEQ ID NO: 232 |
| PosControl_117m2 | GGGAAGCGGAAGTCGGAGTCAACGGATTTGGTCGTATTGGGCGCCTGGTCACCAGGGCTG | SEQ ID NO: 233 |
| PosControl_117m3 | GGAAAGCGGAAGCCGGAGTCAACGGATTTGGTCGTATTGGGCGCCTGGTCACCAGGGCTG | SEQ ID NO: 234 |
| PosControl_117m4 | GGAAGGCGGAAGCCAGAGTCAACGGATTTGGTCGTATTGGGCGCCTGGTCACCAGGGCTG | SEQ ID NO: 235 |
| PosControl_117m5 | GGAAGGTGGAAGCCAGGGTCAACGGATTTGGTCGTATTGGGCGCCTGGTCACCAGGGCTG | SEQ ID NO: 236 |
| PosControl_118 | TGCTCCTCCTGTTCGACAGTCAGCCGCATCTTCTTTTGCGTCGCCAGCCGAGCCACATCG | SEQ ID NO: 237 |
| PosControl_118m1 | TGCTCCTCCTCAGCCGCATCTTCTTTTGCGTCGCCAGCCGAGCCACATCGTTTTTTTTTT | SEQ ID NO: 238 |
| PosControl_118m2 | TGCTCCTCCTGTTCGACAGTTTCTTTTGCGTCGCCAGCCGAGCCACATCGTTTTTTTTTT | SEQ ID NO: 239 |
| PosControl_118m3 | TGCTCCTCCTGTTCGACAGTCAGCCGCATCTCGCCAGCCGAGCCACATCGTTTTTTTTTT | SEQ ID NO: 240 |
| PosControl_118m4 | TGCTCCTCCTGTTCGACAGTCAGCCGCATCTTCTTTTGCGAGCCACATCGTTTTTTTTTT | SEQ ID NO: 241 |
| PosControl_118m5 | TGCTCCTCCTGTTCGACAGTCAGCCGCATCTTCTTTTGCGTCGCCAGCCGTTTTTTTTTT | SEQ ID NO: 242 |
| PosControl_119 | TTTTGCAAGGGCCTCTGAATCTGTCTGTGTCCCTGTTAGCACAATGTGAGGAGGTAGAGA | SEQ ID NO: 243 |
| PosControl_119m1 | TTTTGCAAGGGCCTCTGAATCTGTCTGTGTCCCTGTTAGCACAATGTGAGTTTTTTTTTT | SEQ ID NO: 244 |
| PosControl_119m2 | TTTTGCAAGGGCCTCTGAATCTGTCTGTGTCCCTGTTAGCTTTTTTTTTTTTTTTTTTTT | SEQ ID NO: 245 |
| PosControl_119m3 | TTTTGCAAGGGCCTCTGAATCTGTCTGTGTCCCTGTTTTTTTTTTTTTTTTTTTTTTTTT | SEQ ID NO: 246 |
| PosControl_119m4 | TTTTGCAAGGGCCTCTGAATCTGTCTGTGTTTTTTTTTTTTTTTTTTTTTTTTTTTTTTT | SEQ ID NO: 247 |
| PosControl_119m5 | TTTTGCAAGGGCCTCTGAATCTGTCCTTTTTTTTTTTTTTTTTTTTTTTTTTTTTTTTTT | SEQ ID NO: 248 |
| PosControl_120 | GGTCAGAGTATTGGGACCGGGAGACACGGAGCGCCAGGGACACCGCACAGATTTTCCGAG | SEQ ID NO: 249 |
| PosControl_120m1 | GGTCAGAGTATTGGGACCGGGAGACACGGAGCGCCAGGGACACCGTTTTTTTTTTTTTTT | SEQ ID NO: 250 |
| PosControl_120m2 | GGTCAGAGTATTGGGACCGGGAGACACGGAGCGCCTTTTTTTTTTTTTTTTTTTTTTTTT | SEQ ID NO: 251 |
| PosControl_120m3 | GGTCAGAGTATTGGGACCGGGAGACACGGATTTTTTTTTTTTTTTTTTTTTTTTTTTTTT | SEQ ID NO: 252 |
| PosControl_120m4 | GGTCAGAGTATTGGGACCGGGAGACTTTTTTTTTTTTTTTTTTTTTTTTTTTTTTTTTTT | SEQ ID NO: 253 |
| PosControl_120m5 | GGTCAGAGTATTGGGACCGGTTTTTTTTTTTTTTTTTTTTTTTTTTTTTTTTTTTTTTTT | SEQ ID NO: 254 |
| PosControl_121 | AGATTACAAGGGCACTTGGATCCACCCAGAAATTGACAACCCCGAGTATTCTCCCGATCC | SEQ ID NO: 255 |
| PosControl_121m1 | TTTTTACAAGGGCACTTGGATCCACCCAGAAATTGACAACCCCGAGTATTCTCCCGATCC | SEQ ID NO: 256 |
| PosControl_121m2 | TTTTTTTTTTGGCACTTGGATCCACCCAGAAATTGACAACCCCGAGTATTCTCCCGATCC | SEQ ID NO: 257 |
| PosControl_121m3 | TTTTTTTTTTTTTTTTTGGATCCACCCAGAAATTGACAACCCCGAGTATTCTCCCGATCC | SEQ ID NO: 258 |
| PosControl_121m4 | TTTTTTTTTTTTTTTTTTTTTCCACCCAGAAATTGACAACCCCGAGTATTCTCCCGATCC | SEQ ID NO: 259 |
| PosControl_121m5 | TTTTTTTTTTTTTTTTTTTTTTTTTCCAGAAATTGACAACCCCGAGTATTCTCCCGATCC | SEQ ID NO: 260 |
| PosControl_122 | GATGAGTTTACACACCTGTACACACTGATTGTGCGGCCAGACAACACCTATGAGGTGAAG | SEQ ID NO: 261 |
| PosControl_122m1 | GGTAAATCTGCACACCTGTACACACTGATTGTGCGGCCAGACAACACCTATGAGGTGAAG | SEQ ID NO: 262 |
| PosControl_122m2 | GATGAGTTTATATATCCGCACACACTGATTGTGCGGCCAGACAACACCTATGAGGTGAAG | SEQ ID NO: 263 |
| PosControl_122m3 | GATGAGTTTACACACCTGTATATATTAACTGTGCGGCCAGACAACACCTATGAGGTGAAG | SEQ ID NO: 264 |
| PosControl_122m4 | GATGAGTTTACACACCTGTACACACTGATTATACAGTCGGACAACACCTATGAGGTGAAG | SEQ ID NO: 265 |
| PosControl_122m5 | GATGAGTTTACACACCTGTACACACTGATTGTGCGGCCAGGCGATATCCACGAGGTGAAG | SEQ ID NO: 266 |
| PosControl_123 | GCCGGGTGTGGCACAGCTAGTTCCGTCGCAGCCGGGATTTGGGTCGCGGTTCTTGTTTGT | SEQ ID NO: 267 |
| PosControl_123m1 | GCCGGGTGTGGACAGCTAGTTCGTCGCAGCCGGATTTGGGTCGCGTTCTTGTTTGTTTTT | SEQ ID NO: 268 |
| PosControl_123m2 | GCCGGGTGTGCACAGCTATTCCGTCGCGCCGGGATTGGGTCGCGGTCTTGTTTGTTTTTT | SEQ ID NO: 269 |
| PosControl_123m3 | GCCGGGTTGGCACACTAGTTCGTCGCAGCGGGATTGGGTCGCGTTCTTGTTGTTTTTTTT | SEQ ID NO: 270 |
| PosControl_123m4 | GCCGGTGTGGACAGCAGTTCGTCGCGCCGGATTTGGTCCGGTCTTGTTGTTTTTTTTTTT | SEQ ID NO: 271 |
| PosControl_123m5 | GCCGGTTGGACACTATTCGTCCAGCGGATTGGGCGCGTTTTGTTGTTTTTTTTTTTTTTT | SEQ ID NO: 272 |
| PosControl_124 | GTGATCGTCACTTGACAATGCAGATCTTCGTGAAGACTCTGACTGGTAAGACCATCACCC | SEQ ID NO: 273 |
| PosControl_124m1 | GGTGCCTTGACAATGCAGATCTTCGTGAAGACTCTGACTGGTAAGACCATCACCCTTTTT | SEQ ID NO: 274 |
| PosControl_124m2 | GTGATCGTCATGCAGCAGATCTTCGTGAAGACTCTGACTGGTAAGACCATCACCCTTTTT | SEQ ID NO: 275 |
| PosControl_124m3 | GTGATCGTCACTTGACAATGAACTGTGAAGACTCTGACTGGTAAGACCATCACCCTTTTT | SEQ ID NO: 276 |
| PosControl_124m4 | GTGATCGTCACTTGACAATGCAGATCTTCGGAATTGACTGGTAAGACCATCACCCTTTTT | SEQ ID NO: 277 |
| PosControl_124m5 | GTGATCGTCACTTGACAATGCAGATCTTCGTGAAGACTCTATGAGACCATCACCCTTTTT | SEQ ID NO: 278 |
| PosControl_125 | CCAGGAGACTAGACTACTGTTGTCCAGGGTCAATTTGAGTGTAAAGAAAATGTAGACAAG | SEQ ID NO: 279 |
| PosControl_125m1 | CAGGCAATCTGTTGTCCAGGGTCAATTTGAGTGTAAAGAAAATGTAGACAAGTTTTTTTT | SEQ ID NO: 280 |
| PosControl_125m2 | CCAGGAGACTGCATTGCAGGGTCAATTTGAGTGTAAAGAAAATGTAGACAAGTTTTTTTT | SEQ ID NO: 281 |
| PosControl_125m3 | CCAGGAGACTAGACTACTGTGCAGTATTGAGTGTAAAGAAAATGTAGACAAGTTTTTTTT | SEQ ID NO: 282 |
| PosControl_125m4 | CCAGGAGACTAGACTACTGTTGTCCAGGGTATTATTAGAAAATGTAGACAAGTTTTTTTT | SEQ ID NO: 283 |
| PosControl_125m5 | CCAGGAGACTAGACTACTGTTGTCCAGGGTCAATTTGAGTTAGAAGAACAAGTTTTTTTT | SEQ ID NO: 284 |
| PosControl_126 | ATTTATCATAACAAAAATACTACTAATATAACTACTGTTTCCATGTCCCATGATCCCCTC | SEQ ID NO: 285 |
| PosControl_126m1 | CCCCATCATAACAAAAATACTACTAATATAACTACTGTTTCCATGTCCCATGATCCCCTC | SEQ ID NO: 286 |
| PosControl_126m2 | CCCCCCCATAACAAAAATACTACTAATATAACTACTGTTTCCATGTCCCATGATCCCCTC | SEQ ID NO: 287 |
| PosControl_126m3 | CCCCCCTCTAACAAAAATACTACTAATATAACTACTGTTTCCATGTCCCATGATCCCCTC | SEQ ID NO: 288 |
| PosControl_126m4 | CCCCCCCCCCACAAAAATACTACTAATATAACTACTGTTTCCATGTCCCATGATCCCCTC | SEQ ID NO: 289 |
| PosControl_126m5 | CCCCCCCCCCCTAAAAATACTACTAATATAACTACTGTTTCCATGTCCCATGATCCCCTC | SEQ ID NO: 290 |
| PosControl_127 | TGTAGTGTGTTCCATTTAAAATTTTGGCATATGGCATTTTCTAACTTAGGAAGCCACAAT | SEQ ID NO: 291 |
| PosControl_127m1 | CCCCCCGTGTTCCATTTAAAATTTTGGCATATGGCATTTTCTAACTTAGGAAGCCACAAT | SEQ ID NO: 292 |
| PosControl_127m2 | TGTAGTGTGTCTTCCCTAAAATTTTGGCATATGGCATTTTCTAACTTAGGAAGCCACAAT | SEQ ID NO: 293 |
| PosControl_127m3 | TGTAGTGTGTTCCATTTAAACCCCCCGCATATGGCATTTTCTAACTTAGGAAGCCACAAT | SEQ ID NO: 294 |
| PosControl_127m4 | TGTAGTGTGTTCCATTTAAAATTTTGGCATCCCCTCTTTTCTAACTTAGGAAGCCACAAT | SEQ ID NO: 295 |
| PosControl_127m5 | TGTAGTGTGTTCCATTTAAAATTTTGGCATATGGCATTTTTCCCTCTAGGAAGCCACAAT | SEQ ID NO: 296 |
| PosControl_128 | CATTCTTTGAAATTTATTCCTTATTCCCTCTTGGCAGCTAATGGGCTCTTACCAAGTTTA | SEQ ID NO: 297 |
| PosControl_128m1 | CATTTCCCCCAATTTATTCCTTATTCCCTCTTGGCAGCTAATGGGCTCTTACCAAGTTTA | SEQ ID NO: 298 |
| PosControl_128m2 | CATTCTTTGAAATTCCCCTTTTATTCCCTCTTGGCAGCTAATGGGCTCTTACCAAGTTTA | SEQ ID NO: 299 |
| PosControl_128m3 | CATTCTTTGAAATTTATTCCTTATCTTTCTTTGGCAGCTAATGGGCTCTTACCAAGTTTA | SEQ ID NO: 300 |
| PosControl_128m4 | CATTCTTTGAAATTTATTCCTTATTCCCTCTTGGTCCTCCATGGGCTCTTACCAAGTTTA | SEQ ID NO: 301 |
| PosControl_128m5 | CATTCTTTGAAATTTATTCCTTATTCCCTCTTGGCAGCTAATGGCTCTCCACCAAGTTTA | SEQ ID NO: 302 |
| PosControl_129 | TTGATACTTGCCTAACATGCATGTGCTGTAAAAATAGTTAACAGGGAAATAACTTGAGAT | SEQ ID NO: 303 |
| PosControl_129m1 | TTGATACTTGCCTAACATGCATGTGCTGTAAAAACTTCCTACAGGGAAATAACTTGAGAT | SEQ ID NO: 304 |
| PosControl_129m2 | TTGATACTTGCCTAACATGCATGTGCTGTATTTTCTTCCTACAGGGAAATAACTTGAGAT | SEQ ID NO: 305 |
| PosControl_129m3 | TTGATACTTGCCTAACATGCATGTGTCTCTTTTTCTTCCTACAGGGAAATAACTTGAGAT | SEQ ID NO: 306 |
| PosControl_129m4 | TTGATACTTGCCTAACATGCTCTCTTCTCTTTTTCTTCCTACAGGGAAATAACTTGAGAT | SEQ ID NO: 307 |
| PosControl_129m5 | TTGATACTTGCCTAATTCTTTCTCTTCTCTTTTTCTTCCTACAGGGAAATAACTTGAGAT | SEQ ID NO: 308 |
| PosControl_130 | CTGGGAAATCGAAGATTGAGGACTACTTTCCAGAATTTGCTCGCTACACTACTCCTGAGG | SEQ ID NO: 309 |
| PosControl_130m1 | CTGGGAAATCGAAGATTGAGGACTACTTTCCAGAACCCTTTCGCTACACTACTCCTGAGG | SEQ ID NO: 310 |
| PosControl_130m2 | CTGGGAAATCGAAGATTGAGGACTACTTTCTTTTTCCCTTTCGCTACACTACTCCTGAGG | SEQ ID NO: 311 |
| PosControl_130m3 | CTGGGAAATCGAAGATTGAGGACTATCCCTTTTTTCCCTTTCGCTACACTACTCCTGAGG | SEQ ID NO: 312 |
| PosControl_130m4 | CTGGGAAATCGAAGATTGAGTTTCTTCCCTTTTTTCCCTTTCGCTACACTACTCCTGAGG | SEQ ID NO: 313 |
| PosControl_130m5 | CTGGGAAATCGAAGACCTTTTTTCTTCCCTTTTTTCCCTTTCGCTACACTACTCCTGAGG | SEQ ID NO: 314 |
| PosControl_131 | TCCTGGACAAGATCGACGTGATCAAGCAGGCTGACTATGTGCCGAGCGATCAGGACCTGC | SEQ ID NO: 315 |
| PosControl_131m1 | CTTCGGACAAGATCGACGTGATCAAGCAGGCTGACTATGTGCCGAGCGATCAGGACCTGC | SEQ ID NO: 316 |
| PosControl_131m2 | CTTCTTTTAAGATCGACGTGATCAAGCAGGCTGACTATGTGCCGAGCGATCAGGACCTGC | SEQ ID NO: 317 |
| PosControl_131m3 | CTTCTTTTTTTTTCGACGTGATCAAGCAGGCTGACTATGTGCCGAGCGATCAGGACCTGC | SEQ ID NO: 318 |
| PosControl_131m4 | CTTCTTTTTTTTCTTTCGTGATCAAGCAGGCTGACTATGTGCCGAGCGATCAGGACCTGC | SEQ ID NO: 319 |
| PosControl_131m5 | CTTCTTTTTTTTCTTTTTCTATCAAGCAGGCTGACTATGTGCCGAGCGATCAGGACCTGC | SEQ ID NO: 320 |
| PosControl_132 | CCCCAACTTGAGATGTATGAAGGCTTTTGGTCTCCCTGGGAGTGGGTGGAGGCAGCCAGG | SEQ ID NO: 321 |
| PosControl_132m1 | CCCCAACTTGAGATGCTCTTAGGCTTTTGGTCTCCCTGGGAGTGGGTGGAGGCAGCCAGG | SEQ ID NO: 322 |
| PosControl_132m2 | CCCCAACTTGAGACTCTCTTTTGCTTTTGGTCTCCCTGGGAGTGGGTGGAGGCAGCCAGG | SEQ ID NO: 323 |
| PosControl_132m3 | CCCCAACTTGATTCTCTCTTTTTTTTTGGTCTCCCTGGGAGTGGGTGGAGGCAGCCAGG | SEQ ID NO: 324 |
| PosControl_132m4 | CCCCAACTTTTTTCTCTCTTTTTTCCTTGGTCTCCCTGGGAGTGGGTGGAGGCAGCCAGG | SEQ ID NO: 325 |
| PosControl_132m5 | CCCCAACCCTTTTCTCTCTTTTTTCCCCGGTCTCCCTGGGAGTGGGTGGAGGCAGCCAGG | SEQ ID NO: 326 |
| PosControl_133 | ATTTAAAAACTGGAACGGTGAAGGTGACAGCAGTCGGTTGGAGCGAGCATCCCCCAAAGT | SEQ ID NO: 327 |
| PosControl_133m1 | ATTTAAAAACTGGAATTTCTAAGGTGACAGCAGTCGGTTGGAGCGAGCATCCCCCAAAGT | SEQ ID NO: 328 |
| PosControl_133m2 | ATTTAAAAACTGGTTTTTCTTTGGTGACAGCAGTCGGTTGGAGCGAGCATCCCCCAAAGT | SEQ ID NO: 329 |
| PosControl_133m3 | ATTTAAAAACTTTTTTTTCTTTTTTGACAGCAGTCGGTTGGAGCGAGCATCCCCCAAAGT | SEQ ID NO: 330 |
| PosControl_133m4 | ATTTAAAAATCTTTTTTTCTTTTTCTACAGCAGTCGGTTGGAGCGAGCATCCCCCAAAGT | SEQ ID NO: 331 |
| PosControl_133m5 | ATTTAAATTTCTTTTTTTCTTTTTCTTTAGCAGTCGGTTGGAGCGAGCATCCCCCAAAGT | SEQ ID NO: 332 |
| PosControl_134 | AAGAACATCACCCAGCGCGGCAACGTCGCCAAGACCTCGAGAAATGCCCCCGAAGAGAAG | SEQ ID NO: 333 |
| PosControl_134m1 | GTTGACATCACCCAGCGCGGCAACGTCGCCAAGACCTCGAGAAATGCCCCCGAAGAGAAG | SEQ ID NO: 334 |
| PosControl_134m2 | CGTTGCATCACCCAGCGCGGCAACGTCGCCAAGACCTCGAGAAATGCCCCCGAAGAGAAG | SEQ ID NO: 335 |
| PosControl_134m3 | ACGTTGATCACCCAGCGCGGCAACGTCGCCAAGACCTCGAGAAATGCCCCCGAAGAGAAG | SEQ ID NO: 336 |
| PosControl_134m4 | GACGTTGTCACCCAGCGCGGCAACGTCGCCAAGACCTCGAGAAATGCCCCCGAAGAGAAG | SEQ ID NO: 337 |
| PosControl_134m5 | CGACGTTGCACCCAGCGCGGCAACGTCGCCAAGACCTCGAGAAATGCCCCCGAAGAGAAG | SEQ ID NO: 338 |
| PosControl_135 | TATCATGCTTCATGTGTCATTCCAAGGTTTCTTCATGAGTCATTCCAAGTTTTCTAGTCC | SEQ ID NO: 339 |
| PosControl_135m1 | TATCATGCTTCATGTGTCCAAGGTTTCTTCATGAGTCATTCCAAGTTTTCTAGTCCTTTT | SEQ ID NO: 340 |
| PosControl_135m2 | TATCATGCTTCATGTCCAAGGTTTCTTCATGAGTCATTCCAAGTTTTCTAGTCCTTTTTT | SEQ ID NO: 341 |
| PosControl_135m3 | TATCATGCTTCATGCAAGGTTTCTTCATGAGTCATTCCAAGTTTTCTAGTCCTTTTTTTT | SEQ ID NO: 342 |
| PosControl_135m4 | TATCATGCTTCATAAGGTTTCTTCATGAGTCATTCCAAGTTTTCTAGTCCTTTTTTTTTT | SEQ ID NO: 343 |
| PosControl_135m5 | TATCATGCTTCGAGGTTTCTTCATGAGTCATTCCAAGTTTTCTAGTCCTTTTTTTTTTTT | SEQ ID NO: 344 |
| PosControl_136 | CAGGTGCCACAGGCAGCCCTGGGACATAGGAAGCTGGGAGCAAGGAAAGGGTCTTAGTCA | SEQ ID NO: 345 |
| PosControl_136m1 | CAGGTGCCACAGGCAGCCCTGGGACAAAGCTGGGAGCAAGGAAAGGGTCTTAGTCATTTT | SEQ ID NO: 346 |
| PosControl_136m2 | CAGGTGCCACAGGCAGCCCTGGGACAGCTGGGAGCAAGGAAAGGGTCTTAGTCATTTTTT | SEQ ID NO: 347 |
| PosControl_136m3 | CAGGTGCCACAGGCAGCCCTGGGAGCTGGGAGCAAGGAAAGGGTCTTAGTCATTTTTTTT | SEQ ID NO: 348 |
| PosControl_136m4 | CAGGTGCCACAGGCAGCCCTGGGCTGGGAGCAAGGAAAGGGTCTTAGTCATTTTTTTTTT | SEQ ID NO: 349 |
| PosControl_136m5 | CAGGTGCCACAGGCAGCCCTGGTGGGAGCAAGGAAAGGGTCTTAGTCATTTTTTTTTTTT | SEQ ID NO: 350 |
| PosControl_137 | ACATCTACAATGTTGGCTCAAGTGCTGCATTAGACGTGGAACTATCTGATGATTCCTTCC | SEQ ID NO: 351 |
| PosControl_137m1 | ACATCTTGTTGGCTCAAGTGCTGCATTAGACGTGGAACTATCTGATGATTCCTTCCTTTT | SEQ ID NO: 352 |
| PosControl_137m2 | ACATCGTTGGCTCAAGTGCTGCATTAGACGTGGAACTATCTGATGATTCCTTCCTTTTTT | SEQ ID NO: 353 |
| PosControl_137m3 | ACATTTGGCTCAAGTGCTGCATTAGACGTGGAACTATCTGATGATTCCTTCCTTTTTTTT | SEQ ID NO: 354 |
| PosControl_137m4 | ACATGGCTCAAGTGCTGCATTAGACGTGGAACTATCTGATGATTCCTTCCTTTTTTTTTT | SEQ ID NO: 355 |
| PosControl_137m5 | ACGGCTCAAGTGCTGCATTAGACGTGGAACTATCTGATGATTCCTTCCTTTTTTTTTTTT | SEQ ID NO: 356 |
| PosControl_138 | GCTGCAGAATGGCTCCCGCAAAGAAGGGTGGCGAGAAGAAAAAGGGCCGTTCTGCCATCA | SEQ ID NO: 357 |
| PosControl_138m1 | GCTGCAGAATGCCGCAAAGAAGGGTGGCGAGAAGAAAAAGGGCCGTTCTGCCATCATTTT | SEQ ID NO: 358 |
| PosControl_138m2 | GCTGCAGAATCGCAAAGAAGGGTGGCGAGAAGAAAAAGGGCCGTTCTGCCATCATTTTTT | SEQ ID NO: 359 |
| PosControl_138m3 | GCTGCAGAAGCAAAGAAGGGTGGCGAGAAGAAAAAGGGCCGTTCTGCCATCATTTTTTTT | SEQ ID NO: 360 |
| PosControl_138m4 | GCTGCAGACAAAGAAGGGTGGCGAGAAGAAAAAGGGCCGTTCTGCCATCATTTTTTTTTT | SEQ ID NO: 361 |
| PosControl_138m5 | GCTGCAGAAAGAAGGGTGGCGAGAAGAAAAAGGGCCGTTCTGCCATCATTTTTTTTTTTT | SEQ ID NO: 362 |
| PosControl_139 | ATGTGCCATACCGAATCCGTGTGCGGCTGTCCAGAAAACGTAATGAGGATGAAGATTCAC | SEQ ID NO: 363 |
| PosControl_139m1 | ATGTGCCCGAATCCGTGTGCGGCTGTCCAGAAAACGTAATGAGGATGAAGATTCACTTTT | SEQ ID NO: 364 |
| PosControl_139m2 | ATGTGCGAATCCGTGTGCGGCTGTCCAGAAAACGTAATGAGGATGAAGATTCACTTTTTT | SEQ ID NO: 365 |
| PosControl_139m3 | ATGTGAATCCGTGTGCGGCTGTCCAGAAAACGTAATGAGGATGAAGATTCACTTTTTTTT | SEQ ID NO: 366 |
| PosControl_139m4 | ATGAATCCGTGTGCGGCTGTCCAGAAAACGTAATGAGGATGAAGATTCACTTTTTTTTTT | SEQ ID NO: 367 |
| PosControl_139m5 | ATATCCGTGTGCGGCTGTCCAGAAAACGTAATGAGGATGAAGATTCACTTTTTTTTTTTT | SEQ ID NO: 368 |
| PosControl_140 | GAGTGGGCTTCAAGAAGCGTGCACCTCGGGCACTCAAAGAGATTCGGAAATTTGCCATGA | SEQ ID NO: 369 |
| PosControl_140m1 | GAGTGGGCTTCAGCGTGCACCTCGGGCACTCAAAGAGATTCGGAAATTTGCCATGATTTT | SEQ ID NO: 370 |
| PosControl_140m2 | GAGTGGGCTTGCGTGCACCTCGGGCACTCAAAGAGATTCGGAAATTTGCCATGATTTTTT | SEQ ID NO: 371 |
| PosControl_140m3 | GAGTGGGCTCGTGCACCTCGGGCACTCAAAGAGATTCGGAAATTTGCCATGATTTTTTTT | SEQ ID NO: 372 |
| PosControl_140m4 | GAGTGGGCGTGCACCTCGGGCACTCAAAGAGATTCGGAAATTTGCCATGATTTTTTTTTT | SEQ ID NO: 373 |
| PosControl_140m5 | GAGTGGGTGCACCTCGGGCACTCAAAGAGATTCGGAAATTTGCCATGATTTTTTTTTTTT | SEQ ID NO: 374 |
| PosControl_141 | GTCTGAAAAAGGTATTGCAGTCAGAACTGTGTACTGATGATAAAAGCCTCTGGTAGCAAT | SEQ ID NO: 375 |
| PosControl_141m1 | GTCTGAAAAAGGTATTTCAGAACTGTGTACTGATGATAAAAGCCTCTGGTAGCAATTTTT | SEQ ID NO: 376 |
| PosControl_141m2 | GTCTGAAAAAGGTATCAGAACTGTGTACTGATGATAAAAGCCTCTGGTAGCAATTTTTTT | SEQ ID NO: 377 |
| PosControl_141m3 | GTCTGAAAAAGGTAAGAACTGTGTACTGATGATAAAAGCCTCTGGTAGCAATTTTTTTTT | SEQ ID NO: 378 |
| PosControl_141m4 | GTCTGAAAAAGGTGAACTGTGTACTGATGATAAAAGCCTCTGGTAGCAATTTTTTTTTTT | SEQ ID NO: 379 |
| PosControl_141m5 | GTCTGAAAAAGGAACTGTGTACTGATGATAAAAGCCTCTGGTAGCAATTTTTTTTTTTTT | SEQ ID NO: 380 |
| PosControl_142 | GTAAACTAAGGTAGACTACTTTGGGAATGAGAACATGCAAAATCAGGAAAGGCTGTAGAA | SEQ ID NO: 381 |
| PosControl_142m1 | GTAAACTAAGGTAGACTACTTAATGAGAACATGCAAAATCAGGAAAGGCTGTAGAATTTT | SEQ ID NO: 382 |
| PosControl_142m2 | GTAAACTAAGGTAGACTACTATGAGAACATGCAAAATCAGGAAAGGCTGTAGAATTTTTT | SEQ ID NO: 383 |
| PosControl_142m3 | GTAAACTAAGGTAGACTACTGAGAACATGCAAAATCAGGAAAGGCTGTAGAATTTTTTTT | SEQ ID NO: 384 |
| PosControl_142m4 | GTAAACTAAGGTAGACTAGAGAACATGCAAAATCAGGAAAGGCTGTAGAATTTTTTTTTT | SEQ ID NO: 385 |
| PosControl_142m5 | GTAAACTAAGGTAGACTAGAACATGCAAAATCAGGAAAGGCTGTAGAATTTTTTTTTTTT | SEQ ID NO: 386 |
| PosControl_143 | GCAGCCATGGCCCCAATCAAGGTGGGAGATGCCATCCCAGCAGTGGAGGTGTTTGAAGGG | SEQ ID NO: 387 |
| PosControl_143m1 | GCAGCCATGGCCCCAATCAAGGTGGGGCCATCCCAGCAGTGGAGGTGTTTGAAGGGTTTT | SEQ ID NO: 388 |
| PosControl_143m2 | GCAGCCATGGCCCCAATCAAGGTGGCCATCCCAGCAGTGGAGGTGTTTGAAGGGTTTTTT | SEQ ID NO: 389 |
| PosControl_143m3 | GCAGCCATGGCCCCAATCAAGGTGCATCCCAGCAGTGGAGGTGTTTGAAGGGTTTTTTTT | SEQ ID NO: 390 |
| PosControl_143m4 | GCAGCCATGGCCCCAATCAAGGTATCCCAGCAGTGGAGGTGTTTGAAGGGTTTTTTTTTT | SEQ ID NO: 391 |
| PosControl_143m5 | GCAGCCATGGCCCCAATCAAGGTCCCAGCAGTGGAGGTGTTTGAAGGGTTTTTTTTTTTT | SEQ ID NO: 392 |
| PosControl_144 | AGGGAGTCCAGGTGGTGGCCTGTCTGAGTGTTAATGATGCCTTTGTGACTGGCGAGTGGG | SEQ ID NO: 393 |
| PosControl_144m1 | GACAAGTCCAGGTGGTGGCCTGTCTGAGTGTTAATGATGCCTTTGTGACTGGCGAGTGGG | SEQ ID NO: 394 |
| PosControl_144m2 | AGACAGTCCAGGTGGTGGCCTGTCTGAGTGTTAATGATGCCTTTGTGACTGGCGAGTGGG | SEQ ID NO: 395 |
| PosControl_144m3 | CAGACATCCAGGTGGTGGCCTGTCTGAGTGTTAATGATGCCTTTGTGACTGGCGAGTGGG | SEQ ID NO: 396 |
| PosControl_144m4 | TCAGACACCAGGTGGTGGCCTGTCTGAGTGTTAATGATGCCTTTGTGACTGGCGAGTGGG | SEQ ID NO: 397 |
| PosControl_144m5 | CTCAGACACAGGTGGTGGCCTGTCTGAGTGTTAATGATGCCTTTGTGACTGGCGAGTGGG | SEQ ID NO: 398 |
| PosControl_145 AGGCACATCATTGGAATACAGGAAGTAGCCCTGCACCTGCCAGTGAGCTCGCCATTCACT | SEQ ID NO: 399 | |
| PosControl_145m1 | CCTGACATCATTGGAATACAGGAAGTAGCCCTGCACCTGCCAGTGAGCTCGCCATTCACT | SEQ ID NO: 400 |
| PosControl_145m2 | TCCTGCATCATTGGAATACAGGAAGTAGCCCTGCACCTGCCAGTGAGCTCGCCATTCACT | SEQ ID NO: 401 |
| PosControl_145m3 | TTCCTGATCATTGGAATACAGGAAGTAGCCCTGCACCTGCCAGTGAGCTCGCCATTCACT | SEQ ID NO: 402 |
| PosControl_145m4 | CTTCCTGTCATTGGAATACAGGAAGTAGCCCTGCACCTGCCAGTGAGCTCGCCATTCACT | SEQ ID NO: 403 |
| PosControl_145m5 | ACTTCCTGCATTGGAATACAGGAAGTAGCCCTGCACCTGCCAGTGAGCTCGCCATTCACT | SEQ ID NO: 404 |
| PosControl_146 | TGTGTCCACATAATCCACGCTCATCTTGCAAAGCGCTATTTCAGGCACATCATTGGAATA | SEQ ID NO: 405 |
| PosControl_146m1 | TGAGACGAGATAATCCACGCTCATCTTGCAAAGCGCTATTTCAGGCACATCATTGGAATA | SEQ ID NO: 406 |
| PosControl_146m2 | TGTGACGAGAAAATCCACGCTCATCTTGCAAAGCGCTATTTCAGGCACATCATTGGAATA | SEQ ID NO: 407 |
| PosControl_146m3 | TGTGTCGAGAAACTCCACGCTCATCTTGCAAAGCGCTATTTCAGGCACATCATTGGAATA | SEQ ID NO: 408 |
| PosControl_146m4 | TGTGTCCAGAAACTGCACGCTCATCTTGCAAAGCGCTATTTCAGGCACATCATTGGAATA | SEQ ID NO: 409 |
| PosControl_146m5 | TGTGTCCACAAACTGCCCGCTCATCTTGCAAAGCGCTATTTCAGGCACATCATTGGAATA | SEQ ID NO: 410 |
| PosControl_147 | GGATTGTTTATACTCCAGTGTACATAGTGTAATGTAGCGTGTTTACATGTGTAGCCTATG | SEQ ID NO: 411 |
| PosControl_147m1 | GGCTAGATAAAACTCCAGTGTACATAGTGTAATGTAGCGTGTTTACATGTGTAGCCTATG | SEQ ID NO: 412 |
| PosControl_147m2 | GGATTGATAAAAGTGCAGTGTACATAGTGTAATGTAGCGTGTTTACATGTGTAGCCTATG | SEQ ID NO: 413 |
| PosControl_147m3 | GGATTGTTTAAAGTGCCGAGTACATAGTGTAATGTAGCGTGTTTACATGTGTAGCCTATG | SEQ ID NO: 414 |
| PosControl_147m4 | GGATTGTTTATACTGCCGAGAAGATAGTGTAATGTAGCGTGTTTACATGTGTAGCCTATG | SEQ ID NO: 415 |
| PosControl_147m5 | GGATTGTTTATACTCCAGAGAAGAAATTGTAATGTAGCGTGTTTACATGTGTAGCCTATG | SEQ ID NO: 416 |
| PosControl_148 | GGCGCCGATTCCTACAAAGATGCTGTCCGGAAAGCCATGTTTGCCCGGTTCACTGAGATG | SEQ ID NO: 417 |
| PosControl_148m1 | GGCTAATCGGCCTACAAAGATGCTGTCCGGAAAGCCATGTTTGCCCGGTTCACTGAGATG | SEQ ID NO: 418 |
| PosControl_148m2 | GGCGCCGATGAAGCGCAAGATGCTGTCCGGAAAGCCATGTTTGCCCGGTTCACTGAGATG | SEQ ID NO: 419 |
| PosControl_148m3 | GGCGCCGATTCCTACCCCTCATCTGTCCGGAAAGCCATGTTTGCCCGGTTCACTGAGATG | SEQ ID NO: 420 |
| PosControl_148m4 | GGCGCCGATTCCTACAAAGATTAGTGAAGGAAAGCCATGTTTGCCCGGTTCACTGAGATG | SEQ ID NO: 421 |
| PosControl_148m5 | GGCGCCGATTCCTACAAAGATGCTGTCCTTCCCTACATGTTTGCCCGGTTCACTGAGATG | SEQ ID NO: 422 |
| PosControl_149 | TCATCCCAGCTGGTGTAATAATGAATTGTTTAAAAAACAGCTCATAATTGATGCCAAATT | SEQ ID NO: 423 |
| PosControl_149m1 | TCATGGTGTAATAATGAATTGTTTAAAAAACAGCTCATAATTGATGCCAAATTTTTTTTT | SEQ ID NO: 424 |
| PosControl_149m2 | TCATCCCAGAATAATGAATTGTTTAAAAAACAGCTCATAATTGATGCCAAATTTTTTTTT | SEQ ID NO: 425 |
| PosControl_149m3 | TCATCCCAGCTGGTGGAATTGTTTAAAAAACAGCTCATAATTGATGCCAAATTTTTTTTT | SEQ ID NO: 426 |
| PosControl_149m4 | TCATCCCAGCTGGTGTAATAATTTAAAAAACAGCTCATAATTGATGCCAAATTTTTTTTT | SEQ ID NO: 427 |
| PosControl_149m5 | TCATCCCAGCTGGTGTAATAATGAATTAAACAGCTCATAATTGATGCCAAATTTTTTTTT | SEQ ID NO: 428 |
| PosControl_150 | AAGGGTACCTGAAGCGAATTGGCACCAAAGCAGCAGCTGTATTGCCGCAGTTCTAGCTTC | SEQ ID NO: 429 |
| PosControl_150m1 | AAGGGTACCTGAAGCTTTCCTTCACCAAAGCAGCAGCTGTATTGCCGCAGTTCTAGCTTC | SEQ ID NO: 430 |
| PosControl_150m2 | AAGGGTACCTGAAGCCCCCCCCCACCAAAGCAGCAGCTGTATTGCCGCAGTTCTAGCTTC | SEQ ID NO: 431 |
| PosControl_150m3 | AAGGGTACCTGAAGCCTTAACCCACCAAAGCAGCAGCTGTATTGCCGCAGTTCTAGCTTC | SEQ ID NO: 432 |
| PosControl_150m4 | AAGGGTACCTGAAGCTCCGGTTCACCAAAGCAGCAGCTGTATTGCCGCAGTTCTAGCTTC | SEQ ID NO: 433 |
| PosControl_150m5 | AAGGGTACCTGAAGCCACCAAAGCAGCAGCTGTATTGCCGCAGTTCTAGCTTCTTTTTTT | SEQ ID NO: 434 |
| PosControl_151 | TCCAACGACAAGACGAGGATTCTCAACGTCCAGTCCAGGCTCACAGACGCCAAACGCATT | SEQ ID NO: 435 |
| PosControl_151m1 | TCCAATTTTTTTACGAGGATTCTCAACGTCCAGTCCAGGCTCACAGACGCCAAACGCATT | SEQ ID NO: 436 |
| PosControl_151m2 | TCCAACCCCCCCACGAGGATTCTCAACGTCCAGTCCAGGCTCACAGACGCCAAACGCATT | SEQ ID NO: 437 |
| PosControl_151m3 | TCCAAGCTGTTCACGAGGATTCTCAACGTCCAGTCCAGGCTCACAGACGCCAAACGCATT | SEQ ID NO: 438 |
| PosControl_151m4 | TCCAAATCACCTACGAGGATTCTCAACGTCCAGTCCAGGCTCACAGACGCCAAACGCATT | SEQ ID NO: 439 |
| PosControl_151m5 | TCCAAACGAGGATTCTCAACGTCCAGTCCAGGCTCACAGACGCCAAACGCATTTTTTTTT | SEQ ID NO: 440 |
| PosControl_152 | TTCTACTCCGATGATCGGCTGAATGTAACAGAGGAACTAACGTCCAACGACAAGACGAGG | SEQ ID NO: 441 |
| PosControl_152m1 | TTCTACTCCGTCTTCTTGCTGAATGTAACAGAGGAACTAACGTCCAACGACAAGACGAGG | SEQ ID NO: 442 |
| PosControl_152m2 | TTCTACTCCGCCCCCTCGCTGAATGTAACAGAGGAACTAACGTCCAACGACAAGACGAGG | SEQ ID NO: 443 |
| PosControl_152m3 | TTCTACTCCGTACTAGCGCTGAATGTAACAGAGGAACTAACGTCCAACGACAAGACGAGG | SEQ ID NO: 444 |
| PosControl_152m4 | TTCTACTCCGCGTCGATGCTGAATGTAACAGAGGAACTAACGTCCAACGACAAGACGAGG | SEQ ID NO: 445 |
| PosControl_152m5 | TTCTACTCCGGCTGAATGTAACAGAGGAACTAACGTCCAACGACAAGACGAGGTTTTTTT | SEQ ID NO: 446 |
| PosControl_153 | GGCCACCCATTCAGGGCATATCTGGAATCTGAAGTTGCTATATCTGAGGAGTTGGTTCAG | SEQ ID NO: 447 |
| PosControl_153m1 | GGCCTTTTTCCTTGGGCATATCTGGAATCTGAAGTTGCTATATCTGAGGAGTTGGTTCAG | SEQ ID NO: 448 |
| PosControl_153m2 | GGCCCTTTCCCTCGGGCATATCTGGAATCTGAAGTTGCTATATCTGAGGAGTTGGTTCAG | SEQ ID NO: 449 |
| PosControl_153m3 | GGCCTGGGTAAGTOGGCATATCTGGAATCTGAAGTTGCTATATCTGAGGAGTTGGTTCAG | SEQ ID NO: 450 |
| PosControl_153m4 | GGCCCAAACGGACGGGCATATCTGGAATCTGAAGTTGCTATATCTGAGGAGTTGGTTCAG | SEQ ID NO: 451 |
| PosControl_153m5 | GGCCGGGCATATCTGGAATCTGAAGTTGCTATATCTGAGGAGTTGGTTCAGTTTTTTTTT | SEQ ID NO: 452 |
| PosControl_154 | TACTACCCCGCAGTTCCCGGACATGATCCTCTCCGCCTCTCGAGATAAGACCATCATCAT | SEQ ID NO: 453 |
| PosControl_154m1 | TACTACCCCGCAGTCTTTTTTTTCGATCCTCTCCGCCTCTCGAGATAAGACCATCATCAT | SEQ ID NO: 454 |
| PosControl_154m2 | TACTACCCCGCAGTCTTTCCCTCCGATCCTCTCCGCCTCTCGAGATAAGACCATCATCAT | SEQ ID NO: 455 |
| PosControl_154m3 | TACTACCCCGCAGTAGGGCCTGTAGATCCTCTCCGCCTCTCGAGATAAGACCATCATCAT | SEQ ID NO: 456 |
| PosControl_154m4 | TACTACCCCGCAGTGAAATTCACGGATCCTCTCCGCCTCTCGAGATAAGACCATCATCAT | SEQ ID NO: 457 |
| PosControl_154m5 | TACTACCCCGCAGTGATCCTCTCCGCCTCTCGAGATAAGACCATCATCATTTTTTTTTTT | SEQ ID NO: 458 |
| PosControl_155 | AGCCATTTTCAGTTATTATACCAGAACCTCTTCACATAGACCTATTAGTGCATTTGTAAC | SEQ ID NO: 459 |
| PosControl_155m1 | AGCCATTTTTTTCCTCCTCACCAGAACCTCTTCACATAGACCTATTAGTGCATTTGTAAC | SEQ ID NO: 460 |
| PosControl_155m2 | AGCCATTTTGCTAACAACAACCAGAACCTCTTCACATAGACCTATTAGTGCATTTGTAAC | SEQ ID NO: 461 |
| PosControl_155m3 | AGCCATTTTTCCCCCCCCCACCAGAACCTCTTCACATAGACCTATTAGTGCATTTGTAAC | SEQ ID NO: 462 |
| PosControl_155m4 | AGCCATTTTGTCAATAATAACCAGAACCTCTTCACATAGACCTATTAGTGCATTTGTAAC | SEQ ID NO: 463 |
| PosControl_155m5 | AGCCATTTTACCAGAACCTCTTCACATAGACCTATTAGTGCATTTGTAACTTTTTTTTTT | SEQ ID NO: 464 |
| PosControl_156 | AAAGTCAAATTTCCTGAATTGCTATGTGTCTGGGTTTCATCCATCCGACATTGAAGTTGA | SEQ ID NO: 465 |
| PosControl_156m1 | AAAGTTTTTCCCCCTGAATTGCTATGTGTCTGGGTTTCATCCATCCGACATTGAAGTTGA | SEQ ID NO: 466 |
| PosControl_156m2 | AAAGTTCCCCCCCCTGAATTGCTATGTGTCTGGGTTTCATCCATCCGACATTGAAGTTGA | SEQ ID NO: 467 |
| PosControl_156m3 | AAAGTGTTTAAACCTGAATTGCTATGTGTCTGGGTTTCATCCATCCGACATTGAAGTTGA | SEQ ID NO: 468 |
| PosControl_156m4 | AAAGTGCCCGGGCCTGAATTGCTATGTGTCTGGGTTTCATCCATCCGACATTGAAGTTGA | SEQ ID NO: 469 |
| PosControl_156m5 | AAAGTCCTGAATTGCTATGTGTCTGGGTTTCATCCATCCGACATTGAAGTTGATTTTTTT | SEQ ID NO: 470 |
| PosControl_157 | CATGTGACTTTGTCACAGCCCAAGATAGTTAAGTGGGATCGAGACATGTAAGCAGCATCA | SEQ ID NO: 471 |
| PosControl_157m1 | CATGTTTTCCCTCTTCAGCCCAAGATAGTTAAGTGGGATCGAGACATGTAAGCAGCATCA | SEQ ID NO: 472 |
| PosControl_157m2 | CATGTCCTCCCCCTCCAGCCCAAGATAGTTAAGTGGGATCGAGACATGTAAGCAGCATCA | SEQ ID NO: 473 |
| PosControl_157m3 | CATGTCTGAAACAGTCAGCCCAAGATAGTTAAGTGGGATCGAGACATGTAAGCAGCATCA | SEQ ID NO: 474 |
| PosControl_157m4 | CATGTTCAGGGTGGCCAGCCCAAGATAGTTAAGTGGGATCGAGACATGTAAGCAGCATCA | SEQ ID NO: 475 |
| PosControl_157m5 | CATGTCAGCCCAAGATAGTTAAGTGGGATCGAGACATGTAAGCAGCATCATTTTTTTTTT | SEQ ID NO: 476 |
| PosControl_158 | CTCCAAAGATTCAGGTTTACTCACGTCATCCAGCAGAGAATGGAAAGTCAAATTTCCTGA | SEQ ID NO: 477 |
| PosControl_158m1 | CTCCAAAGTCCTTTTCCCACTCACGTCATCCAGCAGAGAATGGAAAGTCAAATTTCCTGA | SEQ ID NO: 478 |
| PosControl_158m2 | CTCCAAAGCCCTCCCCCCACTCACGTCATCCAGCAGAGAATGGAAAGTCAAATTTCCTGA | SEQ ID NO: 479 |
| PosControl_158m3 | CTCCAAAGTAAGTCCAAAACTCACGTCATCCAGCAGAGAATGGAAAGTCAAATTTCCTGA | SEQ ID NO: 480 |
| PosControl_158m4 | CTCCAAAGCGGACTTGGGACTCACGTCATCCAGCAGAGAATGGAAAGTCAAATTTCCTGA | SEQ ID NO: 481 |
| PosControl_158m5 | CTCCAAAGACTCACGTCATCCAGCAGAGAATGGAAAGTCAAATTTCCTGATTTTTTTTTT | SEQ ID NO: 482 |
| PosControl_159 | CGGATTTGGTCGTATTGGGCGCCTGGTCACCAGGGCTGCTTTTAACTCTGGTAAAGTGGA | SEQ ID NO: 483 |
| PosControl_159m1 | CGGATTTGGTCTCTCCTTTTTCCTGGTCACCAGGGCTGCTTTTAACTCTGGTAAAGTGGA | SEQ ID NO: 484 |
| PosControl_159m2 | CGGATTTGGTCCCCCCCCCTCCCTGGTCACCAGGGCTGCTTTTAACTCTGGTAAAGTGGA | SEQ ID NO: 485 |
| PosControl_159m3 | CGGATTTGGTCCATAACCCGCCCTGGTCACCAGGGCTGCTTTTAACTCTGGTAAAGTGGA | SEQ ID NO: 486 |
| PosControl_159m4 | CGGATTTGGTCTGCGGTTTATCCTCGTCACCAGGGCTGCTTTTAACTCTGGTAAAGTGGA | SEQ ID NO: 487 |
| PosControl_159m5 | CGGATTTGGTCCCTGGTCACCAGGGCTGCTTTTAACTCTGGTAAAGTGGATTTTTTTTTT | SEQ ID NO: 488 |
| PosControl_160 | TGCTCCTCCTGTTCGACAGTCAGCCGCATCTTCTTTTGCGTCGCCAGCCGAGCCACATCG | SEQ ID NO: 489 |
| PosControl_160m1 | TGCTCCTCCTGTTCTTTTTCTTTTCGCATCTTCTTTTGCGTCGCCAGCCGAGCCACATCG | SEQ ID NO: 490 |
| PosControl_160m2 | TGCTCCTCCTGTTCCCCCCCCCCCCGCATCTTCTTTTGCGTCGCCAGCCGAGCCACATCG | SEQ ID NO: 491 |
| PosControl_160m3 | TGCTCCTCCTGTTCCTGTCAGTCGCGCATCTTCTTTTGCGTCGCCAGCCGAGCCACATCG | SEQ ID NO: 492 |
| PosControl_160m4 | TGCTCCTCCTGTTCTCACTGACTACGCATCTTCTTTTGCGTCGCCAGCCGAGCCACATCG | SEQ ID NO: 493 |
| PosControl_160m5 | TGCTCCTCCTGTTCCGCATCTTCTTTTGCGTCGCCAGCCGAGCCACATCGTTTTTTTTTT | SEQ ID NO: 494 |
| PosControl_161 | GTCTGAGGATACCACTGAAGAGACATTAAAGGAGTCATTTGACGGCTCCGTTCGGGCAAG | SEQ ID NO: 495 |
| PosControl_161m1 | GTCTGTTTTCACCACTGAAGTTTTTTTAAAGGAGTCATTTGACGGCTCCGTTCGGGCAAG | SEQ ID NO: 496 |
| PosControl_161m2 | GTCTGCCCCCACCACTGAAGCCCTCTTAAAGGAGTCATTTGACGGCTCCGTTCGGGCAAG | SEQ ID NO: 497 |
| PosControl_161m3 | GTCTGTCCTAACCACTGAAGTCTGTTTAAAGGAGTCATTTGACGGCTCCGTTCGGGCAAG | SEQ ID NO: 498 |
| PosControl_161m4 | GTCTGCTTCGACCACTGAAGCACACTTAAAGGAGTCATTTGACGGCTCCGTTCGGGCAAG | SEQ ID NO: 499 |
| PosControl_161m5 | GTCTGACCACTGAAGTTAAAGGAGTCATTTGACGGCTCCGTTCGGGCAAGTTTTTTTTTT | SEQ ID NO: 500 |
| PosControl_162 | CTCCCTCGACTGTCTTGTCCCAGAACCTTTTCACTCCAAAACAGGAAATTCAGCACCTGT | SEQ ID NO: 501 |
| PosControl_162m1 | CTCCCCTTTTTGTCTTGTCCTTTTTCCTTTTCACTCCAAAACAGGAAATTCAGCACCTGT | SEQ ID NO: 502 |
| PosControl_162m2 | CTCCCCTCCTTGTCTTGTCCTCCCCCCTTTTCACTCCAAAACAGGAAATTCAGCACCTGT | SEQ ID NO: 503 |
| PosControl_162m3 | CTCCCAGCTGTGTCTTGTCCGTCTTCCTTTTCACTCCAAAACAGGAAATTCAGCACCTGT | SEQ ID NO: 504 |
| PosControl_162m4 | CTCCCGATCATGTCTTGTCAACTCACCTTTTCACTCCAAAACAGGAAATTCAGCACCTGT | SEQ ID NO: 505 |
| PosControl_162m5 | CTCCCTGTCTTGTCCCCTTTTCACTCCAAAACAGGAAATTCAGCACCTGTTTTTTTTTTT | SEQ ID NO: 506 |
| PosControl_163 | CTCTGACACTGTGTAAGAAGCTGTGAATATTCCTAACTTACCCAGATGTTGCTTTTGAAA | SEQ ID NO: 507 |
| PosControl_163m1 | CTCTGTTTTCGTGTAAGAAGTCTCTAATATTCCTAACTTACCCAGATGTTGCTTTTGAAA | SEQ ID NO: 508 |
| PosControl_163m2 | CTCTGCTCTCGTGTAAGAAGTCCCCAATATTCCTAACTTACCCAGATGTTGCTTTTGAAA | SEQ ID NO: 509 |
| PosControl_163m3 | CTCTGTGTGAGTGTAAGAAGCTGTGAATATTCCTAACTTACCCAGATGTTGCTTTTGAAA | SEQ ID NO: 510 |
| PosControl_163m4 | CTCTGAACAGGTGTAAGAAGAGTGTAATATTCCTAACTTACCCAGATGTTGCTTTTGAAA | SEQ ID NO: 511 |
| PosControl_163m5 | CTCTGGTGTAAGAAGAATATTCCTAACTTACCCAGATGTTGCTTTTGAAATTTTTTTTTT | SEQ ID NO: 512 |
| PosControl_164 | GCTGGAGTCGATCAACTCTAGGCTCCAACTCGTTATGAAAAGTGGGAAGTACGTCCTGGG | SEQ ID NO: 513 |
| PosControl_164m1 | GCTGGTTCTTATCAACTCTATTTCTCAACTCGTTATGAAAAGTGGGAAGTACGTCCTGGG | SEQ ID NO: 514 |
| PosControl_164m2 | GCTGGCCCTCATCAACTCTACCTCTCAACTCGTTATGAAAAGTGGGAAGTACGTCCTGGG | SEQ ID NO: 515 |
| PosControl_164m3 | GCTGGTCAGCATCAACTCTACCGAGCAACTCGTTATGAAAAGTGGGAAGTACGTCCTGGG | SEQ ID NO: 516 |
| PosControl_164m4 | GCTGGCTGATATCAACTCTATTAGACAACTCGTTATGAAAAGTGGGAAGTACGTCCTGGG | SEQ ID NO: 517 |
| PosControl_164m5 | GCTGGATCAACTCTACAACTCGTTATGAAAAGTGGGAAGTACGTCCTGGGTTTTTTTTTT | SEQ ID NO: 518 |
| PosControl_165 | CAGGTGACTCTGACATCATTAGAAGCATGCCAGAACAGACTGGTGAAAAGTAAACCTTTT | SEQ ID NO: 519 |
| PosControl_165m1 | CAGGTTTTCTTGACATCATTTTTTTCATGCCAGAACAGACTGGTGAAAAGTAAACCTTTT | SEQ ID NO: 520 |
| PosControl_165m2 | CAGGTCCTCTTGACATCATTCCCCCCATGCCAGAACAGACTGGTGAAAAGTAAACCTTTT | SEQ ID NO: 521 |
| PosControl_165m3 | CAGGTCTGAGTGACATCATTTCTTCCATGCCAGAACAGACTGGTGAAAAGTAAACCTTTT | SEQ ID NO: 522 |
| PosControl_165m4 | CAGGTTCAGATGACATCATTCTCCTCATGCCAGAACAGACTGGTGAAAAGTAAACCTTTT | SEQ ID NO: 523 |
| PosControl_165m5 | CAGGTTGACATCATTCATGCCAGAACAGACTGGTGAAAAGTAAACCTTTTTTTTTTTTTT | SEQ ID NO: 524 |

TABLE I
Positive Control Genes

| Gene Symbol | Gene Name |
|---|---|
| TFRC | Transferrin receptor (p90, CD71) |
| SCYE1 | Small inducible cytokine subfamily E, member 1 (endothelial monocyte-activating) |
| RXRB | Retinoid X receptor, beta |
| TBP | TATA box binding protein |
| PPIA | Peptidylprolyl isomerase A (cyclophilin A) |
| ACTB | Actin, beta |
| GUSB | Glucuronidase, beta |
| EIF4G2 | Eukaryotic translation initiation factor 4 gamma, 2 |
| GAPD | Glyceraldehyde-3-phosphate dehydrogenase |
| RPLP0 | Ribosomal protein, large, P0 |
| B2M | Beta-2-microglobulin |
| TK2 | Thymidine kinase 2, mitochondrial |
| PGK1 | Phosphoglycerate kinase 1 |
| ATP7A | ATPase, Cu++ transporting, alpha polypeptide (Menkes syndrome) |
| HPRT1 | Hypoxanthine phosphoribosyltransferase 1 (Lesch-Nyhan syndrome) |
| CALM1 | Calmodulin 1 |
| LGALS3 | Lectin, galactoside-binding, soluble, 3 (galectin 3) |
| VIM | Vimentin |
| EEF2 | Eukaryotic translation elongation factor 2 |
| LDHB | Lactate dehydrogenase B |
| ATP5B | ATP synthase, H+ transporting, mitochondrial F1 complex, beta polypeptide |
| HLA-E | Major histocompatibility complex, class I, E |
| CALR | Calreticulin |
| RPL15 | Ribosomal protein L15 |
| YWHAZ | Tyrosine 3-monooxygenase/tryptophan 5-monooxygenase activation protein, zeta polypeptide |
| GNAS | GNAS complex locus |
| SERP1 | Stress-associated endoplasmic reticulum protein 1; ribosome associated membrane protein 4 |
| RPL13A | Ribosomal protein L13a |
| SSR2 | Signal sequence receptor, beta (translocon-associated protein beta) |
| RPL31 | Ribosomal protein L31 |
| CCNI | Cyclin I |
| PRDX5 | Peroxiredoxin 5 |
| DSTN | Destrin (actin depolymerizing factor) |
| RBM5 | RNA binding motif protein 5 |
| COX7B | Cytochrome c oxidase subunit VIIb |
| OAZ1 | Ornithine decarboxylase antizyme 1 |
| RTN4 | Reticulon 4 |
| GNB2L1 | Guanine nucleotide binding protein (G protein), beta polypeptide 2-like 1 |
| GABARAPL2 | GABA(A) receptor-associated protein-like 2 |
| NCL | Nucleolin |
| RPN2 | Ribophorin II |
| RPL30 | Ribosomal protein L30 |

1. A microarray comprising, an internal control set and at least one control element selected from:
a spatial normalization control, a control ladder, a positive gene mismatch control, a hybridization control, a non-specific background control, a reverse transcription control, a buffer blank control, and an in vitro transcription control.

2. The microarray of claim 1 comprising an internal control set and at least two control elements selected from:
a spatial normalization control, a control ladder, a positive gene mismatch control, a hybridization control, a non-specific background control, a reverse transcription control, a buffer blank control, and an in vitro transcription control.

3. The microarray of claim 1 comprising an internal control set and at least three control elements selected from:
a spatial normalization control, a control ladder, a positive gene mismatch control, a hybridization control, a non-specific background control, a reverse transcription control, a buffer blank control, and an in vitro transcription control.

4. The microarray of claim 1 comprising an internal control set and at least four control elements selected from:
a spatial normalization control, a control ladder, a positive gene mismatch control, a hybridization control, a non-specific background control, a reverse transcription control, a buffer blank control, and an in vitro transcription control.

5. The microarray of claim 1 comprising an internal control set and at least five control elements selected from:
a spatial normalization control, a control ladder, a positive gene mismatch control, a hybridization control, a non-specific background control, a reverse transcription control, a buffer blank control, and an in vitro transcription control.

6. The microarray of claim 1 comprising an internal control set and at least one control element selected from:
a spatial normalization control, a control ladder, and a positive gene mismatch control.

7. The microarray of claim 1 comprising an internal control set and at least two control elements selected from:
a spatial normalization control, a control ladder, and a positive gene mismatch control.

8. The microarray of claim 1 comprising an internal control set and:
a spatial normalization control, a control ladder, and a positive gene mismatch control.

9. The microarray of claim 1 comprising an internal control set and a spatial normalization control.

10. The microarray of claim 1 comprising an internal control set and a control ladder.

11. The microarray of claim 1 comprising an internal control set and a positive gene mismatch control.

12-19. (canceled)

20. A microarray comprising a control ladder.

21. The microarray of claim 20 further comprising at least one control element selected from:
a positive gene mismatch control, a hybridization control, a non-specific background control, a reverse transcription control, a buffer blank control, and an in vitro transcription control.

22. The microarray of claim 20 further comprising at least two control elements selected from:
a positive gene mismatch control, a hybridization control, a non-specific background control, a reverse transcription control, a buffer blank control, and an in vitro transcription control.

23. The microarray of claim 20 further comprising at least three control elements selected from:
a positive gene mismatch control, a hybridization control, a non-specific background control, a reverse transcription control, a buffer blank control, and an in vitro transcription control.

24. The microarray of claim 20 further comprising a positive gene mismatch control.

25. A microarray comprising a positive gene mismatch control.

26. The microarray of claim 25 further comprising at least one control element selected from:
a hybridization control, a non-specific background control, a reverse transcription control, a buffer blank control, and an in vitro transcription control.

27. The microarray of claim 25 further comprising at least two control elements selected from:
a hybridization control, a non-specific background control, a reverse transcription control, a buffer blank control, and an in vitro transcription control.

28-128. (canceled)

129. A microarray comprising, an internal control set and at least one control element selected from:
a spatial normalization control, a control ladder, a positive gene mismatch control, a hybridization control, a non-specific background control, a reverse transcription control, a buffer blank control, an in vitro transcription control, an attachment control, and a contaminant control.

130. A microarray comprising, an internal control set and at least four control elements selected from:
a spatial normalization control, a control ladder, a positive gene mismatch control, a hybridization control, a non-specific background control, a reverse transcription control, a buffer blank control, an in vitro transcription control, an attachment control, and a contaminant control.

131-132. (canceled)

133. A microarray comprising, a control ladder and at least one control element selected from:
a positive gene mismatch control, a hybridization control, a non-specific background control, a reverse transcription control, a buffer blank control, an in vitro transcription control, an attachment control, and a contaminant control.

134. A microarray comprising, a control ladder, and at least four control elements selected from:
a hybridization control, a non-specific background control, a reverse transcription control, a buffer blank control, an in vitro transcription control, an attachment control, and a contaminant control.

135. A microarray comprising, a positive gene mismatch control and at least one control element selected from:
a hybridization control, a non-specific background control, a reverse transcription control, a buffer blank control, an in vitro transcription control, an attachment control, and a contaminant control.

136. A microarray comprising, a positive gene mismatch control, and at least four control elements selected from:
a hybridization control, a non-specific background control, a reverse transcription control, a buffer blank control, an in vitro transcription control, an attachment control, and a contaminant control.


SEQUENCE LISTING (525 sequences)

SEQ ID NO: 1 - DNA, 24 bp, Artificial Sequence
  Synthetic polynucleotide sequence
ttcggctgtgagaacgatcacgca

SEQ ID NO: 2 - DNA, 24 bp, Artificial Sequence
  Synthetic polynucleotide sequence
cgaatcgtttgagcaagacccacg

SEQ ID NO: 3 - DNA, 24 bp, Artificial Sequence
  Synthetic polynucleotide sequence
cgaatcgtttgagcaagacccacg

SEQ ID NO: 4 - DNA, 60 bp, Artificial Sequence
  Synthetic polynucleotide sequence
cgacatgaaacttggtttgtgcccagtagcgacagaatcacgtatcggtttacgccgtca

SEQ ID NO: 5 - DNA, 60 bp, Artificial Sequence
  Synthetic polynucleotide sequence
ctcgagagtaattatgacacgtaaggtttaagagcccgccggacttggatccgtcctact

SEQ ID NO: 6 - DNA, 60 bp, Artificial Sequence
  Synthetic polynucleotide sequence
tgaactggttttgctagcccactcaacggtcacgcatctaagggatatgccgattcagga

SEQ ID NO: 7 - DNA, 60 bp, Artificial Sequence
  Synthetic polynucleotide sequence
cgcggcgtggaagaatccccacgaacgcgatatgccgtacctggaacaaatggtgcaggg

SEQ ID NO: 8 - DNA, 60 bp, Artificial Sequence
  Synthetic polynucleotide sequence
gacacctcgccggagttttacggcaatatcatcaccacacgcacttatcaggaacgcctc

SEQ ID NO: 9 - DNA, 60 bp, Artificial Sequence
  Synthetic polynucleotide sequence
ggtcgcgcggatcatgatgccaacctcttacgtgcgcctttctgccggacgcgagcagat

SEQ ID NO: 10 - DNA, 60 bp, Artificial Sequence
  Synthetic polynucleotide sequence
acctcttacgtgcgcctttctgccggacgcgagcagatgaacgaacagactcaggcgatg

SEQ ID NO: 11 - DNA, 60 bp, Artificial Sequence
  Synthetic polynucleotide sequence
ttgaacaggcgctgatgaccccggacaccgacgaatattacaacgcggcagcattagagg

SEQ ID NO: 12 - DNA, 60 bp, Artificial Sequence
  Synthetic polynucleotide sequence
acgccttactggcaatgcttccacagcgtaaatacacccacgtactggacgcgggttgtg

SEQ ID NO: 13 - DNA, 60 bp, Artificial Sequence
  Synthetic polynucleotide sequence
cgcagaccattatctggcgggagatatcgaatccctgccgttagcgactgcgacgttcga

SEQ ID NO: 14 - DNA, 60 bp, Artificial Sequence
  Synthetic polynucleotide sequence
aactgcatcaggcgtggcaggcggtggacgagcgtccgcatgctaatcgctttttaccgc

SEQ ID NO: 15 - DNA, 60 bp, Artificial Sequence
  Synthetic polynucleotide sequence
ttaccgccagatgaaatcgaacagtcgctgaacggcgtgcattatcaacatcatattcag

SEQ ID NO: 16 - DNA, 60 bp, Artificial Sequence
  Synthetic polynucleotide sequence
gctgtggtttgatgatgcgctcagtgccatgcgttcgctgaaaggcatcggtgccacgca

SEQ ID NO: 17 - DNA, 60 bp, Artificial Sequence
  Synthetic polynucleotide sequence
agtggggaaaactgtcgccagttgtgcacttttacaagccgcaaaggcagcaggctaccg

SEQ ID NO: 18 - DNA, 60 bp, Artificial Sequence
  Synthetic polynucleotide sequence
ggattacgcaacagtaaatccttacaccttcgcagaacccacttcgccgcacatcatcag

SEQ ID NO: 19 - DNA, 60 bp, Artificial Sequence
  Synthetic polynucleotide sequence
gccggtgatactggtagttggtgtgaaactcggctgtattaatcacgcgatgttgactgc

SEQ ID NO: 20 - DNA, 60 bp, Artificial Sequence
  Synthetic polynucleotide sequence
atcacgcgatgttgactgcacaggtaatacaacacgccggactgactctggcgggttggg

SEQ ID NO: 21 - DNA, 60 bp, Artificial Sequence
  Synthetic polynucleotide sequence
gttacgcctccgggaaaacgtcacgctgaatatatgaccacgctcacccgcatgattccc

SEQ ID NO: 22 - DNA, 60 bp, Artificial Sequence
  Synthetic polynucleotide sequence
acatacaaaaattgcattagagcacggagtccgtccagttgtcggaacaaccggtttctc

SEQ ID NO: 23 - DNA, 60 bp, Artificial Sequence
  Synthetic polynucleotide sequence
gaccagaagcttgacgcaccaagcggaactgcgcttaaaacagcggaaatgatttcagaa

SEQ ID NO: 24 - DNA, 60 bp, Artificial Sequence
  Synthetic polynucleotide sequence
attcttataaccgtgcttctttcatgtcaggcgttaaactgtcagtcgaacaagtcatga

SEQ ID NO: 25 - DNA, 60 bp, Artificial Sequence
  Synthetic polynucleotide sequence
caaccggaaccacagggttgaaaattcatgaggcgacaggtcttcaaattgaacgttttc

SEQ ID NO: 26 - DNA, 60 bp, Artificial Sequence
  Synthetic polynucleotide sequence
aggattccgtttctactcctctgacgaatgggtatattgaaatcgttgaagcgagagaaa

SEQ ID NO: 27 - DNA, 60 bp, Artificial Sequence
  Synthetic polynucleotide sequence
ttctttagatgtcgtatccggaggagagctatatacggctgttgcagcaggctttccggc

SEQ ID NO: 28 - DNA, 60 bp, Artificial Sequence
  Synthetic polynucleotide sequence
gaaatcgcgcttcttgaagacctatgtaaagaaacgggtcactccatcgatgttcttctt

SEQ ID NO: 29 - DNA, 60 bp, Artificial Sequence
  Synthetic polynucleotide sequence
tcagctgctgggtgtccattgccatatcggctcgcaaatctttgatacggccggttttgt

SEQ ID NO: 30 - DNA, 60 bp, Artificial Sequence
  Synthetic polynucleotide sequence
aagaaaatgcttcccgttacggttttgacattccggaaatttggatcgaaccgggccgtt

SEQ ID NO: 31 - DNA, 60 bp, Artificial Sequence
  Synthetic polynucleotide sequence
aaaagaagtggataagctgtacaatcgtttcatcattcggcgtgcgaattaaggatccaa

SEQ ID NO: 32 - DNA, 60 bp, Artificial Sequence
  Synthetic polynucleotide sequence
cttaccgcaccattccggagtgtatagatgcagccgttgcaggcgaagttgattttgctt

SEQ ID NO: 33 - DNA, 60 bp, Artificial Sequence
  Synthetic polynucleotide sequence
tttactcacattcacacgcgattgcgcaatgccataaatttcttcatcgacattttcctt

SEQ ID NO: 34 - DNA, 60 bp, Artificial Sequence
  Synthetic polynucleotide sequence
gggacaatcatacaagatttattatcctgtctcccgatgaaaacatatcttttgaagtga

SEQ ID NO: 35 - DNA, 60 bp, Artificial Sequence
  Synthetic polynucleotide sequence
tcaaaaattgagtcacgtccgactaaaaccggattaggccattatttctttattattgat

SEQ ID NO: 36 - DNA, 60 bp, Artificial Sequence
  Synthetic polynucleotide sequence
ccaggggccatgcaggagcttgaagcactcggctgcaaagtgaggcttctgggtgcatac

SEQ ID NO: 37 - DNA, 60 bp, Artificial Sequence
  Synthetic polynucleotide sequence
caacttccttcaacacttgagcggagtcggtgcatccgaacaatggaagcttccacattg

SEQ ID NO: 38 - DNA, 60 bp, Artificial Sequence
  Synthetic polynucleotide sequence
ctcacggtacacaaatccgtgctctaactcgaattcaacacaaggaatccacttgttgcg

SEQ ID NO: 39 - DNA, 60 bp, Artificial Sequence
  Synthetic polynucleotide sequence
gaattttccgcatttgattggttcttgtaagtatgagccggttaggattaggcctcgccg

SEQ ID NO: 40 - DNA, 60 bp, Artificial Sequence
  Synthetic polynucleotide sequence
cggtggatcagttttacttcgatggtagtttattaatggaccaatcagaatgttcttatt

SEQ ID NO: 41 - DNA, 60 bp, Artificial Sequence
  Synthetic polynucleotide sequence
aacgagttttgctgtatgaaatatctatcgatgactcaacaggttttgatcatgatcata

SEQ ID NO: 42 - DNA, 60 bp, Artificial Sequence
  Synthetic polynucleotide sequence
cacgcgtccgcaaaaccatcttaaagcaactacacaaatcttgaaattttctcatatttt

SEQ ID NO: 43 - DNA, 60 bp, Artificial Sequence
  Synthetic polynucleotide sequence
acaagattactctgctagaggagcttcaagaaaagaccgaggaagatgaggagaacaagc

SEQ ID NO: 44 - DNA, 60 bp, Artificial Sequence
  Synthetic polynucleotide sequence
aagatgaggagaacaagcctagtgtcatcgaaaagcttcaccgatccaacagctcttctt

SEQ ID NO: 45 - DNA, 60 bp, Artificial Sequence
  Synthetic polynucleotide sequence
aaaagtcaacgtacgatgacgttttcacttgcgtcactctcatgatttcatttattcttg

SEQ ID NO: 46 - DNA, 60 bp, Artificial Sequence
  Synthetic polynucleotide sequence
ttcaccaatgatgactctgcttgtgctgacagttcgatttagctaaccactcttgttgtg

SEQ ID NO: 47 - DNA, 60 bp, Artificial Sequence
  Synthetic polynucleotide sequence
caggaacttgttatgtcgtgcttcttgatgcagcaatgacagatcattttcgattgggcc

SEQ ID NO: 48 - DNA, 60 bp, Artificial Sequence
  Synthetic polynucleotide sequence
tttcatcacgttcctaacttaaccgggtgccgtatctgaaccaagcggtgctcggacaag

SEQ ID NO: 49 - DNA, 60 bp, Artificial Sequence
  Synthetic polynucleotide sequence
taggccacaacgagtaacactgcggtcgagaaagacctacgaagaacattatatttggaa

SEQ ID NO: 50 - DNA, 60 bp, Artificial Sequence
  Synthetic polynucleotide sequence
gaaccacgatacagctgtcgggcccaccgtgatctttctgggagctggaagctattacca

SEQ ID NO: 51 - DNA, 60 bp, Artificial Sequence
  Synthetic polynucleotide sequence
gttgttctgcgatggtggacaccgtataagccgacaaaggcctcggagggtgctcccggg

SEQ ID NO: 52 - DNA, 60 bp, Artificial Sequence
  Synthetic polynucleotide sequence
ccctaactcatgatgagggtaccataccgtatactaccgtgtaaccccttagttttgatg

SEQ ID NO: 53 - DNA, 60 bp, Artificial Sequence
  Synthetic polynucleotide sequence
tcagaaacatagcgtaataaacgaacagcggtccattatccctatagctctggcgcgttt

SEQ ID NO: 54 - DNA, 60 bp, Artificial Sequence
  Synthetic polynucleotide sequence
ggttcgtgtattgacaagatatgacttacatagttccgtcagtcagtggtgcatggtctg

SEQ ID NO: 55 - DNA, 60 bp, Artificial Sequence
  Synthetic polynucleotide sequence
tggagcgtaggcggtttcttaagctaatcgtctcacctagcgataagattttgcgggcgg

SEQ ID NO: 56 - DNA, 60 bp, Artificial Sequence
  Synthetic polynucleotide sequence
tgtcttttcgacctaatgctatgacgctagtaattagcccacgcctgaatctgtcgccgc

SEQ ID NO: 57 - DNA, 60 bp, Artificial Sequence
  Synthetic polynucleotide sequence
ataaatggtgcagtgtatgaaccttttccaaccagtgctagcaccctacgtctttttgcc

SEQ ID NO: 58 - DNA, 60 bp, Artificial Sequence
  Synthetic polynucleotide sequence
ctgtgattaccgtgcaattcaatcttatggtaaacctacggacgtacgaggaacaagctc

SEQ ID NO: 59 - DNA, 60 bp, Artificial Sequence
  Synthetic polynucleotide sequence
cgtacgaggaacaagctcgactagcttaacacaattgccagtctggaccatacttgtgca

SEQ ID NO: 60 - DNA, 60 bp, Artificial Sequence
  Synthetic polynucleotide sequence
tatgccaggtctatatcctcgttccgtggaaatacaccgccgtagcacaaatgcgagcga

SEQ ID NO: 61 - DNA, 60 bp, Artificial Sequence
  Synthetic polynucleotide sequence
tggtgcaggtactcttctgcaatagcgtcgtatcgtaactagtctatggagcactagaat

SEQ ID NO: 62 - DNA, 60 bp, Artificial Sequence
  Synthetic polynucleotide sequence
cgatgacactgattaaggcaaccgggtggaggtatatgctatccaactaggcatgtacca

SEQ ID NO: 63 - DNA, 60 bp, Artificial Sequence
  Synthetic polynucleotide sequence
ccccataggccatatcctattatcatcgcttacgacgctaacgaccgttaaatcatatcc

SEQ ID NO: 64 - DNA, 60 bp, Artificial Sequence
  Synthetic polynucleotide sequence
gatgctcctatcgttgctgaatgcgaaggctagggtgcacccgacgcggcagtgtaggtt

SEQ ID NO: 65 - DNA, 60 bp, Artificial Sequence
  Synthetic polynucleotide sequence
tagccgcgttgataggagataactatgttacataaagcgaacggttactcttaatgaggg

SEQ ID NO: 66 - DNA, 60 bp, Artificial Sequence
  Synthetic polynucleotide sequence
ggttactcttaatgagggccggcagataatccggaattttggcgctcaagttcttgcacc

SEQ ID NO: 67 - DNA, 60 bp, Artificial Sequence
  Synthetic polynucleotide sequence
cgcggtccctagccgcgtccggaccaatctcacctatgcaagccaattctgtcgcgcgcg

SEQ ID NO: 68 - DNA, 60 bp, Artificial Sequence
  Synthetic polynucleotide sequence
cgaccccggtcttatagccacgattacgcttgcattagtttgataaatagaacgcgcccc

SEQ ID NO: 69 - DNA, 60 bp, Artificial Sequence
  Synthetic polynucleotide sequence
ccataaatgacaagagttcccctcggtaaacagggtatatctgaaactactaggaaaaga

SEQ ID NO: 70 - DNA, 60 bp, Artificial Sequence
  Synthetic polynucleotide sequence
gagcctaactatcgttcatcgagaccgactatgcaacaactcctgcgttgctgagtaggg

SEQ ID NO: 71 - DNA, 60 bp, Artificial Sequence
  Synthetic polynucleotide sequence
tacaatgataggtcgcttattaacagtttgcgagtcgcgtgttgtagtgaacaaagcctt

SEQ ID NO: 72 - DNA, 60 bp, Artificial Sequence
  Synthetic polynucleotide sequence
agcaacgggcctatgcgatatctgatagtcagggttgacacatattcgattatacccttc

SEQ ID NO: 73 - DNA, 60 bp, Artificial Sequence
  Synthetic polynucleotide sequence
tattcgattatacccttcctatcacgtcatctcatgtgccttcgctcatagtatataaac

SEQ ID NO: 74 - DNA, 60 bp, Artificial Sequence
  Synthetic polynucleotide sequence
actaacccgctgtttagagttcagctagtgaccctttgacataccacacatgccaactag

SEQ ID NO: 75 - DNA, 60 bp, Artificial Sequence
  Synthetic polynucleotide sequence
cacacttaggtgcacaaacctgatgattcaagcatagcggactgatctttgtaacacaca

SEQ ID NO: 76 - DNA, 60 bp, Artificial Sequence
  Synthetic polynucleotide sequence
cgaagtcgaatgacagaatgtcaaagcttgtaggcactccgcgactagggttatttagtc

SEQ ID NO: 77 - DNA, 60 bp, Artificial Sequence
  Synthetic polynucleotide sequence
tgcggtatagatgccattgtgcgacttttttgtaaatcgcatgaatgggcaagggtaagc

SEQ ID NO: 78 - DNA, 60 bp, Artificial Sequence
  Synthetic polynucleotide sequence
ccctatagggaccgttgaacgcgtctaggcagcatacgcccagactacatggcgcgcaca

SEQ ID NO: 79 - DNA, 60 bp, Artificial Sequence
  Synthetic polynucleotide sequence
tttatcacggagagttacctcgcgctatagaagcaaatatgtcaggattcgtatgtgtac

SEQ ID NO: 80 - DNA, 60 bp, Artificial Sequence
  Synthetic polynucleotide sequence
gccaaccactgtgtaaaaacattgcgggtaattttagcaccgaaagtccaaagcatctga

SEQ ID NO: 81 - DNA, 60 bp, Artificial Sequence
  Synthetic polynucleotide sequence
ctaatggcgggaccagacccaaaagagttgcaagagtttgactcactgccgttcggtgag

SEQ ID NO: 82 - DNA, 60 bp, Artificial Sequence
  Synthetic polynucleotide sequence
ccgtgaattacggtctgttcgtgtctctgcatgtcagcgatgtgaacacgtggctgatcg

SEQ ID NO: 83 - DNA, 60 bp, Artificial Sequence
  Synthetic polynucleotide sequence
ggtctgttcagataacgctatgcaagtgttggcaatggcgctagtagcaactctgctccg

SEQ ID NO: 84 - DNA, 60 bp, Artificial Sequence
  Synthetic polynucleotide sequence
agtagcaactctgctccggatttcgttgtaggaagtcatgctcgtatcgaactaactaca

SEQ ID NO: 85 - DNA, 60 bp, Artificial Sequence
  Synthetic polynucleotide sequence
gcgctaagaagttaccccatgggttaaaattaggacccgtgttgtagactcaaagaggag

SEQ ID NO: 86 - DNA, 60 bp, Artificial Sequence
  Synthetic polynucleotide sequence
gtcaacagtgacatgccatcctatatactcaacagcagtatcagtcagatgaaaacatga

SEQ ID NO: 87 - DNA, 60 bp, Artificial Sequence
  Synthetic polynucleotide sequence
agtcagatgaaaacatgacgacgtggatcccgacgtagtagcatagggttgtagagcggg

SEQ ID NO: 88 - DNA, 60 bp, Artificial Sequence
  Synthetic polynucleotide sequence
cggctgcatttgtagtagcacccatatctacgatttcagcgggtgtgaacgtccaaacaa

SEQ ID NO: 89 - DNA, 60 bp, Artificial Sequence
  Synthetic polynucleotide sequence
cttgacctgatagctatttcggagttaggtcccaacccgtcagaatgtcacgtgccagtc

SEQ ID NO: 90 - DNA, 60 bp, Artificial Sequence
  Synthetic polynucleotide sequence
ccacactccgtaatcgttgtcgcgcttctgggattgagtcagaaacggcgggctgacact

SEQ ID NO: 91 - DNA, 60 bp, Artificial Sequence
  Synthetic polynucleotide sequence
tcgtagccgataagcaaacttgcgtgcttgaggccattgtacgtgggagacacttcgtac

SEQ ID NO: 92 - DNA, 60 bp, Artificial Sequence
  Synthetic polynucleotide sequence
gagaccagtatgagcgaaacgtcctcagaaatgttacgggtacctagctgcgaaacccca

SEQ ID NO: 93 - DNA, 60 bp, Artificial Sequence
  Synthetic polynucleotide sequence
attatcactctgcgatggttcggagatactgtcatcggtgcccgcatttgtcggaatgga

SEQ ID NO: 94 - DNA, 60 bp, Artificial Sequence
  Synthetic polynucleotide sequence
ttctctaacaatagccgattaatcttgtgcggagctctaggcagctacctagtagtgaga

SEQ ID NO: 95 - DNA, 60 bp, Artificial Sequence
  Synthetic polynucleotide sequence
cgctagtcggccgaaattgtagggattcggaggtatatgataagttcgaaaattcctccc

SEQ ID NO: 96 - DNA, 60 bp, Artificial Sequence
  Synthetic polynucleotide sequence
gcccaatagggtccatagtgccgatgttacgtttggattagggcattctaccttcttgcc

SEQ ID NO: 97 - DNA, 60 bp, Artificial Sequence
  Synthetic polynucleotide sequence
atcccccgataaaatatggctaagcacaggttccatgcggagtgagtctgaggcgttatg

SEQ ID NO: 98 - DNA, 60 bp, Artificial Sequence
  Synthetic polynucleotide sequence
tgagtctgaggcgttatgccttcgattgcctttaccgtacccacgctgccaattgttttg

SEQ ID NO: 99 - DNA, 60 bp, Artificial Sequence
  Synthetic polynucleotide sequence
accggtttacgtcataactacttcgtcagttactctcacgactttcttaatcgctagtag

SEQ ID NO: 100 - DNA, 60 bp, Artificial Sequence
  Synthetic polynucleotide sequence
acttatttgcgtccaaatgactagatacaagaggttaacggcgttgccgactgctaacgc

SEQ ID NO: 101 - DNA, 60 bp, Artificial Sequence
  Synthetic polynucleotide sequence
acggcaattgttctttgtgacgtttaagggccatgcagcagatacgggaatataatacca

SEQ ID NO: 102 - DNA, 60 bp, Artificial Sequence
  Synthetic polynucleotide sequence
tgtgcgtcaggattcacgcgcatgtcacaaagcagttacgtagcggtgtcccaatctgtc

SEQ ID NO: 103 - DNA, 60 bp, Artificial Sequence
  Synthetic polynucleotide sequence
ctgtatatctctgcaaggcatgaacgcaatgtgctctttactcctacccctttaacacct

SEQ ID NO: 104 - DNA, 60 bp, Artificial Sequence
  Synthetic polynucleotide sequence
cctacccctttaacacctgttaccattagttgacgtgagggcggcaaaaagatgattgga

SEQ ID NO: 105 - DNA, 60 bp, Artificial Sequence
  Synthetic polynucleotide sequence
ggccgttgtttgcacactgtagtatctgagtgtcacaatcccaccgtttcacaatattaa

SEQ ID NO: 106 - DNA, 60 bp, Artificial Sequence
  Synthetic polynucleotide sequence
accgtttcacaatattaaagtcccccgaaaatgacctaaatccacatgtcctaatttatt

SEQ ID NO: 107 - DNA, 60 bp, Artificial Sequence
  Synthetic polynucleotide sequence
aagggagtacagactctacccttgtgatccgtgtatcaaattttctaaacgcttaccagc

SEQ ID NO: 108 - DNA, 60 bp, Artificial Sequence
  Synthetic polynucleotide sequence
gttgaggctacataggaacatcggccgtataaacagaggaaacttatcctacatacaggg

SEQ ID NO: 109 - DNA, 60 bp, Artificial Sequence
  Synthetic polynucleotide sequence
ggatttacttaggtctacacgttgtcaccttagtgatagaaacagacactgatgaatata

SEQ ID NO: 110 - DNA, 60 bp, Artificial Sequence
  Synthetic polynucleotide sequence
aaccgttcagtaaaggtaggatagcccatgtgatcaactctatttgcagcaatgcacgtt

SEQ ID NO: 111 - DNA, 60 bp, Artificial Sequence
  Synthetic polynucleotide sequence
tttcagtgtgccattcttgattaatacagcacgagatgaatcgaccgtcttcgcccgtaa

SEQ ID NO: 112 - DNA, 60 bp, Artificial Sequence
  Synthetic polynucleotide sequence
aaccaagtactaagcatagtcaatgtacgagtgttacaggtctcattggtacgggtcggg

SEQ ID NO: 113 - DNA, 60 bp, Artificial Sequence
  Synthetic polynucleotide sequence
aaagggtagccggcacaagttacggactcgaaatatcggtcaaccatcgggcaagtcggg

SEQ ID NO: 114 - DNA, 60 bp, Artificial Sequence
  Synthetic polynucleotide sequence
gacagttgtaacattgcggtaatacttgacttacgactgagctttcaattgagactaacc

SEQ ID NO: 115 - DNA, 60 bp, Artificial Sequence
  Synthetic polynucleotide sequence
accaagaggctcttagaggatcagggttagctatttaagccttcgaatcctagcggcgtg

SEQ ID NO: 116 - DNA, 60 bp, Artificial Sequence
  Synthetic polynucleotide sequence
ccaacggcacgttaacagacggtgaagccttctcgtcagatcttagaggcggattcgaca

SEQ ID NO: 117 - DNA, 60 bp, Artificial Sequence
  Synthetic polynucleotide sequence
accaacctatgtagcacgcgagatttggaaataaggccaacgaatcatcatgttacgaag

SEQ ID NO: 118 - DNA, 60 bp, Artificial Sequence
  Synthetic polynucleotide sequence
gtgacgcgctcagcaattgctatactagcgttctgcaaacctttcaggtgtattagcaga

SEQ ID NO: 119 - DNA, 60 bp, Artificial Sequence
  Synthetic polynucleotide sequence
ctcctttattgagtccgagcgttacaacgacacatttcagttgaagcgcttgacaccaca

SEQ ID NO: 120 - DNA, 60 bp, Artificial Sequence
  Synthetic polynucleotide sequence
ctggatccgactaatagccagcgtcagcggatggcctatctgtgcgttacgttcgccaca

SEQ ID NO: 121 - DNA, 60 bp, Artificial Sequence
  Synthetic polynucleotide sequence
accaggaccttgcccggtctctaaattttcttaatacgtaaactacccaaatgtcccagc

SEQ ID NO: 122 - DNA, 60 bp, Artificial Sequence
  Synthetic polynucleotide sequence
cattcatccaaccgcggctcgatgggcattcctctcatcgttctgtcccatagacctatc

SEQ ID NO: 123 - DNA, 60 bp, Artificial Sequence
  Synthetic polynucleotide sequence
cacccaatcgatgtgtttatgtttgtcctgacgagatccgctgcttgttcaaaagaagat

SEQ ID NO: 124 - DNA, 60 bp, Artificial Sequence
  Synthetic polynucleotide sequence
ctagaagctcgcaatagtcagggacctaccatgttgtgaaggccatacgtatcgcccgcg

SEQ ID NO: 125 - DNA, 60 bp, Artificial Sequence
  Synthetic polynucleotide sequence
ggtagcgttcggtagttataatatctattctcgcgtaaacttgttcccagcgcgcaaggg

SEQ ID NO: 126 - DNA, 60 bp, Artificial Sequence
  Synthetic polynucleotide sequence
taggcgtgtgaaccggatcgccgtaagagccgcacaacactcctaaagcagcgtgtctat

SEQ ID NO: 127 - DNA, 60 bp, Artificial Sequence
  Synthetic polynucleotide sequence
caattcatataaaatcttacgtttcacgcgcttatactagctgaggctagcccaccctca

SEQ ID NO: 128 - DNA, 60 bp, Artificial Sequence
  Synthetic polynucleotide sequence
cggacaaatatactattccaatgtgcctcgtattcaatgtcgttctcagtggaaccacac

SEQ ID NO: 129 - DNA, 60 bp, Artificial Sequence
  Synthetic polynucleotide sequence
gccggttttcactgaacttactctgcctgcagatcaccagtcagtttctacgagggtggg

SEQ ID NO: 130 - DNA, 60 bp, Artificial Sequence
  Synthetic polynucleotide sequence
ccatgtcgcacgtacagctcgtggacaaaatagcttttgcgggcagtaaaaattcattct

SEQ ID NO: 131 - DNA, 60 bp, Artificial Sequence
  Synthetic polynucleotide sequence
gcagtaaaaattcattctgattcggttacgccttagactcctccggcgcgatcaccacaa

SEQ ID NO: 132 - DNA, 60 bp, Artificial Sequence
  Synthetic polynucleotide sequence
caaagaagtctgctatgtttagttacccaatcgtggcaaagacaatccacatttggctgg

SEQ ID NO: 133 - DNA, 60 bp, Artificial Sequence
  Synthetic polynucleotide sequence
gcaaccgtttcgccatggtattctcgggcccgtgacactttggttccaatggattaatac

SEQ ID NO: 134 - DNA, 60 bp, Artificial Sequence
  Synthetic polynucleotide sequence
ccgaggtgcacatctactccgtattcacattagacttcaagactgactggcatacaaggg

SEQ ID NO: 135 - DNA, 60 bp, Artificial Sequence
  Synthetic polynucleotide sequence
caactatgaagaattcgtacagatgatgactgcaaaatgaagacctactttcaactcctt

SEQ ID NO: 136 - DNA, 60 bp, Artificial Sequence
  Synthetic polynucleotide sequence
gaattacgaggacttggtgcatatcataactgcaaaatgaagacctactttcaactcctt

SEQ ID NO: 137 - DNA, 60 bp, Artificial Sequence
  Synthetic polynucleotide sequence
caactatgaagaattcgtacagatgatgacggcgaattgcagtcccacgttgaattcttt

SEQ ID NO: 138 - DNA, 60 bp, Artificial Sequence
  Synthetic polynucleotide sequence
caactatgaagaatttgttcaaatcattacagcgaattgcagacctactttcaactcctt

SEQ ID NO: 139 - DNA, 60 bp, Artificial Sequence
  Synthetic polynucleotide sequence
gaactaagaagagttcgttcagataatgaccgcaaagtgaagccctacattcaattcctt

SEQ ID NO: 140 - DNA, 60 bp, Artificial Sequence
  Synthetic polynucleotide sequence
cactatgagaatcgtacgatgagactgaaaataagactacttcaactctttttttttttt

SEQ ID NO: 141 - DNA, 60 bp, Artificial Sequence
  Synthetic polynucleotide sequence
aacattgctgttcaaagaaattacagtttacgtccattccaagttgtaaatgctagtctt

SEQ ID NO: 142 - DNA, 60 bp, Artificial Sequence
  Synthetic polynucleotide sequence
acttctttaagaaattacagtttacgtccattccaagttgtaaatgctagtctttttttt

SEQ ID NO: 143 - DNA, 60 bp, Artificial Sequence
  Synthetic polynucleotide sequence
aacatgtgtcaaaattacagtttacgtccattccaagttgtaaatgctagtctttttttt

SEQ ID NO: 144 - DNA, 60 bp, Artificial Sequence
  Synthetic polynucleotide sequence
aacattgctttaagaatacgtttacgtccattccaagttgtaaatgctagtctttttttt

SEQ ID NO: 145 - DNA, 60 bp, Artificial Sequence
  Synthetic polynucleotide sequence
aacattgctgttcaagaataagttcgtccattccaagttgtaaatgctagtctttttttt

SEQ ID NO: 146 - DNA, 60 bp, Artificial Sequence
  Synthetic polynucleotide sequence
aacattgctgttcaaagaattcattactcatccaagttgtaaatgctagtcttttttttt

SEQ ID NO: 147 - DNA, 60 bp, Artificial Sequence
  Synthetic polynucleotide sequence
agaacaagagctagagcgattaagaagcgaaaataaggatattgaaaatctgagaagaga

SEQ ID NO: 148 - DNA, 60 bp, Artificial Sequence
  Synthetic polynucleotide sequence
agaacttttttctttgcgattaagaagcgaaaataaggatattgaaaatctgagaagaga

SEQ ID NO: 149 - DNA, 60 bp, Artificial Sequence
  Synthetic polynucleotide sequence
agaacaagagctagattttccttttagcgaaaataaggatattgaaaatctgagaagaga

SEQ ID NO: 150 - DNA, 60 bp, Artificial Sequence
  Synthetic polynucleotide sequence
agaacaagagctagagcgattaagattttttttctaggatattgaaaatctgagaagaga

SEQ ID NO: 151 - DNA, 60 bp, Artificial Sequence
  Synthetic polynucleotide sequence
agaacaagagctagagcgattaagaagcgaaaatattttctccttaaatctgagaagaga

SEQ ID NO: 152 - DNA, 60 bp, Artificial Sequence
  Synthetic polynucleotide sequence
agaacaagagctagagcgattaagaagcgaaaataaggatattgatttctcttttagaga

SEQ ID NO: 153 - DNA, 60 bp, Artificial Sequence
  Synthetic polynucleotide sequence
acacattgttacagctagagtgtgaaaaatacaaatccgtccttgcagaaacagaaggaa

SEQ ID NO: 154 - DNA, 60 bp, Artificial Sequence
  Synthetic polynucleotide sequence
tttttttgttacagctagagtgtgaaaaatacaaatccgtccttgcagaaacagaaggaa

SEQ ID NO: 155 - DNA, 60 bp, Artificial Sequence
  Synthetic polynucleotide sequence
tttttacacattgtttagagtgtgaaaaatacaaatccgtccttgcagaaacagaaggaa

SEQ ID NO: 156 - DNA, 60 bp, Artificial Sequence
  Synthetic polynucleotide sequence
tttttacacattgttacagctagagaaaatacaaatccgtccttgcagaaacagaaggaa

SEQ ID NO: 157 - DNA, 60 bp, Artificial Sequence
  Synthetic polynucleotide sequence
tttttacacattgttacagctagagtgtgaaaaattccgtccttgcagaaacagaaggaa

SEQ ID NO: 158 - DNA, 60 bp, Artificial Sequence
  Synthetic polynucleotide sequence
tttttacacattgttacagctagagtgtgaaaaatacaaatccgtcagaaacagaaggaa

SEQ ID NO: 159 - DNA, 60 bp, Artificial Sequence
  Synthetic polynucleotide sequence
acgccagatgcgtgaaatggaagagaactttgccgttgaagctgctaactaccaagacac

SEQ ID NO: 160 - DNA, 60 bp, Artificial Sequence
  Synthetic polynucleotide sequence
tttttacgcccgtgaaatggaagagaactttgccgttgaagctgctaactaccaagacac

SEQ ID NO: 161 - DNA, 60 bp, Artificial Sequence
  Synthetic polynucleotide sequence
tttttacgccagatgcgtgaaagagaactttgccgttgaagctgctaactaccaagacac

SEQ ID NO: 162 - DNA, 60 bp, Artificial Sequence
  Synthetic polynucleotide sequence
tttttacgccagatgcgtgaaatggaagagtgccgttgaagctgctaactaccaagacac

SEQ ID NO: 163 - DNA, 60 bp, Artificial Sequence
  Synthetic polynucleotide sequence
tttttacgccagatgcgtgaaatggaagagaactttgccggctgctaactaccaagacac

SEQ ID NO: 164 - DNA, 60 bp, Artificial Sequence
  Synthetic polynucleotide sequence
tttttacgccagatgcgtgaaatggaagagaactttgccgttgaagctgcaccaagacac

SEQ ID NO: 165 - DNA, 60 bp, Artificial Sequence
  Synthetic polynucleotide sequence
caggatgttgacaatgcgtctctggcacgtcttgaccttgaacgcaaagtggaatctttg

SEQ ID NO: 166 - DNA, 60 bp, Artificial Sequence
  Synthetic polynucleotide sequence
caggatgttaacaacgcgtttctggcacgtcttgaccttgaacgcaaagtggaatctttg

SEQ ID NO: 167 - DNA, 60 bp, Artificial Sequence
  Synthetic polynucleotide sequence
caggacgtcgataacgcatccctggcacgtcttgaccttgaacgcaaagtggaatctttg

SEQ ID NO: 168 - DNA, 60 bp, Artificial Sequence; identical to SEQ ID NO: 165
  Synthetic polynucleotide sequence

SEQ ID NO: 169 - DNA, 60 bp, Artificial Sequence
  Synthetic polynucleotide sequence
tagaatattaacgatacgcctttgacatgttttaaccttgaacgcaaagtggaatctttg

SEQ ID NO: 170 - DNA, 60 bp, Artificial Sequence
  Synthetic polynucleotide sequence
cggaacgctaatagtacatttttagtatgccttgaccttgaacgcaaagtggaatctttg

SEQ ID NO: 171 - DNA, 60 bp, Artificial Sequence
  Synthetic polynucleotide sequence
cctactttgtcttcagcagttagctaaattacaagatcgagaatggttaacagaactttt

SEQ ID NO: 172 - DNA, 60 bp, Artificial Sequence
  Synthetic polynucleotide sequence
ttcgtcccaccttcagcagttagctaaattacaagatcgagaatggttaacagaactttt

SEQ ID NO: 173 - DNA, 60 bp, Artificial Sequence
  Synthetic polynucleotide sequence
cctactttgttcctgatgactagctaaattacaagatcgagaatggttaacagaactttt

SEQ ID NO: 174 - DNA, 60 bp, Artificial Sequence
  Synthetic polynucleotide sequence
cctactttgtcttcagcagtctatcgggccacaagatcgagaatggttaacagaactttt

SEQ ID NO: 175 - DNA, 60 bp, Artificial Sequence
  Synthetic polynucleotide sequence
cctactttgtcttcagcagttagctaaattgtggagctaggaatggttaacagaactttt

SEQ ID NO: 176 - DNA, 60 bp, Artificial Sequence
  Synthetic polynucleotide sequence
cctactttgtcttcagcagttagctaaattacaagatcgaaggcaaccggcagaactttt

SEQ ID NO: 177 - DNA, 60 bp, Artificial Sequence
  Synthetic polynucleotide sequence
ttccagagtccggaggcagcagacacgccctcttagtagggacttaatgggccggtcggg

SEQ ID NO: 178 - DNA, 60 bp, Artificial Sequence
  Synthetic polynucleotide sequence
ttccttttctcggaggcagcagacacgccctcttagtagggacttaatgggccggtcggg

SEQ ID NO: 179 - DNA, 60 bp, Artificial Sequence
  Synthetic polynucleotide sequence
ttccagagtccggattttttagacacgccctcttagtagggacttaatgggccggtcggg

SEQ ID NO: 180 - DNA, 60 bp, Artificial Sequence
  Synthetic polynucleotide sequence
ttccagagtccggaggcagcagactttttttcttagtagggacttaatgggccggtcggg

SEQ ID NO: 181 - DNA, 60 bp, Artificial Sequence
  Synthetic polynucleotide sequence
ttccagagtccggaggcagcagacacgccctcttttctttgacttaatgggccggtcggg

SEQ ID NO: 182 - DNA, 60 bp, Artificial Sequence
  Synthetic polynucleotide sequence
ttccagagtccggaggcagcagacacgccctcttagtagggactcttcttgccggtcggg

SEQ ID NO: 183 - DNA, 60 bp, Artificial Sequence
  Synthetic polynucleotide sequence
ggtggcggagacccgcaagcgcaagggcctgaaagaaggcatccctgccctggacaactt

SEQ ID NO: 184 - DNA, 60 bp, Artificial Sequence
  Synthetic polynucleotide sequence
ggtggcggagacccagcgcaagggcctgaaagaaggcatccctgccctggacaacttttt

SEQ ID NO: 185 - DNA, 60 bp, Artificial Sequence
  Synthetic polynucleotide sequence
ggtggcggagcccacgcaagggcctgaaagaaggcatccctgccctggacaacttttttt

SEQ ID NO: 186 - DNA, 60 bp, Artificial Sequence
  Synthetic polynucleotide sequence
ggtggcgggcccaccaagggcctgaaagaaggcatccctgccctggacaacttttttttt

SEQ ID NO: 187 - DNA, 60 bp, Artificial Sequence
  Synthetic polynucleotide sequence
ggtggcggcccaccaggcctgaaagaaggcatccctgccctggacaactttttttttttt

SEQ ID NO: 188 - DNA, 60 bp, Artificial Sequence
  Synthetic polynucleotide sequence
ggtgcggcccaccaggcctgaaagaaggcatccctgccctggacaacttttttttttttt

SEQ ID NO: 189 - DNA, 60 bp, Artificial Sequence
  Synthetic polynucleotide sequence
gacatccagaaggacctaaaagacctgtgactagtgagctctaggctgtagaaatttaaa

SEQ ID NO: 190 - DNA, 60 bp, Artificial Sequence
  Synthetic polynucleotide sequence
ttttctcagaaggacctaaaagacctgtgactagtgagctctaggctgtagaaatttaaa

SEQ ID NO: 191 - DNA, 60 bp, Artificial Sequence
  Synthetic polynucleotide sequence
gacatccagttttttctaaaagacctgtgactagtgagctctaggctgtagaaatttaaa

SEQ ID NO: 192 - DNA, 60 bp, Artificial Sequence
  Synthetic polynucleotide sequence
gacatccagaaggacctaatttttttgtgactagtgagctctaggctgtagaaatttaaa

SEQ ID NO: 193 - DNA, 60 bp, Artificial Sequence
  Synthetic polynucleotide sequence
gacatccagaaggacctaaaagacctgtgttcttcgagctctaggctgtagaaatttaaa

SEQ ID NO: 194 - DNA, 60 bp, Artificial Sequence
  Synthetic polynucleotide sequence
gacatccagaaggacctaaaagacctgtgactagtgagcctctttctgtagaaatttaaa

SEQ ID NO: 195 - DNA, 60 bp, Artificial Sequence
  Synthetic polynucleotide sequence
tgaaagtgcctatgaagtcatcaagctaaaaggatataccaactgggctattggattaag

SEQ ID NO: 196 - DNA, 60 bp, Artificial Sequence
  Synthetic polynucleotide sequence
tgcctatgaagtcatcaagctaaaaggatataccaactgggctattggattaagtttttt

SEQ ID NO: 197 - DNA, 60 bp, Artificial Sequence
  Synthetic polynucleotide sequence
tgaaagtgcagtcatcaagctaaaaggatataccaactgggctattggattaagtttttt

SEQ ID NO: 198 - DNA, 60 bp, Artificial Sequence
  Synthetic polynucleotide sequence
tgaaagtgcctatgaagtcctaaaaggatataccaactgggctattggattaagtttttt

SEQ ID NO: 199 - DNA, 60 bp, Artificial Sequence
  Synthetic polynucleotide sequence
tgaaagtgcctatgaagtcatcaagctaaataccaactgggctattggattaagtttttt

SEQ ID NO: 200 - DNA, 60 bp, Artificial Sequence
  Synthetic polynucleotide sequence
tgaaagtgcctatgaagtcatcaagctaaaaggatatagggctattggattaagtttttt

SEQ ID NO: 201 - DNA, 60 bp, Artificial Sequence
  Synthetic polynucleotide sequence
aatctggtgcagagaaatgttaatgtcttcaaattcattattcctcagatcgtcaagtac

SEQ ID NO: 202 - DNA, 60 bp, Artificial Sequence
  Synthetic polynucleotide sequence
aatcagagaaatgttaatgtcttcaaattcattattcctcagatcgtcaagtactttttt

SEQ ID NO: 203 - DNA, 60 bp, Artificial Sequence
  Synthetic polynucleotide sequence
aatctggtgcagagtaatgtcttcaaattcattattcctcagatcgtcaagtactttttt

SEQ ID NO: 204 - DNA, 60 bp, Artificial Sequence
  Synthetic polynucleotide sequence
aatctggtgcagagaaatgttaataaattcattattcctcagatcgtcaagtactttttt

SEQ ID NO: 205 - DNA, 60 bp, Artificial Sequence
  Synthetic polynucleotide sequence
aatctggtgcagagaaatgttaatgtcttcaaatttcctcagatcgtcaagtactttttt

SEQ ID NO: 206 - DNA, 60 bp, Artificial Sequence
  Synthetic polynucleotide sequence
aatctggtgcagagaaatgttaatgtcttcaaattcattattcccgtcaagtactttttt

SEQ ID NO: 207 - DNA, 60 bp, Artificial Sequence
  Synthetic polynucleotide sequence
cagcagattttggcaggtgaatatgaccatctcccagaacaggccttctatatggtggga

SEQ ID NO: 208 - DNA, 60 bp, Artificial Sequence
  Synthetic polynucleotide sequence
tggcaggtgaatatgaccatctcccagaacaggccttctatatggtgggatttttttttt

SEQ ID NO: 209 - DNA, 60 bp, Artificial Sequence
  Synthetic polynucleotide sequence
cagcagatttatatgaccatctcccagaacaggccttctatatggtgggatttttttttt

SEQ ID NO: 210 - DNA, 60 bp, Artificial Sequence
  Synthetic polynucleotide sequence
cagcagattttggcaggtgactcccagaacaggccttctatatggtgggatttttttttt

SEQ ID NO: 211 - DNA, 60 bp, Artificial Sequence
  Synthetic polynucleotide sequence
cagcagattttggcaggtgaatatgaccataggccttctatatggtgggatttttttttt

SEQ ID NO: 212 - DNA, 60 bp, Artificial Sequence
  Synthetic polynucleotide sequence
cagcagattttggcaggtgaatatgaccatctcccagaactatggtgggatttttttttt

SEQ ID NO: 213 - DNA, 60 bp, Artificial Sequence
  Synthetic polynucleotide sequence
gaccgtgtcccgtgcacggaaaatacagcgtttcttgtctcagccattccaggttgctga

SEQ ID NO: 214 - DNA, 60 bp, Artificial Sequence
  Synthetic polynucleotide sequence
gacccgtgcacggaaaatacagcgtttcttgtctcagccattccaggttgctgatttttt

SEQ ID NO: 215 - DNA, 60 bp, Artificial Sequence
  Synthetic polynucleotide sequence
gaccgtgtcccgtgaaatacagcgtttcttgtctcagccattccaggttgctgatttttt

SEQ ID NO: 216 - DNA, 60 bp, Artificial Sequence
  Synthetic polynucleotide sequence
gaccgtgtcccgtgcacggaaaattttcttgtctcagccattccaggttgctgatttttt

SEQ ID NO: 217 - DNA, 60 bp, Artificial Sequence
  Synthetic polynucleotide sequence
gaccgtgtcccgtgcacggaaaatacagcgtttccagccattccaggttgctgatttttt

SEQ ID NO: 218 - DNA, 60 bp, Artificial Sequence
  Synthetic polynucleotide sequence
gaccgtgtcccgtgcacggaaaatacagcgtttcttgtctcagcaggttgctgatttttt

SEQ ID NO: 219 - DNA, 60 bp, Artificial Sequence
  Synthetic polynucleotide sequence
ctgtgccactgtccccccagccattcactcctactgatgagacaagatgcggtgatgaca

SEQ ID NO: 220 - DNA, 60 bp, Artificial Sequence
  Synthetic polynucleotide sequence
ctgtgcccagccattcactcctactgatgagacaagatgcggtgatgacatttttttttt

SEQ ID NO: 221 - DNA, 60 bp, Artificial Sequence
  Synthetic polynucleotide sequence
ctgtgccactgtccccactcctactgatgagacaagatgcggtgatgacatttttttttt

SEQ ID NO: 222 - DNA, 60 bp, Artificial Sequence
  Synthetic polynucleotide sequence
ctgtgccactgtccccccagccattgatgagacaagatgcggtgatgacatttttttttt

SEQ ID NO: 223 - DNA, 60 bp, Artificial Sequence
  Synthetic polynucleotide sequence
ctgtgccactgtccccccagccattcactcctactgatgcggtgatgacatttttttttt

SEQ ID NO: 224 - DNA, 60 bp, Artificial Sequence
  Synthetic polynucleotide sequence
ctgtgccactgtccccccagccattcactcctactgatgagacaatgacatttttttttt

SEQ ID NO: 225 - DNA, 60 bp, Artificial Sequence
  Synthetic polynucleotide sequence
tcgtcatgggtgtgaaccatgagaagtatgacaacagcctcaagatcatcagcaatgcct

SEQ ID NO: 226 - DNA, 60 bp, Artificial Sequence
  Synthetic polynucleotide sequence
tcgtcatgggtgtggaccatgagaagtatgacaacagcctcaagatcatcagcaatgcct

SEQ ID NO: 227 - DNA, 60 bp, Artificial Sequence
  Synthetic polynucleotide sequence
tcgtcatgggtgcggatcatgagaagtatgacaacagcctcaagatcatcagcaatgcct

SEQ ID NO: 228 - DNA, 60 bp, Artificial Sequence
  Synthetic polynucleotide sequence
tcgtcatgagcgcggatcatgagaagtatgacaacagcctcaagatcatcagcaatgcct

SEQ ID NO: 229 - DNA, 60 bp, Artificial Sequence
  Synthetic polynucleotide sequence
tcgtcacgagcgcggatcgtgagaagtatgacaacagcctcaagatcatcagcaatgcct

SEQ ID NO: 230 - DNA, 60 bp, Artificial Sequence
  Synthetic polynucleotide sequence
tcgttacgagcgcggatcgtaagaagtatgacaacagcctcaagatcatcagcaatgcct

SEQ ID NO: 231 - DNA, 60 bp, Artificial Sequence
  Synthetic polynucleotide sequence
ggaaggtgaaggtcggagtcaacggatttggtcgtattgggcgcctggtcaccagggctg

SEQ ID NO: 232 - DNA, 60 bp, Artificial Sequence
  Synthetic polynucleotide sequence
aggaagcggaggtcggagtcaacggatttggtcgtattgggcgcctggtcaccagggctg

SEQ ID NO: 233 - DNA, 60 bp, Artificial Sequence
  Synthetic polynucleotide sequence
gggaagcggaagtcggagtcaacggatttggtcgtattgggcgcctggtcaccagggctg

SEQ ID NO: 234 - DNA, 60 bp, Artificial Sequence
  Synthetic polynucleotide sequence
ggaaagcggaagccggagtcaacggatttggtcgtattgggcgcctggtcaccagggctg

SEQ ID NO: 235 - DNA, 60 bp, Artificial Sequence
  Synthetic polynucleotide sequence
ggaaggcggaagccagagtcaacggatttggtcgtattgggcgcctggtcaccagggctg

SEQ ID NO: 236 - DNA, 60 bp, Artificial Sequence
  Synthetic polynucleotide sequence
ggaaggtggaagccagggtcaacggatttggtcgtattgggcgcctggtcaccagggctg

SEQ ID NO: 237 - DNA, 60 bp, Artificial Sequence
  Synthetic polynucleotide sequence
tgctcctcctgttcgacagtcagccgcatcttcttttgcgtcgccagccgagccacatcg

SEQ ID NO: 238 - DNA, 60 bp, Artificial Sequence
  Synthetic polynucleotide sequence
tgctcctcctcagccgcatcttcttttgcgtcgccagccgagccacatcgtttttttttt

SEQ ID NO: 239 - DNA, 60 bp, Artificial Sequence
  Synthetic polynucleotide sequence
tgctcctcctgttcgacagtttcttttgcgtcgccagccgagccacatcgtttttttttt

SEQ ID NO: 240 - DNA, 60 bp, Artificial Sequence
  Synthetic polynucleotide sequence
tgctcctcctgttcgacagtcagccgcatctcgccagccgagccacatcgtttttttttt

SEQ ID NO: 241 - DNA, 60 bp, Artificial Sequence
  Synthetic polynucleotide sequence
tgctcctcctgttcgacagtcagccgcatcttcttttgcgagccacatcgtttttttttt

SEQ ID NO: 242 - DNA, 60 bp, Artificial Sequence
  Synthetic polynucleotide sequence
tgctcctcctgttcgacagtcagccgcatcttcttttgcgtcgccagccgtttttttttt

SEQ ID NO: 243 - DNA, 60 bp, Artificial Sequence
  Synthetic polynucleotide sequence
ttttgcaagggcctctgaatctgtctgtgtccctgttagcacaatgtgaggaggtagaga

SEQ ID NO: 244 - DNA, 60 bp, Artificial Sequence
  Synthetic polynucleotide sequence
ttttgcaagggcctctgaatctgtctgtgtccctgttagcacaatgtgagtttttttttt

SEQ ID NO: 245 - DNA, 60 bp, Artificial Sequence
  Synthetic polynucleotide sequence
ttttgcaagggcctctgaatctgtctgtgtccctgttagctttttttttttttttttttt

SEQ ID NO: 246 - DNA, 60 bp, Artificial Sequence
  Synthetic polynucleotide sequence
ttttgcaagggcctctgaatctgtctgtgtccctgttttttttttttttttttttttttt

SEQ ID NO: 247 - DNA, 60 bp, Artificial Sequence
  Synthetic polynucleotide sequence
ttttgcaagggcctctgaatctgtctgtgttttttttttttttttttttttttttttttt

SEQ ID NO: 248 - DNA, 60 bp, Artificial Sequence
  Synthetic polynucleotide sequence
ttttgcaagggcctctgaatctgtcctttttttttttttttttttttttttttttttttt

SEQ ID NO: 249 - DNA, 60 bp, Artificial Sequence
  Synthetic polynucleotide sequence
ggtcagagtattgggaccgggagacacggagcgccagggacaccgcacagattttccgag

SEQ ID NO: 250 - DNA, 60 bp, Artificial Sequence
  Synthetic polynucleotide sequence
ggtcagagtattgggaccgggagacacggagcgccagggacaccgttttttttttttttt

SEQ ID NO: 251 - DNA, 60 bp, Artificial Sequence
  Synthetic polynucleotide sequence
ggtcagagtattgggaccgggagacacggagcgccttttttttttttttttttttttttt

SEQ ID NO: 252 - DNA, 60 bp, Artificial Sequence
  Synthetic polynucleotide sequence
ggtcagagtattgggaccgggagacacggatttttttttttttttttttttttttttttt

SEQ ID NO: 253 - DNA, 60 bp, Artificial Sequence
  Synthetic polynucleotide sequence
ggtcagagtattgggaccgggagacttttttttttttttttttttttttttttttttttt

SEQ ID NO: 254 - DNA, 60 bp, Artificial Sequence
  Synthetic polynucleotide sequence
ggtcagagtattgggaccggtttttttttttttttttttttttttttttttttttttttt

SEQ ID NO: 255 - DNA, 60 bp, Artificial Sequence
  Synthetic polynucleotide sequence
agattacaagggcacttggatccacccagaaattgacaaccccgagtattctcccgatcc

SEQ ID NO: 256 - DNA, 60 bp, Artificial Sequence
  Synthetic polynucleotide sequence
tttttacaagggcacttggatccacccagaaattgacaaccccgagtattctcccgatcc

SEQ ID NO: 257 - DNA, 60 bp, Artificial Sequence
  Synthetic polynucleotide sequence
ttttttttttggcacttggatccacccagaaattgacaaccccgagtattctcccgatcc

SEQ ID NO: 258 - DNA, 60 bp, Artificial Sequence
  Synthetic polynucleotide sequence
tttttttttttttttttggatccacccagaaattgacaaccccgagtattctcccgatcc

SEQ ID NO: 259 - DNA, 60 bp, Artificial Sequence
  Synthetic polynucleotide sequence
tttttttttttttttttttttccacccagaaattgacaaccccgagtattctcccgatcc

SEQ ID NO: 260 - DNA, 60 bp, Artificial Sequence
  Synthetic polynucleotide sequence
tttttttttttttttttttttttttccagaaattgacaaccccgagtattctcccgatcc

SEQ ID NO: 261 - DNA, 60 bp, Artificial Sequence
  Synthetic polynucleotide sequence
gatgagtttacacacctgtacacactgattgtgcggccagacaacacctatgaggtgaag

SEQ ID NO: 262 - DNA, 60 bp, Artificial Sequence
  Synthetic polynucleotide sequence
ggtaaatctgcacacctgtacacactgattgtgcggccagacaacacctatgaggtgaag

SEQ ID NO: 263 - DNA, 60 bp, Artificial Sequence
  Synthetic polynucleotide sequence
gatgagtttatatatccgcacacactgattgtgcggccagacaacacctatgaggtgaag

SEQ ID NO: 264 - DNA, 60 bp, Artificial Sequence
  Synthetic polynucleotide sequence
gatgagtttacacacctgtatatattaactgtgcggccagacaacacctatgaggtgaag

SEQ ID NO: 265 - DNA, 60 bp, Artificial Sequence
  Synthetic polynucleotide sequence
gatgagtttacacacctgtacacactgattatacagtcggacaacacctatgaggtgaag

SEQ ID NO: 266 - DNA, 60 bp, Artificial Sequence
  Synthetic polynucleotide sequence
gatgagtttacacacctgtacacactgattgtgcggccaggcgatatccacgaggtgaag

SEQ ID NO: 267 - DNA, 60 bp, Artificial Sequence
  Synthetic polynucleotide sequence
gccgggtgtggcacagctagttccgtcgcagccgggatttgggtcgcggttcttgtttgt

SEQ ID NO: 268 - DNA, 60 bp, Artificial Sequence
  Synthetic polynucleotide sequence
gccgggtgtggacagctagttcgtcgcagccggatttgggtcgcgttcttgtttgttttt

SEQ ID NO: 269 - DNA, 60 bp, Artificial Sequence
  Synthetic polynucleotide sequence
gccgggtgtgcacagctattccgtcgcgccgggattgggtcgcggtcttgtttgtttttt

SEQ ID NO: 270 - DNA, 60 bp, Artificial Sequence
  Synthetic polynucleotide sequence
gccgggttggcacactagttcgtcgcagcgggattgggtcgcgttcttgttgtttttttt

SEQ ID NO: 271 - DNA, 60 bp, Artificial Sequence
  Synthetic polynucleotide sequence
gccggtgtggacagcagttcgtcgcgccggatttggtccggtcttgttgttttttttttt

SEQ ID NO: 272 - DNA, 60 bp, Artificial Sequence
  Synthetic polynucleotide sequence
gccggttggacactattcgtccagcggattgggcgcgttttgttgttttttttttttttt

SEQ ID NO: 273 - DNA, 60 bp, Artificial Sequence
  Synthetic polynucleotide sequence
gtgatcgtcacttgacaatgcagatcttcgtgaagactctgactggtaagaccatcaccc

SEQ ID NO: 274 - DNA, 60 bp, Artificial Sequence
  Synthetic polynucleotide sequence
ggtgccttgacaatgcagatcttcgtgaagactctgactggtaagaccatcacccttttt

SEQ ID NO: 275 - DNA, 60 bp, Artificial Sequence
  Synthetic polynucleotide sequence
gtgatcgtcatgcagcagatcttcgtgaagactctgactggtaagaccatcacccttttt

SEQ ID NO: 276 - DNA, 60 bp, Artificial Sequence
  Synthetic polynucleotide sequence
gtgatcgtcacttgacaatgaactgtgaagactctgactggtaagaccatcacccttttt

SEQ ID NO: 277 - DNA, 60 bp, Artificial Sequence
  Synthetic polynucleotide sequence
gtgatcgtcacttgacaatgcagatcttcggaattgactggtaagaccatcacccttttt

SEQ ID NO: 278 - DNA, 60 bp, Artificial Sequence
  Synthetic polynucleotide sequence
gtgatcgtcacttgacaatgcagatcttcgtgaagactctatgagaccatcacccttttt

SEQ ID NO: 279 - DNA, 60 bp, Artificial Sequence
  Synthetic polynucleotide sequence
ccaggagactagactactgttgtccagggtcaatttgagtgtaaagaaaatgtagacaag

SEQ ID NO: 280 - DNA, 60 bp, Artificial Sequence
  Synthetic polynucleotide sequence
caggcaatctgttgtccagggtcaatttgagtgtaaagaaaatgtagacaagtttttttt

SEQ ID NO: 281 - DNA, 60 bp, Artificial Sequence
  Synthetic polynucleotide sequence
ccaggagactgcattgcagggtcaatttgagtgtaaagaaaatgtagacaagtttttttt

SEQ ID NO: 282 - DNA, 60 bp, Artificial Sequence
  Synthetic polynucleotide sequence
ccaggagactagactactgtgcagtattgagtgtaaagaaaatgtagacaagtttttttt

SEQ ID NO: 283 - DNA, 60 bp, Artificial Sequence
  Synthetic polynucleotide sequence
ccaggagactagactactgttgtccagggtattattagaaaatgtagacaagtttttttt

SEQ ID NO: 284 - DNA, 60 bp, Artificial Sequence
  Synthetic polynucleotide sequence
ccaggagactagactactgttgtccagggtcaatttgagttagaagaacaagtttttttt

SEQ ID NO: 285 - DNA, 60 bp, Artificial Sequence
  Synthetic polynucleotide sequence
atttatcataacaaaaatactactaatataactactgtttccatgtcccatgatcccctc

SEQ ID NO: 286 - DNA, 60 bp, Artificial Sequence
  Synthetic polynucleotide sequence
ccccatcataacaaaaatactactaatataactactgtttccatgtcccatgatcccctc

SEQ ID NO: 287 - DNA, 60 bp, Artificial Sequence
  Synthetic polynucleotide sequence
cccccccataacaaaaatactactaatataactactgtttccatgtcccatgatcccctc

SEQ ID NO: 288 - DNA, 60 bp, Artificial Sequence
  Synthetic polynucleotide sequence
cccccctctaacaaaaatactactaatataactactgtttccatgtcccatgatcccctc

SEQ ID NO: 289 - DNA, 60 bp, Artificial Sequence
  Synthetic polynucleotide sequence
ccccccccccacaaaaatactactaatataactactgtttccatgtcccatgatcccctc

SEQ ID NO: 290 - DNA, 60 bp, Artificial Sequence
  Synthetic polynucleotide sequence
ccccccccccctaaaaatactactaatataactactgtttccatgtcccatgatcccctc

SEQ ID NO: 291 - DNA, 60 bp, Artificial Sequence
  Synthetic polynucleotide sequence
tgtagtgtgttccatttaaaattttggcatatggcattttctaacttaggaagccacaat

SEQ ID NO: 292 - DNA, 60 bp, Artificial Sequence
  Synthetic polynucleotide sequence
ccccccgtgttccatttaaaattttggcatatggcattttctaacttaggaagccacaat

SEQ ID NO: 293 - DNA, 60 bp, Artificial Sequence
  Synthetic polynucleotide sequence
tgtagtgtgtcttccctaaaattttggcatatggcattttctaacttaggaagccacaat

SEQ ID NO: 294 - DNA, 60 bp, Artificial Sequence
  Synthetic polynucleotide sequence
tgtagtgtgttccatttaaaccccccgcatatggcattttctaacttaggaagccacaat

SEQ ID NO: 295 - DNA, 60 bp, Artificial Sequence
  Synthetic polynucleotide sequence
tgtagtgtgttccatttaaaattttggcatcccctcttttctaacttaggaagccacaat

SEQ ID NO: 296 - DNA, 60 bp, Artificial Sequence
  Synthetic polynucleotide sequence
tgtagtgtgttccatttaaaattttggcatatggcatttttccctctaggaagccacaat

SEQ ID NO: 297 - DNA, 60 bp, Artificial Sequence
  Synthetic polynucleotide sequence
cattctttgaaatttattccttattccctcttggcagctaatgggctcttaccaagttta

SEQ ID NO: 298 - DNA, 60 bp, Artificial Sequence
  Synthetic polynucleotide sequence
catttcccccaatttattccttattccctcttggcagctaatgggctcttaccaagttta

SEQ ID NO: 299 - DNA, 60 bp, Artificial Sequence
  Synthetic polynucleotide sequence
cattctttgaaattccccttttattccctcttggcagctaatgggctcttaccaagttta

SEQ ID NO: 300 - DNA, 60 bp, Artificial Sequence
  Synthetic polynucleotide sequence
cattctttgaaatttattccttatctttctttggcagctaatgggctcttaccaagttta

SEQ ID NO: 301 - DNA, 60 bp, Artificial Sequence
  Synthetic polynucleotide sequence
cattctttgaaatttattccttattccctcttggtcctccatgggctcttaccaagttta

SEQ ID NO: 302 - DNA, 60 bp, Artificial Sequence
  Synthetic polynucleotide sequence
cattctttgaaatttattccttattccctcttggcagctaatggctctccaccaagttta

SEQ ID NO: 303 - DNA, 60 bp, Artificial Sequence
  Synthetic polynucleotide sequence
ttgatacttgcctaacatgcatgtgctgtaaaaatagttaacagggaaataacttgagat

SEQ ID NO: 304 - DNA, 60 bp, Artificial Sequence
  Synthetic polynucleotide sequence
ttgatacttgcctaacatgcatgtgctgtaaaaacttcctacagggaaataacttgagat

SEQ ID NO: 305 - DNA, 60 bp, Artificial Sequence
  Synthetic polynucleotide sequence
ttgatacttgcctaacatgcatgtgctgtattttcttcctacagggaaataacttgagat

SEQ ID NO: 306 - DNA, 60 bp, Artificial Sequence
  Synthetic polynucleotide sequence
ttgatacttgcctaacatgcatgtgtctctttttcttcctacagggaaataacttgagat

SEQ ID NO: 307 - DNA, 60 bp, Artificial Sequence
  Synthetic polynucleotide sequence
ttgatacttgcctaacatgctctcttctctttttcttcctacagggaaataacttgagat

SEQ ID NO: 308 - DNA, 60 bp, Artificial Sequence
  Synthetic polynucleotide sequence
ttgatacttgcctaattctttctcttctctttttcttcctacagggaaataacttgagat

SEQ ID NO: 309 - DNA, 60 bp, Artificial Sequence
  Synthetic polynucleotide sequence
ctgggaaatcgaagattgaggactactttccagaatttgctcgctacactactcctgagg

SEQ ID NO: 310 - DNA, 60 bp, Artificial Sequence
  Synthetic polynucleotide sequence
ctgggaaatcgaagattgaggactactttccagaaccctttcgctacactactcctgagg

SEQ ID NO: 311 - DNA, 60 bp, Artificial Sequence
  Synthetic polynucleotide sequence
ctgggaaatcgaagattgaggactactttctttttccctttcgctacactactcctgagg

SEQ ID NO: 312 - DNA, 60 bp, Artificial Sequence
  Synthetic polynucleotide sequence
ctgggaaatcgaagattgaggactatcccttttttccctttcgctacactactcctgagg

SEQ ID NO: 313 - DNA, 60 bp, Artificial Sequence
  Synthetic polynucleotide sequence
ctgggaaatcgaagattgagtttcttcccttttttccctttcgctacactactcctgagg

SEQ ID NO: 314 - DNA, 60 bp, Artificial Sequence
  Synthetic polynucleotide sequence
ctgggaaatcgaagaccttttttcttcccttttttccctttcgctacactactcctgagg

SEQ ID NO: 315 - DNA, 60 bp, Artificial Sequence
  Synthetic polynucleotide sequence
tcctggacaagatcgacgtgatcaagcaggctgactatgtgccgagcgatcaggacctgc

SEQ ID NO: 316 - DNA, 60 bp, Artificial Sequence
  Synthetic polynucleotide sequence
cttcggacaagatcgacgtgatcaagcaggctgactatgtgccgagcgatcaggacctgc

SEQ ID NO: 317 - DNA, 60 bp, Artificial Sequence
  Synthetic polynucleotide sequence
cttcttttaagatcgacgtgatcaagcaggctgactatgtgccgagcgatcaggacctgc

SEQ ID NO: 318 - DNA, 60 bp, Artificial Sequence
  Synthetic polynucleotide sequence
cttctttttttttcgacgtgatcaagcaggctgactatgtgccgagcgatcaggacctgc

SEQ ID NO: 319 - DNA, 60 bp, Artificial Sequence
  Synthetic polynucleotide sequence
cttcttttttttctttcgtgatcaagcaggctgactatgtgccgagcgatcaggacctgc

SEQ ID NO: 320 - DNA, 60 bp, Artificial Sequence
  Synthetic polynucleotide sequence
cttcttttttttctttttctatcaagcaggctgactatgtgccgagcgatcaggacctgc

SEQ ID NO: 321 - DNA, 60 bp, Artificial Sequence
  Synthetic polynucleotide sequence
ccccaacttgagatgtatgaaggcttttggtctccctgggagtgggtggaggcagccagg

SEQ ID NO: 322 - DNA, 60 bp, Artificial Sequence
  Synthetic polynucleotide sequence
ccccaacttgagatgctcttaggcttttggtctccctgggagtgggtggaggcagccagg

SEQ ID NO: 323 - DNA, 60 bp, Artificial Sequence
  Synthetic polynucleotide sequence
ccccaacttgagactctcttttgcttttggtctccctgggagtgggtggaggcagccagg

SEQ ID NO: 324 - DNA, 59 bp, Artificial Sequence
  Synthetic polynucleotide sequence
ccccaacttgattctctctttttttttggtctccctgggagtgggtggaggcagccagg

SEQ ID NO: 325 - DNA, 60 bp, Artificial Sequence
  Synthetic polynucleotide sequence
ccccaacttttttctctcttttttccttggtctccctgggagtgggtggaggcagccagg

SEQ ID NO: 326 - DNA, 60 bp, Artificial Sequence
  Synthetic polynucleotide sequence
ccccaacccttttctctcttttttccccggtctccctgggagtgggtggaggcagccagg

SEQ ID NO: 327 - DNA, 60 bp, Artificial Sequence
  Synthetic polynucleotide sequence
atttaaaaactggaacggtgaaggtgacagcagtcggttggagcgagcatcccccaaagt

SEQ ID NO: 328 - DNA, 60 bp, Artificial Sequence
  Synthetic polynucleotide sequence
atttaaaaactggaatttctaaggtgacagcagtcggttggagcgagcatcccccaaagt

SEQ ID NO: 329 - DNA, 60 bp, Artificial Sequence
  Synthetic polynucleotide sequence
atttaaaaactggtttttctttggtgacagcagtcggttggagcgagcatcccccaaagt

SEQ ID NO: 330 - DNA, 60 bp, Artificial Sequence
  Synthetic polynucleotide sequence
atttaaaaacttttttttcttttttgacagcagtcggttggagcgagcatcccccaaagt

SEQ ID NO: 331 - DNA, 60 bp, Artificial Sequence
  Synthetic polynucleotide sequence
atttaaaaatctttttttctttttctacagcagtcggttggagcgagcatcccccaaagt

SEQ ID NO: 332 - DNA, 60 bp, Artificial Sequence
  Synthetic polynucleotide sequence
atttaaatttctttttttctttttctttagcagtcggttggagcgagcatcccccaaagt

SEQ ID NO: 333 - DNA, 60 bp, Artificial Sequence
  Synthetic polynucleotide sequence
aagaacatcacccagcgcggcaacgtcgccaagacctcgagaaatgcccccgaagagaag

SEQ ID NO: 334 - DNA, 60 bp, Artificial Sequence
  Synthetic polynucleotide sequence
gttgacatcacccagcgcggcaacgtcgccaagacctcgagaaatgcccccgaagagaag

SEQ ID NO: 335 - DNA, 60 bp, Artificial Sequence
  Synthetic polynucleotide sequence
cgttgcatcacccagcgcggcaacgtcgccaagacctcgagaaatgcccccgaagagaag

SEQ ID NO: 336 - DNA, 60 bp, Artificial Sequence
  Synthetic polynucleotide sequence
acgttgatcacccagcgcggcaacgtcgccaagacctcgagaaatgcccccgaagagaag

SEQ ID NO: 337 - DNA, 60 bp, Artificial Sequence
  Synthetic polynucleotide sequence
gacgttgtcacccagcgcggcaacgtcgccaagacctcgagaaatgcccccgaagagaag

SEQ ID NO: 338 - DNA, 60 bp, Artificial Sequence
  Synthetic polynucleotide sequence
cgacgttgcacccagcgcggcaacgtcgccaagacctcgagaaatgcccccgaagagaag

SEQ ID NO: 339 - DNA, 60 bp, Artificial Sequence
  Synthetic polynucleotide sequence
tatcatgcttcatgtgtcattccaaggtttcttcatgagtcattccaagttttctagtcc

SEQ ID NO: 340 - DNA, 60 bp, Artificial Sequence
  Synthetic polynucleotide sequence
tatcatgcttcatgtgtccaaggtttcttcatgagtcattccaagttttctagtcctttt

SEQ ID NO: 341 - DNA, 60 bp, Artificial Sequence
  Synthetic polynucleotide sequence
tatcatgcttcatgtccaaggtttcttcatgagtcattccaagttttctagtcctttttt

SEQ ID NO: 342 - DNA, 60 bp, Artificial Sequence
  Synthetic polynucleotide sequence
tatcatgcttcatgcaaggtttcttcatgagtcattccaagttttctagtcctttttttt

SEQ ID NO: 343 - DNA, 60 bp, Artificial Sequence
  Synthetic polynucleotide sequence
tatcatgcttcataaggtttcttcatgagtcattccaagttttctagtcctttttttttt

SEQ ID NO: 344 - DNA, 60 bp, Artificial Sequence
  Synthetic polynucleotide sequence
tatcatgcttcgaggtttcttcatgagtcattccaagttttctagtcctttttttttttt

SEQ ID NO: 345 - DNA, 60 bp, Artificial Sequence
  Synthetic polynucleotide sequence
caggtgccacaggcagccctgggacataggaagctgggagcaaggaaagggtcttagtca

SEQ ID NO: 346 - DNA, 60 bp, Artificial Sequence
  Synthetic polynucleotide sequence
caggtgccacaggcagccctgggacaaagctgggagcaaggaaagggtcttagtcatttt

SEQ ID NO: 347 - DNA, 60 bp, Artificial Sequence
  Synthetic polynucleotide sequence
caggtgccacaggcagccctgggacagctgggagcaaggaaagggtcttagtcatttttt

SEQ ID NO: 348 - DNA, 60 bp, Artificial Sequence
  Synthetic polynucleotide sequence
caggtgccacaggcagccctgggagctgggagcaaggaaagggtcttagtcatttttttt

SEQ ID NO: 349 - DNA, 60 bp, Artificial Sequence
  Synthetic polynucleotide sequence
caggtgccacaggcagccctgggctgggagcaaggaaagggtcttagtcatttttttttt

SEQ ID NO: 350 - DNA, 60 bp, Artificial Sequence
  Synthetic polynucleotide sequence
caggtgccacaggcagccctggtgggagcaaggaaagggtcttagtcatttttttttttt

SEQ ID NO: 351 - DNA, 60 bp, Artificial Sequence
  Synthetic polynucleotide sequence
acatctacaatgttggctcaagtgctgcattagacgtggaactatctgatgattccttcc

SEQ ID NO: 352 - DNA, 60 bp, Artificial Sequence
  Synthetic polynucleotide sequence
acatcttgttggctcaagtgctgcattagacgtggaactatctgatgattccttcctttt

SEQ ID NO: 353 - DNA, 60 bp, Artificial Sequence
  Synthetic polynucleotide sequence
acatcgttggctcaagtgctgcattagacgtggaactatctgatgattccttcctttttt

SEQ ID NO: 354 - DNA, 60 bp, Artificial Sequence
  Synthetic polynucleotide sequence
acatttggctcaagtgctgcattagacgtggaactatctgatgattccttcctttttttt

SEQ ID NO: 355 - DNA, 60 bp, Artificial Sequence
  Synthetic polynucleotide sequence
acatggctcaagtgctgcattagacgtggaactatctgatgattccttcctttttttttt

SEQ ID NO: 356 - DNA, 60 bp, Artificial Sequence
  Synthetic polynucleotide sequence
acggctcaagtgctgcattagacgtggaactatctgatgattccttcctttttttttttt

SEQ ID NO: 357 - DNA, 60 bp, Artificial Sequence
  Synthetic polynucleotide sequence
gctgcagaatggctcccgcaaagaagggtggcgagaagaaaaagggccgttctgccatca

SEQ ID NO: 358 - DNA, 60 bp, Artificial Sequence
  Synthetic polynucleotide sequence
gctgcagaatgccgcaaagaagggtggcgagaagaaaaagggccgttctgccatcatttt

SEQ ID NO: 359 - DNA, 60 bp, Artificial Sequence
  Synthetic polynucleotide sequence
gctgcagaatcgcaaagaagggtggcgagaagaaaaagggccgttctgccatcatttttt

SEQ ID NO: 360 - DNA, 60 bp, Artificial Sequence
  Synthetic polynucleotide sequence
gctgcagaagcaaagaagggtggcgagaagaaaaagggccgttctgccatcatttttttt

SEQ ID NO: 361 - DNA, 60 bp, Artificial Sequence
  Synthetic polynucleotide sequence
gctgcagacaaagaagggtggcgagaagaaaaagggccgttctgccatcatttttttttt

SEQ ID NO: 362 - DNA, 60 bp, Artificial Sequence
  Synthetic polynucleotide sequence
gctgcagaaagaagggtggcgagaagaaaaagggccgttctgccatcatttttttttttt

SEQ ID NO: 363 - DNA, 60 bp, Artificial Sequence
  Synthetic polynucleotide sequence
atgtgccataccgaatccgtgtgcggctgtccagaaaacgtaatgaggatgaagattcac

SEQ ID NO: 364 - DNA, 60 bp, Artificial Sequence
  Synthetic polynucleotide sequence
atgtgcccgaatccgtgtgcggctgtccagaaaacgtaatgaggatgaagattcactttt

SEQ ID NO: 365 - DNA, 60 bp, Artificial Sequence
  Synthetic polynucleotide sequence
atgtgcgaatccgtgtgcggctgtccagaaaacgtaatgaggatgaagattcactttttt

SEQ ID NO: 366 - DNA, 60 bp, Artificial Sequence
  Synthetic polynucleotide sequence
atgtgaatccgtgtgcggctgtccagaaaacgtaatgaggatgaagattcactttttttt

SEQ ID NO: 367 - DNA, 60 bp, Artificial Sequence
  Synthetic polynucleotide sequence
atgaatccgtgtgcggctgtccagaaaacgtaatgaggatgaagattcactttttttttt

SEQ ID NO: 368 - DNA, 60 bp, Artificial Sequence
  Synthetic polynucleotide sequence
atatccgtgtgcggctgtccagaaaacgtaatgaggatgaagattcactttttttttttt

SEQ ID NO: 369 - DNA, 60 bp, Artificial Sequence
  Synthetic polynucleotide sequence
gagtgggcttcaagaagcgtgcacctcgggcactcaaagagattcggaaatttgccatga

SEQ ID NO: 370 - DNA, 60 bp, Artificial Sequence
  Synthetic polynucleotide sequence
gagtgggcttcagcgtgcacctcgggcactcaaagagattcggaaatttgccatgatttt

SEQ ID NO: 371 - DNA, 60 bp, Artificial Sequence
  Synthetic polynucleotide sequence
gagtgggcttgcgtgcacctcgggcactcaaagagattcggaaatttgccatgatttttt

SEQ ID NO: 372 - DNA, 60 bp, Artificial Sequence
  Synthetic polynucleotide sequence
gagtgggctcgtgcacctcgggcactcaaagagattcggaaatttgccatgatttttttt

SEQ ID NO: 373 - DNA, 60 bp, Artificial Sequence
  Synthetic polynucleotide sequence
gagtgggcgtgcacctcgggcactcaaagagattcggaaatttgccatgatttttttttt

SEQ ID NO: 374 - DNA, 60 bp, Artificial Sequence
  Synthetic polynucleotide sequence
gagtgggtgcacctcgggcactcaaagagattcggaaatttgccatgatttttttttttt

SEQ ID NO: 375 - DNA, 60 bp, Artificial Sequence
  Synthetic polynucleotide sequence
gtctgaaaaaggtattgcagtcagaactgtgtactgatgataaaagcctctggtagcaat

SEQ ID NO: 376 - DNA, 60 bp, Artificial Sequence
  Synthetic polynucleotide sequence
gtctgaaaaaggtatttcagaactgtgtactgatgataaaagcctctggtagcaattttt

SEQ ID NO: 377 - DNA, 60 bp, Artificial Sequence
  Synthetic polynucleotide sequence
gtctgaaaaaggtatcagaactgtgtactgatgataaaagcctctggtagcaattttttt

SEQ ID NO: 378 - DNA, 60 bp, Artificial Sequence
  Synthetic polynucleotide sequence
gtctgaaaaaggtaagaactgtgtactgatgataaaagcctctggtagcaattttttttt

SEQ ID NO: 379 - DNA, 60 bp, Artificial Sequence
  Synthetic polynucleotide sequence
gtctgaaaaaggtgaactgtgtactgatgataaaagcctctggtagcaattttttttttt

SEQ ID NO: 380 - DNA, 60 bp, Artificial Sequence
  Synthetic polynucleotide sequence
gtctgaaaaaggaactgtgtactgatgataaaagcctctggtagcaattttttttttttt

SEQ ID NO: 381 - DNA, 60 bp, Artificial Sequence
  Synthetic polynucleotide sequence
gtaaactaaggtagactactttgggaatgagaacatgcaaaatcaggaaaggctgtagaa

SEQ ID NO: 382 - DNA, 60 bp, Artificial Sequence
  Synthetic polynucleotide sequence
gtaaactaaggtagactacttaatgagaacatgcaaaatcaggaaaggctgtagaatttt

SEQ ID NO: 383 - DNA, 60 bp, Artificial Sequence
  Synthetic polynucleotide sequence
gtaaactaaggtagactactatgagaacatgcaaaatcaggaaaggctgtagaatttttt

SEQ ID NO: 384 - DNA, 60 bp, Artificial Sequence
  Synthetic polynucleotide sequence
gtaaactaaggtagactactgagaacatgcaaaatcaggaaaggctgtagaatttttttt

SEQ ID NO: 385 - DNA, 60 bp, Artificial Sequence
  Synthetic polynucleotide sequence
gtaaactaaggtagactagagaacatgcaaaatcaggaaaggctgtagaatttttttttt

SEQ ID NO: 386 - DNA, 60 bp, Artificial Sequence
  Synthetic polynucleotide sequence
gtaaactaaggtagactagaacatgcaaaatcaggaaaggctgtagaatttttttttttt

SEQ ID NO: 387 - DNA, 60 bp, Artificial Sequence
  Synthetic polynucleotide sequence
gcagccatggccccaatcaaggtgggagatgccatcccagcagtggaggtgtttgaaggg

SEQ ID NO: 388 - DNA, 60 bp, Artificial Sequence
  Synthetic polynucleotide sequence
gcagccatggccccaatcaaggtggggccatcccagcagtggaggtgtttgaagggtttt

SEQ ID NO: 389 - DNA, 60 bp, Artificial Sequence
  Synthetic polynucleotide sequence
gcagccatggccccaatcaaggtggccatcccagcagtggaggtgtttgaagggtttttt

SEQ ID NO: 390 - DNA, 60 bp, Artificial Sequence
  Synthetic polynucleotide sequence
gcagccatggccccaatcaaggtgcatcccagcagtggaggtgtttgaagggtttttttt

SEQ ID NO: 391 - DNA, 60 bp, Artificial Sequence
  Synthetic polynucleotide sequence
gcagccatggccccaatcaaggtatcccagcagtggaggtgtttgaagggtttttttttt

SEQ ID NO: 392 - DNA, 60 bp, Artificial Sequence
  Synthetic polynucleotide sequence
gcagccatggccccaatcaaggtcccagcagtggaggtgtttgaagggtttttttttttt

SEQ ID NO: 393 - DNA, 60 bp, Artificial Sequence
  Synthetic polynucleotide sequence
agggagtccaggtggtggcctgtctgagtgttaatgatgcctttgtgactggcgagtggg

SEQ ID NO: 394 - DNA, 60 bp, Artificial Sequence
  Synthetic polynucleotide sequence
gacaagtccaggtggtggcctgtctgagtgttaatgatgcctttgtgactggcgagtggg

SEQ ID NO: 395 - DNA, 60 bp, Artificial Sequence
  Synthetic polynucleotide sequence
agacagtccaggtggtggcctgtctgagtgttaatgatgcctttgtgactggcgagtggg

SEQ ID NO: 396 - DNA, 60 bp, Artificial Sequence
  Synthetic polynucleotide sequence
cagacatccaggtggtggcctgtctgagtgttaatgatgcctttgtgactggcgagtggg

SEQ ID NO: 397 - DNA, 60 bp, Artificial Sequence
  Synthetic polynucleotide sequence
tcagacaccaggtggtggcctgtctgagtgttaatgatgcctttgtgactggcgagtggg

SEQ ID NO: 398 - DNA, 60 bp, Artificial Sequence
  Synthetic polynucleotide sequence
ctcagacacaggtggtggcctgtctgagtgttaatgatgcctttgtgactggcgagtggg

SEQ ID NO: 399 - DNA, 60 bp, Artificial Sequence
  Synthetic polynucleotide sequence
aggcacatcattggaatacaggaagtagccctgcacctgccagtgagctcgccattcact

SEQ ID NO: 400 - DNA, 60 bp, Artificial Sequence
  Synthetic polynucleotide sequence
cctgacatcattggaatacaggaagtagccctgcacctgccagtgagctcgccattcact

SEQ ID NO: 401 - DNA, 60 bp, Artificial Sequence
  Synthetic polynucleotide sequence
tcctgcatcattggaatacaggaagtagccctgcacctgccagtgagctcgccattcact

SEQ ID NO: 402 - DNA, 60 bp, Artificial Sequence
  Synthetic polynucleotide sequence
ttcctgatcattggaatacaggaagtagccctgcacctgccagtgagctcgccattcact

SEQ ID NO: 403 - DNA, 60 bp, Artificial Sequence
  Synthetic polynucleotide sequence
cttcctgtcattggaatacaggaagtagccctgcacctgccagtgagctcgccattcact

SEQ ID NO: 404 - DNA, 60 bp, Artificial Sequence
  Synthetic polynucleotide sequence
acttcctgcattggaatacaggaagtagccctgcacctgccagtgagctcgccattcact

SEQ ID NO: 405 - DNA, 60 bp, Artificial Sequence
  Synthetic polynucleotide sequence
tgtgtccacataatccacgctcatcttgcaaagcgctatttcaggcacatcattggaata

SEQ ID NO: 406 - DNA, 60 bp, Artificial Sequence
  Synthetic polynucleotide sequence
tgagacgagataatccacgctcatcttgcaaagcgctatttcaggcacatcattggaata

SEQ ID NO: 407 - DNA, 60 bp, Artificial Sequence
  Synthetic polynucleotide sequence
tgtgacgagaaaatccacgctcatcttgcaaagcgctatttcaggcacatcattggaata

SEQ ID NO: 408 - DNA, 60 bp, Artificial Sequence
  Synthetic polynucleotide sequence
tgtgtcgagaaactccacgctcatcttgcaaagcgctatttcaggcacatcattggaata

SEQ ID NO: 409 - DNA, 60 bp, Artificial Sequence
  Synthetic polynucleotide sequence
tgtgtccagaaactgcacgctcatcttgcaaagcgctatttcaggcacatcattggaata

SEQ ID NO: 410 - DNA, 60 bp, Artificial Sequence
  Synthetic polynucleotide sequence
tgtgtccacaaactgcccgctcatcttgcaaagcgctatttcaggcacatcattggaata

SEQ ID NO: 411 - DNA, 60 bp, Artificial Sequence
  Synthetic polynucleotide sequence
ggattgtttatactccagtgtacatagtgtaatgtagcgtgtttacatgtgtagcctatg

SEQ ID NO: 412 - DNA, 60 bp, Artificial Sequence
  Synthetic polynucleotide sequence
ggctagataaaactccagtgtacatagtgtaatgtagcgtgtttacatgtgtagcctatg

SEQ ID NO: 413 - DNA, 60 bp, Artificial Sequence
  Synthetic polynucleotide sequence
ggattgataaaagtgcagtgtacatagtgtaatgtagcgtgtttacatgtgtagcctatg

SEQ ID NO: 414 - DNA, 60 bp, Artificial Sequence
  Synthetic polynucleotide sequence
ggattgtttaaagtgccgagtacatagtgtaatgtagcgtgtttacatgtgtagcctatg

SEQ ID NO: 415 - DNA, 60 bp, Artificial Sequence
  Synthetic polynucleotide sequence
ggattgtttatactgccgagaagatagtgtaatgtagcgtgtttacatgtgtagcctatg

SEQ ID NO: 416 - DNA, 60 bp, Artificial Sequence
  Synthetic polynucleotide sequence
ggattgtttatactccagagaagaaattgtaatgtagcgtgtttacatgtgtagcctatg

SEQ ID NO: 417 - DNA, 60 bp, Artificial Sequence
  Synthetic polynucleotide sequence
ggcgccgattcctacaaagatgctgtccggaaagccatgtttgcccggttcactgagatg

SEQ ID NO: 418 - DNA, 60 bp, Artificial Sequence
  Synthetic polynucleotide sequence
ggctaatcggcctacaaagatgctgtccggaaagccatgtttgcccggttcactgagatg

SEQ ID NO: 419 - DNA, 60 bp, Artificial Sequence
  Synthetic polynucleotide sequence
ggcgccgatgaagcgcaagatgctgtccggaaagccatgtttgcccggttcactgagatg

SEQ ID NO: 420 - DNA, 60 bp, Artificial Sequence
  Synthetic polynucleotide sequence
ggcgccgattcctacccctcatctgtccggaaagccatgtttgcccggttcactgagatg

SEQ ID NO: 421 - DNA, 60 bp, Artificial Sequence
  Synthetic polynucleotide sequence
ggcgccgattcctacaaagattagtgaaggaaagccatgtttgcccggttcactgagatg

SEQ ID NO: 422 - DNA, 60 bp, Artificial Sequence
  Synthetic polynucleotide sequence
ggcgccgattcctacaaagatgctgtccttccctacatgtttgcccggttcactgagatg

SEQ ID NO: 423 - DNA, 60 bp, Artificial Sequence
  Synthetic polynucleotide sequence
tcatcccagctggtgtaataatgaattgtttaaaaaacagctcataattgatgccaaatt

SEQ ID NO: 424 - DNA, 60 bp, Artificial Sequence
  Synthetic polynucleotide sequence
tcatggtgtaataatgaattgtttaaaaaacagctcataattgatgccaaattttttttt

SEQ ID NO: 425 - DNA, 60 bp, Artificial Sequence
  Synthetic polynucleotide sequence
tcatcccagaataatgaattgtttaaaaaacagctcataattgatgccaaattttttttt

SEQ ID NO: 426 - DNA, 60 bp, Artificial Sequence
  Synthetic polynucleotide sequence
tcatcccagctggtggaattgtttaaaaaacagctcataattgatgccaaattttttttt

SEQ ID NO: 427 - DNA, 60 bp, Artificial Sequence
  Synthetic polynucleotide sequence
tcatcccagctggtgtaataatttaaaaaacagctcataattgatgccaaattttttttt

SEQ ID NO: 428 - DNA, 60 bp, Artificial Sequence
  Synthetic polynucleotide sequence
tcatcccagctggtgtaataatgaattaaacagctcataattgatgccaaattttttttt

SEQ ID NO: 429 - DNA, 60 bp, Artificial Sequence
  Synthetic polynucleotide sequence
aagggtacctgaagcgaattggcaccaaagcagcagctgtattgccgcagttctagcttc

SEQ ID NO: 430 - DNA, 60 bp, Artificial Sequence
  Synthetic polynucleotide sequence
aagggtacctgaagctttccttcaccaaagcagcagctgtattgccgcagttctagcttc

SEQ ID NO: 431 - DNA, 60 bp, Artificial Sequence
  Synthetic polynucleotide sequence
aagggtacctgaagcccccccccaccaaagcagcagctgtattgccgcagttctagcttc

SEQ ID NO: 432 - DNA, 60 bp, Artificial Sequence
  Synthetic polynucleotide sequence
aagggtacctgaagccttaacccaccaaagcagcagctgtattgccgcagttctagcttc

SEQ ID NO: 433 - DNA, 60 bp, Artificial Sequence
  Synthetic polynucleotide sequence
aagggtacctgaagctccggttcaccaaagcagcagctgtattgccgcagttctagcttc

SEQ ID NO: 434 - DNA, 60 bp, Artificial Sequence
  Synthetic polynucleotide sequence
aagggtacctgaagccaccaaagcagcagctgtattgccgcagttctagcttcttttttt

SEQ ID NO: 435 - DNA, 60 bp, Artificial Sequence
  Synthetic polynucleotide sequence
tccaacgacaagacgaggattctcaacgtccagtccaggctcacagacgccaaacgcatt

SEQ ID NO: 436 - DNA, 60 bp, Artificial Sequence
  Synthetic polynucleotide sequence
tccaatttttttacgaggattctcaacgtccagtccaggctcacagacgccaaacgcatt

SEQ ID NO: 437 - DNA, 60 bp, Artificial Sequence
  Synthetic polynucleotide sequence
tccaacccccccacgaggattctcaacgtccagtccaggctcacagacgccaaacgcatt

SEQ ID NO: 438 - DNA, 60 bp, Artificial Sequence
  Synthetic polynucleotide sequence
tccaagctgttcacgaggattctcaacgtccagtccaggctcacagacgccaaacgcatt

SEQ ID NO: 439 - DNA, 60 bp, Artificial Sequence
  Synthetic polynucleotide sequence
tccaaatcacctacgaggattctcaacgtccagtccaggctcacagacgccaaacgcatt

SEQ ID NO: 440 - DNA, 60 bp, Artificial Sequence
  Synthetic polynucleotide sequence
tccaaacgaggattctcaacgtccagtccaggctcacagacgccaaacgcattttttttt

SEQ ID NO: 441 - DNA, 60 bp, Artificial Sequence
  Synthetic polynucleotide sequence
ttctactccgatgatcggctgaatgtaacagaggaactaacgtccaacgacaagacgagg

SEQ ID NO: 442 - DNA, 60 bp, Artificial Sequence
  Synthetic polynucleotide sequence
ttctactccgtcttcttgctgaatgtaacagaggaactaacgtccaacgacaagacgagg

SEQ ID NO: 443 - DNA, 60 bp, Artificial Sequence
  Synthetic polynucleotide sequence
ttctactccgccccctcgctgaatgtaacagaggaactaacgtccaacgacaagacgagg

SEQ ID NO: 444 - DNA, 60 bp, Artificial Sequence
  Synthetic polynucleotide sequence
ttctactccgtactagcgctgaatgtaacagaggaactaacgtccaacgacaagacgagg

SEQ ID NO: 445 - DNA, 60 bp, Artificial Sequence
  Synthetic polynucleotide sequence
ttctactccgcgtcgatgctgaatgtaacagaggaactaacgtccaacgacaagacgagg

SEQ ID NO: 446 - DNA, 60 bp, Artificial Sequence
  Synthetic polynucleotide sequence
ttctactccggctgaatgtaacagaggaactaacgtccaacgacaagacgaggttttttt

SEQ ID NO: 447 - DNA, 60 bp, Artificial Sequence
  Synthetic polynucleotide sequence
ggccacccattcagggcatatctggaatctgaagttgctatatctgaggagttggttcag

SEQ ID NO: 448 - DNA, 60 bp, Artificial Sequence
  Synthetic polynucleotide sequence
ggcctttttccttgggcatatctggaatctgaagttgctatatctgaggagttggttcag

SEQ ID NO: 449 - DNA, 60 bp, Artificial Sequence
  Synthetic polynucleotide sequence
ggccctttccctcgggcatatctggaatctgaagttgctatatctgaggagttggttcag

SEQ ID NO: 450 - DNA, 60 bp, Artificial Sequence
  Synthetic polynucleotide sequence
ggcctgggtaagtgggcatatctggaatctgaagttgctatatctgaggagttggttcag

SEQ ID NO: 451 - DNA, 60 bp, Artificial Sequence
  Synthetic polynucleotide sequence
ggcccaaacggacgggcatatctggaatctgaagttgctatatctgaggagttggttcag

SEQ ID NO: 452 - DNA, 60 bp, Artificial Sequence
  Synthetic polynucleotide sequence
ggccgggcatatctggaatctgaagttgctatatctgaggagttggttcagttttttttt

SEQ ID NO: 453 - DNA, 60 bp, Artificial Sequence
  Synthetic polynucleotide sequence
tactaccccgcagttcccggacatgatcctctccgcctctcgagataagaccatcatcat

SEQ ID NO: 454 - DNA, 60 bp, Artificial Sequence
  Synthetic polynucleotide sequence
tactaccccgcagtcttttttttcgatcctctccgcctctcgagataagaccatcatcat

SEQ ID NO: 455 - DNA, 60 bp, Artificial Sequence
  Synthetic polynucleotide sequence
tactaccccgcagtctttccctccgatcctctccgcctctcgagataagaccatcatcat

SEQ ID NO: 456 - DNA, 60 bp, Artificial Sequence
  Synthetic polynucleotide sequence
tactaccccgcagtagggcctgtagatcctctccgcctctcgagataagaccatcatcat

SEQ ID NO: 457 - DNA, 60 bp, Artificial Sequence
  Synthetic polynucleotide sequence
tactaccccgcagtgaaattcacggatcctctccgcctctcgagataagaccatcatcat

SEQ ID NO: 458 - DNA, 60 bp, Artificial Sequence
  Synthetic polynucleotide sequence
tactaccccgcagtgatcctctccgcctctcgagataagaccatcatcattttttttttt

SEQ ID NO: 459 - DNA, 60 bp, Artificial Sequence
  Synthetic polynucleotide sequence
agccattttcagttattataccagaacctcttcacatagacctattagtgcatttgtaac

SEQ ID NO: 460 - DNA, 60 bp, Artificial Sequence
  Synthetic polynucleotide sequence
agccatttttttcctcctcaccagaacctcttcacatagacctattagtgcatttgtaac

SEQ ID NO: 461 - DNA, 60 bp, Artificial Sequence
  Synthetic polynucleotide sequence
agccattttgctaacaacaaccagaacctcttcacatagacctattagtgcatttgtaac

SEQ ID NO: 462 - DNA, 60 bp, Artificial Sequence
  Synthetic polynucleotide sequence
agccatttttcccccccccaccagaacctcttcacatagacctattagtgcatttgtaac

SEQ ID NO: 463 - DNA, 60 bp, Artificial Sequence
  Synthetic polynucleotide sequence
agccattttgtcaataataaccagaacctcttcacatagacctattagtgcatttgtaac

SEQ ID NO: 464 - DNA, 60 bp, Artificial Sequence
  Synthetic polynucleotide sequence
agccattttaccagaacctcttcacatagacctattagtgcatttgtaactttttttttt

SEQ ID NO: 465 - DNA, 60 bp, Artificial Sequence
  Synthetic polynucleotide sequence
aaagtcaaatttcctgaattgctatgtgtctgggtttcatccatccgacattgaagttga

SEQ ID NO: 466 - DNA, 60 bp, Artificial Sequence
  Synthetic polynucleotide sequence
aaagtttttccccctgaattgctatgtgtctgggtttcatccatccgacattgaagttga

SEQ ID NO: 467 - DNA, 60 bp, Artificial Sequence
  Synthetic polynucleotide sequence
aaagttcccccccctgaattgctatgtgtctgggtttcatccatccgacattgaagttga

SEQ ID NO: 468 - DNA, 60 bp, Artificial Sequence
  Synthetic polynucleotide sequence
aaagtgtttaaacctgaattgctatgtgtctgggtttcatccatccgacattgaagttga

SEQ ID NO: 469 - DNA, 60 bp, Artificial Sequence
  Synthetic polynucleotide sequence
aaagtgcccgggcctgaattgctatgtgtctgggtttcatccatccgacattgaagttga

SEQ ID NO: 470 - DNA, 60 bp, Artificial Sequence
  Synthetic polynucleotide sequence
aaagtcctgaattgctatgtgtctgggtttcatccatccgacattgaagttgattttttt

SEQ ID NO: 471 - DNA, 60 bp, Artificial Sequence
  Synthetic polynucleotide sequence
catgtgactttgtcacagcccaagatagttaagtgggatcgagacatgtaagcagcatca

SEQ ID NO: 472 - DNA, 60 bp, Artificial Sequence
  Synthetic polynucleotide sequence
catgttttccctcttcagcccaagatagttaagtgggatcgagacatgtaagcagcatca

SEQ ID NO: 473 - DNA, 60 bp, Artificial Sequence
  Synthetic polynucleotide sequence
catgtcctccccctccagcccaagatagttaagtgggatcgagacatgtaagcagcatca

SEQ ID NO: 474 - DNA, 60 bp, Artificial Sequence
  Synthetic polynucleotide sequence
catgtctgaaacagtcagcccaagatagttaagtgggatcgagacatgtaagcagcatca

SEQ ID NO: 475 - DNA, 60 bp, Artificial Sequence
  Synthetic polynucleotide sequence
catgttcagggtggccagcccaagatagttaagtgggatcgagacatgtaagcagcatca

SEQ ID NO: 476 - DNA, 60 bp, Artificial Sequence
  Synthetic polynucleotide sequence
catgtcagcccaagatagttaagtgggatcgagacatgtaagcagcatcatttttttttt

SEQ ID NO: 477 - DNA, 60 bp, Artificial Sequence
  Synthetic polynucleotide sequence
ctccaaagattcaggtttactcacgtcatccagcagagaatggaaagtcaaatttcctga

SEQ ID NO: 478 - DNA, 60 bp, Artificial Sequence
  Synthetic polynucleotide sequence
ctccaaagtccttttcccactcacgtcatccagcagagaatggaaagtcaaatttcctga

SEQ ID NO: 479 - DNA, 60 bp, Artificial Sequence
  Synthetic polynucleotide sequence
ctccaaagccctccccccactcacgtcatccagcagagaatggaaagtcaaatttcctga

SEQ ID NO: 480 - DNA, 60 bp, Artificial Sequence
  Synthetic polynucleotide sequence
ctccaaagtaagtccaaaactcacgtcatccagcagagaatggaaagtcaaatttcctga

SEQ ID NO: 481 - DNA, 60 bp, Artificial Sequence
  Synthetic polynucleotide sequence
ctccaaagcggacttgggactcacgtcatccagcagagaatggaaagtcaaatttcctga

SEQ ID NO: 482 - DNA, 60 bp, Artificial Sequence
  Synthetic polynucleotide sequence
ctccaaagactcacgtcatccagcagagaatggaaagtcaaatttcctgatttttttttt

SEQ ID NO: 483 - DNA, 60 bp, Artificial Sequence
  Synthetic polynucleotide sequence
cggatttggtcgtattgggcgcctggtcaccagggctgcttttaactctggtaaagtgga

SEQ ID NO: 484 - DNA, 60 bp, Artificial Sequence
  Synthetic polynucleotide sequence
cggatttggtctctcctttttcctggtcaccagggctgcttttaactctggtaaagtgga

SEQ ID NO: 485 - DNA, 60 bp, Artificial Sequence
  Synthetic polynucleotide sequence
cggatttggtccccccccctccctggtcaccagggctgcttttaactctggtaaagtgga

SEQ ID NO: 486 - DNA, 60 bp, Artificial Sequence
  Synthetic polynucleotide sequence
cggatttggtccataacccgccctggtcaccagggctgcttttaactctggtaaagtgga

SEQ ID NO: 487 - DNA, 60 bp, Artificial Sequence
  Synthetic polynucleotide sequence
cggatttggtctgcggtttatcctggtcaccagggctgcttttaactctggtaaagtgga

SEQ ID NO: 488 - DNA, 60 bp, Artificial Sequence
  Synthetic polynucleotide sequence
cggatttggtccctggtcaccagggctgcttttaactctggtaaagtggatttttttttt

SEQ ID NO: 489 - DNA, 60 bp, Artificial Sequence; identical to SEQ ID NO: 237
  Synthetic polynucleotide sequence

SEQ ID NO: 490 - DNA, 60 bp, Artificial Sequence
  Synthetic polynucleotide sequence
tgctcctcctgttctttttcttttcgcatcttcttttgcgtcgccagccgagccacatcg

SEQ ID NO: 491 - DNA, 60 bp, Artificial Sequence
  Synthetic polynucleotide sequence
tgctcctcctgttccccccccccccgcatcttcttttgcgtcgccagccgagccacatcg

SEQ ID NO: 492 - DNA, 60 bp, Artificial Sequence
  Synthetic polynucleotide sequence
tgctcctcctgttcctgtcagtcgcgcatcttcttttgcgtcgccagccgagccacatcg

SEQ ID NO: 493 - DNA, 60 bp, Artificial Sequence
  Synthetic polynucleotide sequence
tgctcctcctgttctcactgactacgcatcttcttttgcgtcgccagccgagccacatcg

SEQ ID NO: 494 - DNA, 60 bp, Artificial Sequence
  Synthetic polynucleotide sequence
tgctcctcctgttccgcatcttcttttgcgtcgccagccgagccacatcgtttttttttt

SEQ ID NO: 495 - DNA, 60 bp, Artificial Sequence
  Synthetic polynucleotide sequence
gtctgaggataccactgaagagacattaaaggagtcatttgacggctccgttcgggcaag

SEQ ID NO: 496 - DNA, 60 bp, Artificial Sequence
  Synthetic polynucleotide sequence
gtctgttttcaccactgaagtttttttaaaggagtcatttgacggctccgttcgggcaag

SEQ ID NO: 497 - DNA, 60 bp, Artificial Sequence
  Synthetic polynucleotide sequence
gtctgcccccaccactgaagccctcttaaaggagtcatttgacggctccgttcgggcaag

SEQ ID NO: 498 - DNA, 60 bp, Artificial Sequence
  Synthetic polynucleotide sequence
gtctgtcctaaccactgaagtctgtttaaaggagtcatttgacggctccgttcgggcaag

SEQ ID NO: 499 - DNA, 60 bp, Artificial Sequence
  Synthetic polynucleotide sequence
gtctgcttcgaccactgaagcacacttaaaggagtcatttgacggctccgttcgggcaag

SEQ ID NO: 500 - DNA, 60 bp, Artificial Sequence
  Synthetic polynucleotide sequence
gtctgaccactgaagttaaaggagtcatttgacggctccgttcgggcaagtttttttttt

SEQ ID NO: 501 - DNA, 60 bp, Artificial Sequence
  Synthetic polynucleotide sequence
ctccctcgactgtcttgtcccagaaccttttcactccaaaacaggaaattcagcacctgt

SEQ ID NO: 502 - DNA, 60 bp, Artificial Sequence
  Synthetic polynucleotide sequence
ctcccctttttgtcttgtcctttttccttttcactccaaaacaggaaattcagcacctgt

SEQ ID NO: 503 - DNA, 60 bp, Artificial Sequence
  Synthetic polynucleotide sequence
ctcccctccttgtcttgtcctccccccttttcactccaaaacaggaaattcagcacctgt

SEQ ID NO: 504 - DNA, 60 bp, Artificial Sequence
  Synthetic polynucleotide sequence
ctcccagctgtgtcttgtccgtcttccttttcactccaaaacaggaaattcagcacctgt

SEQ ID NO: 505 - DNA, 60 bp, Artificial Sequence
  Synthetic polynucleotide sequence
ctcccgatcatgtcttgtcaactcaccttttcactccaaaacaggaaattcagcacctgt

SEQ ID NO: 506 - DNA, 60 bp, Artificial Sequence
  Synthetic polynucleotide sequence
ctccctgtcttgtccccttttcactccaaaacaggaaattcagcacctgttttttttttt

SEQ ID NO: 507 - DNA, 60 bp, Artificial Sequence
  Synthetic polynucleotide sequence
ctctgacactgtgtaagaagctgtgaatattcctaacttacccagatgttgcttttgaaa

SEQ ID NO: 508 - DNA, 60 bp, Artificial Sequence
  Synthetic polynucleotide sequence
ctctgttttcgtgtaagaagtctctaatattcctaacttacccagatgttgcttttgaaa

SEQ ID NO: 509 - DNA, 60 bp, Artificial Sequence
  Synthetic polynucleotide sequence
ctctgctctcgtgtaagaagtccccaatattcctaacttacccagatgttgcttttgaaa

SEQ ID NO: 510 - DNA, 60 bp, Artificial Sequence
  Synthetic polynucleotide sequence
ctctgtgtgagtgtaagaagctgtgaatattcctaacttacccagatgttgcttttgaaa

SEQ ID NO: 511 - DNA, 60 bp, Artificial Sequence
  Synthetic polynucleotide sequence
ctctgaacaggtgtaagaagagtgtaatattcctaacttacccagatgttgcttttgaaa

SEQ ID NO: 512 - DNA, 60 bp, Artificial Sequence
  Synthetic polynucleotide sequence
ctctggtgtaagaagaatattcctaacttacccagatgttgcttttgaaatttttttttt

SEQ ID NO: 513 - DNA, 60 bp, Artificial Sequence
  Synthetic polynucleotide sequence
gctggagtcgatcaactctaggctccaactcgttatgaaaagtgggaagtacgtcctggg

SEQ ID NO: 514 - DNA, 60 bp, Artificial Sequence
  Synthetic polynucleotide sequence
gctggttcttatcaactctatttctcaactcgttatgaaaagtgggaagtacgtcctggg

SEQ ID NO: 515 - DNA, 60 bp, Artificial Sequence
  Synthetic polynucleotide sequence
gctggccctcatcaactctacctctcaactcgttatgaaaagtgggaagtacgtcctggg

SEQ ID NO: 516 - DNA, 60 bp, Artificial Sequence
  Synthetic polynucleotide sequence
gctggtcagcatcaactctaccgagcaactcgttatgaaaagtgggaagtacgtcctggg

SEQ ID NO: 517 - DNA, 60 bp, Artificial Sequence
  Synthetic polynucleotide sequence
gctggctgatatcaactctattagacaactcgttatgaaaagtgggaagtacgtcctggg

SEQ ID NO: 518 - DNA, 60 bp, Artificial Sequence
  Synthetic polynucleotide sequence
gctggatcaactctacaactcgttatgaaaagtgggaagtacgtcctgggtttttttttt

SEQ ID NO: 519 - DNA, 60 bp, Artificial Sequence
  Synthetic polynucleotide sequence
caggtgactctgacatcattagaagcatgccagaacagactggtgaaaagtaaacctttt

SEQ ID NO: 520 - DNA, 60 bp, Artificial Sequence
  Synthetic polynucleotide sequence
caggttttcttgacatcatttttttcatgccagaacagactggtgaaaagtaaacctttt

SEQ ID NO: 521 - DNA, 60 bp, Artificial Sequence
  Synthetic polynucleotide sequence
caggtcctcttgacatcattccccccatgccagaacagactggtgaaaagtaaacctttt

SEQ ID NO: 522 - DNA, 60 bp, Artificial Sequence
  Synthetic polynucleotide sequence
caggtctgagtgacatcatttcttccatgccagaacagactggtgaaaagtaaacctttt

SEQ ID NO: 523 - DNA, 60 bp, Artificial Sequence
  Synthetic polynucleotide sequence
caggttcagatgacatcattctcctcatgccagaacagactggtgaaaagtaaacctttt

SEQ ID NO: 524 - DNA, 60 bp, Artificial Sequence
  Synthetic polynucleotide sequence
caggttgacatcattcatgccagaacagactggtgaaaagtaaacctttttttttttttt

SEQ ID NO: 525 - DNA, 24 bp, Artificial Sequence
  Synthetic polynucleotide sequence
tgcgtgatcgttctcacagccgaa